(19) United States
(12) Patent Application Publication
(10) Pub. No.: US 2026/0116882 A1
(43) Pub. Date: Apr. 30, 2026

(54) KRAS G12D MODULATING COMPOUNDS

(71) Applicant: Gilead Sciences, Inc., Foster City, CA (US)

(72) Inventors: Chaodi Dai, Copenhagen (DK); Hongyan Guo, San Mateo, CA (US); Darryl Kato, San Francisco, CA (US); Scott E. Lazerwith, San Francisco, CA (US); Jonathan William Medley, San Bruno, CA (US); Thomas C.J. Mies, Burlingame, CA (US); Hyung-Jung Pyun, Fremont, CA (US); William J. Watkins, Mountain View, CA (US); Jennifer R. Zhang, Union City, CA (US)

(21) Appl. No.: 19/342,216

(22) Filed: Sep. 26, 2025

Related U.S. Application Data
(60) Provisional application No. 63/701,240, filed on Sep. 30, 2024.

(51) Int. Cl. C07D 471/22; A61K 31/519; A61K 31/55; A61P 35/00; C07B 59/00; C07D 487/22

(52) U.S. Cl. CPC C07D 471/22; A61K 31/519; A61K 31/55; A61P 35/00; C07B 59/002; C07D 487/22

(57) ABSTRACT

Provided herein are compounds, and pharmaceutically acceptable salts thereof, useful as KRAS G12D and/or KRAS G12C inhibitors, methods of making and using the same (singly or in combination with additional agents), and pharmaceutical compositions thereof.

### CROSS-REFERENCE TO RELATED APPLICATIONS

This application claims the benefit under 35 U.S.C. § 119(e) of U.S. Provisional Application No. 63/701,240, filed on Sep. 30, 2024, which is incorporated herein by reference in its entirety for all purposes.

### BACKGROUND

The KRAS protein, Kirsten Rat Sarcoma 2 Viral Oncogene Homolog (“KRAS”), is a GTPase. KRAS gene mutations have been observed in a number of conditions including, for instance, pancreatic cancer, endometrial cancer, lung adenocarcinoma, colorectal cancer, rectal carcinoma, gall bladder cancer, thyroid cancer, bile duct cancer, small cell lung cancer, and non-small cell lung cancer (NSCLC). Accordingly, there is a need for compounds, pharmaceutical compositions, and methods for inhibiting KRAS (e.g., KRAS G12C and/or KRAS G12D) and treating associated cancers.

### SUMMARY

In one embodiment, the present disclosure provides a compound of Formula (I):

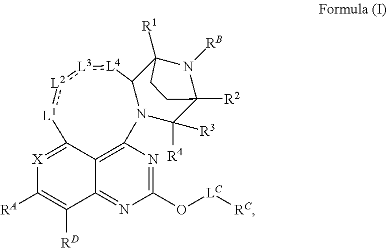

or a pharmaceutically acceptable salt thereof,
wherein
X is N, CH, or CR^{x};
R^{x} is C₁₋₃ alkyl, C₂₋₄ alkenyl, C₁₋₃ alkoxy, C₁₋₃ thioalkyl, —CN, (CH₂)ₘCN, halo, C₁₋₃ haloalkyl, or C₃₋₆ cycloalkyl;
m is 0, 1, 2 or 3;
R¹, R², R³, and R⁴ are each independently H or C₁-C₃ alkyl;
L¹ is O, S, CR^{1a}R^{1b}, C(═CR^{1c}R^{1d}), C(═O), or —C(R^{1e})═;
R^{1a} and R^{1b} are each independently H, C₁-C₃ alkyl, C₁-C₃ alkoxy, halo, C₁-C₆ haloalkyl, C₁-C₆ haloalkoxy, —OH, —CN, C₁-C₃ cyanoalkyl, or C₃-C₆ cycloalkyl;
alternatively, R^{1a} and R^{1b} can combine with the atom to which they are attached to form a C₃-C₆ cycloalkyl or a 3 to 6 membered heterocyclyl having 1 heteroatom that is O, wherein each cycloalkyl and heterocyclyl is substituted with 0, 1, 2, or 3 R^{1x};
R^{1c} and R^{1d} are each independently H, C₁-C₃ alkyl, halo, C₁-C₆ haloalkyl, —CN, C₁-C₃ cyanoalkyl, or C₃-C₆ cycloalkyl;
alternatively, R^{1c} and R^{1d} can combine with the atom to which they are attached to form a C₃-C₆ cycloalkyl or a 3 to 6 membered heterocyclyl having 1 heteroatom that is O, wherein each cycloalkyl and heterocyclyl is substituted with 0, 1, 2, or 3 R^{1x};
R^{1e} is H, C₁-C₃ alkyl, halo, C₁-C₆ haloalkyl, —CN, C₁-C₃ cyanoalkyl, or C₃-C₆ cycloalkyl;
each R^{1x} is independently C₁-C₃ alkyl, C₁-C₃ alkoxy, halo, C₁-C₆ haloalkyl, C₁-C₆ haloalkoxy, or —OH;
L² is absent, O, S, CR^{2a}R^{2b}, C(═CR^{2c}R^{2d}), C(═O), or =C(R^{2e})—, such that when L² is O or S, then L¹ is CR^{1a}R^{1b};
R^{2a} and R^{2b} are each independently H, C₁-C₃ alkyl, C₁-C₃ alkoxy, halo, C₁-C₆ haloalkyl, C₁-C₆ haloalkoxy, —OH, —CN, C₁-C₃ cyanoalkyl, or C₃-C₆ cycloalkyl;
alternatively, R^{2a} and R^{2b} can combine with the atom to which they are attached to form a C₃-C₆ cycloalkyl;
alternatively, R^{1b} and R^{2b} can combine with the atoms to which they are attached to form a C₃-C₆ cycloalkyl, 4- to 10-membered heterocyclyl, C₆-C₁₀ aryl, or 5- to 14-membered heteroaryl, wherein each cycloalkyl, heterocyclyl, aryl and heteroaryl is substituted with 0, 1, 2, or 3 R^{2x};
R^{2c} and R^{2d} are each independently H, C₁-C₃ alkyl, halo, C₁-C₆ haloalkyl, —CN, C₁-C₃ cyanoalkyl, or C₃-C₆ cycloalkyl;
alternatively, R^{2c} and R^{2d} can combine with the atom to which they are attached to form a C₃-C₆ cycloalkyl or a 3 to 6 membered heterocyclyl having 1 heteroatom that is O, wherein each cycloalkyl and heterocyclyl is substituted with 0, 1, 2, or 3 R^{2x};
R^{2e} is H, C₁-C₃ alkyl, halo, C₁-C₆ haloalkyl, —CN, C₁-C₃ cyanoalkyl, or C₃-C₆ cycloalkyl;
alternatively, R^{1e} and R^{2e} can combine with the atoms to which they are attached to form a C₅-C₆ cycloalkyl, 5- to 10-membered heterocyclyl, C₆-C₁₀ aryl, or 5- to 14-membered heteroaryl, wherein each cycloalkyl, heterocyclyl, aryl and heteroaryl is substituted with 0, 1, 2, or 3 R^{2x};
each R^{2x} is independently C₁-C₃ alkyl, C₁-C₃ alkoxy, halo, C₁-C₆ haloalkyl, C₁-C₆ haloalkoxy, or —OH;
alternatively L¹ and L² can combine to form;

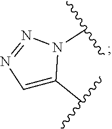 [SMILES: *c1cnnn1*]

L³ is absent, CR^{3a}R^{3b}, C(═CR^{3c}R^{3d}), C(═O), or =C(R^{3e})—;
R^{3a} and R^{3b} are each independently H, C₁-C₃ alkyl, C₁-C₃ alkoxy, halo, C₁-C₆ haloalkyl, C₁-C₆ haloalkoxy, —OH, —CN, C₁-C₃ cyanoalkyl, or C₃-C₆ cycloalkyl;
alternatively, R^{3a} and R^{3b} can combine with the atom to which they are attached to form a C₃-C₆ cycloalkyl;
alternatively, R^{2b} and R^{3b} can combine with the atoms to which they are attached to form a C₃-C₆ cycloalkyl;
R^{3c} and R^{3d} are each independently H, C₁-C₃ alkyl, halo, C₁-C₆ haloalkyl, —CN, C₁-C₃ cyanoalkyl, or C₃-C₆ cycloalkyl;
alternatively, R^{3c} and R^{3d} can combine with the atom to which they are attached to form a C₃-C₆ cycloalkyl or a 3 to 6 membered heterocyclyl having 1 heteroatom that is O, wherein each cycloalkyl and heterocyclyl is substituted with 0, 1, 2, or 3 R^{3x};
each R^{3x} is independently C₁-C₃ alkyl, C₁-C₃ alkoxy, halo, C₁-C₆ haloalkyl, C₁-C₆ haloalkoxy, or —OH;
R^{3e} is H, C₁-C₃ alkyl, halo, C₁-C₆ haloalkyl, —CN, C₁-C₃ cyanoalkyl, or C₃-C₆ cycloalkyl;
alternatively, R^{2e} and R^{3e} can combine with the atoms to which they are attached to form a C₅-C₆ cycloalkyl, 5- to 10-membered heterocyclyl, C₆-C₁₀ aryl, or 5- to 14-membered heteroaryl;
L⁴ is absent, CR^{4a}R^{4b}, C(═CR^{4c}R^{4d}), C(═O), or =C(R^{4e})—;
R^{4a} and R^{4b} are each independently H, C₁-C₃ alkyl, C₁-C₃ alkoxy, halo, C₁-C₆ haloalkyl, C₁-C₆ haloalkoxy, —OH, —CN, C₁-C₃ cyanoalkyl, or C₃-C₆ cycloalkyl;
alternatively, R^{4a} and R^{4b} can combine with the atom to which they are attached to form a C₃-C₆ cycloalkyl;
alternatively, R^{3b} and R^{4b} can combine with the atoms to which they are attached to form a C₃-C₆ cycloalkyl;
R^{4c} and R^{4d} are each independently H, C₁-C₃ alkyl, halo, C₁-C₆ haloalkyl, —CN, C₁-C₃ cyanoalkyl, or C₃-C₆ cycloalkyl;
alternatively, R^{4c} and R^{4d} can combine with the atom to which they are attached to form a C₃-C₆ cycloalkyl or a 3 to 6 membered heterocyclyl having 1 heteroatom that is O, wherein each cycloalkyl and heterocyclyl is substituted with 0, 1, 2, or 3 R^{4x};
each R^{4x} is independently C₁-C₃ alkyl, C₁-C₃ alkoxy, halo, C₁-C₆ haloalkyl, C₁-C₆ haloalkoxy, or —OH;
R^{4e} is H, C₁-C₃ alkyl, halo, C₁-C₆ haloalkyl, —CN, C₁-C₃ cyanoalkyl, or C₃-C₆ cycloalkyl;
alternatively, R^{3e} and R^{4e} can combine with the atoms to which they are attached to form a C₅-C₆ cycloalkyl;
such that when L² is =C(R^{2e})— then L¹ is —C(R^{1e})═ or L³ is =C(R^{3e})—, and when L³ is =C(R^{3e})— then L² is =C(R^{2e})— or L⁴ is =C(R^{4e})—;
R^{A} is phenyl, naphthyl, or 5- to 14-membered heteroaryl, wherein the phenyl, naphthyl, and heteroaryl is substituted with 0, 1, 2, 3, 4, or 5 R^{A2};
each R^{A2} is independently —OH, C₁-C₁₀ alkyl, C₂-C₆ alkenyl, C₂-C₆ alkynyl, C₁-C₁₀ alkoxy, C₁-C₁₀ hydroxyalkyl, C₂-C₁₀ alkoxyalkyl, C₁-C₆ alkyl-N(R^{A2a})(R^{A2b}), C₁-C₁₀ thioalkyl, halo, C₁-C₆ haloalkyl, —CN, —C(O)R^{A2a}, —C(O)OR^{A2a}, —OC(O)R^{A2a}, —OC(O)OR^{A2a}, —C(O)N(R^{A2a})(R^{A2b}) N(R^{A2a})C(O)(R^{A2b}), —OC(O)N(R^{A2a})(R^{A2b}), —N(R^{A2a})C(O)(OR^{A2b}), oxo, —OR^{A2a}, —OR^{A2x}, —SR^{A2a}, —S(O)₂R^{A2a}, —S(O)₂OR^{A2a}, —N(R^{A2a})(R^{A2b}), —(C₀-C₃ alkyl)-SF₅, —OP(O)(OR^{A2a})(OR^{A2b}), C₃-C₈ cycloalkyl, —(C₁-C₆ alkyl)-(C₃-C₈ cycloalkyl), 3- to 14-membered heterocyclyl, —(C₁-C₆ alkyl)-(3- to 14-membered heterocyclyl), C₆-C₁₄ aryl, —(C₁-C₆ alkyl)-(C₆-C₁₄ aryl), 5- to 14-membered heteroaryl, or —(C₁-C₆ alkyl)-(5- to 14-membered heteroaryl), wherein each alkyl, alkenyl, alkynyl, alkoxy, hydroxyalkyl, and haloalkyl is substituted with 0, 1, 2, or 3 R^{A3}, and wherein each cycloalkyl, alkyl-cycloalkyl, heterocyclyl, alkyl-heterocyclyl, aryl, alkyl-aryl, heteroaryl, and alkyl-heteroaryl is substituted with 0, 1, 2, or 3 R^{A4};
each R^{A2a} and R^{A2b} is independently H, C₁-C₁₀ alkyl, C₁-C₆ haloalkyl, or C₃-C₅ cycloalkyl;
each R^{A3} is independently halo, —CN, —OR^{A3a}, SR^{A3a}, —N(R^{A3a})(R^{A3b}) C₃-C₅ cycloalkyl, or 5- to 14-membered heteroaryl;
each R^{A3a} and R^{A3b} is independently H, C₁-C₁₀ alkyl, C₁-C₆ haloalkyl, or C₃-C₅ cycloalkyl;
each R^{A4} is independently C₁-C₆ alkoxy, C₁-C₆ hydroxyalkyl, C₂-C₆ alkoxyalkyl, C₁-C₆ haloalkyl, C₁-C₆ haloalkoxy, C₁-C₆ haloalkylthio, C₃-C₅ cycloalkyl, —(C₁-C₆ alkyl)-(C₆-C₁₀ aryl), halo, —CN, —OH, or —N(R^{A4a})(R^{A4b});
each R^{A4a} and R^{A4b} is independently H or C₁-C₆ alkyl;
alternatively, two R^{A2} can combine to form a C₃-C₁₀ cycloalkyl, C₆-C₁₀ aryl, a 3- to 10-membered heterocyclyl, or 5- to 14-membered heteroaryl on two adjacent atoms on R^{A}, wherein each cycloalkyl, aryl, heterocyclyl, and heteroaryl is substituted with 0, 1, 2, or 3 R^{A5};
each R^{A5} is independently C₁-C₁₀ alkyl, C₂-C₁₀ alkenyl, C₂-C₁₀ alkynyl, halo, C₁-C₆ haloalkyl, —CN, or C₃-C₅ cycloalkyl;
R^{B} is H or R^{B1};
R^{B1} and R^{A2x} are each independently:

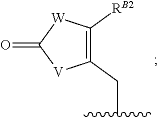

W and V are each independently NR^{B2}, O, or S;
each R^{B2} is independently C₁-C₁₂ alkyl, C₁-C₆ alkoxy, C₂-C₆ alkoxyalkyl, C₁-C₆ alkyl-N(R^{B2a})(R^{B2b}), C₁-C₁₀ thioalkyl, C₁-C₆haloalkyl, C₁-C₆haloalkoxy, C₃-C₈ cycloalkyl, C₆-C₁₄ aryl, or 5- to 14-membered heteroaryl, wherein the cycloalkyl, aryl, and heteroaryl are substituted with 0, 1, 2, or 3 R^{B2c};
each R^{B2a} and R^{B2b} is independently H, C₁-C₆ alkyl, C₁-C₆ haloalkyl, or C₃-C₈ cycloalkyl; each R^{B2c} is independently C₁-C₆ alkyl, C₁-C₆ alkoxy, C₂-C₆ alkoxyalkyl, halo, C₁-C₆ haloalkyl, C₁-C₆ haloalkoxy, oxo, —OH, —CN, or C₃-C₁₀ cycloalkyl;
L^{C} is a bond or

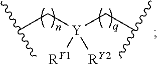

Y is C or Si;
n is 0, 1, 2, or 3;
q is 0, 1, 2, or 3;
R^{Y1} is H or C₁-C₃ alkyl;
R^{Y2} is H or C₁-C₃ alkyl;
alternatively, R^{Y1} and R^{Y2} combine to form a C₃-C₁₀ cycloalkyl or a 3- to 10-membered heterocyclyl;
R^{C} is 3- to 14-membered heterocyclyl substituted with ═C(R^{C3e})(R^{C3f}), wherein the heterocyclyl is further substituted with 0, 1, 2, 3, or 4 R^{C3};
R^{C3e} is H, C₁, C₁-C₆ alkyl, C₁-C₆ haloalkyl, or C₃-C₆ cycloalkyl;
R^{C31} is F, C₁, C₁-C₆ alkyl, C₁-C₆ haloalkyl, or C₃-C₆ cycloalkyl;
each R^{C3} is independently C₁-C₆ alkyl, C₂-C₆ alkenyl, C₂-C₅ alkynyl, C₁-C₆ alkoxyalkyl, C₁-C₆ hydroxyalkyl, halo, C₁-C₆ haloalkyl, C₁-C₆ heteroalkyl, —(C₁-C₆ alkyl)-N(R^{C3a})(R^{C3b}), —CN, —C(O)R^{C3a}, —C(O)OR^{C3a}, —C(O)N(R^{3a})(R^{C3b}), —N(R^{C3a})C(O)(R^{C3b}), —OC(O)N(R^{C3a})(R^{C3b}), N(R^{C3a})C(O)(OR^{C3b}), =CH₂, =CHF, =CF₂, oxo, —OR^{C3a}, SR^{C3a}, —N(R^{C3a})(R^{C3b}), —N₃, SF₅, C₃-C₈ cycloalkyl, —(C₁-C₆ alkyl)-(C₃-C₈ cycloalkyl), 3- to 10-membered heterocyclyl, —(C₁-C₆ alkyl)-(3- to 10-membered heterocyclyl), C₆-C₁₀ aryl, —(C₁-C₆ alkyl)-(C₆-C₁₀ aryl), 5- to 10-membered heteroaryl, or —(C₁-C₆ alkyl)-(5- to 10-membered heteroaryl),
wherein each alkyl is substituted with 0, 1, 2, or 3-CN, —C(O)OR^{C3a1}, —C(O)N(R^{C3a1})(R^{C3a2}), —N(R^{C3a1})C(O)(R^{C3a2}), —OC(O)N(R^{C3a1})(R^{C3a2}), —OR^{C3a1}, —SR^{C3a1}, N₃, SF₅, or 3- to 10-membered heterocyclyl substituted with 0, 1, 2, or 3 R^{C3a2}, each cycloalkyl, alkyl-cycloalkyl, heterocyclyl, alkyl-heterocyclyl, aryl, alkyl-aryl, heteroaryl, and alkyl-heteroaryl is substituted with 0, 1, 2, or 3 halo, —CN, or R^{C3a2}, each alkenyl is substituted with 0, 1, 2, or 3 halo, and each alkoxyalkyl and alkynyl is substituted with 0, 1, 2, or 3 C₁-C₆ alkyl, C₁-C₆ haloalkyl, C₃-C₅ cycloalkyl substituted with 0 or 1 C₁-C₆ haloalkyl, C₆-C₁₀ aryl, or 5- to 10-membered heteroaryl;
each R^{C3a} and R^{C3b} is independently H, C₁-C₁₀ alkyl, C₁-C₆ haloalkyl, C₆-C₁₀ aryl, C₃-C₆ cycloalkyl, 3- to 6-membered heterocyclyl, or 5- to 10-membered heteroaryl, wherein each aryl and heteroaryl is substituted with 0, 1, 2, or 3 halo, —CN, or R^{C3a2};
alternatively, R^{C3a} and R^{C3b} together with the N to which they are attached form a 3- to 8-membered heterocycle;
each R^{C3a1} and R^{C3a2} is independently C₁-C₃ alkyl, halo, C₁-C₆ haloalkyl, C₃-C₈ cycloalkyl, —(C₁-C₃ alkyl)-(C₃-C₈ cycloalkyl), 3- to 10-membered heterocyclyl, —(C₁-C₃ alkyl)-(3- to 10-membered heterocyclyl), C₆-C₁₀ aryl, —(C₁-C₃ alkyl)-(C₆-C₁₀ aryl), —(C₂-C₄ alkynyl)-(C₆-C₁₀ aryl), 5- to 10-membered heteroaryl, —(C₁-C₃ alkyl)-(5- to 10-membered heteroaryl), or SF₅, wherein each cycloalkyl, alkyl-cycloalkyl, heterocyclyl, alkyl-heterocyclyl, aryl, alkyl-aryl, alkynyl-aryl, heteroaryl, and alkyl-heteroaryl is substituted with 0, 1, 2, or 3 halo, C₁-C₃ haloalkyl, C₁-C₃ haloalkoxy, or SF₅;
alternatively, R^{C3a1} and R^{C3a2} together with the N to which they are attached form a 3- to 8-membered heterocycle;
R^{D} is halo;
each heterocyclyl has 1, 2, 3, or 4 heteroatoms selected from N, O, S, and Si; and each heteroaryl has 1, 2, 3, or 4 heteroatoms selected from N, O, and S.

In another embodiment, the present disclosure provides a pharmaceutical composition comprising a compound of the present disclosure, and a pharmaceutically acceptable excipient.

In another embodiment, the present disclosure provides a method of inhibiting KRAS G12D protein in a subject in need thereof, the method comprising administering to the subject a therapeutically effective amount of a compound of the present disclosure, or a pharmaceutically acceptable salt thereof, or a pharmaceutical composition of the present disclosure.

In another embodiment, the present disclosure provides a method of treating cancer in a subject in need thereof, the method comprising administering to the subject a therapeutically effective amount of a compound of the present disclosure, or a pharmaceutically acceptable salt thereof, or a pharmaceutical composition of the present disclosure.

In another embodiment, the present disclosure provides a method for manufacturing a medicament for treating cancer in a subject in need thereof, characterized in that a compound of the present disclosure, or a pharmaceutically acceptable salt thereof, is used.

In another embodiment, the present disclosure provides a method for manufacturing a medicament for inhibiting cancer metastasis in a subject in need thereof, characterized in that a compound of the present invention, or a pharmaceutically acceptable salt thereof, is used.

In another embodiment, the present disclosure provides use of the compound of the present disclosure, or a pharmaceutically acceptable salt thereof, for the manufacture of a medicament for the treatment of cancer in a subject.

In another embodiment, the present disclosure provides use of the compound of the present disclosure, or a pharmaceutically acceptable salt thereof, for the manufacture of a medicament for inhibiting cancer metastasis in a subject.

In another embodiment, the present disclosure provides the compound of the present disclosure, or a pharmaceutically acceptable salt thereof, for use in the treatment of cancer in a subject in need thereof.

In another embodiment, the present disclosure provides the compound of the present disclosure, or a pharmaceutically acceptable salt thereof, for use in inhibiting cancer metastasis in a subject in need thereof.

In another embodiment, the present disclosure provides the compound of the present disclosure, or a pharmaceutically acceptable salt thereof, for use in therapy.

Also disclosed herein are compounds and pharmaceutically acceptable salts thereof of sub-formulas of Formula (I), such as Formula (Ia), (Ib), (Ic), or (Id).

### DETAILED DESCRIPTION

### I. General

The disclosure relates generally to methods and compounds, and pharmaceutically acceptable salts thereof, for inhibiting KRAS^{G12D} and/or KRAS^{G12C}. The following description sets forth exemplary methods, parameters and the like. It should be recognized, however, that such description is not intended as a limitation on the scope of the present disclosure but is instead provided as a description of exemplary embodiments.

### II. Definitions

As used in the present specification, the following words, phrases and symbols are generally intended to have the meanings as set forth below, except to the extent that the context in which they are used indicates otherwise.

A dash (“-”) that is not between two letters or symbols is used to indicate a point of attachment for a substituent. For example, —CONH₂ is attached through the carbon atom. A dash at the front or end of a chemical group is a matter of convenience; chemical groups can be depicted with or without one or more dashes without losing their ordinary meaning. A wavy line drawn through a line in a structure indicates a point of attachment of a group. Unless chemically or structurally required, no directionality is indicated or implied by the order in which a chemical group is written or named.

A squiggly line on a chemical group as shown below, for example,

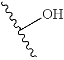 [SMILES: *O]

indicates a point of attachment, i.e., it shows the broken bond by which the group is connected to another described group.

As used herein, “a compound of the disclosure” can mean a compound of any of Formula (I), (Ia), (Ib), (Ic), or (Id), or a pharmaceutically acceptable salt thereof. Similarly, the phrase “a compound of Formula (number)” means a compound of that formula and pharmaceutically acceptable salts thereof.

The prefix “Cᵤ-Cᵥ” indicates that the following group has from u to v carbon atoms. For example, “C₁-C₈ alkyl” indicates that the alkyl group has from 1 to 8 carbon atoms.

“Alkyl” refers to an unbranched or branched saturated hydrocarbon chain. For example, an alkyl group can have 1 to 20 carbon atoms (i.e., C₁-C₂₀ alkyl), 1 to 8 carbon atoms (i.e., C₁-C₈ alkyl), 1 to 6 carbon atoms (i.e., C₁-C₆ alkyl), or 1 to 3 carbon atoms (i.e., C₁-C₃ alkyl). Examples of suitable alkyl groups include, but are not limited to, methyl (Me, —CH₃), ethyl (Et, —CH₂CH₃), 1-propyl (n-Pr, n-propyl, —CH₂CH₂CH₃), 2-propyl (i-Pr, i-propyl, —CH(CH₃)₂), 1-butyl (n-Bu, n-butyl, —CH₂CH₂CH₂CH₃), 2-methyl-1-propyl (i-Bu, i-butyl, —CH₂CH(CH₃)₂), 2-butyl (s-Bu, s-butyl, —CH(CH₃)CH₂CH₃), 2-methyl-2-propyl (t-Bu, t-butyl, —C(CH₃)₃), 1-pentyl (n-pentyl, —CH₂CH₂CH₂CH₂CH₃), 2-pentyl (—CH(CH₃)CH₂CH₂CH₃), 3-pentyl (—CH(CH₂CH₃)₂), 2-methyl-2-butyl (—C(CH₃)₂CH₂CH₃), 3-methyl-2-butyl (—CH(CH₃)CH(CH₃)₂), 3-methyl-1-butyl (—CH₂CH₂CH(CH₃)₂), 2-methyl-1-butyl (—CH₂CH(CH₃)CH₂CH₃), 1-hexyl (—CH₂CH₂CH₂CH₂CH₂CH₃), 2-hexyl (—CH(CH₃)CH₂CH₂CH₂CH₃), 3-hexyl (—CH(CH₂CH₃)(CH₂CH₂CH₃)), 2-methyl-2-pentyl (—C(CH₃)₂CH₂CH₂CH₃), 3-methyl-2-pentyl (—CH(CH₃)CH(CH₃)CH₂CH₃), 4-methyl-2-pentyl (—CH(CH₃)CH₂CH(CH₃)₂), 3-methyl-3-pentyl (—C(CH₃)(CH₂CH₃)₂), 2-methyl-3-pentyl (—CH(CH₂CH₃)CH(CH₃)₂), 2,3-dimethyl-2-butyl (—C(CH₃)₂CH(CH₃)₂), and 3,3-dimethyl-2-butyl (—CH(CH₃)C(CH₃)₃. Other alkyl groups include, but are not limited to, heptyl, octyl, nonyl, decyl, undecyl, dodecyl, pentadecyl, hexadecyl, heptadecyl and octadecyl.

“Alkenyl” refers to an unbranched or branched hydrocarbon chain containing at least two carbon atoms and at least one carbon-carbon double bond. As used herein, alkenyl can have from 2 to 20 carbon atoms (i.e., C₂₋₂₀ alkenyl), 2 to 8 carbon atoms (i.e., C₂₋₈ alkenyl), 2 to 6 carbon atoms (i.e., C₂₋₆ alkenyl), or 2 to 4 carbon atoms (i.e., C₂₋₄ alkenyl). Alkenyl can include any number of carbons, such as C₂, C₃, C₄, C₅, C₆, C₇, C₈, C₉, C₁₀, C₁₁, C₁₂, C₁₃, C₁₋₄, C₁₅, C₁₆, C₁₇, C18, C₁₉, C₂₀, or any range therein. Alkenyl groups can have any suitable number of double bonds, including, but not limited to, 1, 2, 3, 4, 5 or more. Examples of alkenyl groups include, but are not limited to, vinyl (ethenyl), propenyl, isopropenyl, 1-butenyl, 2-butenyl, isobutenyl, butadienyl, 1-pentenyl, 2-pentenyl, isopentenyl, 1,3-pentadienyl, 1,4-pentadienyl, 1-hexenyl, 2-hexenyl, 3-hexenyl, 1,3-hexadienyl, 1,4-hexadienyl, 1,5-hexadienyl, 2,4-hexadienyl, or 1,3,5-hexatrienyl.

“Alkynyl” refers to an unbranched or branched hydrocarbon chain containing at least one carbon-carbon triple bond. For example, an alkynyl group can have from 2 to 20 carbon atoms (i.e., C₂₋₂₀ alkynyl), 2 to 8 carbon atoms (i.e., C₂₋₈ alkynyl), 2 to 6 carbon atoms (i.e., C₂₋₆ alkynyl), or 2 to 4 carbon atoms (i.e., C₂₋₄ alkynyl). The term “alkynyl” also includes those groups having one triple bond and one double bond. Examples of C₂₋₆alkynyl include, but are not limited to, ethynyl, prop-1-ynyl, but-1-ynyl, pent-1-ynyl, pent-4-ynyl and penta-1,4-diynyl.

“Alkoxy” means a group having the formula —O-alkyl, in which an alkyl group, as defined above, is attached to the parent molecule via an oxygen atom. The alkyl portion of an alkoxy group can have 1 to 20 carbon atoms (i.e., C₁-C₂₀ alkoxy), 1 to 12 carbon atoms (i.e., C₁-C₁₂ alkoxy), 1 to 8 carbon atoms (i.e., C₁-C₈ alkoxy), 1 to 6 carbon atoms (i.e., C₁-C₆ alkoxy) or 1 to 3 carbon atoms (i.e., C₁-C₃ alkoxy). Examples of suitable alkoxy groups include, but are not limited to, methoxy (—O—CH₃ or —OMe), ethoxy (—OCH₂CH₃ or -OEt), isopropoxy (—O—CH(CH₃)₂), t-butoxy (—O—C(CH₃)₃ or -OtBu) and the like. Other examples of suitable alkoxy groups include, but are not limited to, sec-butoxy, tert-butoxy, pentoxy, hexoxy, and the like.

“Alkoxyalkyl” refers an alkoxy group linked to an alkyl group which is linked to the remainder of the compound. Alkoxyalkyl can have any suitable number of carbon, such as from 2 to 6 (C₂₋₆ alkoxyalkyl), 2 to 5 (C₂₋₅ alkoxyalkyl), 2 to 4 (C₂_₄ alkoxyalkyl), or 2 to 3 (C₂₋₃ alkoxyalkyl). Alkoxy and alkyl are as defined above. Examples of “alkoxyalkyl” include, but are not limited to, methoxymethyl (CH₃OCH₂—), and methoxyethyl (CH₃OCH₂CH₂).

“Bridged” means a ring system in which non-adjacent atoms on a ring are connected by a divalent substituent, such as an alkylenyl or heteroalkylenyl group or a single heteroatom.

“Hydroxyalkyl” refers to a hydroxy group, —OH, linked to an alkyl group which is linked to the remainder of the compound such that the alkyl group is divalent. Hydroxyalkyl can have any suitable number of carbons, such as from 1 to 8 (C₁₋₈ hydroxyalkyl), 1 to 6 (C₁₋₆ hydroxyalkyl), 2 to 6 (C₂_₆ hydroxyalkyl), 2 to 4 (C₂₋₄ hydroxyalkyl), or 2 to 3 (C₂₋₃ hydroxyalkyl). Alkyl is as defined above where the alkyl is divalent.

“Halo” or “halogen” as used herein refers to fluoro (—F), chloro (—Cl), bromo (—Br) and iodo (—I).

“Haloalkyl” is an alkyl group, as defined above, in which one or more hydrogen atoms of the alkyl group is replaced with a halogen atom. The alkyl portion of a haloalkyl group can have 1 to 20 carbon atoms (i.e., C₁-C₂₀ haloalkyl), 1 to 12 carbon atoms (i.e., C₁-C₁₂ haloalkyl), 1 to 8 carbon atoms (i.e., C₁-C₅ haloalkyl), 1 to 6 carbon atoms (i.e., C₁-C₆ alkyl) or 1 to 3 carbon atoms (i.e., C₁-C₃ alkyl). The alkyl groups can be substituted with 1, 2, 3, 4, 5, 6, 7, 8, 9 or more halogens. Examples of suitable haloalkyl groups include, but are not limited to, —CF₃, —CHF₂, —CFH₂, —CH₂CF₃, fluorochloromethyl, difluorochloromethyl, 1,1,1-trifluoroethyl and pentafluoroethyl.

“Haloalkoxy” refers to an alkoxy group where some or all of the hydrogen atoms are substituted with halogen atoms. As for an alkyl group, haloalkoxy groups can have any suitable number of carbon atoms, such as C₁₋₆. The alkoxy groups can be substituted with 1, 2, 3, 4, 5, 6, 7, 8, 9 or more halogens. When all the hydrogens are replaced with a halogen, for example by fluorine, the compounds are per-substituted, for example, perfluorinated. Haloalkoxy includes, but is not limited to, trifluoromethoxy, 2,2,2,-trifluoroethoxy, perfluoroethoxy, etc.

“Thioalkyl” refers to a thio group, —SH, linked to an alkyl group which is linked to the remainder of the compound such that the alkyl group is divalent. Thioalkyl can have any suitable number of carbons, such as from 1 to 8 (C₁₋₈ thioalkyl), 1 to 6 (C₁₋₆ thioalkyl), 2 to 6 (C₂₋₆ thioalkyl), 2 to 4 (C₂₋₄ thioalkyl), or 2 to 3 (C₂₋₃ thioalkyl). Alkyl is as defined above where the alkyl is divalent.

“Haloalkylthio” is an alkylthio group, as defined above, in which one or more hydrogen atoms of the alkyl group is replaced with a halogen atom. The alkyl portion of a haloalkylthio group can have 1 to 20 carbon atoms (i.e., C₁-C₂₀ haloalkylthio), 1 to 12 carbon atoms (i.e., C₁-C₁₂ haloalkylthio), 1 to 8 carbon atoms (i.e., C₁-C₈ haloalkylthio), 1 to 6 carbon atoms (i.e., C₁-C₆ alkylthio) or 1 to 3 carbon atoms (i.e., C₁-C₃ alkylthio). The alkythio groups can be substituted with 1, 2, 3, 4, 5, 6, 7, 8, 9 or more halogens.

“Heteroalkyl” refers to an unbranched or branched saturated hydrocarbon chain containing from 1 to 4 heteroatoms.

“Cyanoalkyl” refers to a cyano group, —CN, linked to an alkyl group which is linked to the remainder of the compound such that the alkyl group is divalent. Cyanoalkyl can have any suitable number of carbons, such as from 1 to 8 (C₁₋₈ cyanoalkyl), 1 to 6 (C₁₋₆ cyanoalkyl), 2 to 6 (C₂₋₆ cyanoalkyl), 2 to 4 (C₂₋₄ cyanoalkyl), or 2 to 3 (C₂₋₃ cyanoalkyl). Alkyl is as defined above where the alkyl is divalent.

“Cycloalkyl” refers to a saturated or partially saturated cyclic alkyl group having a single ring or multiple rings, such as 2, 3, 4 or more, wherein the multiple rings can be fused, bridged, spiro, or any combination thereof. As used herein, cycloalkyl has from 3 to 20 ring carbon atoms (i.e., C₃₋₂₀ cycloalkyl), 3 to 12 ring carbon atoms (i.e., C₃₋₁₂ cycloalkyl), 3 to 10 ring carbon atoms (i.e., C₃₋₁₀ cycloalkyl), 3 to 8 ring carbon atoms (i.e., C₃₋₈ cycloalkyl), or 3 to 6 ring carbon atoms (i.e., C₃₋₆ cycloalkyl). Examples of cycloalkyl groups include, but are not limited to, cyclopropyl, cyclobutyl, cyclopentyl, cyclohexyl, cycloheptyl, and cyclooctyl. Cycloalkyl groups also include partially unsaturated ring systems containing one or more double bonds, including fused ring systems with one aromatic ring and one non-aromatic ring, but not fully aromatic ring systems.

The term “fused” refers to a ring system in which two or more rings in the system share a pair of adjacent ring atoms.

“Spiro” refers to at least two rings are linked together by one common atom. “Spiro” also refers to a ring substituent which is joined by two bonds at the same carbon atom. Examples of spiro groups include, but are not limited to, 1,1-diethylcyclopentane, dimethyl-dioxolane, and 4-benzyl-4-methylpiperidine, wherein the cyclopentane and piperidine, respectively, are the spiro substituents.

“Alkyl-cycloalkyl” refers to a radical having an alkyl component and a cycloalkyl component, where the alkyl component links the cycloalkyl component to the point of attachment.

The alkyl component is as defined above, except that the alkyl component is at least divalent, an alkylene, to link to the cycloalkyl component and to the point of attachment. In some instances, the alkyl component can be absent. The alkyl component can include any number of carbons, such as C₁₋₆, C₁₋₂, C₁₋₃, C₁₋₄, C₁₋₅, C₂₋₃, C₂₋₄, C₂₋₅, C₂₋₆, C₃₋₄, C₃₋₅, C₃₋₆, C₄₋₅, C₄₋₆ and C₅₋₆. The cycloalkyl component is as defined within. Exemplary alkyl-cycloalkyl groups include, but are not limited to, methyl-cyclopropyl, methyl-cyclobutyl, methyl-cyclopentyl and methyl-cyclohexyl.

“Heterocycle” or “heterocyclyl” or “heterocycloalkyl” refer to a saturated or unsaturated cyclic alkyl group, with one or more ring heteroatoms independently selected from nitrogen, oxygen, sulfur and silicon. A heterocyclyl can be a single ring or multiple rings, such as 2, 3, 4 or more, wherein the multiple rings can be fused, bridged, spiro, or any combination thereof. As used herein, heterocyclyl has 3 to 20 ring atoms (i.e., 3 to 20 membered heterocyclyl), 3 to 12 ring atoms (i.e., 3 to 12 membered heterocyclyl), 3 to 10 ring atoms (i.e., 3 to 10 membered heterocyclyl), 3 to 8 ring atoms (i.e., 3 to 8 membered heterocyclyl), 4 to 12 ring carbon atoms (i.e., 4 to 12 membered heterocyclyl), 4 to 8 ring atoms (i.e., 4 to 8 membered heterocyclyl), or 4 to 6 ring atoms (i.e., 4 to 6 membered heterocyclyl). Examples of heterocyclyl groups include pyrrolidinyl, piperidinyl, piperazinyl, oxetanyl, dioxolanyl, azetidinyl, and morpholinyl.

“Alkyl-heterocycloalkyl” refers to a radical having an alkyl component and a heterocycloalkyl component, where the alkyl component links the heterocycloalkyl component to the point of attachment. The alkyl component is as defined above, except that the alkyl component is at least divalent, an alkylene, to link to the heterocycloalkyl component and to the point of attachment. The alkyl component can include any number of carbons, such as C₀₋₆, C₁₋₂, C₁₋₃, C₁₋₄, C₁₋₅, C₁₋₆, C₂₋₃, C₂₋₄, C₂₋₅, C₂₋₆, C₃₋₄, C₃₋₅, C₃₋₆, C₄₋₅, C₄₋₆ and C₅₋₆. In some instances, the alkyl component can be absent. The heterocycloalkyl component is as defined above.

“Aryl” means an aromatic hydrocarbon radical derived by the removal of one hydrogen atom from a single carbon atom of a parent aromatic ring system. For example, an aryl group can have 6 to 20 carbon atoms, 6 to 14 carbon atoms, or 6 to 10 carbon atoms. Exemplary aryl groups include, but are not limited to, radicals derived from benzene (e.g., phenyl), naphthalene, anthracene, biphenyl, and the like.

“Alkyl-aryl” refers to a radical having an alkyl component and an aryl component, where the alkyl component links the aryl component to the point of attachment. The alkyl component is as defined above, except that the alkyl component is at least divalent, an alkylene, to link to the aryl component and to the point of attachment. The alkyl component can include any number of carbons, such as C₀₋₆, C₁₋₂, C₁₋₃, C₁₋₄, C₁₋₅, C₁₋₆, C₂₋₃, C₂₋₄, C₂₋₅, C₂₋₆, C₃₋₄, C₃₋₅, C₃₋₆, C₄₋₅, C₄₋₆ and C₅₋₆. In some instances, the alkyl component can be absent. The aryl component is as defined above. Examples of alkyl-aryl groups include, but are not limited to, benzyl and ethyl-benzene.

“Heteroaryl” refers to an aromatic group, including groups having an aromatic tautomer or resonance structure, having a single ring, multiple rings, or multiple fused rings, with at least one heteroatom in the ring, i.e., one or more ring heteroatoms independently selected from nitrogen, oxygen, and sulfur, wherein the nitrogen or sulfur can be oxidized. Thus, the term includes rings having one or more annular O, N, S, S(O), S(O)₂, and N-oxide groups. The term includes rings having one or more annular C(O) groups. As used herein, heteroaryl include 5 to 20 ring atoms (i.e., 5- to 20-membered heteroaryl), 5 to 12 ring atoms (i.e., 5- to 12-membered heteroaryl), or 5 to 10 ring atoms (i.e., 5- to 10-membered heteroaryl), and 1 to 5 heteroatoms independently selected from nitrogen, oxygen, and sulfur, and oxidized forms of the heteroatoms. Examples of heteroaryl groups include, but are not limited to, pyridin-2(1H)-one, pyridazin-3(2H)-one, pyrimidin-4(3H)-one, quinolin-2(1H)-one, pyrimidinyl, purinyl, pyridyl, pyridazinyl, benzothiazolyl, and pyrazolyl. Heteroaryl does not encompass or overlap with aryl as defined above.

“Alkyl-heteroaryl” refers to a radical having an alkyl component and a heteroaryl component, where the alkyl component links the heteroaryl component to the point of attachment. The alkyl component is as defined above, except that the alkyl component is at least divalent, an alkylene, to link to the heteroaryl component and to the point of attachment. The alkyl component can include any number of carbons, such as C₀₋₆, C₁₋₂, C₁₋₃, C₁₋₄, C₁₋₅, C₁₋₆, C₂₋₃, C₂₋₄, C₂₋₅, C₂₋₆, C₃₋₄, C₃₋₅, C₃₋₆, C₄₋₅, C₄₋₆ and C₅₋₆. In some instances, the alkyl component can be absent. The heteroaryl component is as defined within.

“KRAS G12D” refers to the G12D mutation of the KRAS protein, where aspartic acid replaces glycine at amino acid position 12.

“KRAS G12D inhibitor” refers to compounds of the present disclosure, including compounds of Formula (I), (Ia), (Ib), (Ic), or (Id), or a pharmaceutically acceptable salt thereof. The compounds modulate or inhibit some or all of the activity of KRAS G12D.

“KRAS G12D-associated disease or disorder” refers to diseases or disorders associated with or mediated by or having a KRAS G12D mutation. Representative diseases or disorders include, but are not limited to, KRAS G12D-associated cancer.

“Oxo” refers to the group (═O) or (O).

Provided are also pharmaceutically acceptable salts, hydrates, solvates, tautomeric forms, polymorphs, and prodrugs of the compounds described herein. “Pharmaceutically acceptable” or “physiologically acceptable” refer to compounds, salts, formulations, dosage forms and other materials which are useful in preparing a pharmaceutical composition that is suitable for veterinary or human pharmaceutical use.

The compounds described herein can be prepared and/or formulated as pharmaceutically acceptable salts or when appropriate as a free base. Pharmaceutically acceptable salts are non-toxic salts of a free base form of a compound that possess the desired pharmacological activity of the free base. These salts can be derived from inorganic or organic acids or bases. For example, a compound that contains a basic nitrogen can be prepared as a pharmaceutically acceptable salt by contacting the compound with an inorganic or organic acid. Non-limiting examples of pharmaceutically acceptable salts include sulfates, pyrosulfates, bisulfates, sulfites, bisulfites, phosphates, monohydrogen-phosphates, dihydrogenphosphates, metaphosphates, pyrophosphates, chlorides, bromides, iodides, acetates, propionates, decanoates, caprylates, acrylates, formates, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxybenzoates, phthalates, sulfonates, methylsulfonates, propylsulfonates, besylates, xylenesulfonates, naphthalene-1-sulfonates, naphthalene-2-sulfonates, phenylacetates, phenylpropionates, phenylbutyrates, citrates, lactates, γ-hydroxybutyrates, glycolates, tartrates, and mandelates. Lists of other suitable pharmaceutically acceptable salts are found in REMINGTON: THE SCIENCE AND PRACTICE OF PHARMACY, 21^{st} Edition, Lippincott Wiliams and Wilkins, Philadelphia, Pa., 2006.

Examples of “pharmaceutically acceptable salts” of the compounds disclosed herein also include salts derived from an appropriate base, such as an alkali metal (for example, sodium, potassium), an alkaline earth metal (for example, magnesium), ammonium and NX₄+(wherein X is C₁-C₄ alkyl). Also included are base addition salts, such as sodium or potassium salts.

Provided are also compounds described herein or pharmaceutically acceptable salts, isomers, or a mixture thereof, in which from 1 to n hydrogen atoms attached to a carbon atom can be replaced by a deuterium atom or D, in which n is the number of hydrogen atoms in the molecule.

As known in the art, the deuterium atom is a non-radioactive isotope of the hydrogen atom. Such compounds can increase resistance to metabolism, and thus can be useful for increasing the half-life of the compounds described herein or pharmaceutically acceptable salts, isomer, or a mixture thereof when administered to a mammal. See, e.g., Foster, “Deuterium Isotope Effects in Studies of Drug Metabolism,” TRENDS PHARMACOL. SCI., 5(12):524-527 (1984). Such compounds are synthesized by means well known in the art, for example by employing starting materials in which one or more hydrogen atoms have been replaced by deuterium.

Examples of isotopes that can be incorporated into the disclosed compounds also include isotopes of hydrogen, carbon, nitrogen, oxygen, phosphorous, fluorine, chlorine, and iodine, such as ²H, ³H, ¹¹C, ¹³C, ¹⁴C, ¹³N, ¹⁵N, ¹⁵O, ¹⁷O ¹⁸O, ³¹P, ³²P, ³⁵S, ¹⁸F, ³⁶Cl, ¹²³I, and ¹²⁵I, respectively. Substitution with positron emitting isotopes, such as ¹¹C, ¹⁸F, ¹⁵O and ¹³N, can be useful in Positron Emission Topography (PET) studies for examining substrate receptor occupancy.

Isotopically-labeled compounds of Formula (I), (Ia), (Ib), (Ic), or (Id) can generally be prepared by conventional techniques known to those skilled in the art or by processes analogous to those described in the Examples as set out below using an appropriate isotopically-labeled reagent in place of the non-labeled reagent previously employed.

The compounds of the embodiments disclosed herein, or their pharmaceutically acceptable salts can contain one or more asymmetric centers and can thus give rise to enantiomers, diastereomers, and other stereoisomeric forms that can be defined, in terms of absolute stereochemistry, as (R)- or (S)- or, as (D)- or (L)- for amino acids. The present disclosure is meant to include all such possible isomers, as well as their racemic and optically pure forms. Optically active (+) and (−), (R)- and (S)-, or (D)- and (L)- isomers can be prepared using chiral synthons or chiral reagents, or resolved using conventional techniques, for example, chromatography and fractional crystallization. Conventional techniques for the preparation/isolation of individual enantiomers include chiral synthesis from a suitable optically pure precursor or resolution of the racemate (or the racemate of a salt or derivative) using, for example, chiral high-pressure liquid chromatography (HPLC). When the compounds described herein contain olefinic double bonds or other centers of geometric asymmetry, and unless specified otherwise, it is intended that the compounds include both E and Z geometric isomers. Likewise, all tautomeric forms are also intended to be included. Where compounds are represented in their chiral form, it is understood that the embodiment encompasses, but is not limited to, the specific diastereomerically or enantiomerically enriched form. Where chirality is not specified but is present, it is understood that the embodiment is directed to either the specific diastereorerically or enantiomerically enriched form; or a racemic or scalemic mixture of such compound(s). As used herein, “scalemic mixture” is a mixture of stereoisomers at a ratio other than 1:1.

“Racemates” refers to a mixture of enantiomers. The mixture can comprise equal or unequal amounts of each enantiomer.

“Stereoisomer” and “stereoisomers” refer to compounds that differ in the chirality of one or more stereocenters. Stereoisomers include enantiomers and diastereomers. The compounds can exist in stereoisomeric form if they possess one or more asymmetric centers or a double bond with asymmetric substitution and, therefore, can be produced as individual stereoisomers or as mixtures. Unless otherwise indicated, the description is intended to include individual stereoisomers as well as mixtures. The methods for the determination of stereochemistry and the separation of stereoisomers are well-known in the art (see, e.g., Chapter 4 of ADVANCED ORGANIC CHEMISTRY, 4th ed., J. March, John Wiley & Sons, New York, 1992).

A “subject” or “patient” is meant to describe a human or vertebrate animal including a dog, cat, pocket pet, marmoset, horse, cow, pig, sheep, goat, elephant, giraffe, chicken, lion, monkey, owl, rat, squirrel, slender loris, and mouse. A “pocket pet” refers to a group of vertebrate animals capable of fitting into a commodious coat pocket such as, for example, hamsters, chinchillas, ferrets, rats, guinea pigs, gerbils, rabbits and sugar gliders.

Unless defined otherwise, all technical and scientific terms used herein have the same meaning as commonly understood by one of ordinary skill in the art. A dash at the front or end of a chemical group is a matter of convenience; chemical groups can be depicted with or without one or more dashes without losing their ordinary meaning. A wavy line drawn through a line in a structure indicates a point of attachment of a group. A dashed line indicates an optional bond. Unless chemically or structurally required, no directionality is indicated or implied by the order in which a chemical group is written or the point at which it is attached to the remainder of the molecule. For instance, the group “—SO₂CH₂—” is equivalent to “—CH₂SO₂—” and both can be connected in either direction. Similarly, an “arylalkyl” group, for example, can be attached to the remainder of the molecule at either an aryl or an alkyl portion of the group. A prefix such as “Cᵤ-Cᵥ” or “(Cᵤ-Cᵥ)” indicates that the following group has from u to v carbon atoms. For example, “C₁₋₆ alkyl” and “C₁-C₆ alkyl” both indicate that the alkyl group has from 1 to 6 carbon atoms.

Unless otherwise specified, the carbon atoms of the compound of Formula (I), (Ia), (Ib), (Ic), or (Id), or a pharmaceutically acceptable salt thereof, are intended to have a valence of four. If in some chemical structure representations, carbon atoms do not have a sufficient number of variables attached to produce a valence of four, the remaining carbon substituents needed to provide a valence of four should be assumed to be hydrogen.

“Treatment” or “treating” is an approach for obtaining beneficial or desired results including clinical results. Beneficial or desired clinical results may include one or more of the following: (a) inhibiting the disease or condition (e.g., decreasing one or more symptoms resulting from the disease or condition, and/or diminishing the extent of the disease or condition); (b) slowing or arresting the development of one or more clinical symptoms associated with the disease or condition (e.g., stabilizing the disease or condition, preventing or delaying the worsening or progression of the disease or condition, and/or preventing or delaying the spread (e.g., metastasis) of the disease or condition); and/or (c) relieving the disease, that is, causing the regression of clinical symptoms (e.g., ameliorating the disease state, providing partial or total remission of the disease or condition, enhancing effect of another medication, delaying the progression of the disease, increasing the quality of life, and/or prolonging survival.

The term “therapeutically effective amount,” as used herein, is the amount of compound disclosed herein present in a formulation described herein that is needed to provide a desired level of drug in the secretions and tissues of the airways and lungs, or alternatively, in the bloodstream of a subject to be treated to give an anticipated physiological response or desired biological effect when such a formulation is administered by the chosen route of administration. The precise amount will depend upon numerous factors, for example the particular compound disclosed herein, the specific activity of the formulation, the delivery device employed, the physical characteristics of the formulation, its intended use, as well as subject considerations such as severity of the disease state, subject cooperation, etc., and can readily be determined by one skilled in the art based upon the information provided herein. The term “therapeutically effective amount” or “effective amount” also means amounts that eliminate or reduce the subject's viral burden and/or viral reservoir.

“Administering” refers to oral administration, administration as a suppository, topical contact, parenteral, intravenous, intraperitoneal, intramuscular, intralesional, intranasal or subcutaneous administration, intrathecal administration, or the implantation of a slow-release device e.g., a mini-osmotic pump, to the subject. The administration can be carried out according to a schedule specifying frequency of administration, dose for administration, and other factors.

“Co-administration” as used herein refers to administration of unit dosages of the compounds disclosed herein before or after administration of unit dosages of one or more additional therapeutic agents, for example, administration of the compound disclosed herein within seconds, minutes, or hours of the administration of one or more additional therapeutic agents. For example, in some embodiments, a unit dose of a compound of the present disclosure is administered first, followed within seconds or minutes by administration of a unit dose of one or more additional therapeutic agents. Alternatively, in other embodiments, a unit dose of one or more additional therapeutic agents is administered first, followed by administration of a unit dose of a compound of the present disclosure within seconds or minutes. In some embodiments, a unit dose of a compound of the present disclosure is administered first, followed, after a period of hours (e.g., 1-12 hours), by administration of a unit dose of one or more additional therapeutic agents. In other embodiments, a unit dose of one or more additional therapeutic agents is administered first, followed, after a period of hours (e.g., 1-12 hours), by administration of a unit dose of a compound of the present disclosure. Co-administration of a compound disclosed herein with one or more additional therapeutic agents generally refers to simultaneous or sequential administration of a compound disclosed herein and one or more additional therapeutic agents, such that therapeutically effective amounts of each agent are present in the body of the patient.

“Subject” refers to animals such as mammals, including, but not limited to, primates (e.g., humans), cows, sheep, goats, horses, dogs, cats, rabbits, rats, mice and the like. In some embodiments, the subject is a human.

“Disease” or “condition” refer to a state of being or health status of a patient or subject capable of being treated with a compound, pharmaceutical composition, or method provided herein. The disease may be an autoimmune, inflammatory, cancer, infectious (e.g., a viral infection), metabolic, developmental, cardiovascular, liver, intestinal, endocrine, neurological, or other disease. In some embodiments, the disease is cancer (e.g. lung cancer, ovarian cancer, osteosarcoma, bladder cancer, cervical cancer, liver cancer, kidney cancer, skin cancer (e.g., Merkel cell carcinoma), testicular cancer, leukemia, lymphoma, head and neck cancer, colorectal cancer, prostate cancer, pancreatic cancer, melanoma, breast cancer, neuroblastoma).

“Cancer” refers to all types of cancer, neoplasm or malignant tumors found in mammals, including leukemias, lymphomas, melanomas, neuroendocrine tumors, carcinomas and sarcomas.

Exemplary cancers that may be treated with a compound, pharmaceutical composition, or method provided herein include lymphoma, sarcoma, bladder cancer, bone cancer, brain tumor, cervical cancer, colon cancer, esophageal cancer, gastric cancer, head and neck cancer, kidney cancer, myeloma, thyroid cancer, leukemia, prostate cancer, breast cancer (e.g. triple negative, ER positive, ER negative, chemotherapy resistant, herceptin resistant, HER2 positive, doxorubicin resistant, tamoxifen resistant, ductal carcinoma, lobular carcinoma, primary, metastatic), ovarian cancer, pancreatic cancer, liver cancer (e.g. hepatocellular carcinoma), lung cancer (e.g. non-small cell lung carcinoma, squamous cell lung carcinoma, adenocarcinoma, large cell lung carcinoma, small cell lung carcinoma, carcinoid, sarcoma), glioblastoma multiforme, glioma, melanoma, prostate cancer, castration-resistant prostate cancer, breast cancer, triple negative breast cancer, glioblastoma, ovarian cancer, lung cancer, squamous cell carcinoma (e.g., head, neck, or esophagus), colorectal cancer, leukemia, acute myeloid leukemia, lymphoma, B cell lymphoma, or multiple myeloma.

Additional examples include, cancer of the thyroid, endocrine system, brain, breast, cervix, colon, head & neck, esophagus, liver, kidney, lung, non-small cell lung, melanoma, mesothelioma, ovary, sarcoma, stomach, uterus or Medulloblastoma, Hodgkin's Disease, Non-Hodgkin's Lymphoma, multiple myeloma, neuroblastoma, glioma, glioblastoma multiforme, ovarian cancer, rhabdomyosarcoma, primary thrombocytosis, primary macroglobulinemia, primary brain tumors, cancer, malignant pancreatic insulanoma, malignant carcinoid, urinary bladder cancer, premalignant skin lesions, testicular cancer, lymphomas, thyroid cancer, neuroblastoma, esophageal cancer, genitourinary tract cancer, malignant hypercalcemia, endometrial cancer, adrenal cortical cancer, neoplasms of the endocrine or exocrine pancreas, medullary thyroid cancer, medullary thyroid carcinoma, melanoma, colorectal cancer, papillary thyroid cancer, hepatocellular carcinoma, Paget's Disease of the Nipple, Phyllodes Tumors, Lobular Carcinoma, Ductal Carcinoma, cancer of the pancreatic stellate cells, cancer of the hepatic stellate cells, or prostate cancer.

“Leukemia” refers broadly to progressive, malignant diseases of the blood-forming organs and is generally characterized by a distorted proliferation and development of leukocytes and their precursors in the blood and bone marrow. Leukemia is generally clinically classified on the basis of (1) the duration and character of the disease-acute or chronic; (2) the type of cell involved; myeloid (myelogenous), lymphoid (lymphogenous), or monocytic; and (3) the increase or non-increase in the number abnormal cells in the blood-leukemic or aleukemic (subleukemic).

Exemplary leukemias that may be treated with a compound, pharmaceutical composition, or method provided herein include, for example, acute nonlymphocytic leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophylic leukemia, blast cell leukemia, bovine leukemia, chronic myelocytic leukemia, leukemia cutis, embryonal leukemia, eosinophilic leukemia, Gross' leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocyte leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, multiple myeloma, plasmacytic leukemia, promyelocytic leukemia, Rieder cell leukemia, Schilling's leukemia, stem cell leukemia, subleukemic leukemia, or undifferentiated cell leukemia.

“Sarcoma” generally refers to a tumor which is made up of a substance like the embryonic connective tissue and is generally composed of closely packed cells embedded in a fibrillar or homogeneous substance. Sarcomas that may be treated with a compound, pharmaceutical composition, or method provided herein include a chondrosarcoma, fibrosarcoma, lymphosarcoma, melanosarcoma, myxosarcoma, osteosarcoma, Abemethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilms' tumor sarcoma, endometrial sarcoma, stromal sarcoma, Ewing's sarcoma, fascial sarcoma, fibroblastic sarcoma, giant cell sarcoma, granulocytic sarcoma, Hodgkin's sarcoma, idiopathic multiple pigmented hemorrhagic sarcoma, immunoblastic sarcoma of B cells, lymphoma, immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, or telangiectaltic sarcoma.

“Melanoma” is taken to mean a tumor arising from the melanocytic system of the skin and other organs. Melanomas that may be treated with a compound, pharmaceutical composition, or method provided herein include, for example, acral-lentiginous melanoma, amelanotic melanoma, benign juvenile melanoma, Cloudman's melanoma, S91 melanoma, Harding-Passey melanoma, juvenile melanoma, lentigo maligna melanoma, malignant melanoma, nodular melanoma, subungal melanoma, or superficial spreading melanoma.

“Carcinoma” refers to a malignant new growth made up of epithelial cells tending to infiltrate the surrounding tissues and give rise to metastases. Exemplary carcinomas that may be treated with a compound, pharmaceutical composition, or method provided herein include, for example, medullary thyroid carcinoma, familial medullary thyroid carcinoma, acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basal oid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, ductal carcinoma, carcinoma durum, embryonal carcinoma, encephaloid carcinoma, epiermoid carcinoma, carcinoma epitheliale adenoides, exophytic carcinoma, carcinoma ex ulcere, carcinoma fibrosum, gelatiniforni carcinoma, gelatinous carcinoma, giant cell carcinoma, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypernephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lobular carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidermoid carcinoma, carcinoma mucosum, mucous carcinoma, carcinoma myxomatodes, nasopharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, carcinoma scroti, signet- ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tubular carcinoma, tuberous carcinoma, verrucous carcinoma, or carcinoma villosum.

“Metastasis,” “metastatic,” and “metastatic cancer” can be used interchangeably and refer to the spread of a proliferative disease or disorder, e.g., cancer, from one organ or another non-adjacent organ or body part. Cancer occurs at an originating site, e.g., breast, which site is referred to as a primary tumor, e.g., primary breast cancer. Some cancer cells in the primary tumor or originating site acquire the ability to penetrate and infiltrate surrounding normal tissue in the local area and/or the ability to penetrate the walls of the lymphatic system or vascular system circulating through the system to other sites and tissues in the body. A second clinically detectable tumor formed from cancer cells of a primary tumor is referred to as a metastatic or secondary tumor.

When cancer cells metastasize, the metastatic tumor and its cells are presumed to be similar to those of the original tumor. Thus, if lung cancer metastasizes to the breast, the secondary tumor at the site of the breast consists of abnormal lung cells and not abnormal breast cells. The secondary tumor in the breast is referred to a metastatic lung cancer. Thus, the phrase metastatic cancer refers to a disease in which a subject has or had a primary tumor and has one or more secondary tumors. The phrases non-metastatic cancer or subjects with cancer that is not metastatic refers to diseases in which subjects have a primary tumor but not one or more secondary tumors.

For example, metastatic lung cancer refers to a disease in a subject with or with a history of a primary lung tumor and with one or more secondary tumors at a second location or multiple locations, e.g., in the breast.

“Associated” or “associated with” in the context of a substance or substance activity or function associated with a disease (e.g., diabetes, cancer (e.g. prostate cancer, renal cancer, metastatic cancer, melanoma, castration-resistant prostate cancer, breast cancer, triple negative breast cancer, glioblastoma, ovarian cancer, lung cancer, squamous cell carcinoma (e.g., head, neck, or esophagus), colorectal cancer, leukemia, acute myeloid leukemia, lymphoma, B cell lymphoma, or multiple myeloma)) means that the disease (e.g. lung cancer, ovarian cancer, osteosarcoma, bladder cancer, cervical cancer, liver cancer, kidney cancer, skin cancer (e.g., Merkel cell carcinoma), testicular cancer, leukemia, lymphoma, head and neck cancer, colorectal cancer, prostate cancer, pancreatic cancer, melanoma, breast cancer, neuroblastoma) is caused by (in whole or in part), or a symptom of the disease is caused by (in whole or in part) the substance or substance activity or function.

The term “adjacent carbons” as used herein refers to consecutive carbons atoms that are directly attached to each other. For example, in

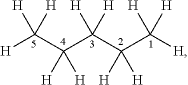

C₁ and C₂ are adjacent carbons, C₂ and C₃ are adjacent carbons, C₃ and C₄ are adjacent carbons, and C₄ and C₅ are adjacent carbons. Similarly, in

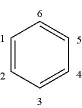

C₁ and C₂ are adjacent carbons, C₂ and C₃ are adjacent carbons, C₃ and C₄ are adjacent carbons, and C₄ and C₅ are adjacent carbons, C₅ and C₆ are adjacent carbons and C₆ and C₁ are adjacent carbons.

“Solvate” as used herein refers to the result of the interaction of a solvent and a compound. Solvates of salts of the compounds described herein are also provided. Hydrates of the compounds described herein are also provided.

“Prodrug” as used herein refers to a derivative of a drug that upon administration to the human body is converted to the parent drug according to some chemical or enzymatic pathway.

As used herein, “pharmaceutically acceptable carrier” or “pharmaceutically acceptable excipient” includes, but is not limited to, any and all solvents, dispersion media, coatings, antibacterial and antifungal agents, isotonic and absorption delaying agents, and combinations thereof. The use of pharmaceutically acceptable carriers and pharmaceutically acceptable excipients for pharmaceutically active substances is well known in the art. Except insofar as any conventional media or agent is incompatible with the active ingredient, its use in the therapeutic formulations is contemplated. Supplementary active ingredients can also be incorporated into the formulations. The carrier(s) must be “acceptable” in the sense of being compatible with the other ingredients of the formulation and physiologically innocuous to the recipient thereof.

### III. Compounds

Disclosed herein are, among other things, compounds of Formula (I), (Ia), (Tb), (Ic), and (Id), or a pharmaceutically acceptable salt thereof. In some embodiments, the compounds of Formulas (I), (Ia), (Tb), (Ic), and (Id) are prodrugs.

In some embodiments, the present disclosure provides a compound of Formula (I):

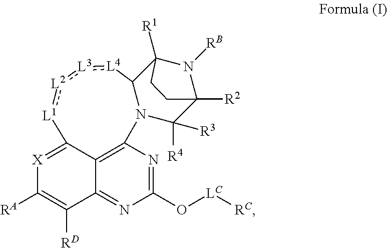

or a pharmaceutically acceptable salt thereof,
wherein
X is N, CH, or CR^{x};
R^{x} is C₁₋₃ alkyl, C₂₋₄ alkenyl, C₁₋₃ alkoxy, C₁₋₃ thioalkyl, —CN, (CH₂)ₘCN, halo, C₁₋₃ haloalkyl, or C₃₋₆ cycloalkyl;
m is 0, 1, 2 or 3;
R¹, R², R³, and R⁴ are each independently H or C₁-C₃ alkyl;
L¹ is O, S, CR^{1a}R^{1b}, C(═CR^{1c}R^{1d}), C(═O), or —C(R^{1e})=;
R^{1a} and R^{1b} are each independently H, C₁-C₃ alkyl, C₁-C₃ alkoxy, halo, C₁-C₆ haloalkyl, C₁-C₆ haloalkoxy, —OH, —CN, C₁-C₃ cyanoalkyl, or C₃-C₆ cycloalkyl;
alternatively, R^{1a} and R^{1b} can combine with the atom to which they are attached to form a C₃-C₆ cycloalkyl or a 3 to 6 membered heterocyclyl having 1 heteroatom that is O, wherein each cycloalkyl and heterocyclyl is substituted with 0, 1, 2, or 3 R^{1x};
R^{1c} and R^{1d} are each independently H, C₁-C₃ alkyl, halo, C₁-C₆ haloalkyl, —CN, C₁-C₃ cyanoalkyl, or C₃-C₆ cycloalkyl;
alternatively, R^{1c} and R^{1d} can combine with the atom to which they are attached to form a C₃-C₆ cycloalkyl or a 3 to 6 membered heterocyclyl having 1 heteroatom that is O, wherein each cycloalkyl and heterocyclyl is substituted with 0, 1, 2, or 3 R^{1x};
R^{1e} is H, C₁-C₃ alkyl, halo, C₁-C₆ haloalkyl, —CN, C₁-C₃ cyanoalkyl, or C₃-C₆ cycloalkyl;
each R^{1x} is independently C₁-C₃ alkyl, C₁-C₃ alkoxy, halo, C₁-C₆ haloalkyl, C₁-C₆ haloalkoxy, or —OH;
L² is absent, O, S, CR^{2a}R^{2b}, C(═CR^{2c}R^{2d}), C(═O), or =C(R^{2c})—, such that when L² is O or S, then L¹ is CR^{1a}R^{1b};
R^{2a} and R^{2b} are each independently H, C₁-C₃ alkyl, C₁-C₃ alkoxy, halo, C₁-C₆ haloalkyl, C₁-C₆ haloalkoxy, —OH, —CN, C₁-C₃ cyanoalkyl, or C₃-C₆ cycloalkyl;
alternatively, R^{2a} and R^{2b} can combine with the atom to which they are attached to form a C₃-C₆ cycloalkyl;
alternatively, R^{1b} and R^{2b} can combine with the atoms to which they are attached to form a C₃-C₆ cycloalkyl, 4- to 10-membered heterocyclyl, C₆-C₁₀ aryl, or 5- to 14-membered heteroaryl, wherein each cycloalkyl, heterocyclyl, aryl and heteroaryl is substituted with 0, 1, 2, or 3 R^{2x};
R^{2c} and R^{2d} are each independently H, C₁-C₃ alkyl, halo, C₁-C₆ haloalkyl, —CN, C₁-C₃ cyanoalkyl, or C₃-C₆ cycloalkyl;
alternatively, R^{2c} and R^{2d} can combine with the atom to which they are attached to form a C₃-C₆ cycloalkyl or a 3 to 6 membered heterocyclyl having 1 heteroatom that is O, wherein each cycloalkyl and heterocyclyl is substituted with 0, 1, 2, or 3 R^{2x};
R^{2e} is H, C₁-C₃ alkyl, halo, C₁-C₆ haloalkyl, —CN, C₁-C₃ cyanoalkyl, or C₃-C₆ cycloalkyl;
alternatively, R^{1e} and R^{2e} can combine with the atoms to which they are attached to form a C₅-C₆ cycloalkyl, 5- to 10-membered heterocyclyl, C₆-C₁₀ aryl, or 5- to 14-membered heteroaryl, wherein each cycloalkyl, heterocyclyl, aryl and heteroaryl is substituted with 0, 1, 2, or 3 R^{2x};
each R^{2x} is independently C₁-C₃ alkyl, C₁-C₃ alkoxy, halo, C₁-C₆ haloalkyl, C₁-C₆ haloalkoxy, or —OH;
alternatively L¹ and L² can combine to form

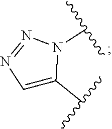 [SMILES: *c1cnnn1*]

L³ is absent, CR^{3a}R^{3b}, C(═CR^{3c}R^{3d}), C(═O), or =C(R^{3e});
R^{3a} and R^{3b} are each independently H, C₁-C₃ alkyl, C₁-C₃ alkoxy, halo, C₁-C₆ haloalkyl, C₁-C₆ haloalkoxy, —OH, —CN, C₁-C₃ cyanoalkyl, or C₃-C₆ cycloalkyl;
alternatively, R^{3a} and R^{3b} can combine with the atom to which they are attached to form a C₃-C₆ cycloalkyl;
alternatively, R^{2b} and R^{3b} can combine with the atoms to which they are attached to form a C₃-C₆ cycloalkyl;
R^{3c} and R^{3d} are each independently H, C₁-C₃ alkyl, halo, C₁-C₆ haloalkyl, —CN, C₁-C₃ cyanoalkyl, or C₃-C₆ cycloalkyl;
alternatively, R^{3c} and R^{3d} can combine with the atom to which they are attached to form a C₃-C₆ cycloalkyl or a 3 to 6 membered heterocyclyl having 1 heteroatom that is O, wherein each cycloalkyl and heterocyclyl is substituted with 0, 1, 2, or 3 R^{3x};
each R^{3x} is independently C₁-C₃ alkyl, C₁-C₃ alkoxy, halo, C₁-C₆ haloalkyl, C₁-C₆ haloalkoxy, or —OH;
R^{3e} is H, C₁-C₃ alkyl, halo, C₁-C₆ haloalkyl, —CN, C₁-C₃ cyanoalkyl, or C₃-C₆ cycloalkyl;
alternatively, R^{2e} and R^{3e} can combine with the atoms to which they are attached to form a C₅-C₆ cycloalkyl, 5- to 10-membered heterocyclyl, C₆-C₁₀ aryl, or 5- to 14-membered heteroaryl;
L⁴ is absent, CR^{4a}R^{4b}, C(═CR^{4c}R^{4d}), C(═O), or =C(R^{4c})—;
R^{4a} and R^{4b} are each independently H, C₁-C₃ alkyl, C₁-C₃ alkoxy, halo, C₁-C₆ haloalkyl, C₁-C₆ haloalkoxy, —OH, —CN, C₁-C₃ cyanoalkyl, or C₃-C₆ cycloalkyl;
alternatively, R^{4a} and R^{4b} can combine with the atom to which they are attached to form a C₃-C₆ cycloalkyl;
alternatively, R^{3b} and R^{4b} can combine with the atoms to which they are attached to form a C₃-C₆ cycloalkyl;
R^{4c} and R^{4d} are each independently H, C₁-C₃ alkyl, halo, C₁-C₆ haloalkyl, —CN, C₁-C₃ cyanoalkyl, or C₃-C₆ cycloalkyl;
alternatively, R^{4c} and R^{4d} can combine with the atom to which they are attached to form a C₃-C₆ cycloalkyl or a 3 to 6 membered heterocyclyl having 1 heteroatom that is O, wherein each cycloalkyl and heterocyclyl is substituted with 0, 1, 2, or 3 R^{4X};
each R^{4x} is independently C₁-C₃ alkyl, C₁-C₃ alkoxy, halo, C₁-C₆ haloalkyl, C₁-C₆ haloalkoxy, or —OH;
R^{4e} is H, C₁-C₃ alkyl, halo, C₁-C₆ haloalkyl, —CN, C₁-C₃ cyanoalkyl, or C₃-C₆ cycloalkyl;
alternatively, R^{3e} and R^{4e} can combine with the atoms to which they are attached to form a C₅-C₆ cycloalkyl;
such that when L² is =C(R^{2e})— then L¹ is —C(R^{1e})═ or L³ is =C(R^{3e})—, and when L³ is =C(R^{3e})— then L² is =C(R^{2e})— or L⁴ is =C(R^{4e})—;
R^{A} is phenyl, naphthyl, or 5- to 14-membered heteroaryl, wherein the phenyl, naphthyl, and heteroaryl is substituted with 0, 1, 2, 3, 4, or 5 R^{A2};
each R^{A2} is independently —OH, C₁-C₁₀ alkyl, C₂-C₆ alkenyl, C₂-C₆ alkynyl, C₁-C₁₀ alkoxy, C₁-C₁₀ hydroxyalkyl, C₂-C₁₀ alkoxyalkyl, C₁-C₆ alkyl-N(R^{A2a})(R^{A2b}), C₁-C₁₀ thioalkyl, halo, C₁-C₆haloalkyl, —CN, —C(O)R^{A2a}, —C(O)OR^{A2a}, —OC(O)R^{A2a}, —OC(O)OR^{A2a}, —C(O)N(R^{A2a})(R^{A2b}), —N(R^{A2a})C(O)(R^{A2b}), —OC(O)N(R^{A2a})(R^{A2b}), —N(R^{A2a})C(O)(OR^{A2b}), oxo, —OR^{A2a}, —OR^{A2x}, —SR^{A2a}, —S(O)₂R^{A2a}, —S(O)₂OR^{A2a}, —N(R^{A2a})(R^{A2b}), —(C₀-C₃ alkyl)-SF₅, —OP(O)(OR^{A2a})(OR^{A2b}), C₃-C₅ cycloalkyl, —(C₁-C₆ alkyl)-(C₃-C₈ cycloalkyl), 3- to 14-membered heterocyclyl, —(C₁-C₆ alkyl)-(3- to 14-membered heterocyclyl), C₆-C₁₄ aryl, —(C₁-C₆ alkyl)-(C₆-C₁₄ aryl), 5- to 14-membered heteroaryl, or —(C₁-C₆ alkyl)-(5- to 14-membered heteroaryl), wherein each alkyl, alkenyl, alkynyl, alkoxy, hydroxyalkyl, and haloalkyl is substituted with 0, 1, 2, or 3 R^{A3}, and wherein each cycloalkyl, alkyl-cycloalkyl, heterocyclyl, alkyl-heterocyclyl, aryl, alkyl-aryl, heteroaryl, and alkyl-heteroaryl is substituted with 0, 1, 2, or 3 R^{A4};
each R^{A2a} and R^{A2b} is independently H, C₁-C₁₀ alkyl, C₁-C₆ haloalkyl, or C₃-C₅ cycloalkyl;
each R^{A3} is independently halo, —CN, —OR^{A3a}, —SR^{A3a}—N(R^{A3a})(R^{A3b}) C₃-C₅ cycloalkyl, or 5- to 14-membered heteroaryl;
each R^{A3a} and R^{A3b} is independently H, C₁-C₁₀ alkyl, C₁-C₆ haloalkyl, or C₃-C₈ cycloalkyl; each R^{A4} is independently C₁-C₆ alkoxy, C₁-C₆ hydroxyalkyl, C₂-C₆ alkoxyalkyl, C₁-C₆ haloalkyl, C₁-C₆ haloalkoxy, C₁-C₆ haloalkylthio, C₃-C₈ cycloalkyl, —(C₁-C₆ alkyl)-(C₆-C₁₀ aryl), halo, —CN, —OH, or —N(R^{A4a})(R_{A4b});
each R^{A4a} and R^{A4b} is independently H or C₁-C₆ alkyl;
alternatively, two R^{A2} can combine to form a C₃-C₁₀ cycloalkyl, C₆-C₁₀ aryl, a 3- to 10-membered heterocyclyl, or 5- to 14-membered heteroaryl on two adjacent atoms on R^{A}, wherein each cycloalkyl, aryl, heterocyclyl, and heteroaryl is substituted with 0, 1, 2, or 3 R^{A5};
each R^{A5} is independently C₁-C₁₀ alkyl, C₂-C₁₀ alkenyl, C₂-C₁₀ alkynyl, halo, C₁-C₆ haloalkyl, —CN, or C₃-C₅ cycloalkyl;
R^{B} is H, Me, or R^{B1};
R^{B1} and R^{A2x} are each independently:

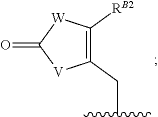

W and V are each independently NR^{B2}, O, or S;
each R^{B2} is independently C₁-C₁₂ alkyl, C₁-C₆ alkoxy, C₂-C₆ alkoxyalkyl, C₁-C₆ alkyl-N(R^{B2a})(R^{B2b}), C₁-C₁₀ thioalkyl, C₁-C₆haloalkyl, C₁-C₆haloalkoxy, C₃-C₅ cycloalkyl, C₆-C₁₄ aryl, or 5- to 14-membered heteroaryl, wherein the cycloalkyl, aryl, and heteroaryl are substituted with 0, 1, 2, or 3 R^{B2c};
each R^{B2a} and R^{B2b} is independently H, C₁-C₆ alkyl, C₁-C₆ haloalkyl, or C₃-C₅ cycloalkyl;
each R^{B2c} is independently C₁-C₆ alkyl, C₁-C₆ alkoxy, C₂-C₆ alkoxyalkyl, halo, C₁-C₆ haloalkyl, C₁-C₆ haloalkoxy, oxo, —OH, —CN, or C₃-C₁₀ cycloalkyl;
L^{C} is a bond or

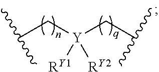

Y is C or Si;
n is 0, 1, 2, or 3;
q is 0, 1, 2, or 3;
R^{Y1} is H or C₁-C₃ alkyl;
R^{Y2} is H or C₁-C₃ alkyl;
alternatively, R^{Y1} and R^{Y2} combine to form a C₃-C₁₀ cycloalkyl or a 3- to 10-membered heterocyclyl;
R^{C} is 3- to 14-membered heterocyclyl substituted with =C(R^{C3e})(R^{C3f}), wherein the heterocyclyl is further substituted with 0, 1, 2, 3, or 4 R^{C3};
R^{C3e} is H, C₁, C₁-C₆ alkyl, C₁-C₆ haloalkyl, or C₃-C₆ cycloalkyl;
R^{C3f} is F, C₁, C₁-C₆ alkyl, C₁-C₆ haloalkyl, or C₃-C₆ cycloalkyl;
each R^{C3} is independently C₁-C₆ alkyl, C₂-C₆ alkenyl, C₂-C₈ alkynyl, C₁-C₆ alkoxyalkyl, C₁-C₆ hydroxyalkyl, halo, C₁-C₆ haloalkyl, C₁-C₆ heteroalkyl, —(C₁-C₆ alkyl)-N(R^{C3a})(R^{C3b}), —CN, —C(O)R^{C3a}, —C(O)OR^{C3a}, —C(O)N(R^{C3a})(R^{C3b}), —N(R^{C3a})C(O)(R^{C3b}), —OC(O)N(R^{C3a})(R^{C3b}), N(R^{C3a})C(O)(OR^{C3b}), =CH₂, =CHF, =CF₂, oxo, —OR^{C3a}, —SR^{C3a}, —N(R^{C3a})(R^{C3b}), —N₃, SF₅, C₃-C₈ cycloalkyl, —(C₁-C₆ alkyl)-(C₃-C₈ cycloalkyl), 3- to 10-membered heterocyclyl, —(C₁-C₆ alkyl)-(3- to 10-membered heterocyclyl), C₆-C₁₀ aryl, —(C₁-C₆ alkyl)-(C₆-C₁₀ aryl), 5- to 10-membered heteroaryl, or —(C₁-C₆ alkyl)-(5- to 10-membered heteroaryl),
wherein each alkyl is substituted with 0, 1, 2, or 3-CN, —C(O)OR^{C3a1}, —C(O)N(R^{C3a1})(R^{C3a2}), —N(R^{C3a1})C(O)(R^{C3a2}), —OC(O)N(R^{C3a1})(R^{C3a2}), —OR^{C3a1}, —SR^{C3a1}, N₃, SF₅, or 3- to 10-membered heterocyclyl substituted with 0, 1, 2, or 3 R^{C3a2}, each cycloalkyl, alkyl-cycloalkyl, heterocyclyl, alkyl-heterocyclyl, aryl, alkyl-aryl, heteroaryl, and alkyl-heteroaryl is substituted with 0, 1, 2, or 3 halo, —CN, or R^{C3a2}, each alkenyl is substituted with 0, 1, 2, or 3 halo, and
each alkoxyalkyl and alkynyl is substituted with 0, 1, 2, or 3 C₁-C₆ alkyl, C₁-C₆ haloalkyl, C₃-C₅ cycloalkyl substituted with 0 or 1 C₁-C₆ haloalkyl, C₆-C₁₀ aryl, or 5- to 10-membered heteroaryl; each R^{C3a} and R^{C3b} is independently H, C₁-C₁₀ alkyl, C₁-C₆ haloalkyl, C₆—CIO aryl, C₃-C₆ cycloalkyl, 3- to 6-membered heterocyclyl, or 5- to 10-membered heteroaryl, wherein each aryl and heteroaryl is substituted with 0, 1, 2, or 3 halo, —CN, or R^{C3a2};
alternatively, R^{C3a} and R^{C3b} together with the N to which they are attached form a 3- to 8-membered heterocycle;
each R^{C3a1} and R^{C3a2} is independently C₁-C₃ alkyl, halo, C₁-C₆ haloalkyl, C₃-C₈ cycloalkyl, —(C₁-C₃ alkyl)-(C₃-C₈ cycloalkyl), 3- to 10-membered heterocyclyl, —(C₁-C₃ alkyl)-(3- to 10-membered heterocyclyl), C₆-C₁₀ aryl, —(C₁-C₃ alkyl)-(C₆-C₁₀ aryl), —(C₂-C₄ alkynyl)-(C₆-C₁₀ aryl), 5- to 10-membered heteroaryl, —(C₁-C₃ alkyl)-(5- to 10-membered heteroaryl), or SF₅, wherein each cycloalkyl, alkyl-cycloalkyl, heterocyclyl, alkyl-heterocyclyl, aryl, alkyl-aryl, alkynyl-aryl, heteroaryl, and alkyl-heteroaryl is substituted with 0, 1, 2, or 3 halo, C₁-C₃ haloalkyl, C₁-C₃ haloalkoxy, or SF₅;
alternatively, R^{C3a1} and R^{C3a2} together with the N to which they are attached form a 3- to 8-membered heterocycle;
R^{D} is halo;
each heterocyclyl has 1, 2, 3, or 4 heteroatoms selected from N, O, S, and Si; and
each heteroaryl has 1, 2, 3, or 4 heteroatoms selected from N, O, and S.

In some embodiments, the present disclosure provides the compound of Formula (I), wherein R¹, R², R³, and R⁴ are each independently H or methyl. In some embodiments, the present disclosure provides the compound of Formula (I), wherein one of two of R¹, R², R³, and R⁴ are methyl. In some embodiments, the present disclosure provides the compound of Formula (I), wherein R¹ and R² are methyl. In some embodiments, the present disclosure provides the compound of Formula (I), wherein R¹, R², R³, and R⁴ are each H.

In some embodiments, the present disclosure provides the compound of Formula (I), or a pharmaceutically acceptable salt thereof, wherein X is N. In some embodiments, the present disclosure provides the compound of Formula (I), or a pharmaceutically acceptable salt thereof, wherein X is CH. In some embodiments, the present disclosure provides the compound of Formula (I), or a pharmaceutically acceptable salt thereof, wherein X is CR^{x}, wherein R^{x} is (CH₂)ₘCN, and m is 0, 1, 2 or 3. In some embodiments, the present disclosure provides the compound of Formula (I), or a pharmaceutically acceptable salt thereof, wherein X is CR^{x}, wherein R^{x} is CH₂CN. In some embodiments, the present disclosure provides the compound of Formula (I), or a pharmaceutically acceptable salt thereof, wherein X is CR^{x}, wherein R^{x} is halo. In some embodiments, the present disclosure provides the compound of Formula (I), or a pharmaceutically acceptable salt thereof, wherein X is C—F. In some embodiments, the present disclosure provides the compound of Formula (I), or a pharmaceutically acceptable salt thereof, wherein X is C—Cl.

In some embodiments, the present disclosure provides the compound of Formula (I), or a pharmaceutically acceptable salt thereof, wherein L¹ is CHR^{1b}. In some embodiments, the present disclosure provides the compound of Formula (I), or a pharmaceutically acceptable salt thereof, wherein L¹ is CH₂. In some embodiments, the present disclosure provides the compound of Formula (I), or a pharmaceutically acceptable salt thereof, wherein L¹ is CH(CH₃). In some embodiments, the present disclosure provides the compound of Formula (I), or a pharmaceutically acceptable salt thereof, wherein R^{1a} and R^{1b} are each independently H, C₁-C₃ alkyl, or halo. In some embodiments, the present disclosure provides the compound of Formula (I), or a pharmaceutically acceptable salt thereof, wherein R^{1b} is C₁-C₃ alkyl or halo. In some embodiments, the present disclosure provides the compound of Formula (I), or a pharmaceutically acceptable salt thereof, wherein R^{1b} is H or methyl. In some embodiments, the present disclosure provides the compound of Formula (I), or a pharmaceutically acceptable salt thereof, wherein R^{1b} is H. In some embodiments, the present disclosure provides the compound of Formula (I), or a pharmaceutically acceptable salt thereof, wherein R^{1b} is methyl.

In some embodiments, the present disclosure provides the compound of Formula (I), or a pharmaceutically acceptable salt thereof, wherein R^{2a} and R^{2b} are each independently H, C₁-C₃ alkyl, halo, or C₁-C₆ haloalkyl. In some embodiments, the present disclosure provides the compound of Formula (I), or a pharmaceutically acceptable salt thereof, wherein L² is CHR^{2b}. In some embodiments, the present disclosure provides the compound of Formula (I), or a pharmaceutically acceptable salt thereof, wherein R^{2b} is H or C₁-C₃ alkyl. In some embodiments, the present disclosure provides the compound of Formula (I), or a pharmaceutically acceptable salt thereof, wherein R^{2b} is H. In some embodiments, the present disclosure provides the compound of Formula (I), or a pharmaceutically acceptable salt thereof, wherein R^{2b} is C₁-C₃ alkyl. In some embodiments, the present disclosure provides the compound of Formula (I), or a pharmaceutically acceptable salt thereof, wherein R^{2b} is methyl.

In some embodiments, the present disclosure provides the compound of Formula (I), or a pharmaceutically acceptable salt thereof, wherein L³ is a bond. In some embodiments, the present disclosure provides the compound of Formula (I), or a pharmaceutically acceptable salt thereof, wherein L³ is CR^{3a}R^{3b}.

In some embodiments, the present disclosure provides the compound of Formula (I), or a pharmaceutically acceptable salt thereof, wherein R^{3a} and R^{3b} are H.

In some embodiments, the present disclosure provides the compound of Formula (I), or a pharmaceutically acceptable salt thereof, wherein R^{A} is phenyl substituted with 0, 1, 2, 3, 4, or 5 R^{A2}. In some embodiments, the present disclosure provides the compound of Formula (I), or a pharmaceutically acceptable salt thereof, wherein R^{A} is phenyl substituted with 0, 1, or 2 R^{A2}. In some embodiments, the present disclosure provides the compound of Formula (I), or a pharmaceutically acceptable salt thereof, wherein R^{A} is naphthyl substituted with 0, 1, 2, 3, 4, or 5 R^{A2}. In some embodiments, the present disclosure provides the compound of Formula (I), or a pharmaceutically acceptable salt thereof, wherein R^{A} is pyridyl, benzothienyl, benzothiazolyl, or isoquinolinyl, each substituted with 0, 1, 2, 3, 4, or 5 R^{A2}.

In some embodiments, the present disclosure provides the compound of Formula (I), or a pharmaceutically acceptable salt thereof, wherein each R^{A2} is independently C₁-C₆ alkyl, —OH, C₂-C₆ alkenyl, C₂-C₆ alkynyl, halo, C₁-C₆ haloalkyl, —OR^{A2a}, —SR^{A2a}, or —(C₁-C₆ alkyl)-(C₃-C₈ cycloalkyl), wherein each alkenyl is substituted with 0, 1, 2, or 3 R^{A3}; each R^{A2a} is independently C₁-C₆ haloalkyl, or C₃-C₅ cycloalkyl; and each R^{A3} is independently halo. In some embodiments, the present disclosure provides the compound of Formula (I), or a pharmaceutically acceptable salt thereof, wherein each R^{A2} is independently Me, —OH, —C(Cl)=CH₂, —CH═CHF₂, —C—CH, F, Cl, —CH₂CF₃, —OCF₃, —O-cyclopropyl, —SCF₃, or —CH₂-cyclopropyl.

In some embodiments, the present disclosure provides the compound of Formula (I), or a pharmaceutically acceptable salt thereof, wherein R^{A} is

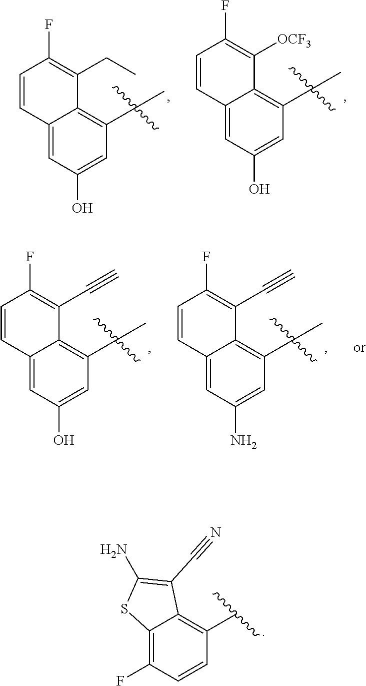 [SMILES: *c1cc(O)cc2ccc(F)c(CC)c12 ; *c1cc(O)cc2ccc(F)c(OC(F)(F)F)c12 ; *c1cc(O)cc2ccc(F)c(C#C)c12 ; *c1cc(N)cc2ccc(F)c(C#C)c12 ; *c1ccc(F)c2sc(N)c(C#N)c12]

In some embodiments, the present disclosure provides the compound of Formula (I), or a pharmaceutically acceptable salt thereof, wherein R^{B} is R^{Bl}.

In some embodiments, the present disclosure provides the compound of Formula (I), or a pharmaceutically acceptable salt thereof, wherein R^{B} is H.

In some embodiments, the present disclosure provides the compound of Formula (I), or a pharmaceutically acceptable salt thereof, wherein R^{B1} is:

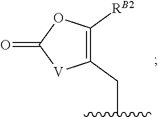

V is each independently NR^{B2} or O.

In some embodiments, the present disclosure provides the compound of Formula (I), or a pharmaceutically acceptable salt thereof, wherein R^{B1} is:

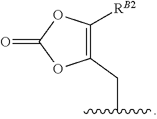

In some embodiments, the present disclosure provides the compound of Formula (I), or a pharmaceutically acceptable salt thereof, wherein each R^{B2} is independently C₁-C₆ alkyl, C₃-C₈ cycloalkyl, C₆-C₁₄ aryl, or 5- to 14-membered heteroaryl, wherein the C₃-C₈ cycloalkyl, C₆-C₁₄ aryl or 5- to 14-membered heteroaryl are substituted with 0, 1, or 2 R^{B2c}; and each R^{B2c} is independently C₁-C₆ alkyl, halo, C₁-C₆ haloalkyl, —CN, or C₃-C₁₀ cycloalkyl. In some embodiments, the present disclosure provides the compound of Formula (I), or a pharmaceutically acceptable salt thereof, wherein each R^{B2} is independently C₁-C₄ alkyl or C₆-C₁₀ aryl. In some embodiments, the present disclosure provides the compound of Formula (I), or a pharmaceutically acceptable salt thereof, wherein each R^{B2} is independently methyl, ethyl, n-propyl, n-butyl, tert-butyl, n-pentyl, n-hexyl, n-octyl, cyclohexyl, or Ph. In some embodiments, the present disclosure provides the compound of Formula (I), or a pharmaceutically acceptable salt thereof, wherein each R^{B2} is independently methyl, ethyl, n-propyl, iso-propyl, tert-butyl, or Ph.

In some embodiments, the present disclosure provides the compound of Formula (I), or a pharmaceutically acceptable salt thereof, wherein R^{B1} is

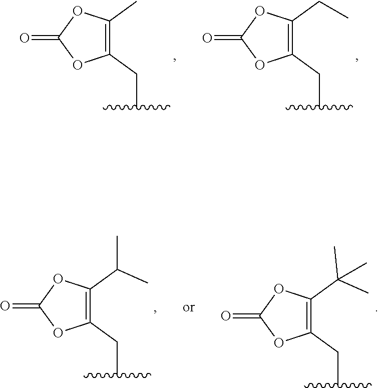

In some embodiments, the present disclosure provides the compound of Formula (I), or a pharmaceutically acceptable salt thereof, wherein R^{B1} is

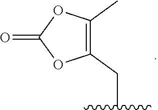 [SMILES: *Cc1oc(=O)oc1C]

In some embodiments, the present disclosure provides the compound of Formula (I), or a pharmaceutically acceptable salt thereof, wherein L^{C} is —CH₂—.

In some embodiments, the present disclosure provides the compound of Formula (I), or a pharmaceutically acceptable salt thereof, wherein R^{C} is 4- to 7-membered heterocyclyl substituted with =C(R^{C3e})(R^{C3H}), wherein the heterocyclyl is further substituted with 2 or 3 R^{C3}.

In some embodiments, the present disclosure provides the compound of Formula (I), or a pharmaceutically acceptable salt thereof, having the structure of Formula (Ia):

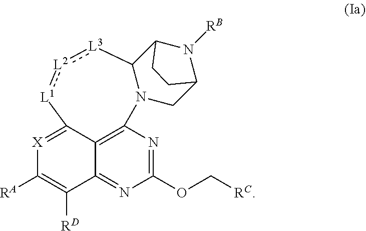

In some embodiments, the present disclosure provides the compound of Formula (I), or a pharmaceutically acceptable salt thereof, wherein R^{C} is

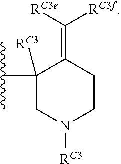

In some embodiments, the present disclosure provides the compound of Formula (I), (Ia), or a pharmaceutically acceptable salt thereof, having the structure of Formula (Ib):

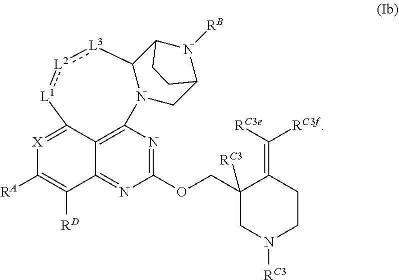

In some embodiments, the present disclosure provides the compound of Formula (I), (Ia), (Ib), or a pharmaceutically acceptable salt thereof, having the structure of Formula (Ic):

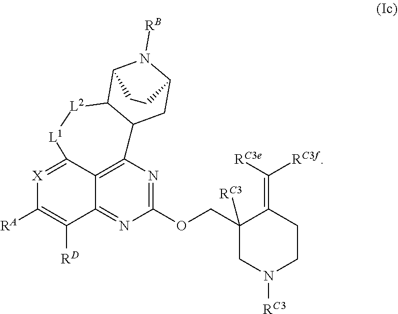

In some embodiments, the present disclosure provides the compound of Formula (I), (Ia), (Ib), or a pharmaceutically acceptable salt thereof, having the structure of Formula (Id):

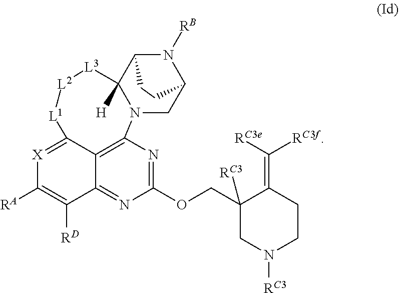

In some embodiments, the present disclosure provides the compound of Formula (I), (Ia), (Ib), (Ic), (Id), or a pharmaceutically acceptable salt thereof, wherein each R^{C3} is C₁-C₆ alkyl. In some embodiments, the present disclosure provides the compound of Formula (I), (Ia), (Ib), (Ic), (Id), or a pharmaceutically acceptable salt thereof, wherein each R^{C3} is methyl.

In some embodiments, the present disclosure provides the compound of Formula (I), (Ia), (Ib), (Ic), (Id), or a pharmaceutically acceptable salt thereof, wherein R^{C3e} is H; and R^{C3f} is F.

In some embodiments, the present disclosure provides the compound of Formula (I), (Ia), or a pharmaceutically acceptable salt thereof, wherein R^{C} is

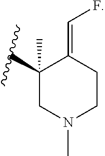 [SMILES: *[C@]1(C)CN(C)CC/C1=C\F]

In some embodiments, the present disclosure provides the compound of Formula (I), (Ia), (Ib), (Ic), (Id), or a pharmaceutically acceptable salt thereof, wherein R^{D} is F or Cl. In some embodiments, the present disclosure provides the compound of Formula (I), (Ia), (Ib), (Ic), (Id), or a pharmaceutically acceptable salt thereof, wherein R^{D} is F.

In some embodiments, the present disclosure provides the compound of Formula (I), (Ia), (Ib), (Ic), (Id), or a pharmaceutically acceptable salt thereof, wherein the compound has the structure:

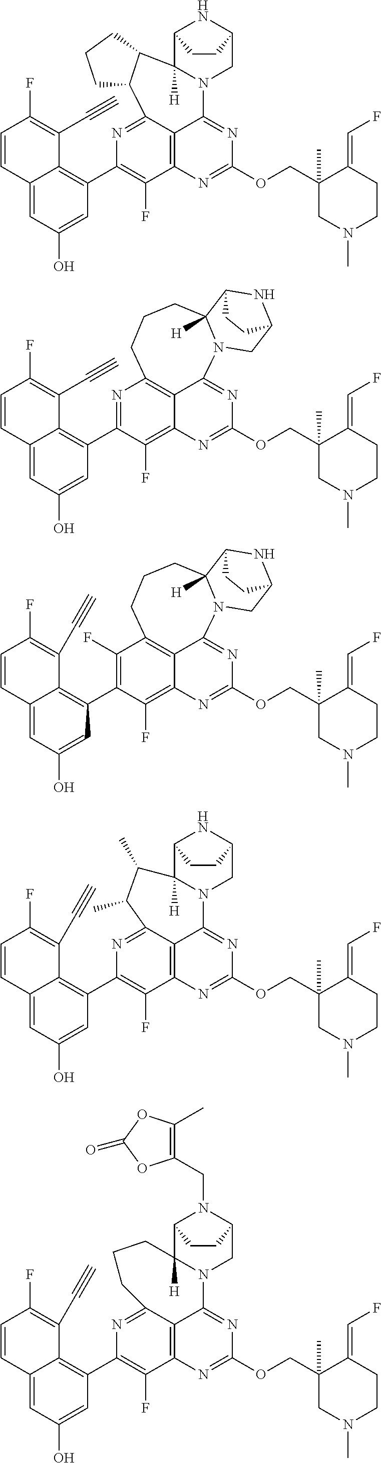

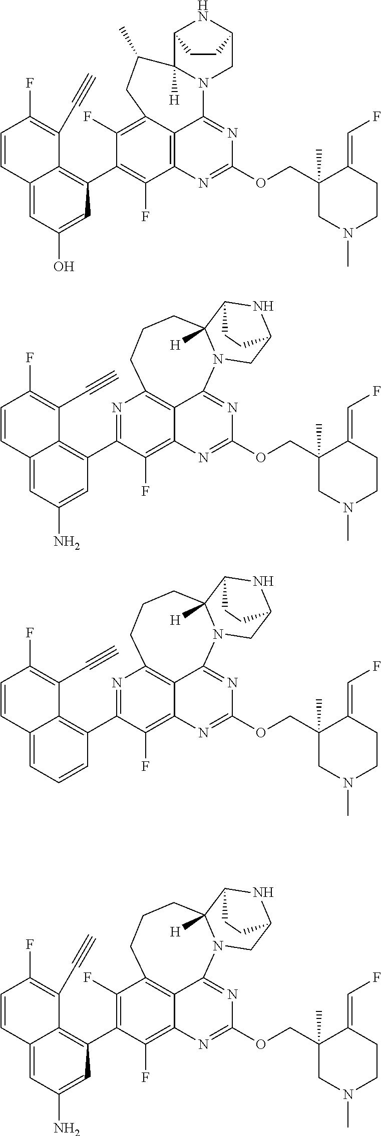

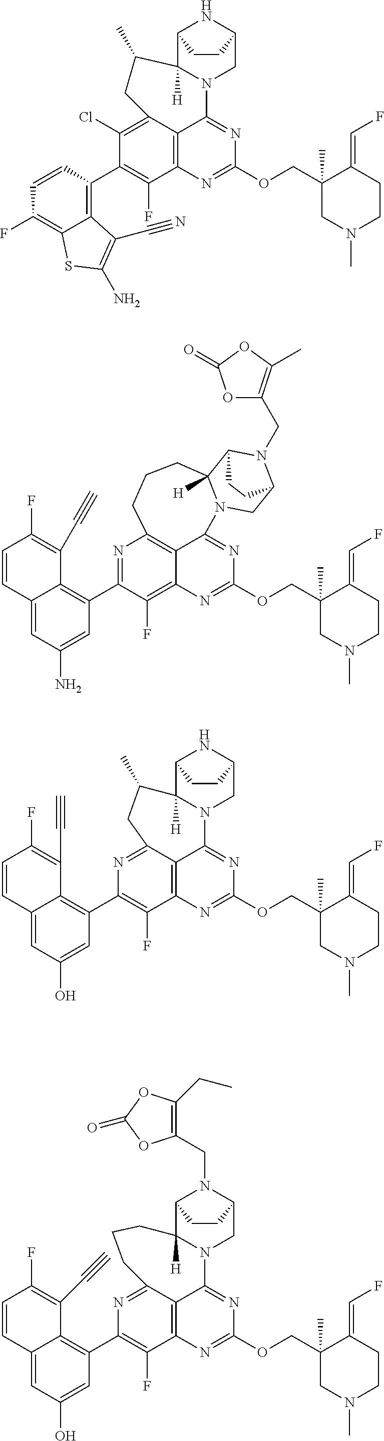

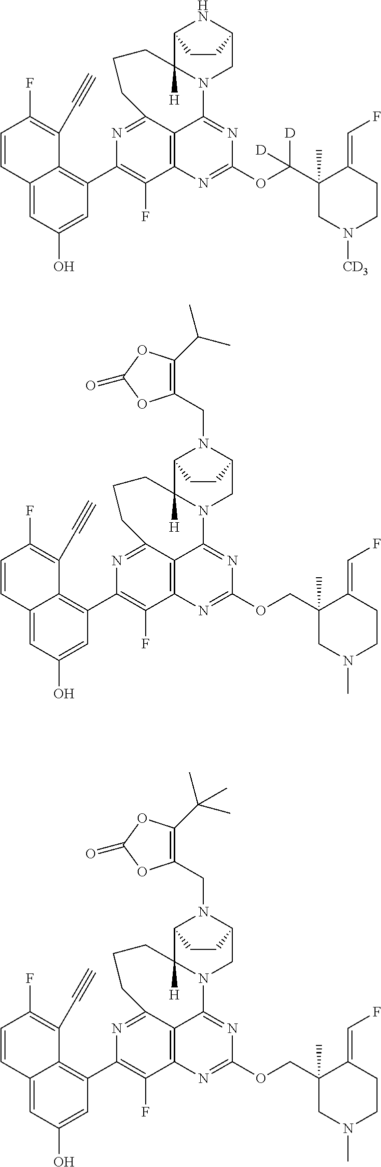

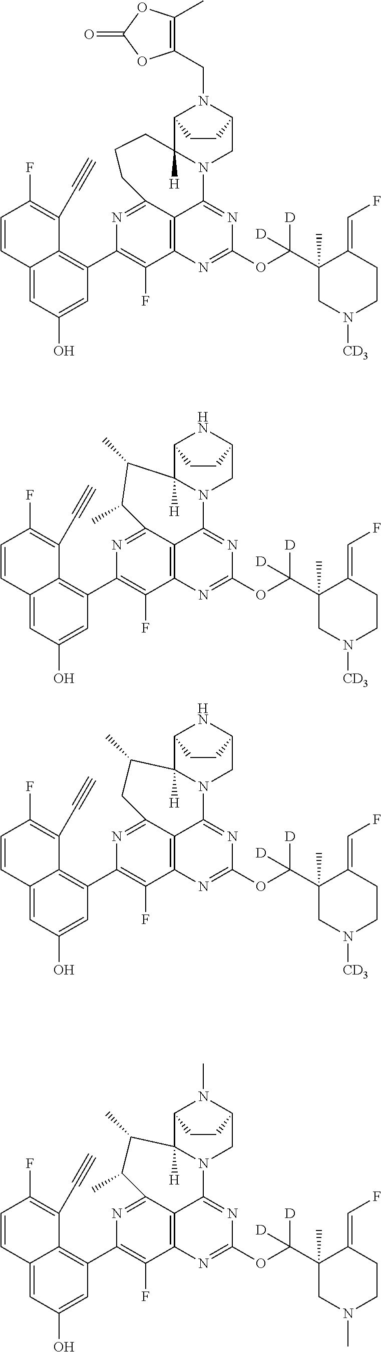

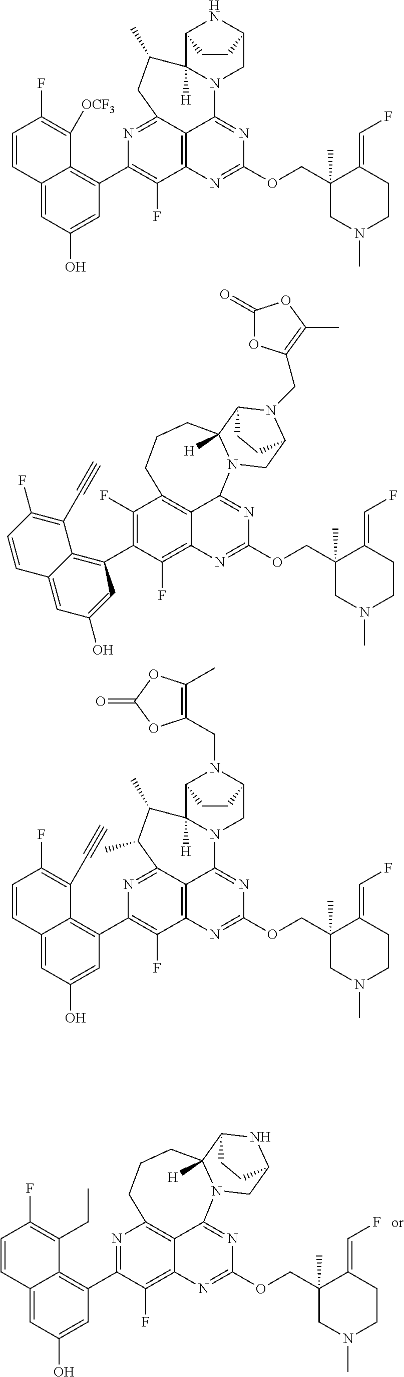

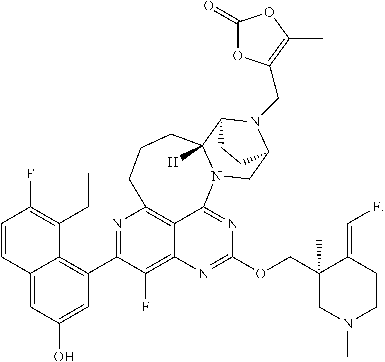

Also falling within the scope herein are the in vivo metabolic products of the compounds described herein. Such products may result for example from the oxidation, reduction, hydrolysis, amidation, esterification and the like of the administered compound, primarily due to enzymatic processes. Accordingly, included are novel and unobvious compounds produced by a process comprising contacting a compound with a mammal for a period of time sufficient to yield a metabolic product thereof. Such products typically are identified by preparing a radiolabelled (e.g., ¹⁴C or ³H) compound, administering it parenterally in a detectable dose (e.g., greater than about 0.5 mg/kg) to an animal such as rat, mouse, guinea pig, monkey, or to man, allowing sufficient time for metabolism to occur (typically about 30 seconds to 30 hours) and isolating its conversion products from the urine, blood or other biological samples. These products are easily isolated since they are labeled (others are isolated by the use of antibodies capable of binding epitopes surviving in the metabolite). The metabolite structures are determined in conventional fashion, e.g., by MS or NMR analysis. In general, analysis of metabolites is done in the same way as conventional drug metabolism studies. The conversion products, so long as they are not otherwise found in vivo, are useful in diagnostic assays for therapeutic dosing of the compounds even if they possess no HSV antiviral activity of their own.

Recipes and methods for determining stability of compounds in surrogate gastrointestinal secretions are known. Compounds are defined herein as stable in the gastrointestinal tract where less than about 50 mole percent of the protected groups are deprotected in surrogate intestinal or gastric juice upon incubation for 1 hour at 37° C. Simply because the compounds are stable to the gastrointestinal tract does not mean that they cannot be hydrolyzed in vivo. The prodrugs typically will be stable in the digestive system but may be substantially hydrolyzed to the parental drug in the digestive lumen, liver, lung or other metabolic organ, or within cells in general. As used herein, a prodrug is understood to be a compound that is chemically designed to efficiently liberate the parent drug after overcoming biological barriers to oral delivery.

In some embodiments, the compound of Formula (I), or pharmaceutically acceptable salt thereof, is a prodrug. In some embodiments, the prodrug as described herein exhibits an improved biological property after in vivo administration in a subject, including enhanced pharmacokinetics, such as higher exposure, higher oral bioavailability, and/or higher maximum blood concentration, and enhanced tissue distribution, compared to in vivo administration of an equivalent amount of the parent drug in the subject.

### IV. Pharmaceutical Compositions

Also disclosed herein are pharmaceutical compositions comprising a pharmaceutically effective amount of a compound of the present disclosure (e.g., a compound of Formula (I), (Ia), (Ib), (Ic), (Id)), or a pharmaceutically acceptable salt thereof, and a pharmaceutically acceptable carrier or excipient. Also provided herein is a pharmaceutical composition comprising a pharmaceutically effective amount of a compound of the present disclosure, or a pharmaceutically acceptable salt thereof, and a pharmaceutically acceptable carrier or excipient. For example, provided herein is a pharmaceutical composition comprising a pharmaceutically effective amount of a compound of the present disclosure, or a pharmaceutically acceptable salt thereof, and a pharmaceutically acceptable excipient.

The compounds disclosed herein can be formulated with conventional carriers and excipients. Tablets can contain, for instance, excipients, glidants, fillers, binders, or a combination thereof. Aqueous formulations are prepared in sterile form, and when intended for delivery by other than oral administration generally will be isotonic. Exemplary excipients include, but are not limited to, those set forth in the “HANDBOOK OF PHARMACEUTICAL EXCIPIENTS” (1986).

In some embodiments, the compounds disclosed herein have pharmacokinetic properties (e.g., oral bioavailability) suitable for oral administration of the compounds. Formulations suitable for oral administration can, for instance, be presented as discrete units such as capsules, cachets or tablets, each containing a predetermined amount of the active ingredient; as a powder or granules; as a solution or a suspension in an aqueous or non-aqueous liquid; or as an oil-in-water liquid emulsion or a water-in-oil liquid emulsion. The active ingredient can also be administered, for instance, as a bolus, electuary, or paste.

A tablet can be made by compression or molding, optionally with at least accessory ingredients. Compressed tablets can be prepared by compressing in a suitable machine the active ingredient in a free-flowing form such as, for instance, a powder or granules, optionally mixed with a binder, lubricant, inert diluent, preservative, surface active, dispersing agent, or a combination thereof. Molded tablets can be made by molding in a suitable machine a mixture of the powdered active ingredient moistened with an inert liquid diluent. The tablets can optionally be coated or scored and optionally are formulated so as to provide slow or controlled release of the active ingredient therefrom.

For infections of the eye or other external tissues (e.g., mouth and skin), the formulations can be applied as a topical ointment or cream containing the active ingredient(s). When formulated in an ointment, the active ingredients can be employed in some embodiments with either a paraffinic or a water-miscible ointment base. Alternatively, the active ingredients can be formulated in a cream with an oil-in-water cream base.

In some embodiments, the cream base can include, for instance, a compound that enhances absorption or penetration of the active ingredient through the skin or other affected areas. In some embodiments, the cream or emulsion does not include water.

The oily phase of the emulsions can be constituted from known ingredients in a known manner. In some embodiments, the phase comprises merely an emulsifier (otherwise known as an emulgent). In some embodiments, the phase comprises a mixture of at least one emulsifier with a fat, an oil, or a combination thereof. In some embodiments, a hydrophilic emulsifier is included together with a lipophilic emulsifier that acts as a stabilizer. Together, the emulsifier(s) with or without stabilizer(s) can make up the so-called emulsifying wax, and the wax together with the oil and fat make up the so-called emulsifying ointment base that can form the oily dispersed phase of the cream formulations.

The choice of suitable oils or fats for the formulation can be based on achieving the desired cosmetic properties.

In some embodiments, the compounds disclosed herein are administered alone. In some embodiments, the compounds disclosed herein are administered in pharmaceutical compositions. In some embodiments, the pharmaceutical compositions are for veterinary use. In some embodiments, the pharmaceutical compositions are for human use. In some embodiments, the pharmaceutical compositions disclosed herein include at least one additional therapeutic agent. In some embodiments, the pharmaceutical compositions disclosed herein include one or more additional therapeutic agent. In some embodiments, the one or more additional therapeutic agents is independently a chemotherapeutic agent, an immunotherapeutic agent, a hormonal agent, an anti-hormonal agent, a targeted therapy agent, or an anti-angiogenesis agent.

Pharmaceutical compositions disclosed herein can be in any form suitable for the intended method of administration. The pharmaceutical compositions disclosed herein can be presented in unit dosage form and can be prepared by any of the methods well known in the art of pharmacy. Exemplary techniques and formulations can be found, for instance, in REMINGTON'S PHARMACEUTICAL SCIENCES (Mack Publishing Co., Easton, PA). Such methods can include the step of bringing into association a compound disclosed herein with the carrier that constitutes at least accessory ingredients. In general, the formulations can be prepared by uniformly and intimately bringing into association the active ingredient with liquid carriers or finely divided solid carriers or both, and then, if necessary, shaping the product.

When used for oral use for example, tablets, troches, lozenges, aqueous or oil suspensions, dispersible powders or granules, emulsions, hard or soft capsules, solutions, syrups or elixirs can be prepared. Formulations intended for oral use can be prepared according to any method known to the art for the manufacture of pharmaceutical compositions and such formulations can contain at least agents including sweetening agents, flavoring agents, coloring agents and preserving agents, in order to provide a palatable preparation. Tablets containing the active ingredient in admixture with non-toxic pharmaceutically acceptable excipient which are suitable for manufacture of tablets are acceptable. Tablets can be uncoated or can be coated by known techniques including microencapsulation to delay disintegration and adsorption in the gastrointestinal tract and thereby provide a sustained action over a longer period.

Formulations for oral use can be also presented as hard gelatin capsules where the active ingredient is mixed with an inert solid diluent, or as soft gelatin capsules wherein the active ingredient is mixed with water or an oil medium.

Aqueous suspensions can contain the active materials in admixture with excipients suitable for the manufacture of aqueous suspensions. The aqueous suspension can also contain, for example, at least preservatives such as ethyl or n-propyl p-hydroxy-benzoate, one or more coloring agents, one or more flavoring agents, one or more sweetening agents, or combinations thereof.

Oil suspensions can be formulated by suspending the active ingredient in a vegetable oil, a mineral oil, or a combination thereof. The oral suspensions can contain, for instance, a thickening agent. In some embodiments, sweetening agents, such as those set forth above, and/or flavoring agents, are added to provide a palatable oral preparation. In some embodiments, the formulations disclosed herein are preserved by the addition of an antioxidant such as ascorbic acid.

Dispersible powders and granules suitable for preparation of an aqueous suspension by the addition of water can provide the active ingredient in admixture with a dispersing or wetting agent, a suspending agent, a preservative, and combinations thereof. Additional excipients, for example sweetening, flavoring and coloring agents, can also be present.

The pharmaceutical compositions can also be in the form of oil-in-water emulsions. The oily phase can be a vegetable oil, a mineral oil, or a mixture of these. The emulsion can also contain sweetening and flavoring agents. Syrups and elixirs can be formulated with sweetening agents. Such formulations can also contain, for instance, a demulcent, a preservative, a flavoring, a coloring agent, or a combination thereof.

The pharmaceutical compositions can be in the form of a sterile injectable or intravenous preparation, such as a sterile injectable aqueous or oleaginous suspension. This suspension can be formulated according to the known art using those suitable dispersing or wetting agents and suspending agents. The sterile injectable or intravenous preparation can also be a sterile injectable solution or suspension in a non-toxic parenterally acceptable diluent or solvent, or prepared as a lyophilized powder.

The amount of active ingredient that can be combined with the carrier material to produce a single dosage form will vary depending upon the host treated and the particular mode of administration. The pharmaceutical composition can be prepared to provide easily measurable amounts for administration.

Formulations suitable for topical administration to the eye also include eye drops wherein the active ingredient is dissolved or suspended in a suitable carrier, especially an aqueous solvent for the active ingredient.

Formulations suitable for topical administration in the mouth include lozenges can comprise an active ingredient (i.e., a compound disclosed herein and/or additional therapeutic agents) in a flavored basis; pastilles comprising the active ingredient in an inert basis; and mouthwashes comprising the active ingredient in a suitable liquid carrier.

Formulations for rectal administration can be presented as a suppository with a suitable base comprising for example cocoa butter or a salicylate.

Formulations suitable for vaginal administration can be presented as pessaries, tampons, creams, gels, pastes, foams or spray formulations containing in addition to the active ingredient such carriers as are known in the art to be appropriate.

Formulations suitable for parenteral administration include aqueous and non-aqueous sterile injection solutions that can contain anti-oxidants, buffers, bacteriostats and solutes that render the formulation isotonic with the blood of the intended recipient; and aqueous and non-aqueous sterile suspensions that can include suspending agents and thickening agents.

The formulations can be presented in unit-dose or multi-dose containers, for example, sealed ampoules and vials, and can be stored in a freeze-dried (lyophilized) condition requiring only the addition of the sterile liquid carrier, immediately before use. Extemporaneous injection solutions and suspensions are prepared from sterile powders, granules and tablets of the kind previously described. Preferred unit-dosage formulations are those containing a daily dose or unit daily sub-dose, as herein above recited, or an appropriate fraction thereof, of the active ingredient.

It should be understood that in addition to the ingredients particularly mentioned above the formulations can include other agents conventional in the art having regard to the type of formulation in question, for example those suitable for oral administration can include flavoring agents.

Further provided are veterinary formulations comprising a compound disclosed herein together with a veterinary carrier therefor.

Veterinary carriers are materials useful for the purpose of administering the formulation and can be solid, liquid or gaseous materials which are otherwise inert or acceptable in the veterinary art and are compatible with the active ingredient. These veterinary formulations can be administered orally, parenterally, or by any other desired route.

Compounds herein are used to provide controlled release pharmaceutical compositions containing as active ingredient one or more of the compounds (“controlled release formulations”) in which the release of the active ingredient can be controlled and regulated to allow less frequency dosing or to improve the pharmacokinetic or toxicity profile of a given active ingredient.

Effective dose of active ingredient depends at least on the nature of the condition being treated, toxicity, whether the compound is being used prophylactically (lower doses) or against an active viral infection, the method of delivery, and the pharmaceutical composition, and will be determined by the clinician using conventional dose escalation studies.

### V. Kits

Also provided herein are kits that includes a compound disclosed herein or a pharmaceutically acceptable salt thereof. In some embodiments the kits described herein can comprise a label and/or instructions for use of the compound in the treatment of a disease or condition in a subject (e.g., human) in need thereof. In some embodiments, the disease or condition is viral infection.

In some embodiments, the kit can also comprise one or more additional therapeutic agents and/or instructions for use of additional therapeutic agents in combination with the compound disclosed herein in the treatment of the disease or condition in a subject (e.g., human) in need thereof.

In some embodiments, the kits provided herein comprise individual dose units of a compound as described herein, or a pharmaceutically acceptable salt, racemate, enantiomer, diastereomer, tautomer, polymorph, pseudopolymorph, amorphous form, hydrate or solvate thereof. Examples of individual dosage units can include pills, tablets, capsules, prefilled syringes or syringe cartridges, IV bags, inhalers, nebulizers etc., each comprising a therapeutically effective amount of the compound in question, or a pharmaceutically acceptable salt, racemate, enantiomer, diastereomer, tautomer, polymorph, pseudopolymorph, amorphous form, hydrate or solvate thereof. In some embodiments, the kit can contain a single dosage unit and in others multiple dosage units are present, such as the number of dosage units required for a specified regimen or period.

Also provided are articles of manufacture that include a compound disclosed herein, or a pharmaceutically acceptable salt, stereoisomer, mixture of stereoisomers or tautomer thereof; and a container. In some embodiments, the container of the article of manufacture is a vial, jar, ampoule, preloaded syringe, blister package, tin, can, bottle, box, an intravenous bag, an inhaler, or a nebulizer.

### VI. Administration

One or more of the compounds of Formula (I), (herein referred to as the active ingredients) are administered by any route appropriate to the condition to be treated. Suitable routes include oral, rectal, nasal, pulmonary, topical (including buccal and sublingual), vaginal and parenteral (including subcutaneous, intramuscular, intravenous, intradermal, intrathecal and epidural), and the like. It will be appreciated that the route may vary with for example the condition of the recipient. An advantage of the compounds herein is that they are orally bioavailable and can be dosed orally.

The compounds of the present disclosure (also referred to herein as the active ingredients), can be administered by any route appropriate to the condition to be treated.

Suitable routes include oral, rectal, nasal, topical (including buccal and sublingual), transdermal, vaginal and parenteral (including subcutaneous, intramuscular, intravenous, intradermal, intrathecal and epidural), and the like. It will be appreciated that the route may vary with for example the condition of the recipient. An advantage of certain compounds disclosed herein is that they are orally bioavailable and can be dosed orally.

A compound of the present disclosure may be administered to an individual in accordance with an effective dosing regimen for a desired period of time or duration.

The dosage or dosing frequency of a compound of the present disclosure may be adjusted over the course of the treatment, based on the judgment of the administering physician.

The compound may be administered to an individual (e.g., a human) in an effective amount. In some embodiments, the compound is administered once daily.

The compound can be administered by any useful route and means, such as by oral or parenteral (e.g., intravenous) administration.

A compound of the present disclosure may be combined with one or more additional therapeutic agents in any dosage amount of the compound of the present disclosure. A single dose can be administered hourly, daily, or weekly.

The frequency of dosage of the compound of the present disclosure can be determined by the needs of the individual patient and can be, for example, once per day or twice, or more times, per day. Administration of the compound continues for as long as necessary to treat the disease or condition.

Administration can be intermittent, with a period of several or more days during which a patient receives a daily dose of the compound of the present disclosure followed by a period of several or more days during which a patient does not receive a daily dose of the compound.

Alternating periods of administration of the compound, followed by non-administration of the compound, can be repeated as clinically required to treat the patient.

In some embodiments, pharmaceutical compositions comprising a compound of the present disclosure, or a pharmaceutically acceptable salt thereof, in combination with one or more (e.g., one, two, three, four, one or two, one to three, or one to four) additional therapeutic agents, and a pharmaceutically acceptable excipient are provided.

In some embodiments, kits comprising a compound of the present disclosure, or a pharmaceutically acceptable salt thereof, in combination with one or more (e.g., one, two, three, four, one or two, one to three, or one to four) additional therapeutic agents are provided.

In some embodiments, a compound of the present disclosure, or a pharmaceutically acceptable salt thereof, is combined with one, two, three, four or more additional therapeutic agents. In some embodiments, a compound of the present disclosure, or a pharmaceutically acceptable salt thereof, is combined with two additional therapeutic agents. In some embodiments, a compound of the present disclosure, or a pharmaceutically acceptable salt thereof, is combined with three additional therapeutic agents. In some embodiments, a compound of the present disclosure, or a pharmaceutically acceptable salt thereof, is combined with four additional therapeutic agents. The one, two, three, four or more additional therapeutic agents can be different therapeutic agents selected from the same class of therapeutic agents, and/or they can be selected from different classes of therapeutic agents.

In some embodiments, when a compound of the present disclosure is combined with one or more additional therapeutic agents as described herein, the components of the composition are administered as a simultaneous or sequential regimen. When administered sequentially, the combination may be administered in two or more administrations.

In some embodiments, a compound of the present disclosure is combined with one or more additional therapeutic agents in a unitary dosage form for simultaneous administration to a patient, for example as a solid dosage form for oral administration.

In some embodiments, a compound of the present disclosure is co-administered with one or more additional therapeutic agents.

In order to prolong the effect of a compound of the present disclosure, it is often desirable to slow the absorption of a compound from subcutaneous or intramuscular injection. This may be accomplished by the use of a liquid suspension of crystalline or amorphous material with poor water solubility. The rate of absorption of the compound then depends upon its rate of dissolution that, in turn, may depend upon crystal size and crystalline form. Alternatively, delayed absorption of a parenterally administered compound form is accomplished by dissolving or suspending a compound in an oil vehicle. Injectable depot forms are made by forming microencapsule matrices of a compound in biodegradable polymers. Depending upon the ratio of compound to polymer and the nature of the particular polymer employed, the rate of compound release can be controlled.

Depot injectable formulations are also prepared by entrapping a compound in liposomes or microemulsions that are compatible with body tissues.

### VII. Methods of Use

The disclosure further relates to the use of compounds disclosed herein for the treatment and/or prophylaxis of diseases and/or conditions through inhibition of KRAS G12D and/or G12C.

Further, the present disclosure relates to the use of said compounds for the preparation of a medicament for the treatment and/or prophylaxis of cancer.

Medicaments as referred to herein can be prepared by conventional processes, including the combination of a compound according to the present disclosure and a pharmaceutically acceptable carrier.

In some embodiments, provided herein is a method of inhibiting KRAS G12D protein in a subject in need thereof, the method comprising administering to the subject a therapeutically effective amount of a compound of Formula (I), (Ia), (Ib), (Ic), or (Id), or a pharmaceutically acceptable salt thereof, or a pharmaceutical composition comprising a compound of Formula (I), (Ia), (Ib), (Ic), or (Id), or a pharmaceutically acceptable salt thereof.

In some embodiments, provided herein is treating cancer in a subject in need thereof, the method comprising administering to the subject a therapeutically effective amount of a compound of Formula (I), (Ia), (Ib), (Ic), or (Id), or a pharmaceutically acceptable salt thereof, or a pharmaceutical composition comprising a compound of Formula (I), (Ia), (Ib), (Ic), or (Id), or a pharmaceutically acceptable salt thereof.

In some embodiments, provided herein is a method of treating and/or preventing a cancer.

In some embodiments, provided herein is a method of treating and/or preventing a KRAS G12D-associated cancer.

In some embodiments, provided herein is a method of reducing the proliferation of a cell comprising contacting the cell with a compound of Formula (I), (Ia), (Ib), (Ic), or (Id), or a pharmaceutically acceptable salt thereof.

In some embodiments, the KRAS G12D associated disease or condition includes cancer.

In some embodiments, the cancer is a hematological cancer. In some embodiments, the cancer includes a solid tumor. In some embodiments, the cancer includes a malignant tumor. In some embodiments the cancer includes a metastatic cancer. In some embodiments, the cancer is resistant or refractory to one or more anticancer therapies. In some embodiments, greater than about 50% of the cancer cells detectably express one or more cell surface immune checkpoint receptors (e.g., so-called “hot” cancer or tumor). In some embodiments, greater than about 1% and less than about 50% of the cancer cells detectably express one or more cell surface immune checkpoint receptors (e.g., so called “warm” cancer or tumor). In some embodiments, less than about 1% of the cancer cells detectably express one or more cell surface immune checkpoint receptors (e.g., so called “cold” cancer or tumor).

In some embodiments, the KRAS G12D associated disease or condition is a hematological cancer, e.g., a leukemia (e.g., Acute Myelogenous Leukemia (AML), Acute Lymphoblastic Leukemia (ALL), B-cell ALL, Myelodysplastic Syndrome (MDS), myeloproliferative disease (MPD), Chronic Myelogenous Leukemia (CML), Chronic Lymphocytic Leukemia (CLL), undifferentiated leukemia), a lymphoma (e.g., small lymphocytic lymphoma (SLL), mantle cell lymphoma (MCL), follicular lymphoma (FL), T-cell lymphoma, B-cell lymphoma, diffuse large B-cell lymphoma (DLBCL), marginal zone lymphoma (MZL), Waldestrom's macroglobulinemia (WM)) and/or a myeloma (e.g., multiple myeloma (MM)).

In some embodiments, the KRAS G12D associated disease or condition is an epithelial tumor (e.g., a carcinoma, a squamous cell carcinoma, a basal cell carcinoma, a squamous intraepithelial neoplasia), a glandular tumor (e.g., an adenocarcinoma, an adenoma, an adenomyoma), a mesenchymal or soft tissue tumor (e.g., a sarcoma, a rhabdomyosarcoma, a leiomyosarcoma, a liposarcoma, a fibrosarcoma, a dermatofibrosarcoma, a neurofibrosarcoma, a fibrous histiocytoma, an angiosarcoma, an angiomyxoma, a leiomyoma, a chondroma, a chondrosarcoma, an alveolar soft-part sarcoma, an epithelioid hemangioendothelioma, a Spitz tumor, a synovial sarcoma), or a lymphoma. In some embodiments, the KRAS G12D associated disease or condition includes a solid tumor in or arising from a tissue or organ, such as bone, lips and oral cavity, esophagus, gastrointestinal tract, pancreas, gall bladder, neuro-endocrine, thyroid, liver, kidney, breast, peritoneum, female sex organ tissues, male sex organ tissues, bladder, brain, eye, head and neck, thymus, heart, lung, lymph, central nervous system (CNS), neuroendocrine tissues, skin, and soft tissues.

In some embodiments, the KRAS G12D associated disease or condition is a cancer selected from a lung cancer, a colorectal cancer, a breast cancer, a prostate cancer, a cervical cancer, a pancreatic cancer and a head and neck cancer. In some embodiments, the cancer is metastatic.

In some embodiments, the KRAS G12D associated disease or condition is a cancer selected from non-small lung cancer (NSCLC), melanoma, triple-negative breast cancer (TNBC), nasopharyngeal cancer (NPC), microsatellite stable colorectal cancer (mssCRC), thymoma, and gastrointestinal stromal tumor (GIST). In some embodiments, the cancer is metastatic.

In some embodiments, the KRAS G12D associated disease or condition is a cancer of pancreatic cancer, bladder cancer, colorectal cancer, breast cancer, prostate cancer, renal cancer, hepatocellular cancer, lung cancer, ovarian cancer, cervical cancer, uterine cancer, gastric cancer, bile duct cancer, testicular cancer, esophageal cancer, head and neck cancer, melanoma, neuroendocrine cancer, CNS cancer, brain cancer, bone cancer, soft tissue sarcoma, non-small cell lung cancer, small-cell lung cancer, myelodysplastic syndrome, thyroid cancer, or colon cancer.

In some embodiments, the KRAS G12D associated disease or condition is a cancer of pancreatic cancer, colorectal cancer, non-small cell lung cancer, endometrial cancer, uterine endometrical carcinoma, cholangio carcinoma, testicular germ cell cancer, cervical squamous carcinoma, or myelodysplastic syndrome.

In some embodiments, the cancer is or myelodysplastic syndrome. In some embodiments, the cancer is high risk myelodysplastic syndrome or low risk myelodysplastic syndrome. In some embodiments, the cancer is high risk myelodysplastic syndrome. In some embodiments, the cancer is high risk myelodysplastic syndrome.

In some embodiments, the cancer is colorectal cancer. In some embodiments, the cancer is non-small cell lung cancer. In some embodiments, the cancer is pancreatic cancer. In some embodiments, the cancer is endometrial cancer. In some embodiments, the cancer is uterine endometrical carcinoma. In some embodiments, the cancer is testicular germ cell cancer. In some embodiments, the cancer is cervical squamous carcinoma. In some embodiments, the cancer is cholangio carcinoma.

The effective dosage of active ingredient employed may vary depending on the particular compound employed, the mode of administration, the condition being treated and the severity of the condition being treated. Such dosage may be ascertained readily by a person skilled in the art.

When treating or preventing a KRAS G12D associated disease or condition for which compounds of the present disclosure are indicated, generally satisfactory results are obtained when the compounds of the present disclosure are administered at a daily dosage of from about 0.1 milligram to about 300 milligram per kilogram of animal body weight.

The compounds of the present application or the compositions thereof may be administered once, twice, three, or four times daily, using any suitable mode described above.

In some embodiments, the compound or pharmaceutically acceptable salt thereof of the present disclosure is administered in combination with one or more additional therapeutic agent or therapeutic modality.

In some embodiments, the present disclosure provides the pharmaceutical composition or the method wherein the one or more additional therapeutic agent or additional therapeutic modality comprises one, two, three, or four additional therapeutic agents and/or therapeutic modalities.

In some embodiments, the present disclosure provides the pharmaceutical composition or the method wherein the additional therapeutic agent or therapeutic modalities are selected from an immune checkpoint modulator, an antibody-drug conjugate (ADC), an antiapoptotic agent, a targeted anticancer therapeutic, a chemotherapeutic agent, surgery, or radiation therapy.

In some embodiments, the present disclosure provides the pharmaceutical composition or the method wherein the immune checkpoint modulator is selected from an anti-PD-(L)₁ antibody, an anti-TIGIT antibody, an anti-CTLA4 antibody, an anti-CCR8 antibody, an anti-TREM1 antibody, an anti-TREM2 antibody, a CD47 inhibitor, a DGKX inhibitor, an HPK1 inhibitor, a FLT3 agonist, an adenosine receptor antagonist, a CD39 inhibitor, a CD73 inhibitor, an IL-2 variant (IL-2v), and a CAR-T cell therapy.

In some embodiments, the present disclosure provides the pharmaceutical composition or the method wherein the anti-PD-(L)₁ antibody is selected from pembrolizumab, nivolumab, cemiplimab, pidilizumab, spartalizumab, atezolizumab, avelumab, durvalumab, cosibelimab, sasanlimab, tislelizumab, retifanlimab, balstilimab, toripalimab, cetrelimab, genolimzumab, prolgolimab, lodapolimab, camrelizumab, budigalimab, avelumab, dostarlimab, envafolimab, sintilimab, and zimberelimab.

In some embodiments, the present disclosure provides the pharmaceutical composition or the method wherein the anti-TIGIT antibody is selected from tiragolumab, vibostolimab, domvanalimab, AB308, AK127, BMS-986207, and etigilimab.

In some embodiments, the present disclosure provides the pharmaceutical composition or the method wherein the anti-CTLA4 antibody is selected from ipilimumab, tremelimumab, and zalifrelimab.

In some embodiments, the present disclosure provides the pharmaceutical composition or the method wherein the CD47 inhibitor is selected from magrolimab, letaplimab, lemzoparlimab, AL-008, RRx-001, CTX-5861, FSI-189 (GS-0189), ES-004, BI-765063, ADU1805, CC-95251, and Q-1801.

In some embodiments, the present disclosure provides the pharmaceutical composition or the method wherein the adenosine receptor antagonist is etrumadenant (AB928), taminadenant, TT-10, TT-4, or M1069.

In some embodiments, the present disclosure provides the pharmaceutical composition or the method wherein the CD39 inhibitor is TTX-030.

In some embodiments, the present disclosure provides the pharmaceutical composition or the method wherein the CD73 inhibitor is quemliclustat (AB680), uliledlimab, mupadolimab, ORIC-533, ATG-037, PT-199, AK131, NZV930, BMS-986179, or oleclumab.

In some embodiments, the present disclosure provides the pharmaceutical composition or the method wherein the IL-2v is aldesleukin (Proleukin), bempegaldesleukin (NKTR-214), nemvaleukin alfa (ALKS-4230), THOR-202 (SAR-444245), BNT-151, ANV-419, XTX-202, RG-6279 (RO-7284755), NL-201, STK-012, SHR-1916, or GS-4528.

In some embodiments, the present disclosure provides the pharmaceutical composition or the method wherein the ADC is selected from sacituzumab govitecan, datopotamab deruxtecan, enfortumab vedotin, and trastuzumab deruxtecan.

In some embodiments, the present disclosure provides the pharmaceutical composition or the method wherein the additional therapeutic agent is selected from idealisib, sacituzumab govitecan, magrolimab, GS-0189, GS-3583, zimberelimab, GS-4224, GS-9716, GS-6451, GS-1811 (JTX-1811), quemliclustat (AB680), etrumadenant (AB928), domvanalimab, AB308, PY159, PY314, AGEN-1223, AGEN-2373, axicabtagene ciloleucel and brexucabtagene autoleucel.

In some embodiments, the method includes administering one or more additional therapeutic agents. The one or more additional therapeutic agents can be one or more therapuetic agents as described below. In some embodiments, the one or more additional therapeutic agents is independently a chemotherapeutic agent, an immunotherapeutic agent, a hormonal agent, an anti-hormonal agent, a targeted therapy agent, or an anti-angiogenesis agent.

In some embodiments, the one or more additional therapeutic agents includes therapeutic agents used to treat high risk myelodysplastic syndrome (HR MDS), low risk myelodyplastic syndrome (LR MDS), colorectal cancer, non-small cell lung cancer (NSCLC), pancreatic cancer, or endometrial cancer. In some embodiments, the one or more additional therapeutic agents includes therapeutic agents used to treat high risk myelodysplastic syndrome (HR MDS).

In some embodiments, the one or more additional therapeutic agents includes therapeutic agents used to treat low risk myelodyplastic syndrome (LR MDS).

In some embodiments, the one or more additional therapeutic agents includes therapeutic agents used to treat colorectal cancer.

In some embodiments, the one or more additional therapeutic agents includes therapeutic agents used to treat non-small cell lung cancer (NSCLC).

In some embodiments, the one or more additional therapeutic agents includes therapeutic agents used to treat pancreatic cancer.

In some embodiments, the one or more additional therapeutic agents includes therapeutic agents used to treat endometrial cancer. In some embodiments, the one or more additional therapeutic agents includes carboplatin, paclitaxel, cisplatin, doxorubicin, ifosfamide, progesterone, anastrozole (Arimidex®), letrozole (Femara®), exemestane (Aromasin®), pembrolizumab (Keytruda®), lenvatinib (Lenvima®), or dostarlimab (Jemperli®).

In some embodiments, the one or more additional therapeutic agents is independently SNS-301, 5-FU+leucovorin+oxaliplatin+irinotecan, 5-FU+nanoliposomal irinotecan, 5-FU, afatinib (Gilotrif©), aflibercept (Zaltrap®), aflibercept+FOLFIRI, albumin-bound paclitaxel, alectinib (Alecensa®), anastrozole (Arimidex®), atezolizumab, avelumab, azacitidine (Vidaza®), bevacizumab (Avastin®), bevacizumab+carboplatin+nab-paclitaxel, bevacizumab+carboplatin +pemetrexed, bevacizumab+FOLFIRI, bevacizumab+FOLFOX, bevacizumab+FOLFOXIRI, bevacizumab+leucovorin+5-FU+oxaliplatin (FOLFOX), bevacizumab+XELOX, bevacizumab, BGB324, binimetinib+encorafenib+cetuximab, brigatinib, cabozantinib, canakinumab, capecitabine, carboplatin+nab-paclitaxel, carboplatin+pemetrexed, carboplatin, cemiplimab, cetuximab (Erbitux®), cetuximab+FOLFIRI, cisplatin+gemcitabine, cisplatin+pemetrexed, cisplatin, crizotinib (Xalkori®), cytarabine+daunorubicin, cytarabine+idarubicin, cytarabine, dabrafenib (Tafinlar®), dabrafenib+trametinib, datopotamab deruxtecan (DS-1062), datopotamab deruxtecan+durvalumab, datopotamab deruxtecan+pembrolizumab, daunorubicin, decitabine (Dacogen®), docetaxel, domvanalimab, dostarlimab (Jemperli®), doxorubicin, DSP-7888, durvalumab+tremelimumab, durvalumab, enasidenib, enfortumab vedotin (Padcev®), entrectinib (Tarceva®), erlotinib, etoposide, exemestane (Aromasin®), fluorouracil, FOLFIRI, FOLFIRINOX, FOLFOXIRI, gefitinib (Iressa®), gemcitabine+nab-paclitaxel, gemcitabine, guadecitabine, idarubicin, ifosfamide, imetelstat, irinotecan, ivosidenib, LB-100, lenalidomide (Revlimid®), lenalidomide, lenvatinib (Lenvima®), letrozole (Femara®), leucovorin+nanoliposomal irinotecan, leucovorin, Lonsurf (Orcantas®), luspatercept, nab-paclitaxel (Abraxane®), napabucasin+FOLFIRI+bevacizumab, nivolumab (Opdivo®), nivolumab+docetaxel, nivolumab+ipilimumab, nogapendekin alfa (N-803)+pembrolizumab, nogapendekin alfa, ociperlimab+tislelizumab, ociperlimab, osimertinib (Tagrisso®), oxaliplatin (FOLFOX), paclitaxel, panitumumab, pembrolizumab (Keytruda®), pembrolizumab+carboplatin +nab-paclitaxel, pembrolizumab+carboplatin+pemetrexed, pembrolizumab+lenvatinib+pemetrexed, pembrolizumab+olaparib, pembrolizumab+pemetrexed+carboplatin, pemetrexed (Alimta®), pemetrexed+cisplatin+carboplatin, pevonedistat, progesterone, ramucirumab (Cyramza®), ramucirumab+docetaxel, regorafenib (Stivarga®), rigosertib, roxadustat, sabatolimab, selinexor, tiragolumab+atezolizumab, tiragolumab, trametinib (Mekinist®), trametinib+dabrafenib+panitumumab, trastuzumab+pertuzumab, trastuzumab deruxtecan (Enhertu®), trastuzumab, vandetanib, vemurafenib, venetoclax, vibostolimab+pembrolizumab, vibostolimab, vinblastine, vinorelbine, XELOX, or ziv-aflibercept.

In another embodiment, the present disclosure provides a method for manufacturing a medicament for treating cancer in a subject in need thereof, characterized in that a compound of the present disclosure, or a pharmaceutically acceptable salt thereof, is used.

In another embodiment, the present disclosure provides a method for manufacturing a medicament for inhibiting cancer metastasis in a subject in need thereof, characterized in that a compound of the present invention, or a pharmaceutically acceptable salt thereof, is used.

In another embodiment, the present disclosure provides use of the compound of the present disclosure, or a pharmaceutically acceptable salt thereof, for the manufacture of a medicament for the treatment of cancer in a subject.

In another embodiment, the present disclosure provides use of the compound of the present disclosure, or a pharmaceutically acceptable salt thereof, for the manufacture of a medicament for inhibiting cancer metastasis in a subject.

In another embodiment, the present disclosure provides the compound of the present disclosure, or a pharmaceutically acceptable salt thereof, for use in the treatment of cancer in a subject in need thereof.

In another embodiment, the present disclosure provides the compound of the present disclosure, or a pharmaceutically acceptable salt thereof, for use in inhibiting cancer metastasis in a subject in need thereof.

In another embodiment, the present disclosure provides the compound of the present disclosure, or a pharmaceutically acceptable salt thereof, for use in therapy.

### VIII. Combination Therapy

In some embodiments, a compound of Formula (I), provided herein, or pharmaceutically acceptable salt thereof, is administered in combination with one or more additional therapeutic agents to treat or prevent a disease or condition disclosed herein. In some embodiments, the one or more additional therapeutic agents are one, two, three, or four additional therapeutic agents. In some embodiments, the one or more additional therapeutic agents are one additional therapeutic agent. In some embodiments, the one or more additional therapeutic agents are two additional therapeutic agents. In some embodiments, the one or more additional therapeutic agents are three additional therapeutic agents. In some embodiments, the one or more additional therapeutic agents are four additional therapeutic agents.

In some embodiments, the pharmaceutical compositions provided herein have a compound of Formula (I), provided herein, or pharmaceutically acceptable salt thereof, and one or more additional therapeutic agents. In some embodiments, the one or more additional therapeutic agents are one, two, three, or four additional therapeutic agents. In some embodiments, the one or more additional therapeutic agents are one additional therapeutic agent. In some embodiments, the one or more additional therapeutic agents are two additional therapeutic agents. In some embodiments, the one or more additional therapeutic agents are three additional therapeutic agents. In some embodiments, the one or more additional therapeutic agents are four additional therapeutic agents.

In some embodiments the additional therapeutic agent includes, e.g., an inhibitory immune checkpoint blocker or inhibitor, a stimulatory immune checkpoint stimulator, agonist or activator, a chemotherapeutic agent, an anti-cancer agent, a radiotherapeutic agent, an anti-neoplastic agent, an anti-proliferation agent, an anti-angiogenic agent, an anti-inflammatory agent, an immunotherapeutic agent, a therapeutic antigen-binding molecule (e.g., a mono- and multi-specific antibody, or fragment thereof, in any format, such as DART®, Duobody®, BiTE®, BiKE, TriKE, XmAb®, TandAb®, scFv, Fab, Fab derivative), a bi-specific antibody, a non-immunoglobulin antibody mimetic (e.g., including adnectin, affibody, affilin, affimer, affitin, alphabody, anticalin, peptide aptamer, armadillo repeat protein (ARM), atrimer, avimer, designed ankyrin repeat protein (DARPin®), fynomer, knottin, Kunitz domain peptide, monobody, and nanoCLAMPs), an antibody-drug conjugate (ADC), antibody-peptide conjugate), an oncolytic virus, a gene modifier or editor, a cell comprising a chimeric antigen receptor (CAR), e.g., including a T-cell immunotherapeutic agent, an NK-cell immunotherapeutic agent, or a macrophage immunotherapeutic agent, a cell comprising an engineered T-cell receptor (TCR-T), or any combination thereof.

### Illustrative Targets

In some embodiments, the one or more additional therapeutic agents include, e.g., an inhibitor, agonist, antagonist, ligand, modulator, stimulator, blocker, activator or suppressor of a target (e.g., polypeptide or polynucleotide), such as: 2′-5′-oligoadenylate synthetase (OAS1; NCBI Gene ID: 4938); 5′-3′ exoribonuclease 1 (XRN1; NCBI Gene ID: 54464); 5′-nucleotidase ecto (NT5E, CD73; NCBI Gene ID: 4907); ABL proto-oncogene 1, non-receptor tyrosine kinase (ABL1, BCR-ABL, c-ABL, v-ABL; NCBI Gene ID: 25); absent in melanoma 2 (AIM2; NCBI Gene ID: 9447); acetyl-CoA acyltransferase 2 (ACAA2; NCBI Gene ID: 10499); acid phosphatase 3 (ACP3; NCBI Gene ID: 55); adenosine deaminase (ADA, ADA1; NCBI Gene ID: 100); adenosine receptors (e.g., ADORA1 (A1), ADORA2A (A2a, A2AR), ADORA2B (A2b, A2BR), ADORA3 (A3); NCBI Gene IDs: 134, 135, 136, 137); AKT serine/threonine kinase 1 (AKT1, AKT, PKB; NCBI Gene ID: 207); alanyl aminopeptidase, membrane (ANPEP, CD13; NCBI Gene ID: 290); ALK receptor tyrosine kinase (ALK, CD242; NCBI Gene ID: 238); alpha fetoprotein (AFP; NCBI Gene ID: 174); amine oxidase copper containing (e.g., AOC1 (DAO1), AOC2, AOC3 (VAP1); NCBI Gene IDs: 26, 314, 8639); androgen receptor (AR; NCBI Gene ID: 367); angiopoietins (ANGPT1, ANGPT2; NCBI Gene IDs: 284, 285); angiotensin II receptor type 1 (AGTR1; NCBI Gene ID: 185); angiotensinogen (AGT; NCBI Gene ID: 183); apolipoprotein A1 (APOA1; NCBI Gene ID: 335); apoptosis inducing factor mitochondria associated 1 (AIFM1, AIF; NCBI Gene ID: 9131); arachidonate 5-lipoxygenase (ALOX5; NCBI Gene ID: 240); asparaginase (ASPG; NCBI Gene ID: 374569); asteroid homolog 1 (ASTE1; NCBI Gene ID: 28990); ATM serine/threonine kinase (ATM; NCBI Gene ID: 472); ATP binding cassette subfamily B member 1 (ABCB1, CD243, GP170; NCBI Gene ID: 5243); ATP-dependent Clp-protease (CLPP; NCBI Gene ID: 8192); ATR serine/threonine kinase (ATR; NCBI Gene ID: 545); AXL receptor tyrosine kinase (AXL; NCBI Gene ID: 558); B and T lymphocyte associated (BTLA, CD272; NCBI Gene ID: 151888); baculoviral IAP repeat containing proteins (BIRC2 (cIAPI), BIRC3 (cIAP2), XIAP (BIRC4, IAP3), BIRC5 (survivin); NCBI Gene IDs: 329, 330, 331, 332); basigin (Ok blood group) (BSG, CD147; NCBI Gene ID: 682); B-cell lymphoma 2 (BCL2; NCBI Gene ID: 596); BCL2 binding component 3 (BBC3, PUMA; NCBI Gene ID: 27113); BCL2 like (e.g., BCL2L1 (Bcl-x), BCL2L2 (BIM); Bcl-x; NCBI Gene IDs: 598, 10018); beta 3-adrenergic receptor (ADRB3; NCBI Gene ID: 155); bone gamma-carboxyglutamate protein (BGLAP; NCBI Gene ID: 632); bone morphogenetic protein-10 ligand (BMP10; NCBI Gene ID: 27302); bradykinin receptors (e.g., BDKRB1, BDKRB2; NCBI Gene IDs: 623, 624); B-RAF (BRAF; NCBI Gene ID: 273); breakpoint cluster region (BCR; NCBI Gene ID: 613); bromodomain and external domain (BET) bromodomain containing proteins (e.g., BRD2, BRD3, BRD4, BRDT; NCBI Gene IDs: 6046, 8019, 23476, 676); Bruton's tyrosine kinase (BTK; NCBI Gene ID: 695); cadherins (e.g., CDH3 (p-cadherin), CDH6 (k-cadherin); NCBI Gene IDs: 1001, 1004); cancer/testis antigens (e.g., CTAG1A, CTAG1B, CTAG2; NCBI Gene IDs: 1485, 30848, 246100); cannabinoid receptors (e.g., CNR1 (CB1), CNR2 (CB2); NCBI Gene IDs: 1268, 1269); carbohydrate sulfotransferase 15 (CHST15; NCBI Gene ID: 51363); carbonic anhydrases (e.g., CA1, CA2, CA3, CA4, CA5A, CA5B, CA6, CA7, CA8, CA9, CA10, CA1l, CA12, CA13, CA14; NCBI Gene IDs: 759, 760, 761, 762, 763, 765, 766, 767, 768, 770, 771, 11238, 23632, 56934, 377677); carcinoembryonic antigen related cell adhesion molecules (e.g., CEACAM3 (CD66d), CEACAM5 (CD66e), CEACAM6 (CD66c); NCBI Gene IDs: 1048, 1084, 4680); casein kinases (e.g., CSNK1A1 (CK1), CSNK2A1 (CK2); NCBI Gene IDs: 1452, 1457); caspases (e.g., CASP3, CASP7, CASP8; NCBI Gene IDs: 836, 840, 841, 864); catenin beta 1 (CTNNB1; NCBI Gene ID: 1499); cathepsin G (CTSG; NCBI Gene ID: 1511); Cbl proto-oncogene B (CBLB, Cbl-b; NCBI Gene ID: 868); C—C motif chemokine ligand 21 (CCL21; NCBI Gene ID: 6366); C—C motif chemokine receptor 2 (CCR2; NCBI Gene ID: 729230); C—C motif chemokine receptors (e.g., CCR3 (CD193), CCR4 (CD194), CCR5 (CD195), CCR8 (CDw198); NCBI Gene IDs: 1232, 1233, 1234, 1237); CCAAT enhancer binding protein alpha (CEBPA, CEBP; NCBI Gene ID: 1050); cell adhesion molecule 1 (CADM1; NCBI Gene ID: 23705); cell division cycle 7 (CDC7; NCBI Gene ID: 8317); cellular communication network factor 2 (CCN2; NCBI Gene ID: 1490); cereblon (CRBN; NCBI Gene ID: 51185); checkpoint kinases (e.g., CHEK1 (CHK1), CHEK2 (CHK2); NCBI Gene IDs: 1111, 11200); cholecystokinin B receptor (CCKBR; NCBI Gene ID: 887); chorionic somatomammotropin hormone 1 (CSH1; NCBI Gene ID: 1442); claudins (e.g., CLDN6, CLDN18; NCBI Gene IDs: 9074, 51208); cluster of differentiation markers (e.g., CD1A, CD1C, CD1D, CD1E, CD2, CD3 alpha (TRA), CD beta (TRB), CD gamma (TRG), CD delta (TRD), CD4, CD8A, CD8B, CD19, CD20 (MS4A1), CD22, CD24, CD25 (IL2RA, TCGFR), CD28, CD33 (SIGLEC3), CD37, CD38, CD39 (ENTPD1), CD40 (TNFRSF5), CD44 (MIC4, PGP1), CD47 (IAP), CD48 (BLAST1), CD52, CD55 (DAF), CD58 (LFA3), CD74,CD79a, CD79b, CD80 (B7-1), CD84, CD86 (B7-2), CD96 (TACTILE), CD99 (MIC2), CD115 (CSF1R), CD116 (GMCSFR, CSF2RA), CD122 (IL2R^{B}), CD123 (IL3RA), CD128 (IL8R1), CD132 (IL2RG), CD135 (FLT3), CD137 (TNFRSF9, 4-1BB), CD142 (TF, TFA), CD152 (CTLA4), CD160, CD182 (IL8R2), CD193 (CCR3), CD194 (CCR4), CD195 (CCR5), CD207, CD221 (IGF1R), CD222 (IGF2R), CD223 (LAG3), CD226 (DNAM1), CD244, CD247, CD248, CD276 (B7-H3), CD331 (FGFR1), CD332 (FGFR2), CD333 (FGFR3), CD334 (FGFR4); NCBI Gene IDs: 909, 911, 912, 913, 914, 919, 920, 923, 925, 926, 930, 931, 933, 940, 941, 942, 945, 951, 952, 953, 958,960, 961, 962, 965, 972, 973, 974, 1043, 1232, 1233, 1234, 1237, 1436, 1438, 1493, 1604, 2152, 2260, 2261, 2263, 2322, 3480, 3482, 3559, 3560, 3561, 3563, 3577, 3579, 3604, 3902, 4267, 6955, 6957, 6964, 6965, 8832, 10666, 11126, 50489, 51744, 80381, 100133941); clusterin (CLU; NCBI Gene ID: 1191); coagulation factors (e.g., F7, FXA; NCBI Gene IDs: 2155, 2159); collagen type IV alpha chains (e.g., COL4A1, COL4A2, COL4A3, COL4A4, COL4A5; NCBI Gene IDs: 1282, 1284, 1285, 1286, 1287); collectin subfamily member 10 (COLEC10; NCBI Gene ID: 10584); colony stimulating factors (e.g., CSF1 (MCSF), CSF2 (GMCSF), CSF3 (GCSF); NCBI Gene IDs: 1435, 1437, 1440); complement factors (e.g., C₃, C₅; NCBI Gene IDs: 718, 727); COP9 signalosome subunit 5 (COPS5; NCBI Gene ID: 10987); C-type lectin domain family member (e.g., CLEC4C (CD303), CLEC9A (CD370), CLEC12A (CD371); CD371; NCBI Gene ID: 160364, 170482, 283420); C—X—C motif chemokine ligand 12 (CXCL12; NCBI Gene ID: 6387); C—X—C motif chemokine receptors (CXCR1 (IL8R1, CD128), CXCR2 (IL8R2, CD182), CXCR3 (CD182, CD183, IP-10R), CXCR4 (CD184); NCBI Gene ID: 2833, 3577, 3579, 7852); cyclin D1 (CCND1, BCL1; NCBI Gene ID: 595); cyclin dependent kinases (e.g., CDK1, CDK2, CDK3, CDK4, CDK5, CDK6, CDK7, CDK8, CDK9, CDK10, CDK12; NCBI Gene ID: 983, 1017, 1018, 1019, 1020, 1021, 1022, 1024, 1025, 8558, 51755); cyclin G1 (CCNG1; NCBI Gene ID: 900); cytochrome P450 family members (e.g., CYP2D6, CYP3A4, CYP11A1, CYP11B2, CYP17A1, CYP19A1, CYP51A1; NCBI Gene IDs: 1565, 1576, 1583, 1585, 1586, 1588, 1595); cytochrome P450 oxidoreductase (POR; NCBI Gene ID: 5447); cytokine inducible SH2 containing protein (CISH; NCBI Gene ID: 1154); cytotoxic T-lymphocyte associated protein 4 (CTLA4, CD152; NCBI Gene ID: 1493); DEAD-box helicases (e.g., DDX5, DDX6, DDX58; NCBI Gene IDs: 1655, 1656, 23586); delta like canonical Notch ligands (e.g., DLL3, DLL4; NCBI Gene IDs: 10683, 54567); diablo IAP-binding mitochondrial protein (DIABLO, SMAC; NCBI Gene ID: 56616); diacylglycerol kinases (e.g., DGKA, DGKZ; NCBI Gene IDs: 1606, 8525); dickkopf WNT signaling pathway inhibitors (e.g., DKK1, DKK3; NCBI Gene ID: 22943, 27122); dihydrofolate reductase (DHFR; NCBI Gene ID: 1719); dihydropyrimidine dehydrogenase (DPYD; NCBI Gene ID: 1806); dipeptidyl peptidase 4 (DPP4; NCBI Gene ID: 1803); discoidin domain receptor tyrosine kinases (e.g., DDR1 (CD167), DDR2; CD167; NCBI Gene ID: 780, 4921); DNA dependent protein kinase (PRKDC; NCBI Gene ID: 5591); DNA topoisomerases (e.g., TOP1, TOP2A, TOP2B, TOP3A, TOP3B; NCBI Gene ID: 7150, 7153, 7155, 7156, 8940); dopachrome tautomerase (DCT; NCBI Gene ID: 1638); dopamine receptor D2 (DRD2; NCBI Gene ID: 1318); DOT1 like histone lysine methyltransferase (DOTIL; NCBI Gene ID: 84444); ectonucleotide pyrophosphatase/phosphodiesterase 3 (ENPP3, CD203c; NCBI Gene ID: 5169); EMAP like 4 (EML4; NCBI Gene ID: 27436); endoglin (ENG; NCBI Gene ID: 2022); endoplasmic reticulum aminopeptidases (e.g., ERAP1, ERAP2; NCBI Gene ID: 51752, 64167); enhancer of zeste 2 polycomb repressive complex 2 subunit (EZH2; NCBI Gene ID: 2146); ephrin receptors (e.g., EPHA1, EPHA2EPHA3, EPHA4, EPHA5, EPHA7, EPHB4; NCBIGene ID:1969, 2041, 2042, 2043, 2044, 2045, 2050); ephrins (e.g., EFNA1, EFNA4, EFNB2; NCBI Gene ID: 1942, 1945, 1948); epidermal growth factor receptors (e.g., ERBB1 (HER1, EGFR), ERBB1 variant III (EGFRvIII), ERBB2 (HER2, NEU, CD340), ERBB3 (HER3), ERBB4 (HER4); NCBI Gene ID: 1956, 2064, 2065, 2066); epithelial cell adhesion molecule (EPCAM; NCBI Gene ID: 4072); epithelial mitogen (EPGN; NCBI Gene ID: 255324); eukaryotic translation elongation factors (e.g., EEF1A2, EEF2; NCBI Gene ID: 1917, 1938); eukaryotic translation initiation factors (e.g., EIF4A1, EIFSA; NCBI Gene ID: 1973, 1984); exportin-1 (XPO1; NCBI Gene ID: 7514); farnesoid X receptor (NR1H4, FXR; NCBI Gene ID: 9971); Fas ligand (FASLG, FASL, CD95L, CD178, TNFSF6; NCBI Gene ID: 356); fatty acid amide hydrolase (FAAH; NCBI Gene ID: 2166); fatty acid synthase (FASN; FAS; NCBI Gene ID: 2194); Fc fragment of Ig receptors (e.g., FCER1A, FCGRT, FCGR3A (CD16); NCBI Gene IDs: 2205, 2214, 2217); Fc receptor like 5 (FCRL5, CD307; NCBI Gene ID: 83416); fibroblast activation protein alpha (FAP; NCBI Gene ID: 2191); fibroblast growth factor receptors (e.g., FGFR1 (CD331), FGFR2 (CD332), FGFR3 (CD333), FGFR4 (CD334); NCBI Gene IDs: 2260, 2261, 2263, 2264); fibroblast growth factors (e.g., FGF1 (FGF alpha), FGF2 (FGF beta), FGF4, FGF5; NCBI Gene IDs: 2246, 2247, 2249, 2250); fibronectin 1 (FN1, MSF; NCBI Gene ID: 2335); fms related receptor tyrosine kinases (e.g., FLT1 (VEGFR1), FLT3 (STK1, CD135), FLT4 (VEGFR2); NCBI Gene IDs: 2321, 2322, 2324); fms related receptor tyrosine kinase 3 ligand (FLT3LG; NCBI Gene ID: 2323); focal adhesion kinase 2 (PTK2, FAK1; NCBI Gene ID: 5747); folate hydrolase 1 (FOLH1, PSMA; NCBI Gene ID: 2346); folate receptor 1 (FOLR1; NCBI Gene ID: 2348); forkhead box protein M1 (FOXM1; NCBI Gene ID: 2305); FURIN (FURIN, PACE; NCBI Gene ID: 5045); FYN tyrosine kinase (FYN, SYN; NCBI Gene ID: 2534); galectins (e.g., LGALS3, LGALS8 (PCTA1), LGALS9; NCBI Gene ID: 3958, 3964, 3965); glucocorticoid receptor (NR3C₁, GR; NCBI Gene ID: 2908); glucuronidase beta (GUSB; NCBI Gene ID: 2990); glutamate metabotropic receptor 1 (GRM1; NCBI Gene ID: 2911); glutaminase (GLS; NCBI Gene ID: 2744); glutathione S-transferase Pi (GSTP1; NCBI Gene ID: 2950); glycogen synthase kinase 3 beta (GSK3B; NCBI Gene ID: 2932); glypican 3 (GPC3; NCBI Gene ID: 2719); gonadotropin releasing hormone 1 (GNRH1; NCBI Gene ID: 2796); gonadotropin releasing hormone receptor (GNRHR; NCBI Gene ID: 2798); GPNMB glycoprotein nmb (GPNMB, osteoactivin; NCBI Gene ID: 10457); growth differentiation factor 2 (GDF2, BMP9; NCBI Gene ID: 2658); growth factor receptor-bound protein 2 (GRB2, ASH; NCBI Gene ID: 2885); guanylate cyclase 2C (GUCY2C, STAR, MECIL, MUCIL, NCBI Gene ID: 2984); H19 imprinted maternally expressed transcript (H19; NCBI Gene ID: 283120); HCK proto-oncogene, Src family tyrosine kinase (HCK; NCBI Gene ID: 3055); heat shock proteins (e.g., HSPA5 (HSP70, BIP, GRP78), HSPB1 (HSP27), HSP90B1 (GP96); NCBI Gene IDs: 3309, 3315, 7184); heme oxygenases (e.g., HMOX1 (HO1), HMOX2 (HO1); NCBI Gene ID: 3162, 3163); heparanase (HPSE; NCBI Gene ID: 10855); hepatitis A virus cellular receptor 2 (HAVCR2, TIM3, CD366; NCBI Gene ID: 84868); hepatocyte growth factor (HGF; NCBI Gene ID: 3082); HERV-H LTR-associating 2 (HHLA2, B7-H7; NCBI Gene ID: 11148); histamine receptor H2 (HRH2; NCBI Gene ID: 3274); histone deacetylases (e.g., HDAC1, HDAC7, HDAC9; NCBI Gene ID: 3065, 9734, 51564); HRas proto-oncogene, GTPase (HRAS; NCBI Gene ID: 3265); hypoxia-inducible factors (e.g., HIF1A, HIF2A (EPAS1); NCBI Gene IDs: 2034, 3091); I-Kappa-B kinase (IKK beta; NCBI Gene IDs: 3551, 3553); IKAROS family zinc fingers (IKZF1 (LYF1), IKZF3; NCBI Gene ID: 10320, 22806); immunoglobulin superfamily member 11 (IGSF11; NCBI Gene ID: 152404); indoleamine 2,3-dioxygenases (e.g., IDO1, ID02; NCBI Gene IDs: 3620, 169355); inducible T cell costimulator (ICOS, CD278; NCBI Gene ID: 29851); inducible T cell costimulator ligand (ICOSLG, B7-H2; NCBI Gene ID: 23308); insulin like growth factor receptors (e.g., IGF1R, IGF2R; NCBI Gene ID: 3480, 3482); insulin like growth factors (e.g., IGF1, IGF2; NCBI Gene IDs: 3479, 3481); insulin receptor (INSR, CD220; NCBI Gene ID: 3643); integrin subunits (e.g., ITGA5 (CD49e), ITGAV (CD51), ITGB1 (CD29), ITGB2 (CD18, LFA1, MAC1), ITGB7; NCBI Gene IDs: 3678, 3685, 3688, 3695, 3698); intercellular adhesion molecule 1 (ICAM1, CD54; NCBI Gene ID: 3383); interleukin 1 receptor associated kinase 4 (IRAK4; NCBI Gene ID: 51135); interleukin receptors (e.g., IL2RA (TCGFR, CD25), IL2R^{B} (CD122), IL2RG (CD132), IL3RA, IL6R, IL13RA2 (CD213A2), IL22RA1; NCBI Gene IDs: 3598, 3559, 3560, 3561, 3563, 3570, 58985); interleukins (e.g., IL1A, IL1B, IL2, IL3, IL6 (HGF), IL7, IL8 (CXCL8), IL10 (TGIF), IL12A, IL12B, IL15, IL17A (CTLA8), IL18, IL23A, IL24, IL-29 (IFNL1); NCBI Gene IDs: 3552, 3553, 3558, 3562, 3565, 3569, 3574, 3586, 3592, 3593, 3600, 3605, 3606, 11009, 51561, 282618); isocitrate dehydrogenases (NADP(+)₁) (e.g., IDH1, IDH2; NCBI Gene IDs: 3417, 3418); Janus kinases (e.g., JAK1, JAK2, JAK3; NCBI Gene IDs: 3716, 3717, 3718); kallikrein related peptidase 3 (KLK3; NCBI Gene ID: 354); killer cell immunoglobulin like receptor, Ig domains and long cytoplasmic tails (e.g., KIR2DL1 (CD158A), KIR2DL2 (CD158B1), KIR2DL3 (CD158B), KIR2DL4 (CD158D), KIR2DL5A (CD158F), KIR2DL5B, KIR3DL1 (CD158E1), KIR3DL2 (CD158K), KIR3DP1 (CD158c), KIR2DS2 (CD158J); NCBI Gene IDs: 3802, 3803, 3804, 3805, 3811, 3812, 57292, 553128, 548594, 100132285); killer cell lectin like receptors (e.g., KLRC1 (CD159A), KLRC2 (CD159c), KLRC3, KLRRC4, KLRD1 (CD94), KLRG1, KLRK1 (NKG2D, CD314); NCBI Gene IDs: 3821, 3822, 3823, 3824, 8302, 10219, 22914); kinase insert domain receptor (KDR, CD309, VEGFR2; NCBI Gene ID: 3791); kinesin family member 11 (KIF11; NCBI Gene ID: 3832); KiSS-1 metastasis suppressor (KISS1; NCBI Gene ID: 3814); KIT proto-oncogene, receptor tyrosine kinase (KIT, C-KIT, CD117; NCBI Gene ID: 3815); KRAS proto-oncogene, GTPase (KRAS; NCBI Gene ID: 3845); lactotransferrin (LTF; NCBI Gene ID: 4057); LCK proto-oncogene, Src family tyrosine kinase (LCK; NCBI Gene ID: 3932); LDL receptor related protein 1 (LRP1, CD91, IGFBP3R; NCBI Gene ID: 4035); leucine rich repeat containing 15 (LRRC15; NCBI Gene ID: 131578); leukocyte immunoglobulin like receptors (e.g., LILRB1 (ILT2, CD85J), LILRB2 (ILT4, CD85D); NCBI Gene ID: 10288, 10859); leukotriene A4 hydrolase (LTA4H; NCBI Gene ID: 4048); linker for activation of T-cells (LAT; NCBI Gene ID: 27040); luteinizing hormone/choriogonadotropin receptor (LHCGR; NCBI Gene ID: 3973); LY6/PLAUR domain containing 3 (LYPD3; NCBI Gene ID: 27076); lymphocyte activating 3 (LAG3; CD223; NCBI Gene ID: 3902); lymphocyte antigens (e.g., LY9 (CD229), LY75 (CD205); NCBI Gene IDs: 4063, 17076); LYN proto-oncogene, Src family tyrosine kinase (LYN; NCBI Gene ID: 4067); lypmphocyte cytosolic protein 2 (LCP2; NCBI Gene ID: 3937); lysine demethylase 1A (KDM1A; NCBI Gene ID: 23028); lysophosphatidic acid receptor 1 (LPAR1, EDG2, LPA1, GPR26; NCBI Gene ID: 1902); lysyl oxidase (LOX; NCBI Gene ID: 4015); lysyl oxidase like 2 (LOXL2; NCBI Gene ID: 4017); macrophage migration inhibitory factor (MIF, GIF; NCBI Gene ID: 4282); macrophage stimulating 1 receptor (MST1R, CD136; NCBI Gene ID: 4486); MAGE family members (e.g., MAGEA1, MAGEA2, MAGEA2B, MAGEA3, MAGEA4, MAGEA5, MAGEA6, MAGEA10, MAGEA11, MAGEC1, MAGEC2, MAGED1, MAGED2; NCBI Gene IDs: 4100, 4101, 4102, 4103, 4104, 4105, 4109, 4110, 9500, 9947, 10916, 51438, 266740); major histocompatibility complexes (e.g., HLA-A, HLA-E, HLA-F, HLA-G; NCBI Gene IDs: 3105, 3133, 3134, 3135); major vault protein (MVP, VAULTI; NCBI Gene ID: 9961); MALT1 paracaspase (MALT1; NCBI Gene ID: 10892); MAPK activated protein kinase 2 (MAPKAPK2; NCBI Gene ID: 9261); MAPK interacting serine/threonine kinases (e.g., MKNK1, MKNK2; NCBI Gene IDs: 2872, 8569); matrix metallopeptidases (e.g., MMP1, MMP2, MMP3, MMP7, MMP8, MMP9, MMP10, MMP11, MMP12, MMP13, MMP14, MMP15, MMP16, MMP17, MMP19, MMP20, MMP21, MMP24, MMP25, MMP26, MMP27, MMP28; NCBI Gene IDs: 4312, 4313, 4314, 4316, 4317, 4318, 4319, 4320, 4321, 4322, 4323, 4324, 4325, 4326, 4327, 9313, 10893, 56547, 64066, 64386, 79148, 118856); MCL1 apoptosis regulator, BCL2 family member (MCL1; NCBI Gene ID: 4170); MDM2 proto-oncogene (MDM2; NCBI Gene ID: 4193); MDM4 regulator of p53 (MDM4; BMFS6; NCBI Gene ID: 4194); mechanistic target of rapamycin kinase (MTOR, FRAPI; NCBI Gene ID: 2475); melan-A (MLANA; NCBI Gene ID: 2315); melanocortin receptors (MC1R, MC2R; NCBI Gene IDs: 4157, 4148); MER proto-oncogene, tyrosine kinase (MERTK; NCBI Gene ID: 10461); mesothelin (MSLN; NCBI Gene ID: 10232); MET proto-oncogene, receptor tyrosine kinase (MET, c-Met, HGFR; NCBI Gene ID: 4233); methionyl aminopeptidase 2 (METAP2, MAP2; NCBI Gene ID: 10988); MHC class I polypeptide-related sequences (e.g., MICA, MICB; NCBI Gene IDs: 4277, 100507436); mitogen activated protein kinases (e.g., MAPK1 (ERK2), MAPK3 (ERK1), MAPK8 (JNK1), MAPK9 (JNK2), MAPK10 (JNK3), MAPK11 (p38 beta), MAPK12; NCBI Gene IDs: 5594, 5595, 5599, 5600, 5601, 5602, 819251); mitogen-activated protein kinase kinase kinases (e.g., MAP3K5 (ASKI), MAP3K8 (TPL2, AURA2); NCBI Gene IDs: 4217, 1326); mitogen-activated protein kinase kinase kinase kinase 1 (MAP4K1, HPK1; NCBI Gene ID: 11184); mitogen-activated protein kinase kinases (e.g., MAP2K1 (MEK1), MAP2K2 (MEK2), MAP2K7 (MEK7); NCBI Gene IDs: 5604, 5605, 5609); MPL proto-oncogene, thrombopoietin receptor (MPL; NCBI Gene ID: 4352); mucins (e.g., MUC1 (including splice variants thereof (e.g., including MUC1/A, C, D, X, Y, Z and REP)), MUCSAC, MUC16 (CA125); NCBI Gene IDs: 4582, 4586, 94025); MYC proto-oncogene, bHLH transcription factor (MYC; NCBI Gene ID: 4609); myostatin (MSTN, GDF8; NCBI Gene ID: 2660); myristoylated alanine rich protein kinase C substrate (MARCKS; NCBI Gene ID: 4082); natriuretic peptide receptor 3 (NPR3; NCBI Gene ID: 4883); natural killer cell cytotoxicity receptor 3 ligand 1 (NCR3LG1, B7-H6; NCBI Gene ID: 374383); necdin, MAGE family member (NDN; NCBI Gene ID: 4692); nectin cell adhesion molecules (e.g., NECTIN2 (CD112, PVRL2), NECTIN4 (PVRL4); NCBI Gene IDs: 5819, 81607); neural cell adhesion molecule 1 (NCAM1, CD56; NCBI Gene ID: 4684); neuropilins (e.g., NRP1 (CD304, VEGF165R), NRP2 (VEGF165R2); NCBI Gene IDs: 8828, 8829); neurotrophic receptor tyrosine kinases (e.g., NTRK1 (TRKA), NTRK2 (TRKB), NTRK3 (TRKC); NCBI Gene IDs: 4914, 4915, 4916); NFKB activating protein (NKAP; NCBI Gene ID: 79576); NIMA related kinase 9 (NEK9; NCBI Gene ID: 91754); NLR family pyrin domain containing 3 (NLRP3, NALP3; NCBI Gene ID: 114548); notch receptors (e.g., NOTCH1, NOTCH2, NOTCH3, NOTCH4; NCBI Gene IDs: 4851, 4853, 4854, 4855); NRAS proto-oncogene, GTPase (NRAS; NCBI Gene ID: 4893); nuclear factor kappa B (NFKB1, NFKB2; NCBI Gene IDs: 4790, 4791); nuclear factor, erythroid 2 like 2 (NFE2L²; NRF2; NCBI Gene ID: 4780); nuclear receptor subfamily 4 group A member 1 (NR4A1; NCBI Gene ID: 3164); nucleolin (NCL; NCBI Gene ID: 4691); nucleophosmin 1 (NPM1; NCBI Gene ID: 4869); nucleotide binding oligomerization domain containing 2 (NOD2; NCBI Gene ID: 64127); nudix hydrolase 1 (NUDT1; NCBI Gene ID: 4521); 0-6-methylguanine-DNA methyltransferase (MGMT; NCBI Gene ID: 4255); opioid receptor delta 1 (OPRD1; NCBI Gene ID: 4985); ornithine decarboxylase 1 (ODC1; NCBI Gene ID: 4953); oxoglutarate dehydrogenase (OGDH; NCBI Gene ID: 4967); parathyroid hormone (PTH; NCBI Gene ID: 5741); PD-L1 (CD274; NCBI Gene ID: 29126); periostin (POSTN; NCBI Gene ID: 10631); peroxisome proliferator activated receptors (e.g., PPARA (PPAR alpha), PPARD (PPAR delta), PPARG (PPAR gamma); NCBI Gene IDs: 5465, 5467, 5468); phosphatase and tensin homolog (PTEN; NCBI Gene ID: 5728); phosphatidylinositol-4,5-bisphosphate 3-kinases (PIK3CA (PI3K alpha), PIK3CB (PI3K beta), PIK3CD (PI3K delta), PIK3CG (PI3K gamma); NCBI Gene IDs: 5290, 5291, 5293, 5294); phospholipases (e.g., PLA2G1B, PLA2G2A, PLA2G2D, PLA2G3, PLA2G4A, PLA2G5, PLA2G7, PLA2G10, PLA2G12A, PLA2G12B, PLA2G15; NCBI Gene IDs: 5319, 5320, 5321, 5322, 7941, 8399, 50487, 23659, 26279, 81579, 84647); Pim proto-oncogene, serine/threonine kinases (e.g., PIM1, PIM2, PIM3; NCBI Gene IDs: 5292, 11040, 415116); placenta growth factor (PGF; NCBI Gene ID: 5228); plasminogen activator, urokinase (PLAU, u-PA, ATF; NCBI Gene ID: 5328); platelet derived growth factor receptors (e.g., PDGFRA (CD140A, PDGFR2), FDGFRB (CD140B, PDGFR1); NCBI Gene IDs: 5156, 5159); plexin Bi (PLXNB1; NCBI Gene ID: 5364); poliovirus receptor (PVR) cell adhesion molecule (PVR, CD155; NCBI Gene ID: 5817); polo like kinase 1 (PLKI; NCBI Gene ID: 5347); poly(ADP-ribose) polymerases (e.g., PARP1, PARP2, PARP3; NCBI Gene IDs: 142, 10038, 10039); polycomb protein EED (EED; NCBI Gene ID: 8726); porcupine O-acyltransferase (PORCN; NCBI Gene ID: 64840); PRAME nuclear receptor transcriptional regulator (PRAME; NCBI Gene ID: 23532); premelanosome protein (PMEL; NCBI Gene ID: 6490); progesterone receptor (PGR; NCBI Gene ID: 5241); programmed cell death 1 (PDCD1, PD-1, CD279; NCBI Gene ID: 5133); programmed cell death 1 ligand 2 (PDCDILG2, CD273, PD-L2; NCBI Gene ID: 80380); prominin 1 (PROM1, CD133; NCBI Gene ID: 8842); promyelocytic leukemia (PML; NCBI Gene ID: 5371); prosaposin (PSAP; NCBI Gene ID: 5660); prostaglandin E receptor 4 (PTGER4; NCBI Gene ID: 5734); prostaglandin E synthase (PTGES; NCBI Gene ID: 9536); prostaglandin-endoperoxide synthases (PTGS1 (COX1), PTGS2 (COX2); NCBI Gene ID: 5742, 5743); proteasome 20S subunit beta 9 (PSMB9; NCBI Gene ID: 5698); protein arginine methyltransferases (e.g., PRMT1, PRMT5; NCBI Gene ID: 3276, 10419); protein kinase N3 (PKN3; NCBI Gene ID: 29941); protein phosphatase 2A (PPP2CA; NCBI Gene ID: 5515); protein tyrosine kinase 7 (inactive) (PTK7; NCBI Gene ID: 5754); protein tyrosine phosphatase receptors (PTPRB (PTPB), PTPRC (CD45R); NCBI Gene ID: 5787, 5788); prothymosin alpha (PTMA; NCBI Gene ID: 5757); purine nucleoside phosphorylase (PNP; NCBI Gene ID: 4860); purinergic receptor P2X 7 (P2R^{x}7; NCBI Gene ID: 5027); PVR related immunoglobulin domain containing (PVRIG, CD112R; NCBI Gene ID: 79037); Raf-1 proto-oncogene, serine/threonine kinase (RAF1, c-Raf; NCBI Gene ID: 5894); RAR-related orphan receptor gamma (RORC; NCBI Gene ID: 6097); ras homolog family member C (RHOC); NCBI Gene ID: 389); Ras homolog, mTORC1 binding (RHEB; NCBI Gene ID: 6009); R^{B} transcriptional corepressor 1 (R^{B1}; NCBI Gene ID: 5925); receptor-interacting serine/threonine protein kinase 1 (RIPK1; NCBI Gene ID: 8737); ret proto-oncogene (RET; NCBI Gene ID: 5979); retinoic acid early transcripts (e.g., RAETIE, RAETIG, RAETIL; NCBI Gene IDs: 135250, 154064, 353091); retinoic acid receptors alpha (e.g., RARA, RARG; NCBI Gene IDs: 5914, 5916); retinoid X receptors (e.g., RXRA, RXRB, RXRG; NCBI Gene IDs: 6256, 6257, 6258); Rho associated coiled-coil containing protein kinases (e.g., ROCK1, ROCK2; NCBI Gene IDs: 6093, 9475); ribosomal protein S6 kinase B1 (RPS6KB1, S6K-beta 1; NCBI Gene ID: 6198); ring finger protein 128 (RNF128, GRAIL; NCBI Gene ID: 79589); ROS proto-oncogene 1, receptor tyrosine kinase (ROS1; NCBI Gene ID: 6098); roundabout guidance receptor 4 (ROBO4; NCBI Gene ID: 54538); RUNX family transcription factor 3 (RUNX3; NCBI Gene ID: 864); S100 calcium binding protein A9 (S100A9; NCBI Gene ID: 6280); secreted frizzled related protein 2 (SFRP2; NCBI Gene ID: 6423); secreted phosphoprotein 1 (SPP1; NCBI Gene ID: 6696); secretoglobin family 1A member 1 (SCGB1A1; NCBI Gene ID: 7356); selectins (e.g., SELE, SELL (CD62L), SELP (CD62); NCBI Gene IDs: 6401, 6402, 6403); semaphorin 4D (SEMA4D; CD100; NCBI Gene ID: 10507); sialic acid binding Ig like lectins (SIGLEC7 (CD328), SIGLEC9 (CD329), SIGLEC10; NCBI Gene ID: 27036, 27180, 89790); signal regulatory protein alpha (SIRPA, CD172A; NCBI Gene ID: 140885); signal transducer and activator of transcription (e.g., STAT1, STAT3, STATSA, STATSB; NCBI Gene IDs: 6772, 6774, 6776, 6777); sirtuin-3 (SIRT3; NCBI Gene ID: 23410); signaling lymphocytic activation molecule (SLAM) family members (e.g., SLAMFI (CD150), SLAMF6 (CD352), SLAMF7 (CD319), SLAMF8 (CD353), SLAMF9; NCBI Gene IDs: 56833, 57823, 89886, 114836); SLIT and NTRK like family member 6 (SLITRK6; NCBI Gene ID: 84189); smoothened, frizzled class receptor (SMO; NCBI Gene ID: 6608); soluble epoxide hydrolase 2 (EPHX2; NCBI Gene ID: 2053); solute carrier family members (e.g., SLC3A2 (CD98), SLC5A5, SLC6A2, SLC10A3, SLC34A2, SLC39A6, SLC43A2 (LAT4), SLC44A4; NCBI Gene IDs: 6520, 6528, 6530, 8273, 10568, 25800, 80736, 124935); somatostatin receptors (e.g., SSTR1, SSTR2, SSTR3, SSTR4, SSTR5; NCBI Gene IDs: 6751, 6752, 6753, 6754, 6755); sonic hedgehog signaling molecule (SHH; NCBI Gene ID: 6469); Sp1 transcription factor (SP1; NCBI Gene ID: 6667); sphingosine kinases (e.g., SPHK1, SPHK2; NCBI Gene IDs: 8877, 56848); sphingosine-1-phosphate receptor 1 (S1PR1, CD363; NCBI Gene ID: 1901); spleen associated tyrosine kinase (SYK; NCBI Gene ID: 6850); splicing factor 3B factor 1 (SF₃B1; NCBI Gene ID: 23451); SRC proto-oncogene, non-receptor tyrosine kinase (SRC; NCBI Gene ID: 6714); stabilin 1 (STAB1, CLEVER-1; NCBI Gene ID: 23166); STEAP family member 1 (STEAPI; NCBI Gene ID: 26872); steroid sulfatase (STS; NCBI Gene ID: 412); stimulator of interferon response cGAMP interactor 1 (STINGI; NCBI Gene ID: 340061); superoxide dismutase 1 (SOD1, ALS1; NCBI Gene ID: 6647); suppressors of cytokine signaling (SOCS1 (CISH1), SOCS3 (CISH3); NCBI Gene ID: 8651, 9021); synapsin 3 (SYN3; NCBI Gene ID: 8224); syndecan 1 (SDC1, CD138, syndecan; NCBI Gene ID: 6382); synuclein alpha (SNCA, PARK1; NCBI Gene ID: 6622); T cell immunoglobulin and mucin domain containing 4 (TIMD4, SMUCKLER; NCBI Gene ID: 91937); T cell immunoreceptor with Ig and ITIM domains (TIGIT; NCBI Gene ID: 201633); tachykinin receptors (e.g., TACR1, TACR3; NCBI Gene ID: 6869, 6870); TANK binding kinase 1 (TBK1; NCBI Gene ID: 29110); tankyrase (TNKS; NCBI Gene ID: 8658); TATA-box binding protein associated factor, RNA polymerase I subunit B (TAF1B; NCBI Gene ID: 9014); T-box transcription factor T (TBXT; NCBI Gene ID: 6862); TCDD inducible poly(ADP-ribose) polymerase (TIPARP, PAPR7; NCBI Gene ID: 25976); tec protein tyrosine kinase (TEC; NCBI Gene ID: 7006); TEK receptor tyrosine kinase (TEK, CD202B, TIE2; NCBI Gene ID: 7010); telomerase reverse transcriptase (TERT; NCBI Gene ID: 7015); tenascin C (TNC; NCBI Gene ID: 3371); three prime repair exonucleases (e.g., TREX1, TREX2; NCBI Gene ID: 11277, 11219); thrombomodulin (THBD, CD141; NCBI Gene ID: 7056); thymidine kinases (e.g., TK1, TK2; NCBI Gene IDs: 7083, 7084); thymidine phosphorylase (TYMP; NCBI Gene ID: 1890); thymidylate synthase (TYMS; NCBI Gene ID: 7298); thyroid hormone receptor (THRA, THRB; NCBI Gene IDs: 7606, 7608); thyroid stimulating hormone receptor (TSHR; NCBI Gene ID: 7253); TNF superfamily members (e.g., TNFSF4 (OX40L, CD252), TNFSF5 (CD40L), TNFSF7 (CD70), TNFSF8 (CD153, CD30L), TNFSF9 (4-1BB-L, CD137L), TNFSF10 (TRAIL, CD253, APO2L), TNFSF11 (CD254, RANKL2, TRANCE), TNFSF13 (APRIL, CD256, TRAIL2), TNFSF13b (BAFF, BLYS, CD257), TNFSF14 (CD258, LIGHT), TNFSF18 (GITRL); NCBI Gene IDs: 944, 959, 970, 7292, 8600, 8740, 8741, 8743, 8744, 8995); toll like receptors (e.g., TLR1 (CD281), TLR2 (CD282), TLR3 (CD283), TLR4 (CD284), TLR5, TLR6 (CD286), TLR7, TLR8 (CD288), TLR9 (CD289), TLR10 (CD290); NCBI Gene IDs: 7096, 7097, 7098, 7099, 10333, 51284, 51311, 54106, 81793); transferrin (TF; NCBI Gene ID: 7018); transferrin receptor (TFRC, CD71; NCBI Gene ID: 7037); transforming growth factors (e.g., TGFA, TGFB1; NCBI Gene ID: 7039, 7040); transforming growth factor receptors (e.g., TGFBR1, TGFBR2, TGFBR3; NCBI Gene ID: 7046, 7048, 7049); transforming protein E7 (E7; NCBI Gene ID: 1489079); transglutaminase 5 (TGM5; NCBI Gene ID: 9333); transient receptor potential cation channel subfamily V member 1 (TRPV1, VR1; NCBI Gene ID: 7442); transmembrane and immunoglobulin domain containing 2 (TMIGD2, CD28H, IGPR1; NCBI Gene ID: 126259); triggering receptors expressed on myeloid cells (e.g., TREM1 (CD354), TREM2; NCBI Gene ID: 54209, 54210); trophinin (TRO, MAGED3; NCBI Gene ID: 7216); trophoblast glycoprotein (TPBG; NCBI Gene ID: 7162); tryptophan 2,3-dioxygenase (TDO2; NCBI Gene ID: 6999); tryptophan hydroxylases (e.g., TPH1, TPH2; NCBI Gene ID: 7166, 121278); tumor associated calcium signal transducer 2 (TACSTD2, TROP2, EGP1; NCBI Gene ID: 4070); tumor necrosis factor (TNF; NCBI Gene ID: 7124); tumor necrosis factor (TNF) receptor superfamily members (e.g., TNFRSF1A (CD120a), TNFRSFIB (CD120b), TNFRSF4 (OX40), TNFRSF5 (CD40), TNFRSF6 (CD95, FAS receptor), TNFRSF7 (CD27), TNFRSF8 (CD30), TNFRSF9 (CD137, 4-1BB), TNFRSF10A (CD261), TNFRSF10B (TRAIL, DR5, CD262), TNFRSF10C, TNFRSF10D, TNFRSF11A, TNFRSF11B (OPG), TNFRSF12A, TNFRSF13B, TNFR13C (, CD268, BAFFR), TNFRSF14 (CD270, LIGHTR), TNFRSF16, TNFRSF17 (CD269, BCMA), TNFRSF18 (GITR, CD357), TNFRSF19, TNFRSF21, TNFRSF25; NCBI Gene IDs: 355, 608, 939, 943, 958, 3604, 4804, 4982, 7132, 7133, 7293, 8718, 8764, 8784, 8792, 8793, 8794, 8795, 8797, 23495, 27242, 51330, 55504); tumor protein p53 (TP53; NCBI Gene ID: 7157); tumor suppressor 2, mitochondrial calcium regulator (TUSC2; NCBI Gene ID: 11334); TYRO3 protein tyrosine kinase (TYRO3; BYK; NCBI Gene ID: 7301); tyrosinase (TYR; NCBI Gene ID: 7299); tyrosine hydroxylase (TH; NCBI Gene ID: 7054); tyrosine kinase with immunoglobulin like and EGF like domains 1 (e.g., TIEl, TIEl; NCBI Gene ID: 7075); tyrosine-protein phosphatase non-receptor type 11 (PTPN11, SHP2; NCBI Gene ID: 5781); ubiquitin conjugating enzyme E2 I (UBE2I, UBC9; NCBI Gene ID: 7329); ubiquitin C-terminal hydrolase L5 (UCHL5; NCBI Gene ID: 51377); ubiquitin specific peptidase 7 (USP7; NCBI Gene ID: 7874); ubiquitin-like modifier activating enzyme 1 (UBA1; NCBI Gene ID: 7317); UL16 binding proteins (e.g., ULBP1, ULBP2, ULBP3; NCBI Gene ID: 79465, 80328, 80328); valosin-containing protein (VCP, CDC48; NCBI Gene ID: 7415); vascular cell adhesion molecule 1 (VCAM1, CD106; NCBI Gene ID: 7412); vascular endothelial growth factors (e.g., VEGFA, VEGFB; NCBI Gene ID: 7422, 7423); vimentin (VIM; NCBI Gene ID: 7431); vitamin D receptor (VDR; NCBI Gene ID: 7421); V-set domain containing T cell activation inhibitor 1 (VTCN1, B7-H4; NCBI Gene ID: 79679); V-set immunoregulatory receptor (VSIR, VISTA, B7-H5; NCBI Gene ID: 64115); WEE1 G2 checkpoint kinase (WEE1; NCBI Gene ID: 7465); WRN RecQ like helicase (WRN; RECQ3; NCBI Gene ID: 7486); WT1 transcription factor (WT1; NCBI Gene ID: 7490); WW domain containing transcription regulator 1 (WWTR1; TAZ; NCBI Gene ID: 25937); X—C motif chemokine ligand 1 (XCL1, ATAC; NCBI Gene ID: 6375); X—C motif chemokine receptor 1 (XCR1, GPR5, CCXCR1; NCBI Gene ID: 2829); Yesl associated transcriptional regulator (YAPi; NCBI Gene ID: 10413); zeta chain associated protein kinase 70 (ZAP70; NCBI Gene ID: 7535).

In some embodiments, the one or more additional therapeutic agents include, e.g., an agent targeting 5′-nucleotidase ecto (NTSE or CD73; NCBI Gene ID: 4907); adenosine A_{2A} receptor (ADORA2A; NCBI Gene ID: 135); adenosine A_{2B} receptor (ADORA2B; NCBI Gene ID: 136); C—C motif chemokine receptor 8 (CCR8, CDwl98; NCBI Gene ID: 1237); cytokine inducible SH2 containing protein (CISH; NCBI Gene ID: 1154); diacylglycerol kinase alpha (DGKA, DAGK, DAGK1 or DGK-alpha; NCBI Gene ID: 1606); fms like tyrosine kinase 3 (FLT3, CD135; NCBI Gene ID: 2322); integrin associated protein (IAP, CD47; NCBI Gene ID: 961); interleukine-2 (IL2; NCBI Gene ID:3558); interleukine 2 receptor (IL2RA, IL2R^{B}, IL2RG; NCBI Gene IDs: 3559, 3560, 3561); Kirsten rat sarcoma virus (KRAS; NCBI Gene ID: 3845; including mutations, such as KRAS G12C or G12D); mitogen-activated protein kinase kinase kinase kinase 1 (MAP4K1) (also called Hematopoietic Progenitor Kinase 1 (HPK1), NCBI Gene ID: 11184); myeloid cell leukemia sequence 1 apoptosis regulator (MCL1; NCBI Gene ID: 4170); phosphatidylinositol-4,5-bisphosphate 3-kinase, catalytic subunit delta (PIK3CD; NCBI Gene ID: 5293); programmed death-ligand 1 (PD-L1, CD274; NCBI Gene ID 29126); programmed cell death protein 1 (PD-1, CD279; NCBI Gene ID: 5133); proto-oncogen c-KIT (KIT, CD117; NCBI Gene ID: 3815); signal-regulatory protein alpha (SIRPA, CD172A; NCBI Gene ID: 140885); TCDD inducible poly(ADP-ribose) polymerase (TIPARP, PARP7; NCBI Gene ID: 25976); T cell immunoreceptor with Ig and ITIM domains (TIGIT; NCBI Gene ID: 201633); triggering receptor expressed on myeloid cells 1 (TREM1; NCBI Gene ID: 54210); triggering receptor expressed on myeloid cells 2 (TREM2; NCBI Gene ID: 54209); tumor-associated calcium signal transducer 2 (TACSTD2, TROP2, EGP1; NCBI Gene ID: 4070); tumor necrosis factor receptor superfamily, member 4 (TNFRSF4, CD134, OX40; NCBI Gene ID:7293); tumor necrosis factor receptor superfamily, member 9 (TNFRSF9, 4-1BB, CD137; NCBI Gene ID: 3604); tumor necrosis factor receptor superfamily, member 18 (TNFRSF18, CD357, GITR; NCBI Gene ID: 8784); WRN RecQ like helicase (WRN; NCBI Gene ID: 7486); zinc finger protein Helios (IKZF2; NCBI Gene ID: 22807).

### Illustrative Mechanisms of Action

### Immune Checkpoint Modulators

In some embodiments a compound provided herein is administered with one or more blockers or inhibitors of inhibitory immune checkpoint proteins or receptors and/or with one or more stimulators, activators or agonists of one or more stimulatory immune checkpoint proteins or receptors. In some embodiments, the immune checkpoint proteins or receptors regulate T cell responses (e.g., reviewed in Xu, et al., J Exp Clin Cancer Res. (2018) 37:110). In some embodiments, the immune checkpoint proteins or receptors regulate NK cell responses (e.g., reviewed in Davis, et al., Semin Immunol. (2017) 31:64-75 and Chiossone, et al., Nat Rev Immunol. (2018) 18(11):671-688). Inhibition of regulatory T-cells (Treg) or Treg depletion can alleviate their suppression of antitumor immune responses and have anticancer effects (e.g., reviewed in Plitas and Rudensky, Annu. Rev. Cancer Biol. (2020) 4:459-77; Tanaka and Sakaguchi, Eur. J. Immunol. (2019) 49:1140-1146).

### Cluster of Differentiation Agonists or Activators

In some embodiments, the compound provided herein is administered with agents targeting a cluster of differentiation (CD) marker.

### SIRPa Targeting Agents

In some embodiments the compound provided herein is administered with a SIRPa targeting agent (NCBI Gene ID: 140885; UniProt P78324).

### FLT3R Agonists

In some embodiments the compound provided herein is administered with a FLT3R agonist. In some embodiments, the compound provided herein is administered with a FLT3 ligand.

In some embodiments, the compound provided herein is administered with a FLT3L-Fc fusion protein.

### TNF Receptor Superfamily (TNFRSF) Member Agonists or Activators

In some embodiments, the compound provided herein is administered with an agonist of one or more TNF receptor superfamily (TNFRSF) members.

### Bi-Specific T-Cell Engagers

In some embodiments compound provided herein is administered with a bi-specific T-cell engager (e.g., not having an Fc) or an anti-CD3 bi-specific antibody (e.g., having an Fc).

### Bi- and Tri-Specific Natural Killer (NK)-Cell Engagers

In some embodiments the compound provided herein is administered with a bi-specific NK-cell engager (BiKE) or a tri-specific NK-cell engager (TriKE) (e.g., not having an Fc) or bi-specific antibody (e.g., having an Fc) against an NK cell activating receptor MCL1 apoptosis regulator, BCL2 family member (MCL1) Inhibitors

In some embodiments the compound provided herein is administered with an inhibitor of MCL1 apoptosis regulator, BCL2 family member (MCL1, TM; EAT; MCL1L; MCL1S; Mel-1; BCL2L³; MCL1-ES; bcl2-L-3; mcl1/EAT; NCBI Gene ID: 4170).

### SHP2 Inhibitors

In some embodiments compound provided herein is administered with an inhibitor of protein tyrosine phosphatase non-receptor type 11 (PTPN11; BPTP3, CFC, JMML, METCDS, NS1, PTP-1D, PTP2C, SH-PTP2, SH-PTP3, SHP2; NCBI Gene ID: 5781).

### Hematopoietic Progenitor Kinase 1 (HPK1) Inhibitors and Degraders

In some embodiments, the compound provided herein is administered with an inhibitor of mitogen-activated protein kinase kinase kinase kinase 1 (MAP4K1, HPK1; NCBI Gene ID: 11184).

### Apoptosis Signal-Regulating Kinase (ASK) Inhibitors

In some embodiments the compound provided herein is administered with an ASK inhibitor, e.g., mitogen-activated protein kinase kinase kinase 5 (MAP3K5; ASK1, MAPKKK5, MEKK5; NCBI Gene ID: 4217).

### Bruton Tyrosine Kinase (BTK) Inhibitors

In some embodiments the compound provided herein is administered with an inhibitor of Bruton tyrosine kinase (BTK, AGMX1, AT, ATK, BPK, IGHD3, IMD1, PSCTK1, XLA; NCBI Gene ID: 695).

### Cyclin-dependent Kinase (CDK) Inhibitors

In some embodiments the compound provided herein is administered with an inhibitor of cyclin dependent kinase 1 (CDK1, CDC2; CDC28A; P34CDC2; NCBI Gene ID: 983); cyclin dependent kinase 2 (CDK2, CDKN2; p33(CDK2); NCBI Gene ID: 1017); cyclin dependent kinase 3 (CDK3; NCBI Gene ID: 1018); cyclin dependent kinase 4 (CDK4, CMM3; PSK-J3; NCBI Gene ID: 1019); cyclin dependent kinase 6 (CDK6, MCPH12; PLSTIRE; NCBI Gene ID: 1021); cyclin dependent kinase 7 (CDK7, CAK; CAKI; HCAK; MO15; STK1; CDKN7; p39MO15; NCBI Gene ID: 1022), or cyclin dependent kinase 9 (CDK9, TAK; C-2k; CTK1; CDC2L⁴; PITALRE; NCBI Gene ID: 1025).

### Discoidin Domain Receptor (DDR) Inhibitors

In some embodiments the compound provided herein is combined with an inhibitor of discoidin domain receptor tyrosine kinase 1 (DDR1, CAK, CD167, DDR, EDDR1, HGK2, MCK10, NEP, NTRK4, PTK3, PTK3A, RTK6, TRKE; NCBI Gene ID: 780); and/or discoidin domain receptor tyrosine kinase 2 (DDR2, MIG20a, NTRKR3, TKT, TYRO10, WRCN; NCBI Gene ID: 4921).

### Targeted E3 Ligase Ligand Conjugates

In some embodiments the compound provided herein is administered with a targeted E3 ligase ligand conjugate. Such conjugates have a target protein binding moiety and an E3 ligase binding moiety (e.g., an inhibitor of apoptosis protein (IAP) (e.g., XIAP, c-IAP1, c-IAP2, NIL-IAP, Bruce, and surviving) E3 ubiquitin ligase binding moiety, Von Hippel-Lindau E3 ubiquitin ligase (VHL) binding moiety, a cereblon E3 ubiquitin ligase binding moiety, mouse double minute 2 homolog (MDM2) E3 ubiquitin ligase binding moiety), and can be used to promote or increase the degradation of targeted proteins, e.g., via the ubiquitin pathway.

### Histone Deacetylase (HDAC) Inhibitors

In some embodiments the compound provided herein is administered with an inhibitor of a histone deacetylase, e.g., histone deacetylase 9 (HDAC9, HD7, HD7b, HD9, HDAC, HDAC7, HDAC7B, HDAC9B, HDAC9FL, HDRP, MITR; Gene ID: 9734).

Indoleamine-pyrrole-2,3-dioxygenase (IDO1) inhibitors

In some embodiments the compound provided herein is administered with an inhibitor of indoleamine 2,3-dioxygenase 1 (IDO1; NCBI Gene ID: 3620). Janus Kinase (JAK) Inhibitors

In some embodiments, the compound provided herein is administered with an inhibitor of Janus kinase 1 (JAK1, JAK1A, JAK1B, JTK3; NCBI Gene ID: 3716); Janus kinase 2 (JAK2, JTK10, THCYT3; NCBI Gene ID: 3717); and/or Janus kinase 3 (JAK3, JAK-3, JAK3_HUMAN, JAKL, L-JAK, LJAK; NCBI Gene ID: 3718).

### Lysyl Oxidase-Like Protein (LOXL) Inhibitors

In some embodiments the compound provided herein is administered with an inhibitor of a LOXL protein, e.g., LOXL1 (NCBI Gene ID: 4016), LOXL2 (NCBI Gene ID: 4017), LOXL3 (NCBI Gene ID: 84695), LOXL4 (NCBI Gene ID: 84171), and/or LOX (NCBI Gene ID: 4015).

### Matrix Metalloprotease (MMP) Inhibitors

In some embodiments the compound provided herein is administered with an inhibitor of a matrix metallopeptidase (MMP), e.g., an inhibitor of MMP1 (NCBI Gene ID: 4312), MMP2 (NCBI Gene ID: 4313), MMP3 (NCBI Gene ID: 4314), MMP7 (NCBI Gene ID: 4316), MMP8 (NCBI Gene ID: 4317), MMP9 (NCBI Gene ID: 4318); MMP10 (NCBI Gene ID: 4319); MMP11 (NCBI Gene ID: 4320); MMP12 (NCBI Gene ID: 4321), MMP13 (NCBI Gene ID: 4322), MMP14 (NCBI Gene ID: 4323), MMP15 (NCBI Gene ID: 4324), MMP16 (NCBI Gene ID: 4325), MMP17 (NCBI Gene ID: 4326), MMP19 (NCBI Gene ID: 4327), MMP20 (NCBI Gene ID: 9313), MMP21 (NCBI Gene ID: 118856), MMP24 (NCBI Gene ID: 10893), MMP25 (NCBI Gene ID: 64386), MMP26 (NCBI Gene ID: 56547), MMP27 (NCBI Gene ID: 64066) and/or MMP28 (NCBI Gene ID: 79148).

### RAS and RAS Pathway Inhibitors

In some embodiments the compound provided herein is administered with an inhibitor of KRAS proto-oncogene, GTPase (KRAS; a.k.a., NS; NS3; CFC2; RALD; K-Ras; KRAS1; KRAS2; RASK2; KI-RAS; C—K-RAS; K-RAS2A; K-RAS2B; K-RAS4A; K-RAS4B; c-Ki-ras2; NCBI Gene ID: 3845); NRAS proto-oncogene, GTPase (NRAS; a.k.a., NS6; CMNS; NCMS; ALPS4; N-ras; NRAS1; NCBI Gene ID: 4893) or HRAS proto-oncogene, GTPase (HRAS; a.k.a., CTLO; KRAS; HAMSV; HRAS1; KRAS2; RASHI; RASK2; Ki-Ras; p2lras; C—H-RAS; c-K-ras; H-RASIDX; c-Ki-ras; C-BAS/HAS; C-HA-RAS1; NCBI Gene ID: 3265). The Ras inhibitors can inhibit Ras at either the polynucleotide (e.g., transcriptional inhibitor) or polypeptide (e.g., GTPase enzyme inhibitor) level. In some embodiments, the inhibitors target one or more proteins in the Ras pathway, e.g., inhibit one or more of EGFR, Ras, Raf (A-Raf, B-Raf, C-Raf), MEK (MEK1, MEK2), ERK, PI3K, AKT and mTOR.

### Mitogen-Activated Protein Kinase (MEK) Inhibitors

In some embodiments the compound provided herein is administered with an inhibitor of mitogen-activated protein kinase kinase 7 (MAP2K7, JNKK2, MAPKK7, MEK, MEK 7, MKK7, PRKMK7, SAPKK-4, SAPKK4; NCBI Gene ID: 5609).

Phosphatidylinositol 3-kinase (PI3K) Inhibitors

In some embodiments compounds provided herein is administered with an inhibitor of a phosphatidylinositol-4,5-bisphosphate 3-kinase catalytic subunit, e.g., phosphatidylinositol-4,5-bisphosphate 3-kinase catalytic subunit alpha (PIK3CA, CLAPO, CLOVE, CWS5, MCAP, MCM, MCMTC, PI3K, PI3K-alpha, p110-alpha; NCBI Gene ID: 5290); phosphatidylinositol-4,5-bisphosphate 3-kinase catalytic subunit beta (PIK3CB, P110BETA, PI3K, PI3KBETA, PIK3C₁; NCBI Gene ID: 5291); phosphatidylinositol-4,5-bisphosphate 3-kinase catalytic subunit gamma (PIK3CG, PI3CG, PI3K, PI3Kgamma, PIK3, p110 gamma, p120-PI3K; Gene ID: 5494); and/or phosphatidylinositol-4,5-bisphosphate 3-kinase catalytic subunit delta (PIK3CD, APDS, IMD14, P110DELTA, PI3K, p¹ 10D, NCBI Gene ID: 5293).

### Spleen Tyrosine Kinase (SYK) Inhibitors

In some embodiments the compound provided herein is administered with an inhibitor of spleen associated tyrosine kinase (SYK, p72-Syk, NCBI Gene ID: 6850).

### Toll-Like Receptor (TLR) Agonists

In some embodiments compound provided herein is administered with an agonist of a toll-like receptor (TLR), e.g., an agonist of TLR1 (NCBI Gene ID: 7096), TLR2 (NCBI Gene ID: 7097), TLR3 (NCBI Gene ID: 7098), TLR4 (NCBI Gene ID: 7099), TLR5 (NCBI Gene ID: 7100), TLR6 (NCBI Gene ID: 10333), TLR7 (NCBI Gene ID: 51284), TLR8 (NCBI Gene ID: 51311), TLR9 (NCBI Gene ID: 54106), and/or TLR10 (NCBI Gene ID: 81793).

### Tyrosine-kinase Inhibitors (TKIs)

In some embodiments the compound provided herein is administered with a tyrosine kinase inhibitor (TKI). TKIs may target epidermal growth factor receptors (EGFRs) and receptors for fibroblast growth factor (FGF), platelet-derived growth factor (PDGF), and vascular endothelial growth factor (VEGF).

### Chemotherapeutic Agents

In some embodiments the compound provided herein is administered with a chemotherapeutic agent or anti-neoplastic agent.

As used herein, the term “chemotherapeutic agent” or “chemotherapeutic” (or “chemotherapy” in the case of treatment with a chemotherapeutic agent) is meant to encompass any non-proteinaceous (e.g., non-peptidic) chemical compound useful in the treatment of cancer.

### Anti-Hormonal Agents

Also included in the definition of “chemotherapeutic agent” are anti-hormonal agents such as anti-estrogens and selective estrogen receptor modulators (SERMs), inhibitors of the enzyme aromatase, anti-androgens, and pharmaceutically acceptable salts, acids or derivatives of any of the above that act to regulate or inhibit hormone action on tumors.

### Anti-Angiogenic Agents

In some embodiments the compound provided herein is administered with an anti-angiogenic agent.

### Anti-Fibrotic Agents

In some embodiments the compound provided herein is administered with an anti-fibrotic agent.

### Anti-Inflammatory Agents

In some embodiments the compound provided herein is administered with an anti-inflammatory agent.

### Tumor Oxygenation Agents

In some embodiments the compound provided herein is administered with an agent that promotes or increases tumor oxygenation or reoxygenation, or prevents or reduces tumor hypoxia.

### Immunotherapeutic Agents

In some embodiments the compound provided herein is administered with an immunotherapeutic agent. In some embodiments the immunotherapeutic agent is an antibody.

In some embodiments, the immunotherapeutic agent is an antibody-drug conjugate (ADC). Illustrative ADCs that can be co-administered include without limitation drug-conjugated antibodies, fragments thereof, or antibody mimetics targeting the proteins or antigens listed above and herein.

### Cancer Gene Therapy and Cell Therapy

In some embodiments the compound provided herein is administered with a cancer gene therapy and cell therapy. Cancer gene therapies and cell therapies include the insertion of a normal gene into cancer cells to replace a mutated or altered gene; genetic modification to silence a mutated gene; genetic approaches to directly kill the cancer cells; including the infusion of immune cells designed to replace most of the patient's own immune system to enhance the immune response to cancer cells, or activate the patient's own immune system (T cells or Natural Killer cells) to kill cancer cells, or find and kill the cancer cells; genetic approaches to modify cellular activity to further alter endogenous immune responsiveness against cancer.

### Cellular Therapies

In some embodiments the compound provided herein is administered with one or more cellular therapies. As appropriate, a cellular therapy can entail the co-administration of cells that are autologous, syngeneic or allogeneic to the subject.

In some embodiments the cellular therapy entails co-administering cells comprising chimeric antigen receptors (CARs). In such therapies, a population of immune effector cells engineered to express a CAR, wherein the CAR comprises a tumor antigen-binding domain. In T cell therapies, the T cell receptors (TCRs) are engineered to target tumor derived peptides presented on the surface of tumor cells.

### Additional Exemplified Combination Therapies

In some embodiments the compound provided herein is administered with one or more therapeutic agents selected from a PI3K inhibitor, a Trop-2 binding agent, CD47 antagonist, a SIRPα antagonist, a FLT3R agonist, a PD-1 antagonist, a PD-L1 antagonist, an MCL1 inhibitor, a CCR8 binding agent, an HPK1 antagonist, a DGKa inhibitor, a CISH inhibitor, a PARP-7 inhibitor, a Cbl-b inhibitor, a KRAS inhibitor (e.g., a KRAS G12C or G12D inhibitor), a KRAS degrader, a beta-catenin degrader, a helios degrader, a CD73 inhibitor, an adenosine receptor antagonist, a TIGIT antagonist, a TREM1 binding agent, a TREM2 binding agent, a CD137 agonist, a GITR binding agent, an OX40 binding agent, and a CAR-T cell therapy.

### IX. Compound Preparation

In some embodiments, the present disclosure provides processes and intermediates useful for preparing the compounds disclosed herein or pharmaceutically acceptable salts thereof.

Compounds disclosed herein can be purified by any of the means known in the art, including chromatographic means, including but not limited to high-performance liquid chromatography (HPLC), preparative thin layer chromatography, flash column chromatography, and ion exchange chromatography. Any suitable stationary phase can be used, including but not limited to, normal and reversed phases as well as ionic resins. In some embodiments, the disclosed compounds are purified via silica gel and/or alumina chromatography.

During any of the processes for preparation of the compounds provided herein, it can be necessary and/or desirable to protect sensitive or reactive groups on any of the molecules concerned. This can be achieved by means of conventional protecting groups as described in standard works, such as T. W. Greene and P. G. M. Wuts, PROTECTIVE GROUPS IN ORGANIC SYNTHESIS, 4^{th} ed., Wiley, New York 2006. The protecting groups can be removed at a convenient subsequent stage using methods known from the art.

Exemplary chemical entities useful in methods of the embodiments will now be described by reference to illustrative synthetic schemes for their general preparation herein and the specific examples that follow. Skilled artisans will recognize that, to obtain the various compounds herein, starting materials can be suitably selected so that the ultimately desired substituents will be carried through the reaction scheme with or without protection as appropriate to yield the desired product. Alternatively, it can be necessary or desirable to employ, in the place of the ultimately desired substituent, a suitable group that can be carried through the reaction scheme and replaced as appropriate with the desired substituent. Furthermore, one of skill in the art will recognize that the transformations shown in the schemes below can be performed in any order that is compatible with the functionality of the particular pendant groups.

The methods of the present disclosure generally provide a specific enantiomer or diastereomer as the desired product, although the stereochemistry of the enantiomer or diastereomer was not determined in all cases. When the stereochemistry of the specific stereocenter in the enantiomer or diastereomer is not determined, the compound is drawn without showing any stereochemistry at that specific stereocenter even though the compound can be substantially enantiomerically or disatereomerically pure.

Compounds disclosed herein can be prepared from commercially available reagents using the synthetic methods and reaction schemes described herein, or using other reagents and conventional methods known to persons of ordinary skill in the art. For instance, representative syntheses of compounds of the present disclosure are described in the schemes below, and the particular examples that follow.

### EXAMPLES

### I. Abbreviations

Certain abbreviations and acronyms are used in describing the experimental details. Although most of these would be understood by one skilled in the art, Table 1 contains a list of many of these abbreviations and acronyms.

TABLE 1
List of Abbreviations and Acronyms

| Abbreviation | Meaning |
|---|---|
| ° C. | degree(s) Celsius |
| μg or ug | microgram(s) |
| μL or uL | microliter(s) |
| μm or um | micron(s) |
| μmol or umol | micromole(s) |
| aq | aqueous |
| Boc | tert-butoxycarbonyl |
| br s | broad singlet |
| cataCXium | mesylate[(di(1-adamantyl)-n-butylphosphine)-2-(2′- |
| A Pd G3 | amino-1,1′-biphenyl)]palladium(II) |
| Cbz | benzyloxycarbonyl |
| CPhos Pd | [(2-dicyclohexylphosphino-2′,6′-bis(N,N- |
| G3 | dimethylamino)-1,1′-biphenyl)-2-(2′-amino- |
| | 1,1′-biphenyl)] palladium(II) |
| | methanesulfonate |
| d | doublet |
| DCM | dichloromethane |
| dd | doublet of doublets |
| ddd | doublet of doublet of doublets |
| ddt | doublet of doublet of triplets |
| DIEA | N,N-diisopropylethylamine |
| DMF | dimethylformamide |
| DMSO | dimethyl sulfoxide |
| dt | doublet of triplets |
| Et | ethyl |
| EtOAc | ethyl acetate |
| g | gram(s) |
| h | hour(s) |
| HATU | hexafluorophosphate azabenzotriazole tetramethyl |
| | uronium |
| HPLC | high-performance liquid chromatography |
| Hz | hertz |
| ⁱPr | isopropyl |
| J | coupling constant |
| KOAc | potassium acetate |
| LAH | lithium aluminum hydride |
| LCMS | liquid chromatography mass spectrometry |
| LDA | lithium diisopropylamide |
| m | multiplet |
| M | molarity |
| Me | methyl |
| MeCN | acetonitrile |
| MeOH | methanol |
| mg | milligram(s) |
| MHz | megahertz |
| min | minute(s) |
| mL | milliliter(s) |
| mm | millimeter(s) |
| mmol | millimole(s) |
| MOMO or OMOM | methoxymethoxy |
| NaOMe | sodium methoxide |
| n-BuLi | n-butyllithium |
| NMR | nuclear magnetic resonance |
| OSEM | 2-(trimethylsilyl)ethoxymethoxy |
| OTf | trifluoromethanesulfonate |
| Piv | pivaloyl |
| qd | quartet of doublets |
| rt | room temperature |
| s | singlet |
| t | triplet |
| TBS | tert-butyldimethylsilyl |
| TBSCl | tert-Butyldimethylsilyl chloride |
| ^{t}Bu | tertbutyl |
| td | triplet of doublets |
| TES | triethylsilane |
| TF₂O or Tf₂O | trifluoromethanesulfonic anhydride |
| TFA | trifluoroacetic acid |
| THF | tetrahydrofuran |
| TIPS | triisopropylsilyl |
| TMS | trimethylsilyl |
| TR-FRET | time-resolved fluorescence energy transfer |
| tt | triplet of triplets |
| v/v | volume/volume |
| wt | weight |
| δ | parts per million referenced to residual |
| | non-deuterated solvent peak |

### II. Intermediates

### Intermediate 1-1: tert-butyl (1S,2S,5R)-2-((S)-hepta-1,6-dien-3-yl)-3,8-diazabicyclo[3.2.1]octane-8-carboxylate

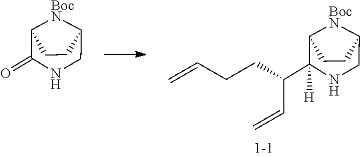 [SMILES: CC(C)(C)OC(=O)N1[C@@H]2CC[C@H]1C(=O)NC2 ; C=CCC[C@@H](C=C)[C@@H]1NC[C@H]2CC[C@@H]1N2C(=O)OC(C)(C)C]

Bis(cyclopentadienyl)zirconium(IV) chloride hydride (5.49 g, 21.2 mmol) was added to a vigorously stirred mixture of tert-butyl (1S,5R)-2-oxo-3,8-diazabicyclo[3.2.1]octane-8-carboxylate (3.00 g, 13.3 mmol) in tetrahydrofuran (22 mL) at 0° C. After 34 min, water (478 μL, 26.5 mmol) and 1-methylmorpholine (2.33 mL, 21.2 mmol) were added sequentially. After 10 min, the resulting mixture was cooled to −40° C. over 17 min. A solution of (E)-2-(hepta-2,6-dien-1-yl)-4,4,5,5-tetramethyl-1,3,2-dioxaborolane (3.98 g, 17.9 mmol) in tetrahydrofuran (5.0 mL) was added over 2 min via cannula, and the resulting mixture was allowed to warm to room temperature over 20 h. Saturated aqueous sodium carbonate solution (70 mL), ethyl ether (200 mL), 2-methyltetrahydrofuran (50 mL), and water (20 mL) were added sequentially. The biphasic mixture was agitated, and the layers were separated. The aqueous layer was extracted with 2-methyltetrahydrofuran (50 mL), and the combined organic layers were dried over anhydrous magnesium sulfate, were filtered, and were concentrated under reduced pressure. The residue was redissolved in tetrahydrofuran (34 mL) and methanol (27 mL), and the resulting mixture was vigorously stirred at room temperature. Aqueous sodium hydroxide solution (6.0 M, 13.3 mL, 80 mmol) was added via syringe. After 230 min, saturated aqueous ammonium chloride solution (20 mL) was added, and the resulting mixture was concentrated under reduced pressure to remove most of the tetrahydrofuran and methanol. Saturated aqueous sodium carbonate solution (15 mL) was added, and the aqueous layer was extracted with a mixture of ethyl ether and ethyl acetate (3:1 v:v, 2×150 mL). The combined organic layers were dried over anhydrous magnesium sulfate, were filtered, and were concentrated under reduced pressure. The residue was purified by flash column chromatography on silica gel (0 to 10% methanol in dichloromethane) to give Intermediate 1-1. LCMS: 307.2.

### Intermediate 1-2: tert-butyl (1S,2S,5R)-3-(5-bromo-7-chloro-2-(ethylthio)-8-fluoropyrido[4,3-d]pyrimidin-4-yl)-2-((S)-hepta-1,6-dien-3-yl)-3,8-diazabicyclo[3.2.1]octane-8-carboxylate

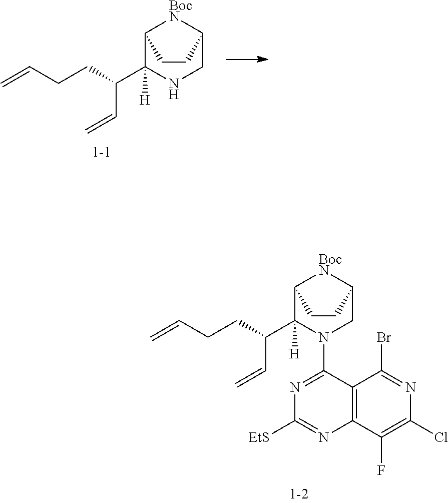

5-Bromo-4,7-dichloro-2-(ethylthio)-8-fluoropyrido[4,3-d]pyrimidine (2.28 g, 6.39 mmol) was added to a vigorously stirred mixture of Intermediate 1-1 (1.78 g, 5.81 mmol), N,N-diisopropylethylamine (1.37 mL, 7.84 mmol), and dichloromethane (8.0 mL) at room temperature. After 5 min, the resulting mixture was heated to 50° C. After 150 min, the resulting mixture was cooled to room temperature and was concentrated under reduced pressure. The residue was purified by flash column chromatography on silica gel (0 to 11% ethyl acetate in hexanes) to give Intermediate 1-2. LCMS: 626.1.

### Intermediate 1-3: tert-butyl (1S,2S,5R)-3-(5-bromo-7-chloro-2-(ethylthio)-8-fluoropyrido[4,3-d]pyrimidin-4-yl)-2-((S)-cyclopent-2-en-1-yl)-3,8-diazabicyclo[3.2.1]octane-8-carboxylate

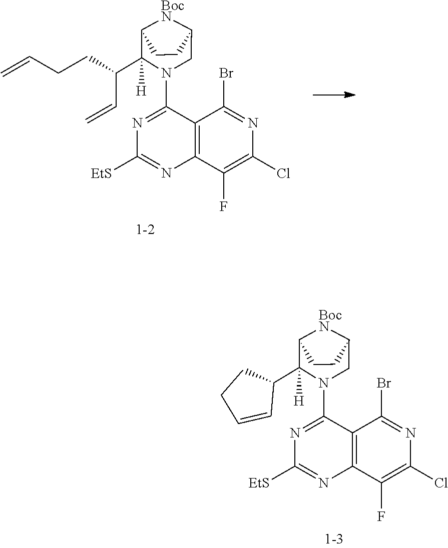

(1,3-Bis-(2,4,6-trimethylphenyl)-2-imidazolidinylidene)dichloro(o-isopropoxyphenylmethylene)ruthenium (246 mg, 392 μmol) was added to a stirred solution of Intermediate 1-2 (12.3 g, 19.6 mmol) in 1,2-dichloroethane (300 mL) at room temperature, and the resulting mixture was heated to 60° C. After 220 min, the resulting mixture was cooled to room temperature and was concentrated under reduced pressure. The residue was purified by flash column chromatography on silica gel (0 to 10% ethyl acetate in hexanes) to give Intermediate 1-3. LCMS: 598.1.

### Intermediate 1-4: tert-butyl (3aS,3bS,4S,7R,14bR)-13-chloro-10-(ethylthio)-12-fluoro-3a,3b,4,5,6,7,8,14b-octahydro-3H-8a,9,11,14,15-pentaaza-4,7-methanocyclopenta[d]naphtho[1,8-ab]heptalene-15-carboxylate

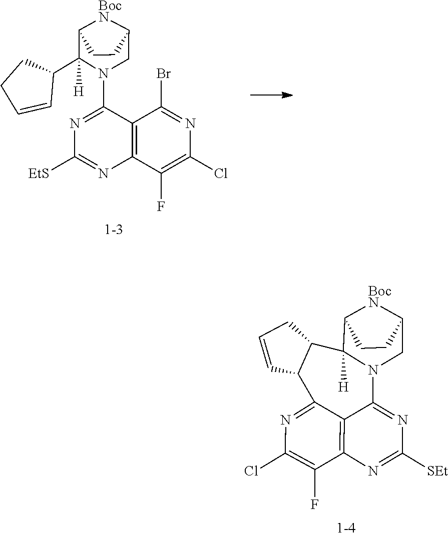

Triethylamine (308 mL, 2.21 mol) was added via syringe to a vigorously stirred mixture of Intermediate 1-3 (9.43 g, 15.7 mmol), bis(tri-tert-butylphosphine)palladium(0) (402 mg, 787 μmol), and toluene (300 mL) at room temperature, and the resulting mixture was heated to 75° C. After 30 min, the resulting mixture was heated to 83° C. After 135 min, the resulting mixture was cooled to room temperature and was concentrated under reduced pressure. The residue was purified by flash column chromatography on silica gel (0 to 25% ethyl acetate in hexanes) to give Intermediate 1-4. LCMS: 518.1.

### Intermediate 1-5: tert-butyl (3aS,3bS,4S,7R,14bR)-13-chloro-10-(ethylthio)-12-fluoro-2,3,3a,3b,4,5,6,7,8,14b-decahydro-1H-8a,9,11,14,15-pentaaza-4,7-methanocyclopenta[d]naphtho[1,8-ab]heptalene-15-carboxylate

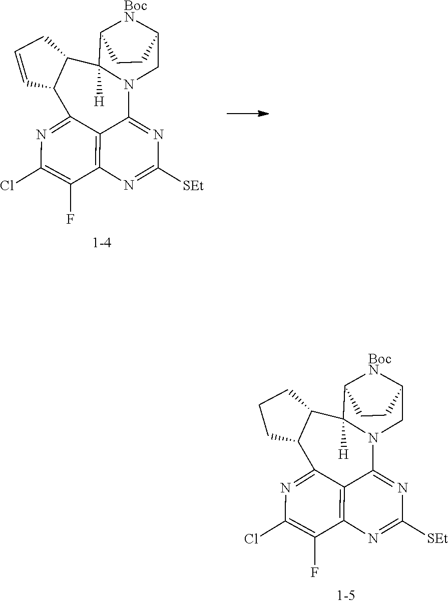

Triethylamine (6.38 mL, 45.8 mmol) was added via syringe to a stirred mixture of Intermediate 1-4 (5.93 g, 11.4 mmol), 2-nitrobenzenesulfonyl hydrazide (4.97 g, 22.9 mmol), tetrahydrofuran (80 mL), and 2-propanol (80 mL) at room temperature. After 375 min, 2-nitrobenzenesulfonyl hydrazide (7.46 g, 34.3 mmol) and triethylamine (9.57 mL, 68.7 mmol) were added sequentially. After 19 h 15 min, the resulting mixture was concentrated under reduced pressure. Ethyl ether (500 mL), ethyl acetate (50 mL), and saturated aqueous sodium carbonate solution (40 mL) were added sequentially. The organic layer was washed with water (300 mL), was dried over anhydrous magnesium sulfate, was filtered, and was concentrated under reduced pressure. The residue was purified by flash column chromatography on silica gel (0 to 15% ethyl acetate in hexanes) to give Intermediate 1-5. LCMS: 520.2.

### Intermediate 1-6: tert-butyl (3aS,3bS,4S,7R,14bR)-10-(ethylthio)-12-fluoro-13-(7-fluoro-3-(methoxymethoxy)-8-((triisopropylsilyl)ethynyl)naphthalen-1-yl)-2,3,3a,3b,4,5,6,7,8,14b-decahydro-1H-8a,9,11,14,15-pentaaza-4,7-methanocyclopenta[d]naphtho[1,8-ab]heptalene-15-carboxylate

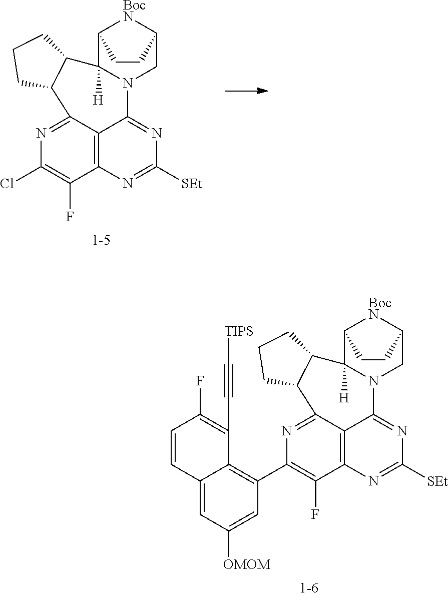

Aqueous potassium phosphate solution (2.0 M, 8.67 mL, 17 mmol) was added via syringe to a vigorously stirred mixture of Intermediate 1-5 (3.00 g, 5.78 mmol), ((2-fluoro-6-(methoxymethoxy)-8-(4,4,5,5-tetramethyl-1,3,2-dioxaborolan-2-yl)naphthalen-1-yl)ethynyl)triisopropylsilane (3.11 g, 6.07 mmol), [(di(1-adamantyl)-butylphosphine)-2-(2′-amino-1,1′-biphenyl)]palladium(II) methanesulfonate (421 mg, 578 μmol), and tetrahydrofuran (10 mL) at room temperature, and the resulting mixture was heated to 70° C. After 120 min, the resulting mixture was cooled to room temperature, and ethyl ether (100 mL) and ethyl acetate (25 mL) were added sequentially. The organic layer was washed with a mixture of water and brine (2:1 v:v, 60 mL), was dried over anhydrous magnesium sulfate, was filtered, and was concentrated under reduced pressure. The residue was purified by flash column chromatography on silica gel (0 to 25% ethyl acetate in hexanes) to give Intermediate 1-6. LCMS: 870.4.

### Intermediate 1-7: tert-butyl (3aS,3bS,4S,7R,14bR)-10-(ethylsulfonyl)-12-fluoro-13-(7-fluoro-3-(methoxymethoxy)-8-((triisopropylsilyl)ethynyl)naphthalen-1-yl)-2,3,3a,3b,4,5,6,7,8,14b-decahydro-1H-8a,9,11,14,15-pentaaza-4,7-methanocyclopenta[d]naphtho[1,8-ab]heptalene-15-carboxylate

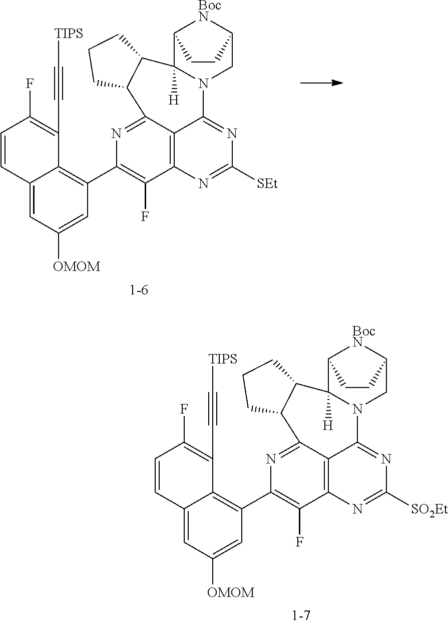

3-Chloroperbenzoic acid (77% wt, 2.53 g, 11 mmol) was added in three equal portions over 5 min to Intermediate 1-6 (4.80 g, 5.52 mmol) in dichloromethane (10 mL) at 0° C. After 2 min, the resulting mixture was warmed to room temperature. After 35 min, the resulting mixture was purified by flash column chromatography on silica gel (0 to 65% ethyl acetate in hexanes) to give Intermediate 1-7. LCMS: 902.3.

### Intermediate 1-8: (R)-1-tert-butyl 3-methyl 3-methyl-4-oxopiperidine-1,3-dicarboxylate

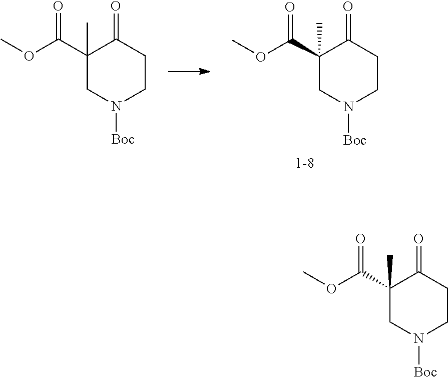 [SMILES: COC(=O)C1(C)CN(C(=O)OC(C)(C)C)CCC1=O ; COC(=O)[C@]1(C)CN(C(=O)OC(C)(C)C)CCC1=O ; COC(=O)[C@@]1(C)CN(C(=O)OC(C)(C)C)CCC1=O]

1-tert-Butyl 3-methyl 3-methyl-4-oxopiperidine-1,3-dicarboxylate (25.0 g, 0.0921 mol) was separated by SFC (SFC:column: DAICEL CHIRALPAK IG(250 mm*50 mm, 10 um); mobile phase: [A: CO₂; B: MeOH (0.1% NH₃H₂O)]; B %: 11.00%-11.00%, 5.00 min). The peak with Rt (Rt =0.635) was assigned as Intermediate 1-8. The peak with Rt (Rt=0.837) was assigned as (S)-1-tert-butyl 3-methyl 3-methyl-4-oxopiperidine-1,3-dicarboxylate.

### Intermediate 1-9: 1-(tert-butyl) 3-methyl (S,E)-4-(fluoromethylene)-3-methylpiperidine-1,3-dicarboxylate

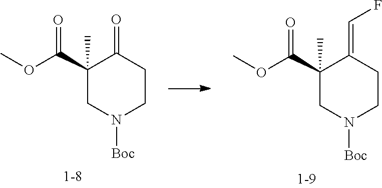 [SMILES: COC(=O)[C@]1(C)CN(C(=O)OC(C)(C)C)CCC1=O ; COC(=O)[C@]1(C)CN(C(=O)OC(C)(C)C)CC/C1=C\F]

To the mixture of fluoromethyl(triphenyl)phosphonium tetrafluoroborate (10.6 g, 0.0276 mol) in tetrahydrofuran (80.0 mL) was added potassium tert-butoxide solution (1000 mmol/L, 27.6 mL, 0.0276 mol) at −70° C. Then a solution of Intermediate 1-8 (5.00 g, 0.0184 mol) in tetrahydrofuran (30.0 mL) was added to the mixture. The mixture was stirred at 20° C. for 12 hrs under N₂. The reaction mixture was quenched by addition saturated aqueous NH₄Cl solution (100 mL) at 0° C., and then extracted with EtOAc (100 mL*3). The combined organic layers were dried over Na₂SO₄, filtered and concentrated under reduced pressure to give a residue. The residue was purified by flash silica gel chromatography (20 g Silica Flash Column, Eluent of 0 to 30% Ethyl acetate/Petroleum ether gradient at 60 mL/min) to give Intermediate 1-9. LCMS: 232.0.

### Intermediate 1-10: methyl (S,E)-4-(fluoromethylene)-3-methylpiperidine-3-carboxylate

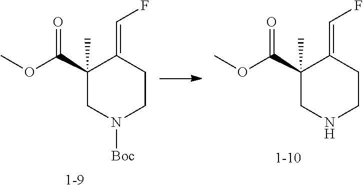 [SMILES: COC(=O)[C@]1(C)CN(C(=O)OC(C)(C)C)CC/C1=C\F ; COC(=O)[C@]1(C)CNCC/C1=C\F]

To a mixture of Intermediate 1-9 (3.20 g, 0.0111 mol) in ethyl acetate (10.0 mL) was added hydrochloric acid (10.0 mL). The mixture was stirred at 25° C. for 0.2 hr. The reaction mixture was concentrated under reduced pressure to give Intermediate 1-10 hydrochloride. LCMS: 188.1.

### Intermediate 1-11: methyl (S,E)-4-(fluoromethylene)-1,3-dimethylpiperidine-3-carboxylate

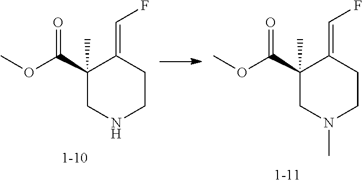

At 25° C., Intermediate 1-10 (2.10 g, 0.0112 mol) was dissolved in methanol (20.0 mL), and triethylamine was added to the reaction solution until pH-6 was obtained, and the resulting mixture was stirred for 10 minutes. Formaldehyde solution (37.0%, 2.73 g, 0.0337 mol) was added into the reaction solution and stirred at 25° C. for 30 mins. Sodium cyanoborohydride (0.775 g, 0.0123 mol) was added to the reaction solution and stirred at 25° C. for 2 hrs. The reaction mixture was concentrated under reduced pressure to give a residue. The residue was purified by flash silica gel chromatography (20 g Silica Flash Column, Eluent of 0 to 10% Ethyl acetate/DCM ether gradient at 80 mL/min) to give Intermediate 1-11. LCMS: 202.1.

### Intermediate 1-12: (S,E)-(4-(fluoromethylene)-1,3-dimethylpiperidin-3-yl)methanol

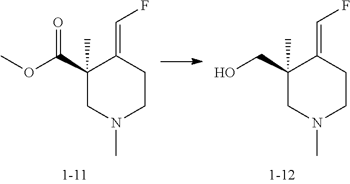

To the mixture of Intermediate 1-11 (0.800 g, 0.00398 mol) in tetrahydrofuran (10.0 mL) was added lithium aluminum hydride (2500 mmol/L, 2.39 mL, 0.00596 mol). The mixture was stirred at 25° C. for 1 hr under N₂. Na₂SO₄·10H₂O (3 g) was added slowly to quench the unreacted hydride reagents, at 0° C., and the resulting inhomogeneous mixture was stirred 12 hrs under N₂. Then the mixture was filtered and collected filtrate was concentrated under reduced pressure to give Intermediate 1-12. LCMS: 174.1.

### Intermediate 1-13: tert-butyl (3aS,3bS,4S,7R,14bR)-13-(8-ethynyl-7-fluoro-3-(methoxymethoxy)naphthalen-1-yl)-12-fluoro-10-(((S,E)-4-(fluoromethylene)-1,3-dimethylpiperidin-3-yl)methoxy)-2,3,3a,3b,4,5,6,7,8,14b-decahydro-1H-8a,9,11,14,15-pentaaza-4,7-methanocyclopenta[d]naphtho[1,8-ab]heptalene-15-carboxylate

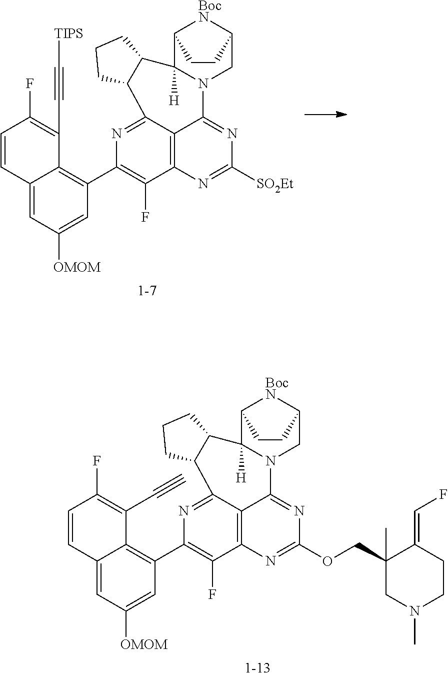

Lithium bis(trimethylsilyl)amidesolution (1.0 M in tetrahydrofuran, 15.4 μL, 15 μmol) was added via syringe to a vigorously stirred mixture of Intermediate 1-7 (11.6 mg, 12.9 μmol), Intermediate 1-12 (2.7 mg, 15 μmol), and 2-methyltetrahydrofuran (0.20 mL) at room temperature, and the resulting mixture was heated to 40° C. After 40 min, the resulting mixture was cooled to room temperature over 4 min. 1,1,1,3,3,3-Hexafluoropropan-2-ol (6.8 μL, 64 μmol), cesium fluoride (29.3 mg, 193 μmol), and N,N-dimethylformamide (1.0 mL) were added sequentially, and the resulting mixture was heated to 50° C. After 30 min, the resulting mixture was cooled to room temperature, and saturated aqueous sodium bicarbonate solution (5 mL), saturated aqueous sodium carbonate solution (5 mL), ethyl ether (40 mL), and ethyl acetate (20 mL) were added sequentially. The organic layer was washed with with water (2×30 mL), was dried over anhydrous magnesium sulfate, was filtered, and was concentrated under reduced pressure to give Intermediate 1-13. LCMS: 825.2.

### Intermediate 3-0: 5-bromo-6,8-difluoro-2-(methylthio)quinazolin-4(3H)-one

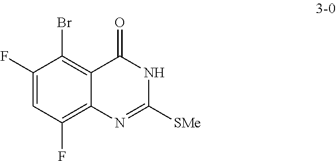 [SMILES: CSc1nc2c(F)cc(F)c(Br)c2c(=O)[nH]1]

Intermediate 3-0 was synthesized in a manner similar to Intermediate 10-3 using 2-amino-6-bromo-3,5-difluorobenzoic acid instead of Intermediate 10-1. LCMS: 307.0.

### Intermediate 3-1: tert-butyl (1S,2R,5R)-2-allyl-3-(5-bromo-6,8-difluoro-2-(methylthio)quinazolin-4-yl)-3,8-diazabicyclo[3.2.1]octane-8-carboxylate

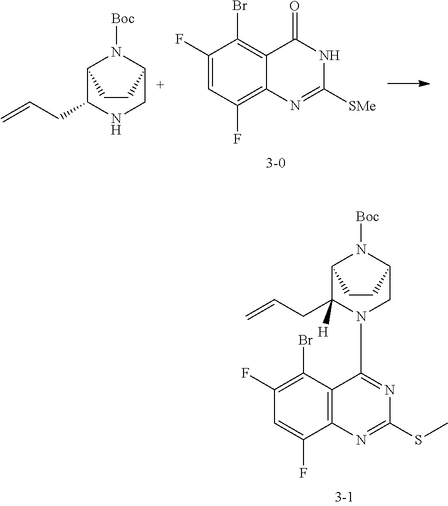

To a reaction mixture of Intermediate 3-0 (1.3 g, 4.2 mmol) and 2-(7-Aza-1H-benzotriazole-1-yl)-1,1,3,3-tetramethyluroniumhexafluorophosphate (HATU) (2.48 g, 6.5 mmol) in 25 ml of acetonitrile was added N,N-diisopropylethylamine (2.2 ml, 13 mmol). After stirring at room temperature for 1 hour, to this mixture was added a solution of tert-butyl (1S,2R,5R)-2-allyl-3,8-diazabicyclo[3.2.1]octane-8-carboxylate (1.07 g, 4.2 mmol) in 5 ml of acetonitrile. The reaction was stirred at room temperature for 1 day. Upon completion, the reaction mixture was partitioned between EtOAc and water, and the aqueous layer was extracted with EtOAc twice. The combined organic layer was washed with brine, filtered, and concentrated to dryness. The residue was purified by silica gel chromatography eluting with EtOAc/hexane to afford the title product. LCMS: 543.1.

### Intermediate 3-2: tert-butyl (1S,4R,15aR)-10,12-difluoro-8-(methylthio)-2,3,4,5,13,14,15,15a-octahydro-1H-1,4-epiminoazepino[1′,2′:1,8]azocino[2,3,4-de]quinazoline-16-carboxylate

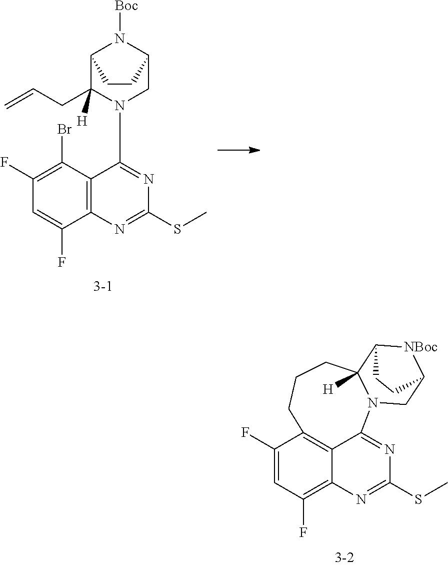

A solution of Intermediate 3-1 (1.0 g, 1.85 mmol; azeotroped with toluene 3 x) dissolved in 0.5 M 9-BBN in THE (11.1 mL, 5.55 mmol). The resulting solution was stirred at room temperature for 1 hour until Intermediate 3-1 was consumed. The reaction solution was cooled to 0° C. as 1.5 M K₃PO₄ (6.2 mL, 9.3 mmol) was added and, after 5 minutes stirring, was added cataCXium A Pd G3 (134.91 mg, 185 μmol) and THF (22.7 mL) at 0° C. The resulting mixture was purged with Argon gas for 15 minutes at room temperature, then stirred at 60° C. in preheated oil bath. After 30 minutes, the reaction mixture was cooled to room temperature and diluted with ethyl acetate (˜120 mL) before washing with brine (120 mL). After the aqueous fraction was extracted with EtOAc (100 mL×1), combined organic layer was dried (MgSO₄), and concentrated. The residue was purified by silica gel chromatography eluting with EtOAc/hexane to afford the title product. LCMS: 463.3.

### Intermediate 3-3: tert-butyl (1S,4R,15aR)-11-bromo-10,12-difluoro-8-(methylthio)-2,3,4,5,13,14,15,15a-octahydro-1H-1,4-epiminoazepino[1′,2′:1,8]azocino[2,3,4-de]quinazoline-16-carboxylate

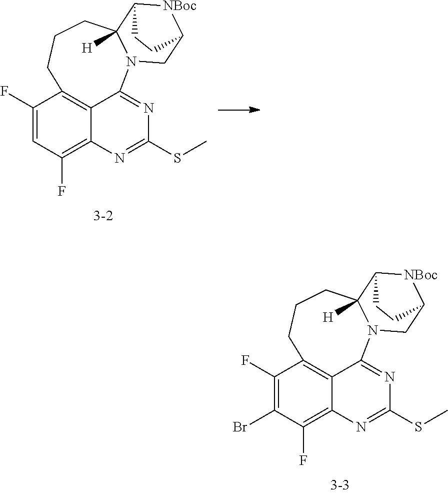

Intermediate 3-2 (1.0 g. 2.16 mmol) in a dry flask was evacuated under vacuum and filled with nitrogen three times. The bis(2,2,6,6-tetramethylpiperidinyl)zinc, lithium chloride, and magnesium chloride complex ((TMP)₂Zn·2 MgCl₂·2 LiCl) (16.6 mL of a 0.26 M solution in THF, 4.32 mmol) was added and the reaction mixture was allowed to stir for 2 hours at room temperature. The mixture was cooled to 0-5° C. under nitrogen. Bromine (2.22 mL of 10% solution in THF, 4.32 mmol) was added dropwise into the reaction mixture and stirred at 0-5° C. for 0.5 hour. Upon completion, the reaction was quenched with half saturated NH₄Cl aqueous solution, and extracted with ethyl acetate three times; and filtered through a pad of celite. The organic layer was combined and washed with brine, dried over Na₂SO₄, filtered and concentrated. The residue was purified by silica gel chromatography eluting with EtOAc/hexane to afford the title product. LCMS: 543.2.

### Intermediate 3-4: tert-butyl (1S,4R,15aR)-10,12-difluoro-11-(7-fluoro-3-(methoxymethoxy)-8-((triisopropylsilyl)ethynyl)naphthalen-1-yl)-8-(methylthio)-2,3,4,5,13,14,15,15a-octahydro-1H-1,4-epiminoazepino[1′,2′:1,8]azocino[2,3,4-de]quinazoline-16-carboxylate

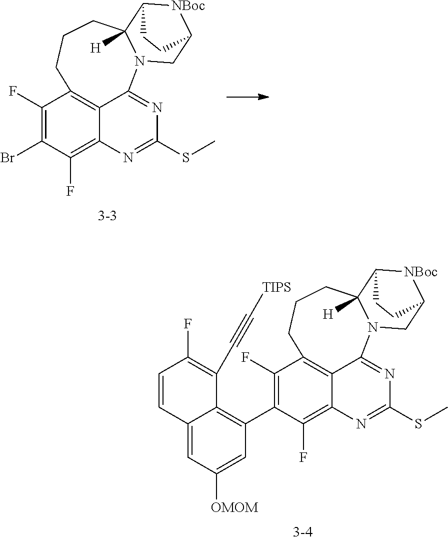

Intermediate 3-4 was synthesized in a manner similar to Intermediate 1-6 using Intermediate 3-3 instead of Intermediate 1-5. LCMS: 847.5.

### Intermediate 3-5B: tert-butyl (1S,4R,15aR)-10,12-difluoro-11-((R)-7-fluoro-3-(methoxymethoxy)-8-((triisopropylsilyl))naphthalen-1-yl)-8-(methylsulfonyl)-2,3,4,5,13,14,15,15a-octahydro-1H-1,4-epiminoazepino[1′,2′:1,8]azocino[2,3,4-de]quinazoline-16-carboxylate

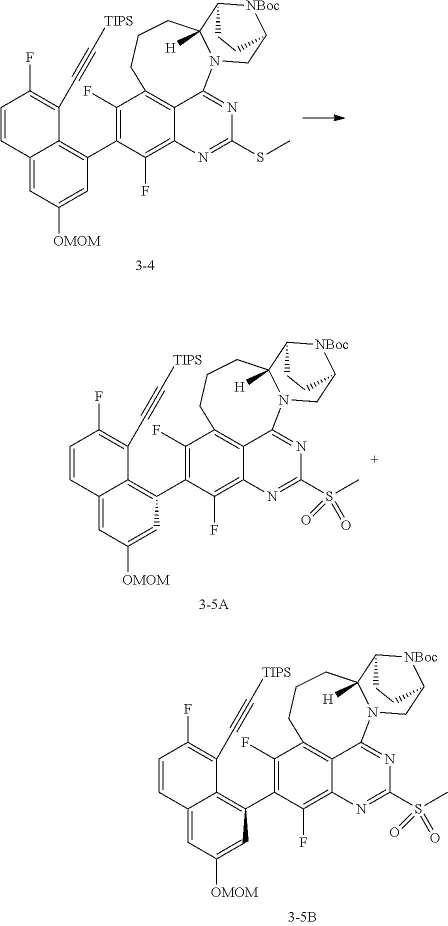

3-Chloroperbenzoic acid (77% wt, 821 mg, 3.66 mmol) was added portion wise to Intermediate 3-4 (1.41 g, 1.66 mmol) in dichloromethane (5 mL) at 0° C. The resulting mixture was stirred for 1 hour. Upon completion, the resulting mixture was purified by flash column chromatography on silica gel (0 to 50% ethyl acetate in hexanes) to give Intermediate 3-5A (early eluent peak from silica gel column) and Intermediate 3-5B (late eluent peak from silica gel column). LCMS: 879.4.

### Intermediate 3-6: tert-butyl (1S,4R,15aR)-10,12-difluoro-11-((R)-7-fluoro-3-(methoxymethoxy)-8-((triisopropylsilyl)ethynyl)naphthalen-1-yl)-8-(((S,E)-4-(fluoromethylene)-1,3-dimethylpiperidin-3-yl)methoxy)-2,3,4,5,13,14,15,15a-octahydro-1H-1,4-epiminoazepino[1′,2′:1,8]azocino[2,3,4-de]quinazoline-16-carboxylate

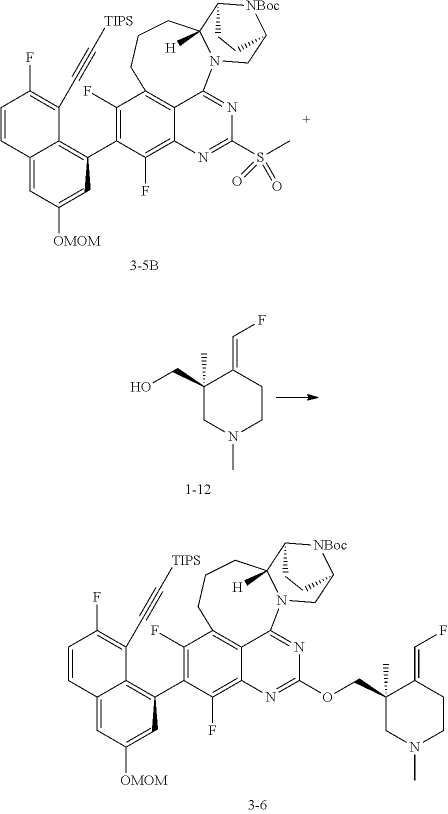

Lithium bis(trimethylsilyl)amide solution (1.0 M in tetrahydrofuran, 114 μL, 114 μmol) was added via syringe to a vigorously stirred mixture of Intermediate 3-5B (50 mg, 56.9 μmol), Intermediate 1-12 (14.8 mg, 85 μmol), and tetrahydrofuran (1 mL) at 0° C., and the resulting mixture was stirred for 1 hour. The reaction was quenched by adding saturated NH₄Cl aqueous solution followed by extraction with EtOAc. The organic layer was washed with brine, filtered, and concentrated to dryness. The residue was purified by alumina basic column eluting with EtOAc/hexane to afford the title product. LCMS: 972.6.

### Intermediate 3-7: tert-butyl (1S,4R,15aR)-11-((R)-8-ethynyl-7-fluoro-3-(methoxymethoxy)naphthalen-1-yl)-10,12-difluoro-8-(((S,E)-4-(fluoromethylene)-1,3-dimethylpiperidin-3-yl)methoxy)-2,3,4,5,13,14,15,15a-octahydro-1H-1,4-epiminoazepino[1′,2′:1,8]azocino[2,3,4-de]quinazoline-16-carboxylate

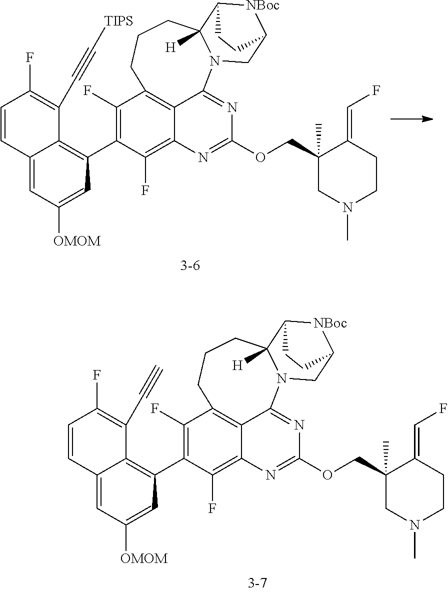

Intermediate 3-6 (50 mg, 51 μmol) was dissolved in N,N-dimethylformamide (0.5 mL), to it was added cesium fluoride (86.4 mg, 569 μmol), and the resulting mixture was stirred at room temperature for 1 hour. Upon completion, the mixture was filtered through a pad of celite and washed with dichloromethane. The filtrate was concentrated to dryness and the residue was purified by RP-HPLC (10% to 80% 0.1% TFA in MeCN/0.1% TFA in H₂O). Fractions containing the product were pooled and lyophilized to yield the title compound. LCMS: 816.4.

### Intermediate 4-1: tert-butyl (1S,2S,5R)-2-((S)-but-3-en-2-yl)-3,8-diazabicyclo[3.2.1]octane-8-carboxylate

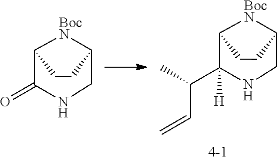 [SMILES: CC(C)(C)OC(=O)N1[C@@H]2CC[C@H]1C(=O)NC2 ; C=C[C@H](C)[C@@H]1NC[C@H]2CC[C@@H]1N2C(=O)OC(C)(C)C]

Intermediate 4-1 was synthesized in a manner similar to Intermediate 1-1 using (E)-2-(but-2-en-1-yl)-4,4,5,5-tetramethyl-1,3,2-dioxaborolane instead of (E)-2-(hepta-2,6-dien-1-yl)-4,4,5,5-tetramethyl-1,3,2-dioxaborolane. LCMS: 267.1.

### Intermediate 4-2: tert-butyl (1S,2S,5R)-3-(5-bromo-7-chloro-2-(ethylthio)-8-fluoropyrido[4,3-d]pyrimidin-4-yl)-2-((S)-but-3-en-2-yl)-3,8-diazabicyclo[3.2.1]octane-8-carboxylate

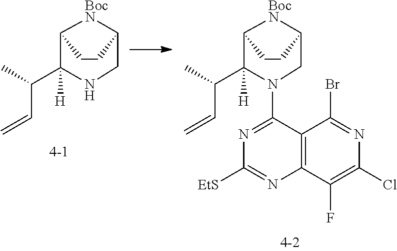

Intermediate 4-2 was synthesized in a manner similar to Intermediate 1-2 using Intermediate 4-1 instead of Intermediate 1-1. LCMS: 586.2.

### Intermediate 4-3: tert-butyl (8S,8aS,9S,12R)-5-chloro-2-(ethylthio)-4-fluoro-8-methyl-7-methylene-7,8,8a,9,10,11,12,13-octahydro-1,3,6,13a,14-pentaaza-9,12-methanonaphtho[1,8-ab]heptalene-14-carboxylate

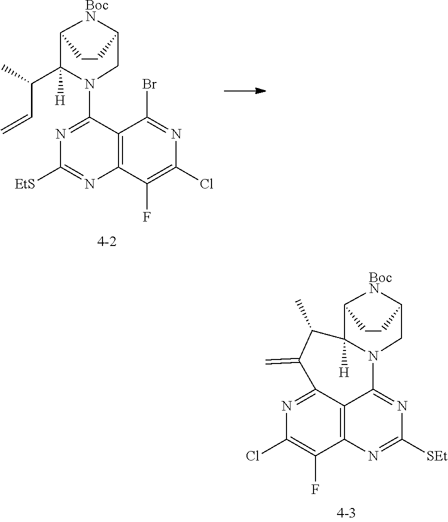

Triethylamine (2.67 mL, 19.2 mmol) was added via syringe to a vigorously stirred mixture of Intermediate 4-2 (6.62 g, 11.3 mmol), bis(tri-tert-butylphosphine)palladium(0) (284 mg, 338 μmol), and heptane (115 mL) at room temperature, and the resulting mixture was heated to 70° C. After 45 min, the resulting mixture was cooled to room temperature, and diethyl ether (150 mL) and hexanes (80 mL) were added sequentially. The organic layer was washed with a mixture of water and brine (2:1 v:v, 75 mL), was dried over anhydrous magnesium sulfate, was filtered, and was concentrated under reduced pressure. The residue was purified by flash column chromatography on silica gel (0 to 3.5% ethyl acetate in toluene) to give Intermediate 4-3. LCMS: 506.2.

### Intermediate 4-4: tert-butyl (7R,8S,8aS,9S,12R)-5-chloro-2-(ethylthio)-4-fluoro-7,8-dimethyl-7,8,8a,9,10,11,12,13-octahydro-1,3,6,13a,14-pentaaza-9,12-methanonaphtho[1,8-ab]heptalene-14-carboxylate

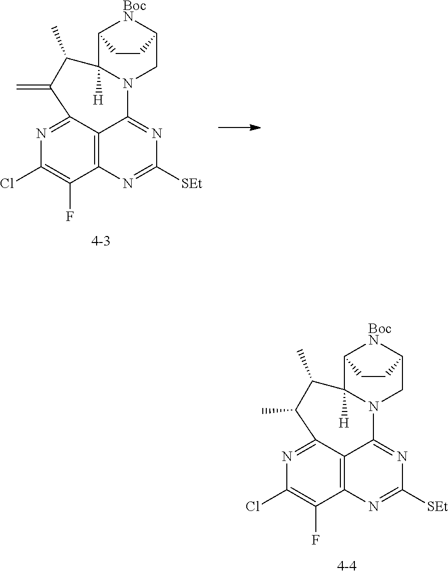

A vigorously stirred mixture of Intermediate 4-3 (3.21 g, 6.34 mmol), p-toluenesulfonyl hydrazide (5.91 g, 31.7 mmol), sodium acetate (5.20 g, 63.4 mmol), 1,4-dioxane (90 mL), and water (45 mL) was heated to 80° C. After 103 min, p-toluenesulfonyl hydrazide (5.91 g, 31.7 mmol) and sodium acetate (3.90 g, 47.6 mmol) were added sequentially. After 255 min, the resulting mixture was cooled to room temperature and was concentrated under reduced pressure to remove most of the 1,4-dioxane. Ethyl ether (200 mL), ethyl acetate (50 mL), and aqueous phosphoric acid solution (85% wt, 12 mL) were added sequentially. The organic layer was washed sequentially with water (300 mL) and a mixture of water and saturated aqueous sodium carbonate solution (300 mL), was dried over anhydrous magnesium sulfate, was filtered, and was concentrated under reduced pressure. The residue was purified by flash column chromatography on silica gel (0 to 8% ethyl acetate in hexanes) to give Intermediate 4-4. LCMS: 508.2.

### Intermediate 4-5: tert-butyl (7R,8S,8aS,9S,12R)-2-(ethylsulfonyl)-4-fluoro-5-(7-fluoro-3-(methoxymethoxy)-8-((triisopropylsilyl)ethynyl)naphthalen-1-yl)-7,8-dimethyl-7,8,8a,9,10,11,12,13-octahydro-1,3,6,13a,14-pentaaza-9,12-methanonaphtho[1,8-ab]heptalene-14-carboxylate

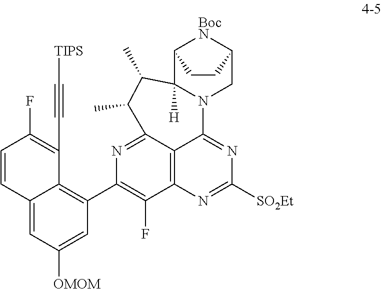

Intermediate 4-5 was synthesized in a manner similar to Intermediate 1-7 using Intermediate 4-4 instead of Intermediate 1-5. LCMS: 890.2.

### Intermediate 5-1: tert-butyl (6aR,7S,10R)-2-(8-ethynyl-7-fluoro-3-(methoxymethoxy)naphthalen-1-yl)-1-fluoro-13-(((S,E)-4-(fluoromethylene)-1,3-dimethylpiperidin-3-yl)methoxy)-5,6,6a,7,8,9,10,11-octahydro-4H-3,11a,12,14,15-pentaaza-7,10-methanocyclohepta[4,5]cycloocta[1,2,3-de]naphthalene-15-carboxylate

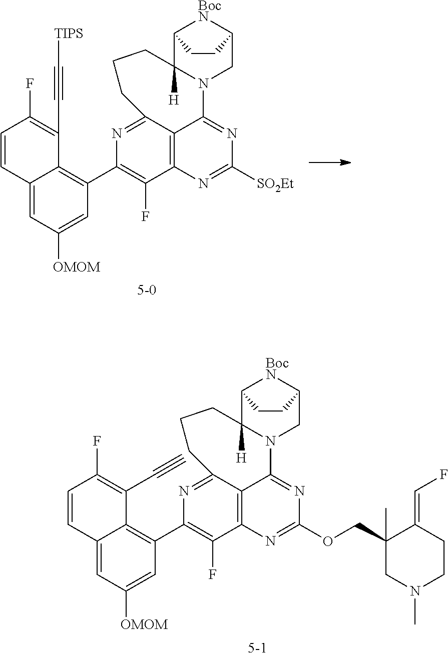

Lithium bis(trimethylsilyl)amide solution (1.0 M in tetrahydrofuran, 120 μL, 120 μmol) was added over 1 min via syringe to a vigorously stirred solution of Intermediate 5-0 (prepared according to US20230374036, 100 mg, 114 μmol) in 2-methyltetrahydrofuran (0.3 mL) at room temperature, and the resulting mixture was heated to 40° C. After 35 min, the resulting mixture was cooled to room temperature over 3 min, and 1,1,1,3,3,3-hexafluoropropan-2-ol (50.5 μL, 479 μmol), cesium fluoride (79.4 mg, 2.28 mmol), and N,N-dimethylformamide (1.0 mL) were added sequentially. The resulting mixture was heated to 60° C. After 30 min, the resulting mixture was cooled to room temperature, and saturated aqueous sodium bicarbonate solution (5.0 mL), saturated aqueous sodium carbonate solution (5.0 mL), ethyl ether (40 mL), and ethyl acetate (20 mL) were added sequentially. The organic layer was washed with water (2×30 mL), was dried over anhydrous magnesium sulfate, was filtered, and was concentrated under reduced pressure. The residue was purified by flash column chromatography on basic alumina (0 to 80% ethyl acetate in hexanes) to give Intermediate 5-1. LCMS: 799.2.

### Intermediate 5-2: (6aR,7S,10R)-2-(8-ethynyl-7-fluoro-3-(methoxymethoxy)naphthalen-1-yl)-1-fluoro-13-(((S,E)-4-(fluoromethylene)-1,3-dimethylpiperidin-3-yl)methoxy)-5,6,6a,7,8,9,10,11-octahydro-4H-3,11a,12,14,15-pentaaza-7,10-methanocyclohepta[4,5]cycloocta[1,2,3-de]naphthalene

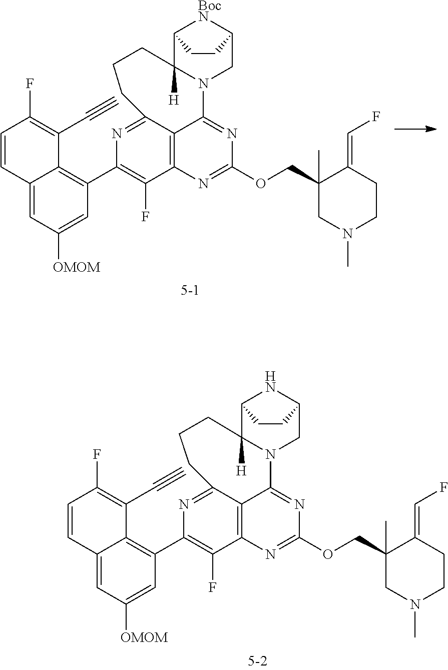

Trimethylsilyl trifluoromethanesulfonate (620 μL, 3.42 mmol) was added over 1 min via syringe to a stirred mixture of Intermediate 5-1 (1.82 g, 2.28 mmol), 2,6-lutidine (425 μL, 3.65 mmol), and dichloromethane (3.0 mL) at 0° C. After 40 min, methanol (3.0 mL) was added via syringe, and the resulting mixture was warmed to room temperature. After 10 min, the resulting mixture was concentrated under reduced pressure. The residue was purified by flash column chromatography on silica gel (0 to 40% [8:1 v:v ethanol:30% wt aqueous ammonia] in dichloromethane) to give Intermediate 5-2. LCMS: 699.2.

### Intermediate 5-3: 4-(((6aR,7S,10R)-2-(8-ethynyl-7-fluoro-3-(methoxymethoxy)naphthalen-1-yl)-1-fluoro-13-(((S,E)-4-(fluoromethylene)-1,3-dimethylpiperidin-3-yl)methoxy)-5,6,6a,7,8,9,10,11-octahydro-4H-3,11a,12,14,15-pentaaza-7,10-methanocyclohepta[4,5]cycloocta[1,2,3-de]naphthalen-15-yl)methyl)-5-methyl-1,3-dioxol-2-one

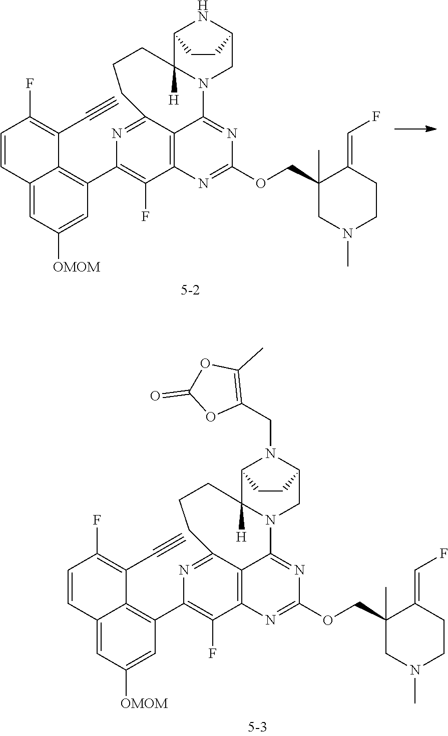

4-(Bromomethyl)-5-methyl-1,3-dioxol-2-one (168 μL, 1.52 mmol) was added over 1 min via syringe to a vigorously stirred mixture of Intermediate 5-2 (1.01 g, 1.45 mmol), potassium carbonate (1.20 g, 8.68 mmol), and N,N-dimethylformamide (3.0 mL) at 0° C. After 100 min, acetic acid (845 microliters), ethyl ether (125 mL), and ethyl acetate (25 mL) were added sequentially, and the resulting mixture was agitated. Saturated aqueous sodium bicarbonate (5 mL) was added, and the organic layer was washed with water (2×90 mL), was dried over anhydrous magnesium sulfate, was filtered, and was concentrated under reduced pressure. The residue was purified by flash column chromatography on silica gel (0 to 100% ethyl acetate in hexanes, each containing 3% triethylamine) to give Intermediate 5-3. LCMS: 811.2.

### Intermediate 6-2: tert-butyl (1S,2S,5R)-2-(prop-1-en-2-yl)-3,8-diazabicyclo[3.2.1]octane-8-carboxylate

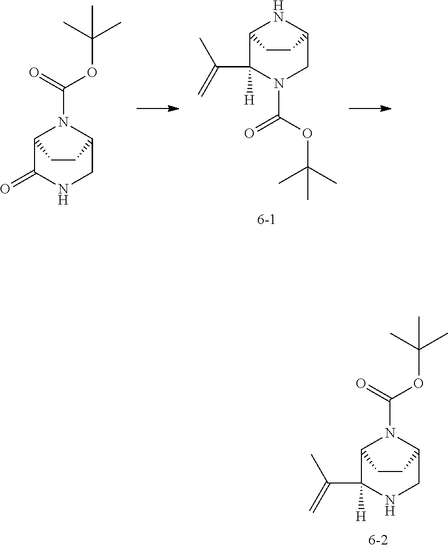

To a solution of tert-butyl (1S,5R)-2-oxo-3,8-diazabicyclo[3.2.1]octane-8-carboxylate (21 g, 92.8 mmol, 1.0 equiv.) in 336 mL of tetrahydrofuran with an inert atmosphere of argon was added bis(cyclopentadienyl)zirconium(IV) chloride hydride (38.2 g, 148.5 mmol, 1.6 equiv) at 0° C. After 15 min, the resulting mixture was cooled to −30° C. and isopropenylmagnesium bromide solution (0.5 M in tetrahydrofuran, 556.8 mL, 278.5 mmol, 3.0 equiv) was added over 10 min. The reaction mixture was stirred for 2.5 h at 0° C. then added isopropenylmagnesium bromide solution (0.5 M in tetrahydrofuran, 92.8 mL, 46.4 mmol, 0.5 equiv.). The reaction mixture was stirred for 2.5 h at 0° C. After 150 min, saturated aqueous ammonium chloride solution (60 mL), aqueous potassium phosphate solution (2.0 M, 400 mL), and ethyl acetate (500 mL) were added sequentially. The resulting mixture was warmed to room temperature and was filtered through celite. The filter cake was extracted with ethyl acetate (200 mL), and the combined filtrates were washed with 2×200 mL brine. The organic layer was dried over anhydrous magnesium sulfate, was filtered, and was concentrated under reduced pressure. The residue was purified applied onto a silica gel column with dichloromethane/methanol (100:1-10:1). 12.3 g (52% yield) of Intermediate 6-1 was obtained as a colorless oil. MS m/z [M+H]⁺ (ESI): 253.30. ¹H NMR (400 MHz, Methanol-d4) δ 5.01-4.99 (m, 1H), 4.68-4.66 (m, 1H), 4.13 (d, J=21.3 Hz, 1H), 3.70-3.57 (m, 2H), 3.45-3.35 (m, 1H), 3.17-3.05 (m, 1H), 2.02-1.89 (m, 1H), 1.88-1.80 (m, 1H), 1.78 (s, 3H), 1.75-1.60 (m, 2H), 1.43 (d, J=19.4 Hz, 9H).

To a solution of Intermediate 6-1 (8.1 g, 32.098 mmol, 1.0 equiv.) in 64.8 mL of Hydrogen chloride solution (4.0 M in 1,4-dioxane, 102.1 mmol) at room temperature. The reaction mixture was stirred for 1 h at 55° C. The resulting mixture was cooled to room temperature and was concentrated under reduced pressure. Dichloromethane (81 mL) and N,N-diisopropylethylamine (16.6 g, 128.4 mmol, 4.0 equiv.) were added sequentially to the residue. The resulting mixture was added Di-tert-butyl dicarbonate (6.3 g, 28.88 mmol, 0.9 equiv.) at −30° C. then stirred for 16 h at 25° C. The resulting mixture was added 40.5 mL of aqueous potassium phosphate solution (2.0 M), 81 mL of brine, and 24.3 mL of water sequentially, the aqueous layer was extracted with 2×250 mL ethyl acetate. The organic layer was dried over anhydrous magnesium sulfate, was filtered, and was concentrated under reduced pressure. The residue was applied onto a silica gel column with dichloromethane/methanol (100:1-15:1) and the fractions were concentrated under reduced pressure. 5.0 g (62% yield) of Intermediate 6-2 was obtained as a light-yellow oil. MS m/z [M+H]⁺ (ESI): 253.25. ¹H NMR (300 MHz, Methanol-d4) δ 5.06 (s, 1H), 4.90 (s, 1H), 4.39 (d, J=7.3 Hz, 1H), 4.14 (s, 1H), 3.24 (d, J=12.4 Hz, 1H), 3.08 (s, 1H), 2.54 (d, J=11.8 Hz, 1H), 2.10-1.94 (m, 2H), 1.92-1.83 (m, 5H), 1.43 (s, 9H).

### Intermediate 6-3: 5-bromo-4-chloro-6,8-difluoro-2-(methylthio)quinazoline

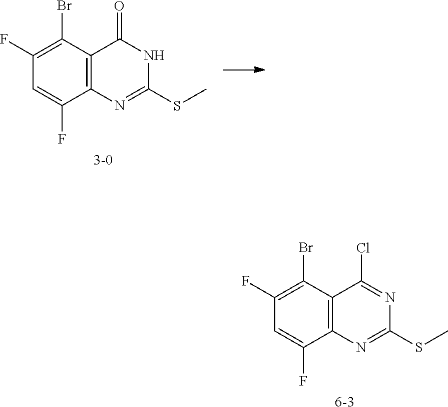

18 mL Phosphorus oxychloride (˜20 eq.) was added to Intermediate 3-0 (3 g, 9.77 mmol) at 0° C. (ice bath). DIEA (3 eq. 5.1 mL) was added dropwise. The reaction was warmed up to room temperature and stirred at room temperature for 40 minutes. The mixture was concentrated under vacuum for 2 hours. The reaction crude Intermediate 6-3 was used directly for next step.

### Intermediate 6-4: tert-butyl (1S,2S,5R)-3-(5-bromo-6,8-difluoro-2-(methylthio) quinazolin-4-yl)-2-(prop-1-en-2-yl)-3,8-diazabicyclo [3.2.1]octane-8-carboxylate

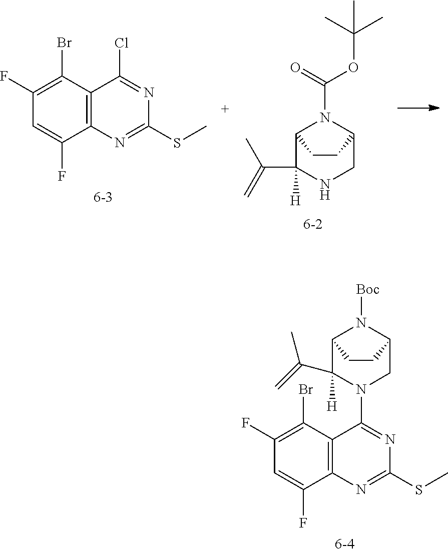

In a round bottom flask, DCM 20 mL was added to Intermediate 6-3 (9.51 mmol) and Intermediate 6-2 (2 g, 7.93 mmol). The mixture was cooled to 0° C., N,N-diisopropylethylamine (6.9 mL, 39.6 mmol) was added, and the resulting mixture allowed to warm to room temperature. After 5 min, the resulting mixture was heated to 40° C. overnight. The resulting mixture was cooled to room temperature and was concentrated under reduced pressure. The residue was purified by flash column chromatography on silica gel (0 to 11% ethyl acetate in hexanes) to give Intermediate 6-4 (3.7 g, yield 86%). LCMS (M+1): 543.1.

### Intermediate 6-5: tert-butyl (1S,4R,14S,14aS)-10,12-difluoro-14-methyl-8-(methylthio)-1,2,3,4,5,13,14,14a-octahydro-1,4-epiminoazepino[1′,2′:1,7] azepino [2,3,4-de]quinazoline-15-carboxylate

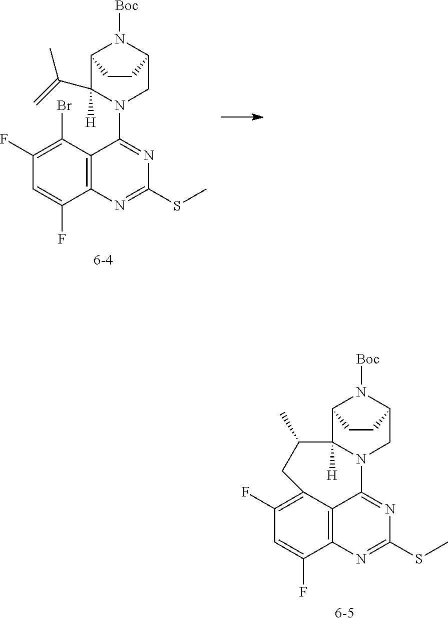

9-Borabicyclo[3.3.1]nonane (0.50 M in tetrahydrofuran, 20.5 mL, 1.5 eq.) was added to Intermediate 6-4 (3.7 g, 6.83 mmol) at room temperature. The resulting solution was stirred at 70° C. for 2 hours before it was cooled to room temperature. To the solution was added degassed 1,4-dioxane (20 ml). After bubbling with argon for 10 minutes, Pd(dppf)Cl₂ (496 mg, 0.678 mmol) and 1.5 N potassium phosphate in water (22.6 mL, 33.9 mmol) were added. The reaction vessel was equipped with a condenser. The reaction mixture was degassed again and filled with argon. The mixture was stirred at 120° C. oil bath for 2 hours. After cooling to room temperature, the reaction mixture was partitioned between EtOAc and brine, and the aqueous layer was extracted with EtOAc two more times. The combined organic layer was washed with brine, dried over magnesium sulfate, filtered, and concentrated to dryness. The crude was purified by silica gel column chromatography (0 to 50% ethyl acetate in hexanes) to give Intermediate 6-5. LCMS (M+1): 463.3.

### Intermediate 6-6: tert-butyl (1S,4R,14S,14aS)-11-bromo-10,12-difluoro-14-methyl-8-(methylthio)-1,2,3,4,5,13,14,14a-octahydro-1,4-epiminoazepino [1′,2′:1,7] azepino[2,3,4-de]quinazoline-15-carboxylate

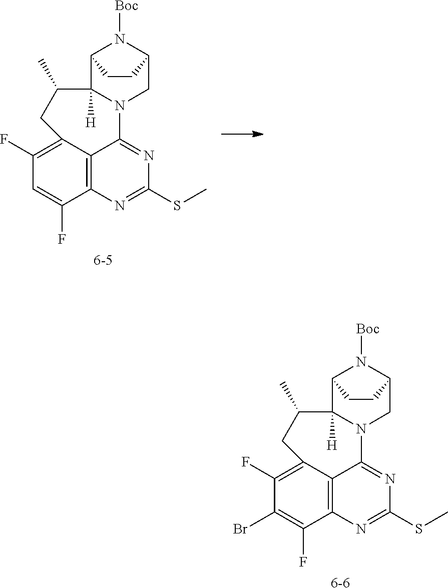

At rt, a mixture of ((TMP)₂Zn·2 MgCl₂·2 LiCl) (prepared by mixing 1:1 volume ratio of TMPMgCl·LiCl (1 M in THF) and ZnCl₂ (0.5 M in THF), stirred at room temperature overnight) (0.25 M, 19 mL, 3 eq.) was added to Intermediate 6-5 (high vac o/n before use) in a 200 mL round bottom flask, which was sealed and incubated at rt for 3 hours. 10% Br₂ in Me-THF (prepared in an ice bath, Br₂ was added dropwise to Me-THF) was added to the reaction at 0° C., and warmed to room temperature for 10 minutes. The reaction mixture was partitioned between EtOAc and brine, the aqueous layer was extracted with EtOAc two more times. The combined organic layer was washed with brine, dried over magnesium sulfate, filtered, and concentrated to dryness. The crude was purified by silica gel column chromatography (0 to 60% ethyl acetate in hexanes) to give Intermediate 6-6. LCMS (M+1): 529.2.

### Intermediate 6-7: tert-butyl (1S,4R,11R,14S,14aS)-10,12-difluoro-11-(7-fluoro-3-(methoxymethoxy)-8-((triisopropylsilyl)ethynyl) naphthalen-1-yl)-14-methyl-8-(methylthio)-1,2,3,4,5,13,14,14a-octahydro-1,4-epiminoazepino [1′,2′:1,7] azepino[2,3,4-de]quinazoline-15-carboxylate

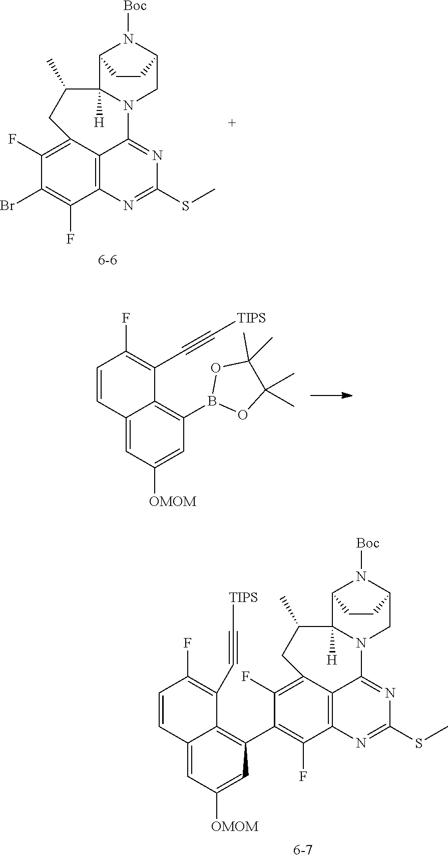

In a 20 mL microwave vial, Intermediate 6-6 (200 mg, 0.369 mmol) and ((2-fluoro-6-(methoxymethoxy)-8-(4,4,5,5-tetramethyl-1,3,2-dioxaborolan-2-yl)naphthalen-1-yl)ethynyl)triisopropylsilane (379 mg, 0.739 mmol) were dissolved in 3 mL THE. The solution was purged under argon for 3 minutes, then cataCXium A Pd G3 (27 mg, 0.037 mmol) was added. The mixture was purged under argon for 2 minutes, and then 1.5N K₃PO₄ aqueous solution (1.23 mL, 1.85 mmol) was added. The mixture was purged under argon for 3 minutes. The sealed vial was heated to 75° C. for 4 hours. The crude reaction mixture was cooled down and partitioned between EtOAc and brine, the aqueous layer was extracted with EtOAc two more times. The combined organic layer was washed with brine, dried over magnesium sulfate, filtered and concentrated to dryness. The crude was purified by silica gel column chromatography (20% ethyl acetate in hexanes) to give Intermediate 6-7 (R-atropisomer as less polar product). LCMS (M+1): 847.4.

### Intermediate 6-8: tert-butyl (1S,4R,11R,14S,14aS)-11-(8-ethynyl-7-fluoro-3-(methoxymethoxy)naphthalen-1-yl)-10,12-difluoro-14-methyl-8-(methylthio)-1,2,3,4,5,13,14,14a-octahydro-1,4-epiminoazepino[1′,2′:1,7]azepino[2,3,4-de]quinazoline-15-carboxylate

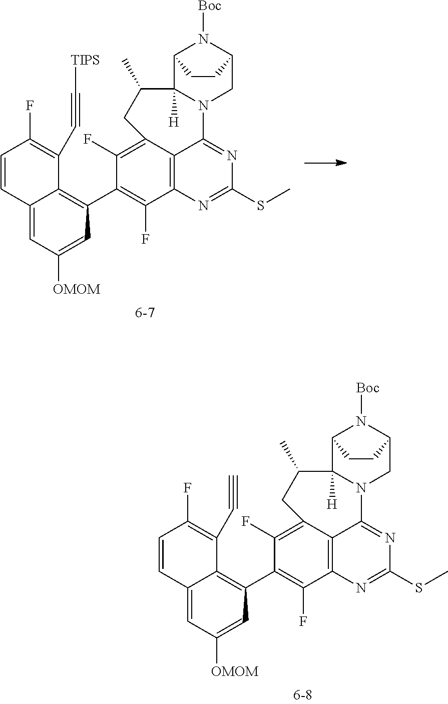

Cesium fluoride (62.8 mg, 0.413 mmol) was added to Intermediate 6-7 (70 mg, 0.0826 mmol) in 2 mL DMF. The reaction was stirred at room temperature for one hour. The reaction crude was filtered and purified via prep HPLC (0.1% TFA in MeCN and water) to give Intermediate 6-8. LCMS (M+1): 691.3.

### Intermediate 6-9: tert-butyl (1S,4R,11R,14S,14aS)-11-(8-ethynyl-7-fluoro-3-(methoxymethoxy)naphthalen-1-yl)-10,12-difluoro-14-methyl-8-(methylsulfonyl)-1,2,3,4,5,13,14,14a-octahydro-1,4-epiminoazepino [1′,2′:1,7] azepino[2,3,4-de]quinazoline-15-carboxylate

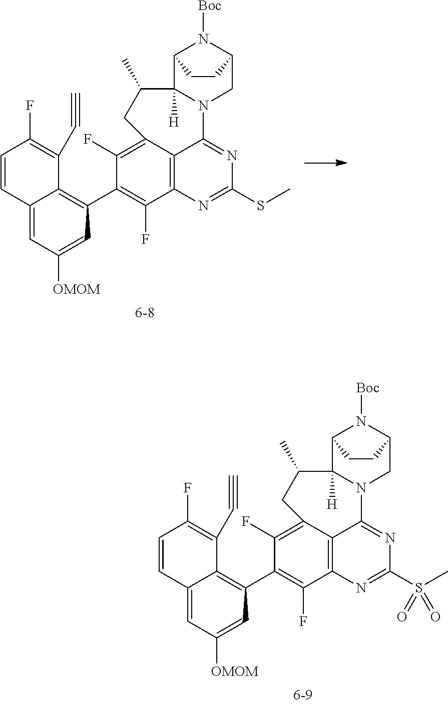

3-Chloroperoxybenzoic acid (50%, 43 mg, 0.124 mmol) was added to Intermediate 6-8 (43 mg, 0.0622 mmol) in 2 mL DCM. The reaction was stirred at room temperature for one hour. The reaction crude was subjected to silica gel column chromatography (0-80% ethyl acetate in hexanes) to give Intermediate 6-9. LCMS (M+1): 723.3.

### Intermediate 6-10: tert-butyl (1S,4R,11R,14S,14aS)-11-(8-ethynyl-7-fluoro-3-(methoxymethoxy)naphthalen-1-yl)-10,12-difluoro-8-(((S,E)-4-(fluoromethylene)-1,3-dimethylpiperidin-3-yl)methoxy)-14-methyl-1,2,3,4,5,13,14,14a-octahydro-1,4-epiminoazepino[1′,2′:1,7]azepino[2,3,4-de]quinazoline-15-carboxylate

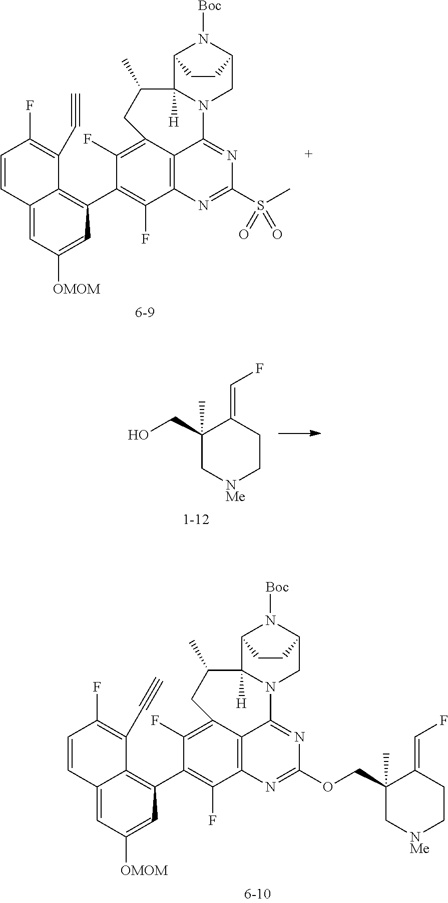

Intermediate 6-9 (20 mg, 0.0277 mmol) and Intermediate 1-12 (9.6 mg, 0.056 mmol) were azeotrope with toluene twice, then dissolved in 1 mL Me-THF. [Bis(trimethylsilyl)amino]lithium (1 M in THF, 0.083 mL) was added at 0° C. The reaction was warmed to room temperature for 15 minutes. The reaction crude was diluted with EtOAc and washed with brine. The organic layer was concentrated and purified via prep HPLC (0.1% TFA in MeCN and water) to give Intermediate 6-10. LCMS (M+1): 816.4.

### Intermediate 7-1: tert-butyl (6aR,7S,10R)-2-chloro-13-(ethylsulfonyl)-1-fluoro-5,6,6a,7,8,9,10,11-octahydro-4H-3,11a,12,14,15-pentaaza-7,10-methanocyclohepta[4,5]cycloocta[1,2,3-de]naphthalene-15-carboxylate

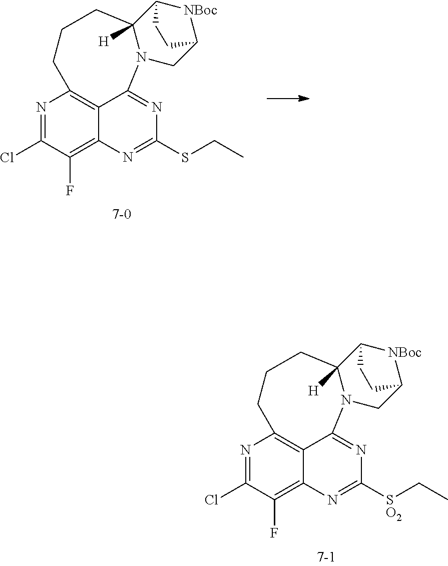

Intermediate 7-1 was synthesized in a manner similar to Intermediate 1-7 using Intermediate 7-0 (prepared according to US patent publication 2023/0374036) instead of Intermediate 1-6. LCMS: 526.2.

### Intermediate 7-2: tert-butyl (6aR,7S,10R)-2-chloro-1-fluoro-13-(((S,E)-4-(fluoromethylene)-1,3-dimethylpiperidin-3-yl)methoxy)-5,6,6a,7,8,9,10,11-octahydro-4H-3,11a,12,14,15-pentaaza-7,10-methanocyclohepta[4,5]cycloocta[1,2,3-de]naphthalene-15-carboxylate

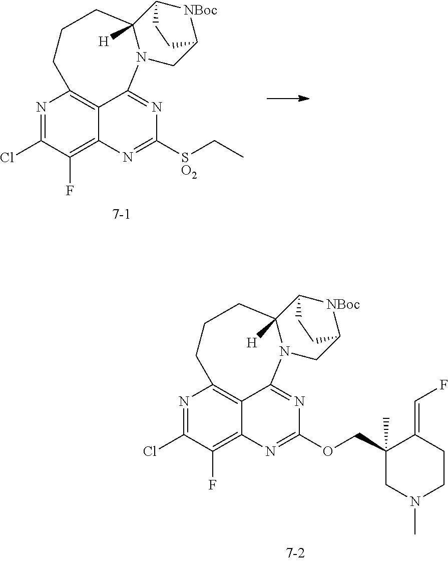

Lithium bis(trimethylsilyl)amide solution (1.0 M in tetrahydrofuran, 111 μL, 111 μmol) was added via syringe to a vigorously stirred mixture of Intermediate 7-1 (48.7 mg, 92.6 μmol), Intermediate 1-12 (17.6 mg, 102 μmol), and 2-methyltetrahydrofuran (0.20 mL) at 0° C. After 40 min, ethyl acetate (5 mL) and saturated ammonium chloride solution (1 mL) were added sequentially. The organic layer was washed with a mixture of water and brine (2:1 v:v, 5 mL), was dried over anhydrous magnesium sulfate, was filtered, and was concentrated under reduced pressure. The residue was purified by flash column chromatography on basic alumina (0 to 100% ethyl acetate in hexanes) to give Intermediate 7-2. LCMS: 526.2.

### Intermediate 7-3: tert-butyl (6aR,7S,10R)-2-(3-((diphenylmethylene)amino)-7-fluoro-8-((triisopropylsilyl)ethynyl)naphthalen-1-yl)-1-fluoro-13-(((S,E)-4-(fluoromethylene)-1,3-dimethylpiperidin-3-Yl)methoxy)-5,6,6a,7,8,9,10,11-octahydro-4H-3,11a,12,14,15-pentaaza-7,10-methanocyclohepta[4,5]cycloocta[1,2,3-de]naphthalene-15-carboxylate

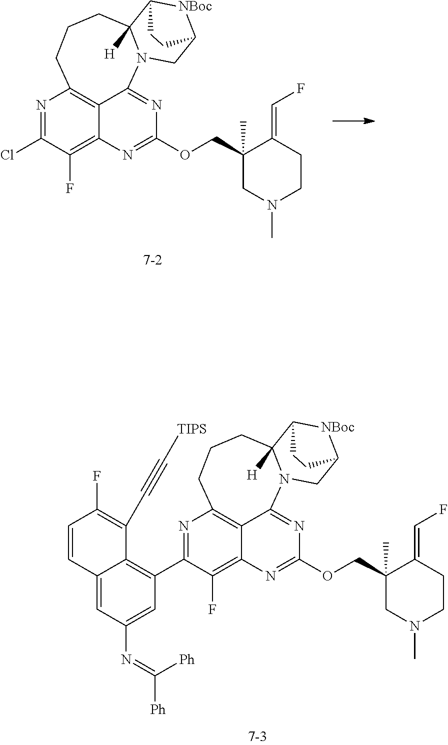

Intermediate 7-3 was synthesized in a manner similar to Intermediate 1-6 using N-(6-fluoro-4-(4,4,5,5-tetramethyl-1,3,2-dioxaborolan-2-yl)-5-((triisopropylsilyl)ethynyl)naphthalen-2-yl)-1,1-diphenylmethanimine (WO2022170999) instead of Intermediate 1-5. LCMS: 1074.6.

### Intermediate 7-4: tert-butyl (6aR,7S,10R)-2-(3-((diphenylmethylene)amino)-8-ethynyl-7-fluoronaphthalen-1-yl)-1-fluoro-13-(((S,E)-4-(fluoromethylene)-1,3-dimethylpiperidin-3-yl)methoxy)-5,6,6a,7,8,9,10,11-octahydro-4H-3,11a,12,14,15-pentaaza-7,10-methanocyclohepta[4,5]cycloocta[1,2,3-de]naphthalene-15-carboxylate

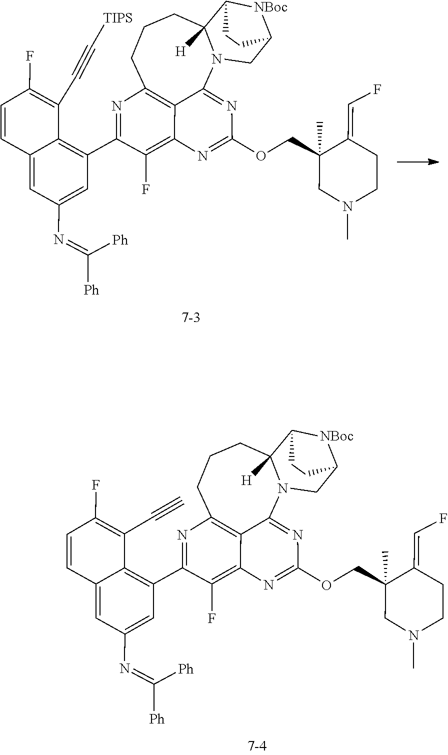

Dimethylformamide (0.58 mL) was added via syringe to a vigorously stirred mixture of Intermediate 7-3 (93.2 mg, 86.7 μmol) and cesium fluoride (132 mg, 867 μmol) at room temperature. After 40 min, ethyl acetate (5 mL) was added. The resulting mixture was filtered, and was concentrated under reduced pressure to give Intermediate 7-4. LCMS: 918.3.

### Intermediate 8-1: tert-butyl (6aR,7S,10R)-2-(8-ethynyl-7-fluoronaphthalen-1-yl)-1-fluoro-13-(((S,E)-4-(fluoromethylene)-1,3-dimethylpiperidin-3-yl)methoxy)-5,6,6a,7,8,9,10,11-octahydro-4H-3,11a,12,14,15-pentaaza-7,10-methanocyclohepta[4,5]cycloocta[1,2,3-de]naphthalene-15-carboxylate

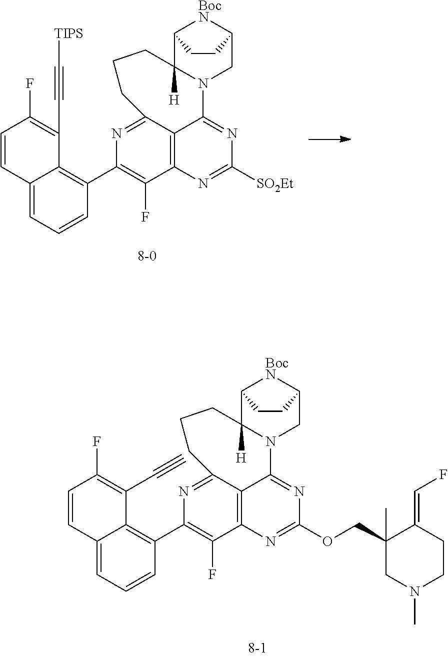

Intermediate 8-1 was synthesized in a manner similar to Intermediate 1-13 using Intermediate 8-0 (prepared according to US patent publication 2023/0374036) instead of Intermediate 1-7. LCMS: 739.2.

### Intermediate 9-1: tert-butyl (1S,4R,15aR)-11-(3-((tert-butoxycarbonyl)amino)-7-fluoro-8-((triisopropylsilyl)ethynyl)naphthalen-1-yl)-10,12-difluoro-8-(methylthio)-2,3,4,5,13,14,15,15a-octahydro-1H-1,4-epiminoazepino[1′,2′:1,8]azocino[2,3,4-de]quinazoline-16-carboxylate

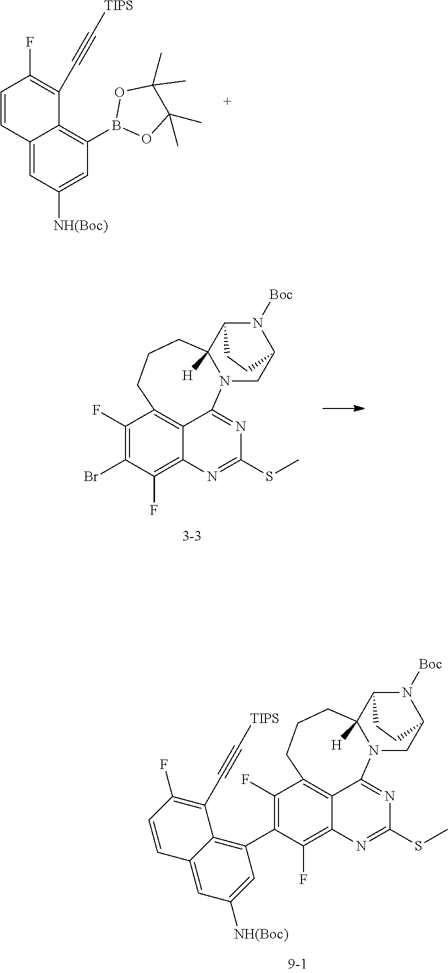

Intermediate 9-1 was synthesized in a manner similar to Intermediate 3-4 using tert-butyl (6-fluoro-4-(4,4,5,5-tetramethyl-1,3,2-dioxaborolan-2-yl)-5-((triisopropylsilyl)ethynyl)naphthalen-2-yl)carbamate instead of ((2-fluoro-6-(methoxymethoxy)-8-(4,4,5,5-tetramethyl-1,3,2-dioxaborolan-2-yl)naphthalen-1-yl)ethynyl)triisopropylsilane. LCMS: 902.5.

### Intermediate 9-2B: tert-butyl (1S,4R,11R,15aR)-11-(3-((tert-butoxycarbonyl)amino)-7-fluoro-8-((triisopropylsilyl)ethynyl)naphthalen-1-yl)-10,12-difluoro-8-(methylsulfonyl)-2,3,4,5,13,14,15,15a-octahydro-1H-1,4-epiminoazepino[1′,2′:1,8]azocino[2,3,4-de]quinazoline-16-carboxylate

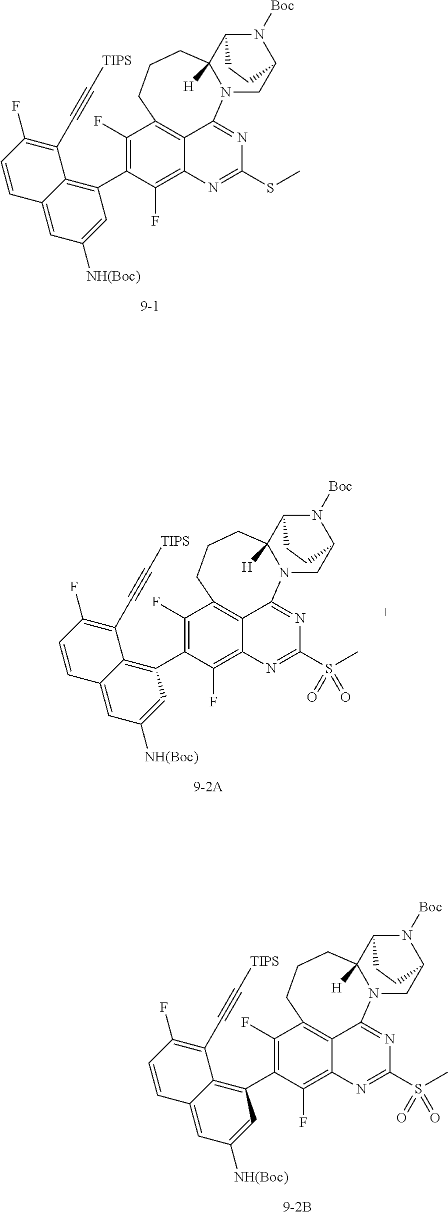

Intermediate 9-2B was synthesized in a manner similar to Intermediate 3-5B using Intermediate 9-1 instead of Intermediate 3-4. LCMS: 934.9.

### Intermediate 9-3: tert-butyl (1S,4R,15aR)-11-((R)-3-((tert-butoxycarbonyl)amino)-7-fluoro-8-((triisopropylsilyl)ethynyl)naphthalen-1-yl)-10,12-difluoro-8-(((S,E)-4-(fluoromethylene)-1,3-dimethylpiperidin-3-yl)methoxy)-2,3,4,5,13,14,15,15a-octahydro-1H-1,4-epiminoazepino[1′,2′:1,8]azocino[2,3,4-de]quinazoline-16-carboxylate

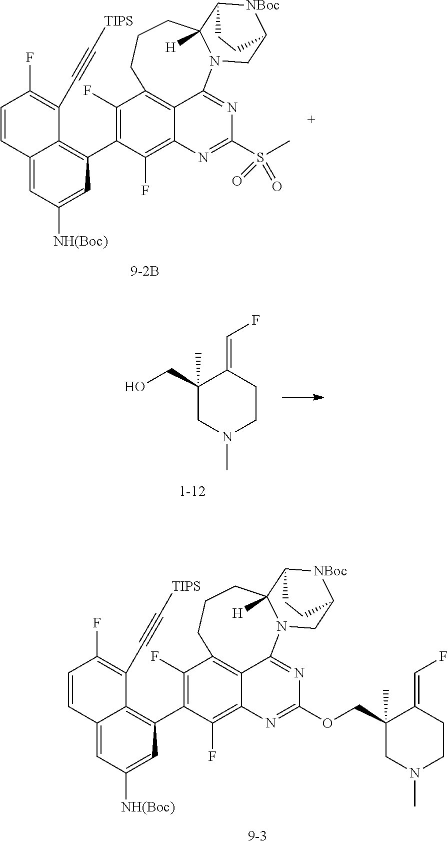

Intermediate 9-3 was synthesized in a manner similar to Intermediate 3-6 using Intermediate 9-2B instead of Intermediate 3-5B. LCMS: 1027.6.

### Intermediate 9-4: tert-butyl (1S,4R,15aR)-11-((R)-3-((tert-butoxycarbonyl)amino)-8-ethynyl-7-fluoronaphthalen-1-yl)-10,12-difluoro-8-(((S,E)-4-(fluoromethylene)-1,3-dimethylpiperidin-3-Yl)methoxy)-2,3,4,5,13,14,15,15a-octahydro-1H-1,4-epiminoazepino[1′,2′:1,8]azocino[2,3,4-de]quinazoline-16-carboxylate

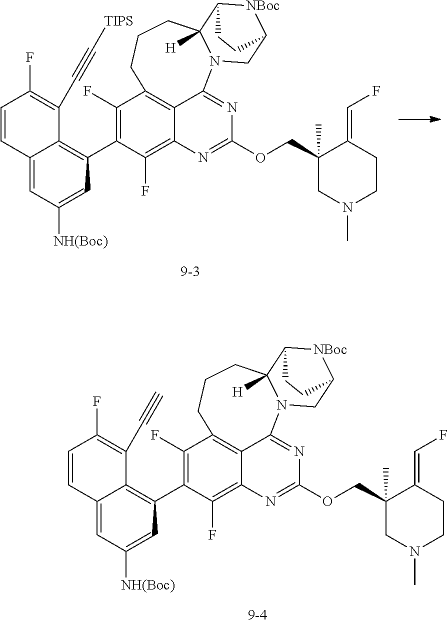

Intermediate 9-4 was synthesized in a manner similar to Intermediate 3-7 using Intermediate 9-3 instead of Intermediate 3-6. LCMS: 871.3.

### Intermediate 10-1: 2-amino-6-bromo-5-chloro-3-fluorobenzoic acid

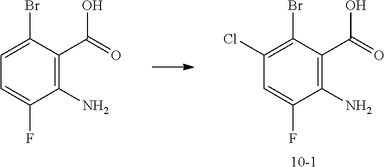

To a stirred solution of 2-amino-6-bromo-3-fluorobenzoic acid (95 g, 406 mmol) in acetonitrile (1 L) was added N-chlorosuccinimide (64.6 g, 406 mmol) at 0° C. Then the mixture was stirred for 16 hrs at 60° C. The reaction mixture was concentrated under reduced pressure to give crude product which was further purified by column chromatography using petroleum ether/ethyl acetate (I/O to 4/1) to afford Intermediate 10-1. LCMS: 266 [M−H]⁻.

### Intermediate 10-2: 5-bromo-6-chloro-8-fluoro-2-thioxo-2,3-dihydroquinazolin-4(1H)-one

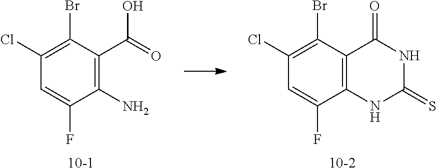

To a stirred solution of Intermediate 10-1 (85.0 g, 285 mmol) in anhydrous DCM (850 mL) was added SOCl₂ (100 mL) at 0° C., and then stirred for 6 hrs at 80° C. The mixture was allowed to cool down to 25° C. The resulting mixture was concentrated under reduced pressure. The residue was dissolved in acetone (1.7 L), and then NH₄SCN (43.4 g, 570 mmol) was added to the mixture, and then stirred for 1 hr at 25° C. The product was precipitated by the addition of ice water (1.7 L). The precipitated solids were collected by filtration and washed with water (2×500 mL) to give Intermediate 10-2. LCMS: 307 [M−H]⁻.

### Intermediate 10-3: 5-bromo-6-chloro-8-fluoro-2-(methylthio)quinazolin-4(3H)-one

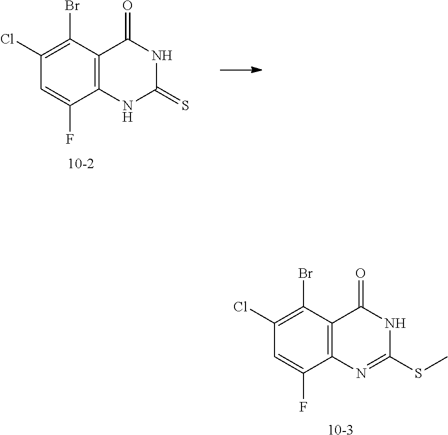

To a stirred solution of Intermediate 10-2 (90 g, 291 mmol) in MeOH (900 mL) and H₂O (450 mL) was added NaOH (23.3 g, 582 mmol) and Mel (82.5 g, 582 mmol) in portions at 25° C. The mixture was stirred for 1 hr, whereupon the reaction mixture was diluted with H₂O (450 mL). The mixture was acidified to pH 6 with HCl (1 M). The precipitated solids were collected by filtration and washed with CS₂ (500 mL) and ACN/MeOH (1/1, 1 L) to afford Intermediate 10-3. LCMS: 323. ¹H NMR (400 MHz, DMSO-d₆) δ: 12.93 (s, 1H), 8.00-7.97 (m, 1H), 2.55 (s, 3H).

### Intermediate 10-4: tert-butyl (1S,2S,5R)-3-(5-bromo-6-chloro-8-fluoro-2-(methylthio)quinazolin-4-yl)-2-(prop-1-en-2-yl)-3,8-diazabicyclo[3.2.1]octane-8-carboxylate

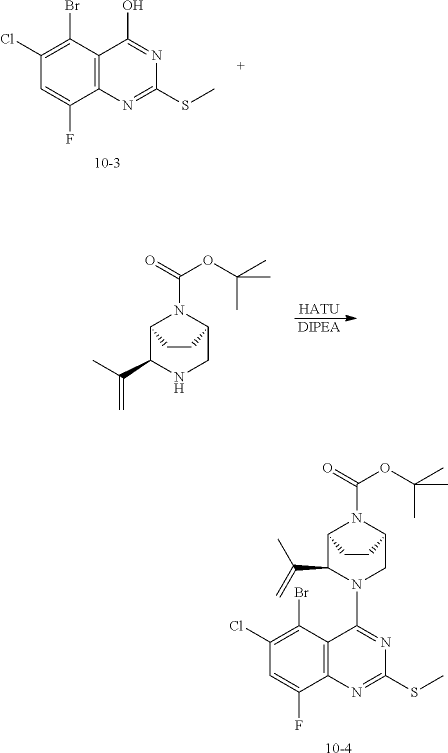

To a stirred suspension of Intermediate 10-3 (2021 mg, 6.24 mmol), tert-butyl (1S,2S,5R)-2-(prop-1-en-2-yl)-3,8-diazabicyclo[3.2.1]octane-8-carboxylate (1657 mg, 6.57 mmol), and 2-(1H-7-azabenzotriazol-1-yl)-1,1,3,3-tetramethyl uronium hexafluorophosphate (3030 mg, 7.97 mmol) in N,N-dimethylformamide (12 mL) was added diisopropylethylamine (5.5 mL, 31.6 mmol) at rt. The resulting mixture was stirred at rt. After 18 h, the reaction mixture was diluted with ethyl acetate (˜100 mL) and washed with saturated aqueous NaHCO₃ (˜100 mL×1+˜50 mL of water) and water (˜100 mL×1). After the aqueous fractions were extracted with ethyl acetate (100 mL×1), the organic fractions were dried (MgSO₄), and concentrated. The residue was purified by silica gel column chromatography eluting 0-15% ethyl acetate in hexane to give Intermediate 10-4. LCMS: 557.1.

### Intermediate 10-5: tert-butyl (1S,4R,14S,14aS)-12-chloro-10-fluoro-14-methyl-8-(methylthio)-1,2,3,4,5,13,14,14a-octahydro-1,4-epiminoazepino[1′,2′:1,7]azepino[2,3,4-de]quinazoline-15-carboxylate

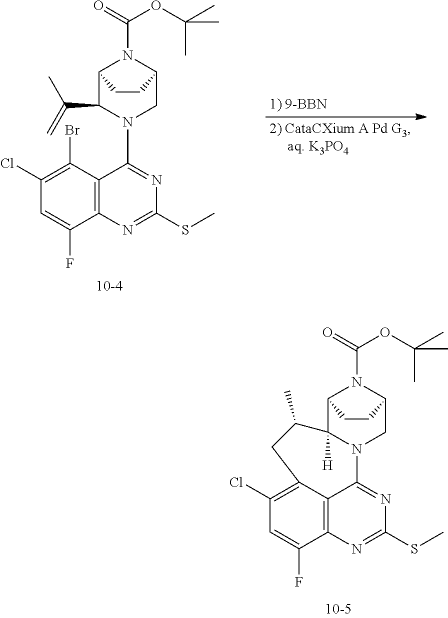

A solution of Intermediate 10-4 (2569 mg, 4.61 mmol; azeotroped with toluene 3 x) in 2-methyltetrahydrofuran (7.7 mL) was stirred at rt as 0.5 M 9-borabicyclo[33,1]nonane in THE (13.8 mL, 6.91 mmol) was added. The resulting solution was heated at 70° C. oil bath for 120 min and then cooled to rt. After the resulting reaction mixture was added 2.0 M aq. potassium phosphate tribasic (6.91 mL, 13.8 mmol) and the mixture was stirred at rt for 10 min, cataCXium A Pd G3 (335.3 mg, 0.461 mmol) and 2-methyltetrahydrofuran (23.9 mL) were added to the mixture. After the mixture was purged with argon gas for 10 min, the resulting mixture was heated at 110° C. heating bath for 45 min with reflux condenser and then cooled to rt. The reaction mixture was diluted with ethyl acetate (˜100 mL), washed with saturated aqueous NaCl (100 mL). After the aqueous fractions were extracted with ethyl acetate (100 mL×1), the combined organic fractions were dried (MgSO₄) and concentrated. The residue was purified by silica gel column chromatography eluting 0-20% ethyl acetate in hexane to give Intermediate 10-5. LCMS: 479.2.

### Intermediate 10-6: tert-butyl (1S,4R,11R,14S,14aS)-11-(2-((tert-butoxycarbonyl)amino)-3-cyano-7-fluorobenzo[b]thiophen-4-yl)-12-chloro-10-fluoro-14-methyl-8-(methylthio)-1,2,3,4,5,13,14,14a-octahydro-1,4-epiminoazepino[1′,2′:1,7]azepino[2,3,4-de]quinazoline-15-carboxylate

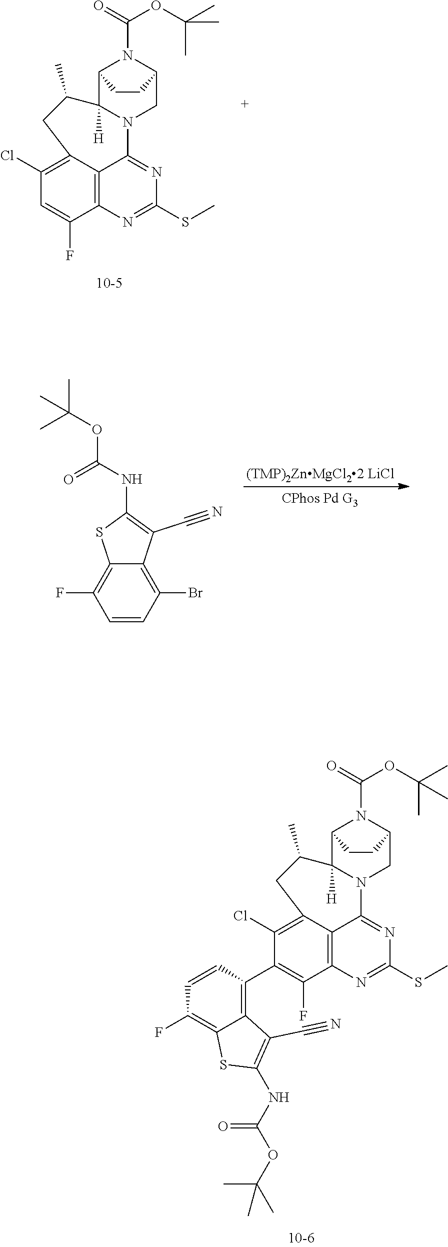

Intermediate 10-5 (202 mg. 422 μmol; co-evaporated with toluene×3) was evacuated under vacuum and filled with argon gas three times. To the vial containing the reactant a solution of bis(2,2,6,6-tetramethylpiperidinyl)zinc, lithium chloride, and magnesium chloride complex ((TMP)₂Zn·2MgCl₂·2LiCl) (5 mL of a 0.247 M solution in THF, 1.23 mmol) was added at rt and the mixture was stirred at 50° C. for 2.5 h and then cooled to rt. To the resulting mixture was added a suspension of tert-butyl N-(4-bromo-3-cyano-7-fluoro-benzothiophen-2-yl)carbamate (214 mg, 576 μmol) and CPhos Pd G3 (51.0 mg, 63.3 μmol) in 1,4-dioxane (2.25 mL), and the resulting suspension was stirred at 60° C. for 6 h and then cooled to rt. After the reaction mixture was diluted with ethyl acetate (˜30 mL) and saturated aqueous NaHCO₃ (˜30 mL), the mixture was filtered through celite pad and the two phases of the filtrate were separated. After the aqueous fraction was extracted with ethyl acetate (˜30 mL), the two organic fractions were combined, dried (MgSO₄), and concentrated. The residue was purified by silica gel column chromatography eluting 0-35% ethyl acetate in hexane to give Intermediate 10-6. LCMS: 769.3.

### Intermediate 10-7: tert-butyl (1S,4R,11R,14S,14aS)-11-(2-((tert-butoxycarbonyl)amino)-3-cyano-7-fluorobenzo[b]thiophen-4-yl)-12-chloro-10-fluoro-14-methyl-8-(methylsulfonyl)-1,2,3,4,5,13,14,14a-octahydro-1,4-epiminoazepino[1′,2′:1,7]azepino[2,3,4-de]quinazoline-15-carboxylate

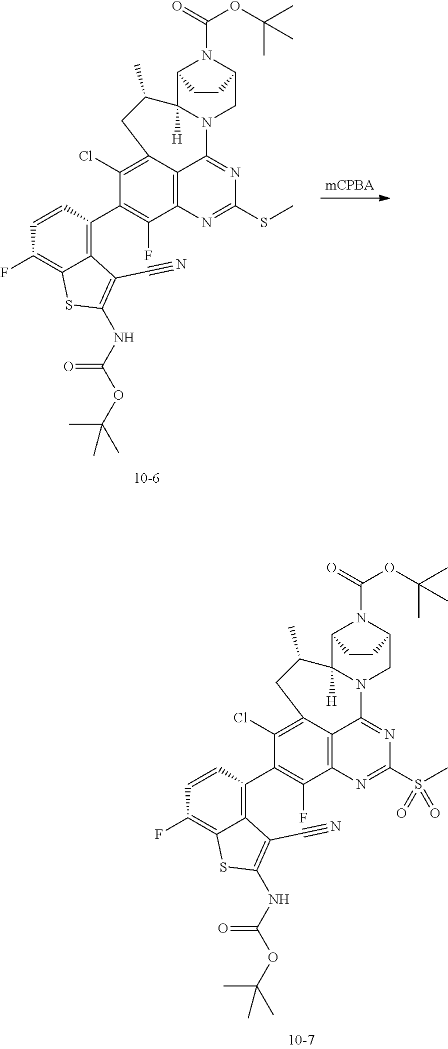

The solution of Intermediate 10-6 (110 mg, 143 μmol) in dichloromethane (5 mL) was stirred at 0° C. bath as m-chloroperbenzoic acid (mCPBA, <77% pure, 90.0 mg, 402 μmol) was added. After 1.5 h at 0° C., additional m-chloroperbenzoic acid (<77% pure, 29.19 mg, 130 μmol) was added at 0° C. and stirred at 0° C. for 1 h. The reaction mixture was diluted with ethyl acetate (˜25 mL) and washed with a mixture of saturated aqueous Na₂S₂O₃ solution (15 mL) and saturated aqueous NaHCO₃ solution (15 mL). After the organic fraction was washed with brine (30 mL×1), the aq. fractions were extracted with ethyl acetate (20 mL×1). The two organic fractions were combined, dried (MgSO₄), concentrated, and purified by silica gel column chromatography eluting 0-60% ethyl acetate in hexane to give Intermediate 10-7. LCMS: 801.3.

### Intermediate 10-8: tert-butyl (1S,4R,11R,14S,14aS)-11-(2-((tert-butoxycarbonyl)amino)-3-cyano-7-fluorobenzo[b]thiophen-4-yl)-12-chloro-10-fluoro-8-(((S,E)-4-(fluoromethylene)-1,3-dimethylpiperidin-3-yl)methoxy)-14-methyl-1,2,3,4,5,13,14,14a-octahydro-1,4-epiminoazepino[1′,2′:1,7]azepino[2,3,4-de]quinazoline-15-carboxylate

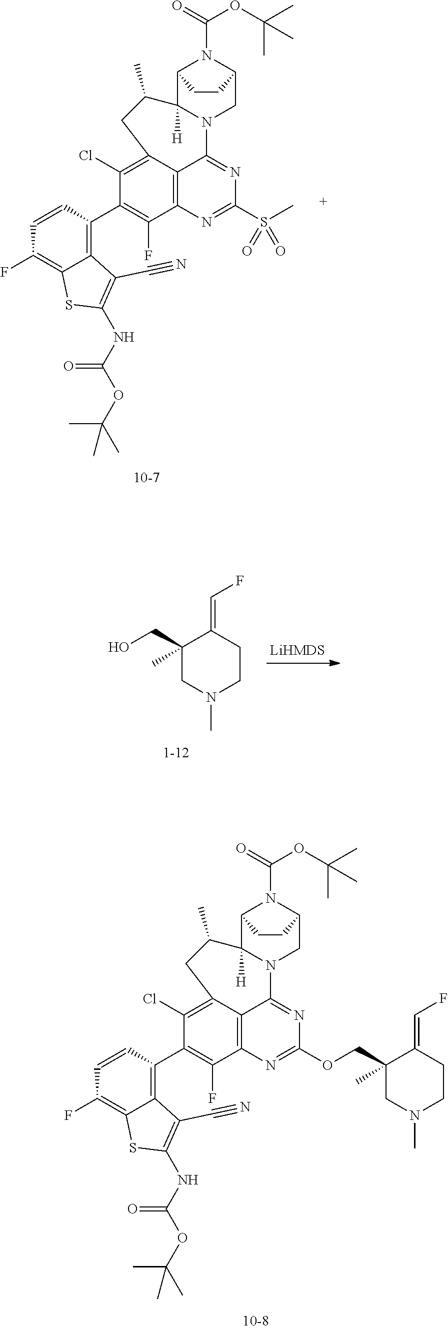

A mixture of Intermediate 10-7 (46.3 mg, 57.8 μmol) and Intermediate 1-12 (30.8 mg, 178 μmol) was co-evaporated with toluene 3 times. The resulting residue was dissolved in 2-methyltetrahydrofuran (1 mL) and stirred at 0° C. as 1 M solution of lithium bis(trimethylsilyl)amide in THE (0.1 mL) was added. The resulting solution was stirred at 0° C. for 0.5 h. After additional 1 M solution of lithium bis(trimethylsilyl)amide in THE (0.1 mL) was added at the reaction mixture at 0° C., the resulting solution was stirred at 0° C. for 1 h. The reaction mixture was diluted with saturated aqueous NaHCO₃ (˜25 mL) and the product was extracted with ethyl acetate (25 mL×2). After the extracts were washed with water (˜25 mL×1), the organic fractions were combined, dried (MgSO₄), and concentrated. The residue was purified by silica gel column chromatography eluting 0-15% methanol in dichloromethane. The collected product was further purified by preparative HPLC (2 injections: Gemini 5 um C18 110A, AX, 100 ×30.00 mm 5 micro column) eluting 5-70% acetonitrile (0.1% TFA) in water (0.1% TFA). The product containing fractions were combined, neutralized by adding saturated aqueous NaHCO₃ solution (˜1 mL), and concentrated to remove organic solvent. The product was extracted with ethyl acetate (˜20 mL×2), washed with water (˜20 mL×1), combined, dried (MgSO₄), and concentrated to give Intermediate 10-8. LCMS: 894.3.

### Intermediate 11-1: N-(5-ethynyl-6-fluoro-4-((6aR,7S,10R)-1-fluoro-13-(((S,E)-4-(fluoromethylene)-1,3-dimethylpiperidin-3-yl)methoxy)-5,6,6a,7,8,9,10,11-octahydro-4H-3,11a,12,14,15-pentaaza-7,10-methanocyclohepta[4,5]cycloocta[1,2,3-de]naphthalen-2-yl)naphthalen-2-yl)-1,1-diphenylmethanimine

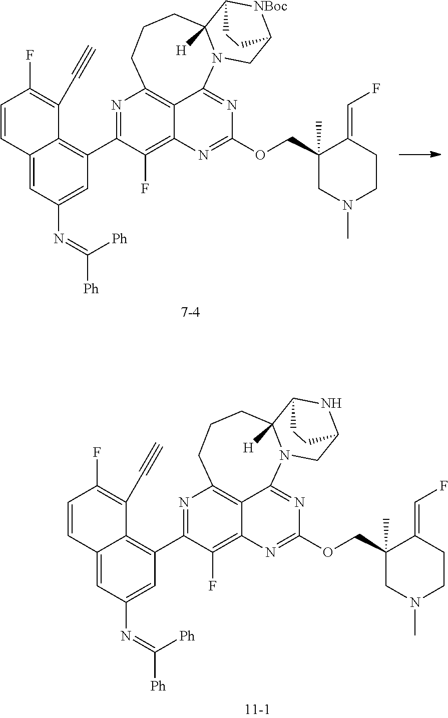

Intermediate 11-1 was synthesized in a manner similar to Intermediate 5-2 using Intermediate 7-4 instead of Intermediate 5-1. LCMS: 818.3.

### Intermediate 11-2: 4-(((6aR,7S,10R)-2-(3-((diphenylmethylene)amino)-8-ethynyl-7-fluoronaphthalen-1-yl)-1-fluoro-13-(((S,E)-4-(fluoromethylene)-1,3-dimethylpiperidin-3-yl)methoxy)-5,6,6a,7,8,9,10,11-octahydro-4H-3,11a,12,14,15-pentaaza-7,10-methanocyclohepta[4,5]cycloocta[1,2,3-de]naphthalen-15-yl)methyl)-5-methyl-1,3-dioxol-2-one

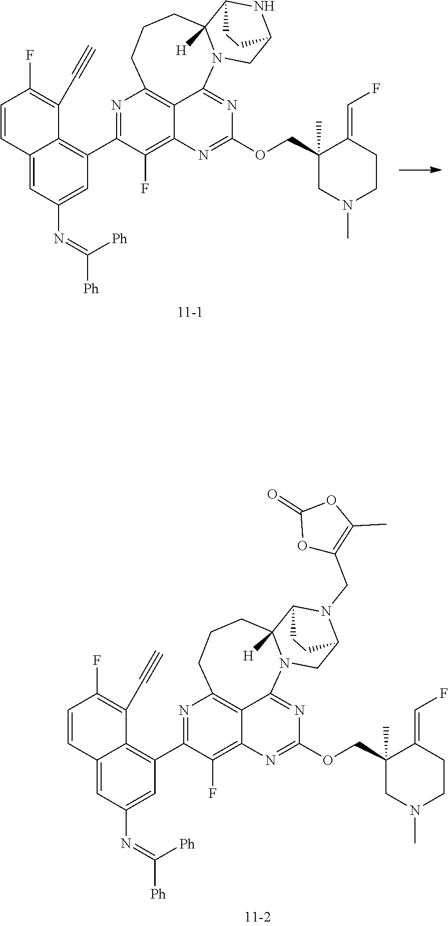

Intermediate 11-2 was synthesized in a manner similar to Intermediate 5-3 using Intermediate 11-1 instead of Intermediate 5-2. LCMS: 930.2.

### Intermediate 12-1: tert-butyl (8S,8aS,9S,12R)-5-(8-ethynyl-7-fluoro-3-(methoxymethoxy)naphthalen-1-yl)-4-fluoro-2-(((S,E)-4-(fluoromethylene)-1,3-dimethylpiperidin-3-yl)methoxy)-8-methyl-7,8,8a,9,10,11,12,13-octahydro-1,3,6,13a,14-pentaaza-9,12-methanonaphtho[1,8-ab]heptalene-14-carboxylate

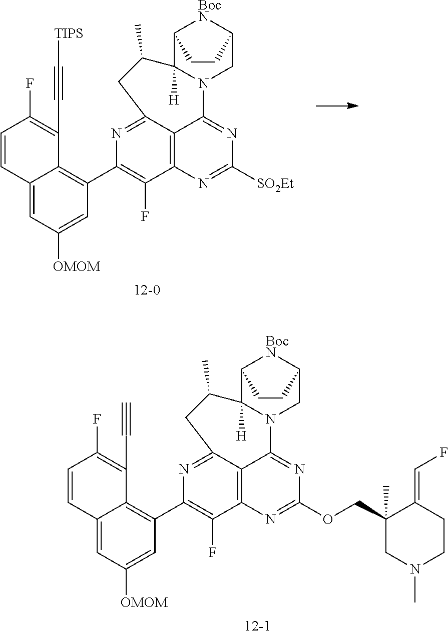

Intermediate 12-1 was synthesized in a manner similar to Intermediate 1-13 using Intermediate 12-0 (prepared according to US patent publication 2023/0374036) instead of Intermediate 1-7. LCMS: 799.5.

### Intermediate 14-1: methyl (S,E)-4-(fluoromethylene)-3-methyl-1-(methyl-d₃)piperidine-3-carboxylate

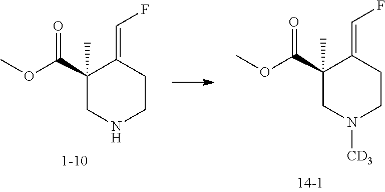 [SMILES: COC(=O)[C@]1(C)CNCC/C1=C\F ; [2H]C([2H])([2H])N1CC/C(=C\F)[C@](C)(C(=O)OC)C1]

To the mixture of Intermediate 1-10 (1.70 g, 0.00845 mol) in acetonitrile (20.0 mL) was added trideuterio(iodo)methane (0.980 g, 0.00676 mol) and potassium carbonate (3.50 g, 0.0253 mol). The mixture was stirred at 90° C. for 2 hrs. The reaction mixture was filtered, and the filtrate was concentrated under reduced pressure. The residue was purified by flash silica gel chromatography (20 g Silica Flash Column, Eluent of 0-30% Ethyl acetate/Petroleum ether gradient at 80 mL/min) to give Intermediate 14-1. LCMS: 205.2.

### Intermediate 14-2: (S,E)-(4-(fluoromethylene)-3-methyl-1-(methyl-d₃)piperidin-3-yl)methan-d₂-ol

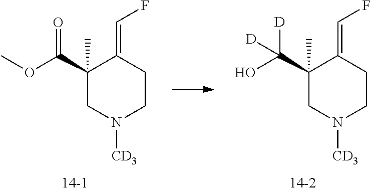 [SMILES: [2H]C([2H])([2H])N1CC/C(=C\F)[C@](C)(C(=O)OC)C1 ; [2H]C([2H])([2H])N1CC/C(=C\F)[C@](C)(C([2H])([2H])O)C1]

Intermediate 14-2 was synthesized in a manner similar to Intermediate 1-12 using Intermediate 14-1 instead of Intermediate 1-11 and using lithium aluminum deuteride instead of lithium aluminum hydride. LCMS: 179.1.

### Intermediate 21-1: tert-butyl (8S,8aS,9S,12R)-2-(ethylthio)-4-fluoro-5-(7-fluoro-3-(methoxymethoxy)-8-(trifluoromethoxy)naphthalen-1-yl)-8-methyl-7,8,8a,9,10,11,12,13-octahydro-1,3,6,13a,14-pentaaza-9,12-methanonaphtho[1,8-ab]heptalene-14-carboxylate

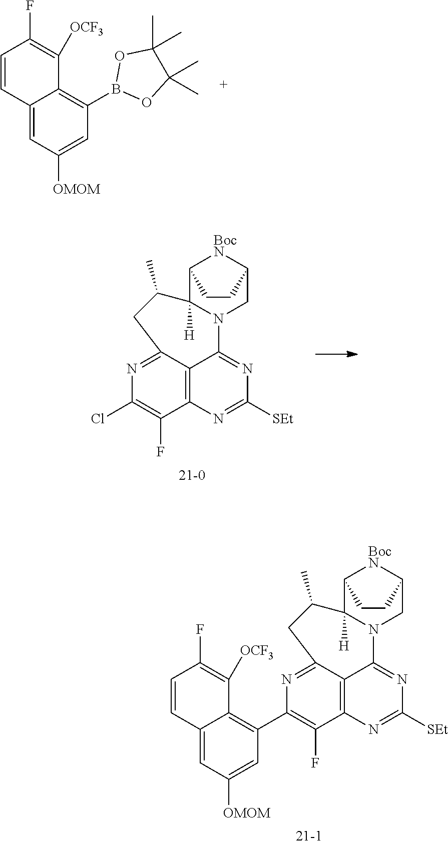

Intermediate 21-1 was synthesized in a manner similar to Intermediate 1-6 using Intermediate 21-0 (prepared according to US20230374036) instead of Intermediate 1-5 and using 2-(7-fluoro-3-(methoxymethoxy)-8-(trifluoromethoxy)naphthalen-1-yl)-4,4,5,5-tetramethyl-1,3,2-dioxaborolane instead of ((2-fluoro-6-(methoxymethoxy)-8-(4,4,5,5-tetramethyl-1,3,2-dioxaborolan-2-yl)naphthalen-1-yl)ethynyl)triisopropylsilane. LCMS: 748.3.

### Intermediate 21-2: tert-butyl (8S,8aS,9S,12R)-2-(ethylsulfonyl)-4-fluoro-5-(7-fluoro-3-(methoxymethoxy)-8-(trifluoromethoxy)naphthalen-1-yl)-8-methyl-7,8,8a,9,10,11,12,13-octahydro-1,3,6,13a,14-pentaaza-9,12-methanonaphtho[1,8-ab]heptalene-14-carboxylate

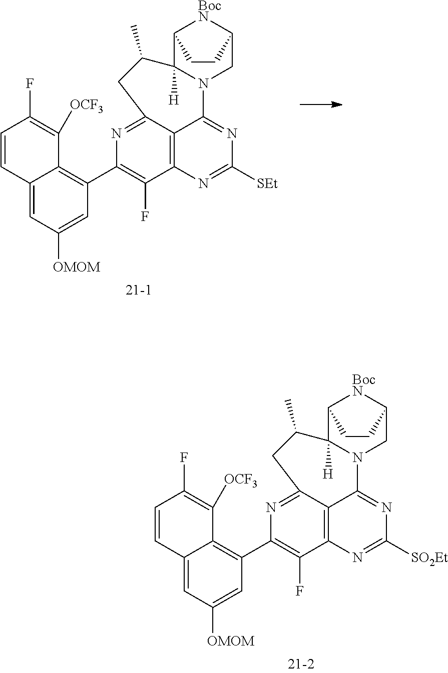

Intermediate 21-2 was synthesized in a manner similar to Intermediate 1-7 using Intermediate 21-1 instead of Intermediate 1-6. LCMS: 780.3.

### Intermediate 21-3: tert-butyl (8S,8aS,9S,12R)-4-fluoro-5-(7-fluoro-3-(methoxymethoxy)-8-(trifluoromethoxy)naphthalen-1-yl)-2-(((S,E)-4-(fluoromethylene)-1,3-dimethylpiperidin-3-yl)methoxy)-8-methyl-7,8,8a,9,10,11,12,13-octahydro-1,3,6,13a,14-pentaaza-9,12-methanonaphtho[1,8-ab]heptalene-14-carboxylate

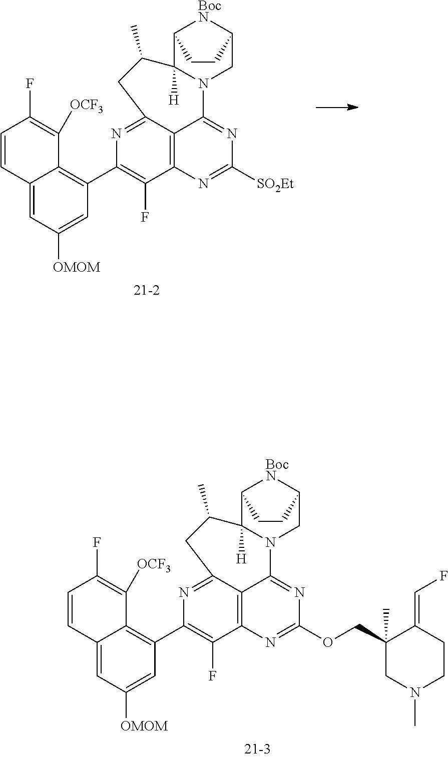

Intermediate 21-3 was synthesized in a manner similar to Intermediate 1-13 using Intermediate 21-2 instead of Intermediate 1-7. LCMS: 859.5.

### Intermediate 22-1: (1S,4R,15aR)-11-((R)-8-ethynyl-7-fluoro-3-(methoxymethoxy)naphthalen-1-yl)-10,12-difluoro-8-(((S,E)-4-(fluoromethylene)-1,3-dimethylpiperidin-3-yl)methoxy)-2,3,4,5,13,14,15,15a-octahydro-1H-1,4-epiminoazepino[1′,2′:1,8]azocino[2,3,4-de]auinazoline

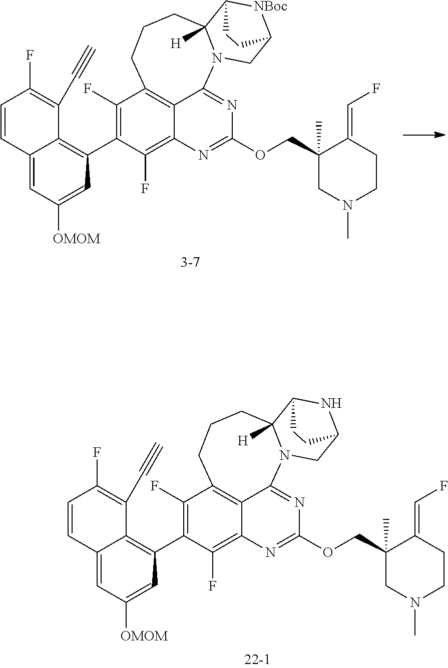

Intermediate 22-1 was synthesized in a manner similar to Intermediate 5-2 using Intermediate 3-7 instead of Intermediate 5-1. LCMS: 716.4.

### Intermediate 22-2: 4-(((1S,4R,11R,15aR)-11-(8-ethynyl-7-fluoro-3-(methoxymethoxy)naphthalen-1-yl)-10,12-difluoro-8-(((S,E)-4-(fluoromethylene)-1,3-dimethylpiperidin-3-yl)methoxy)-2,3,4,5,13,14,15,15a-octahydro-1H-1,4-epiminoazepino[1′,2′:1,8]azocino[2,3,4-de]quinazolin-16-yl)methyl)-5-methyl-1,3-dioxol-2-one

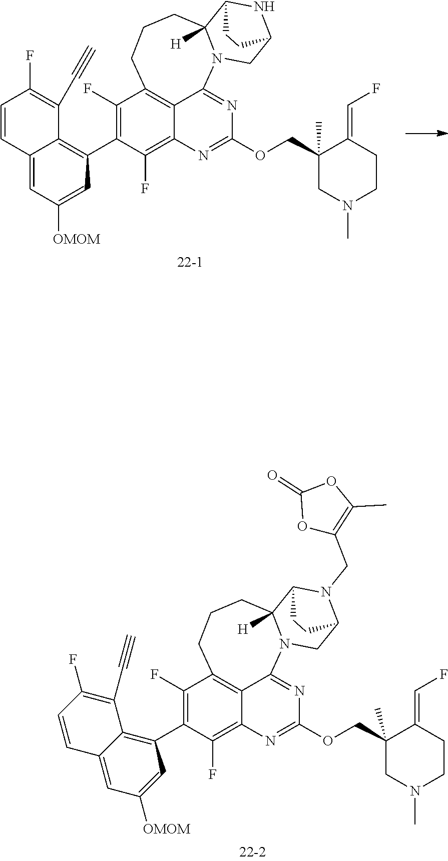

Intermediate 22-2 was synthesized in a manner similar to Intermediate 5-3 using Intermediate 22-1 instead of Intermediate 5-2. LCMS: 828.4.

### Intermediate 23-1: (7R,8S,8aS,9S,12R)-5-(8-ethynyl-7-fluoro-3-(methoxymethoxy)naphthalen-1-yl)-4-fluoro-2-(((S,E)-4-(fluoromethylene)-1,3-dimethylpiperidin-3-yl)methoxy)-7,8-dimethyl-7,8,8a,9,10,11,12,13-octahydro-1,3,6,13a,14-pentaaza-9,12-methanonaphtho[1,8-ab]heptalene

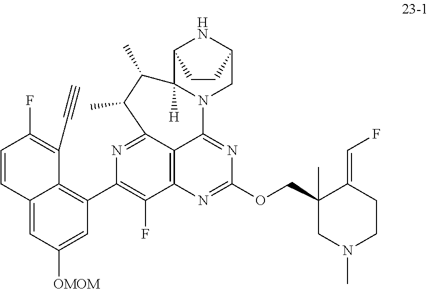

Intermediate 23-1 was synthesized in a manner similar to Intermediate 5-2 using Intermediate 4-5 instead of Intermediate 5-0. LCMS: 713.4.

### Intermediate 23-2: 4-(((7R,8S,8aS,9S,12R)-5-(8-ethynyl-7-fluoro-3-(methoxymethoxy)naphthalen-1-yl)-4-fluoro-2-(((S,E)-4-(fluoromethylene)-1,3-dimethylpiperidin-3-yl)methoxy)-7,8-dimethyl-7,8,8a,9,10,11,12,13-octahydro-1,3,6,13a,14-pentaaza-9,12-methanonaphtho[1,8-ab]heptalen-14-yl)methyl)-5-methyl-1,3-dioxol-2-one

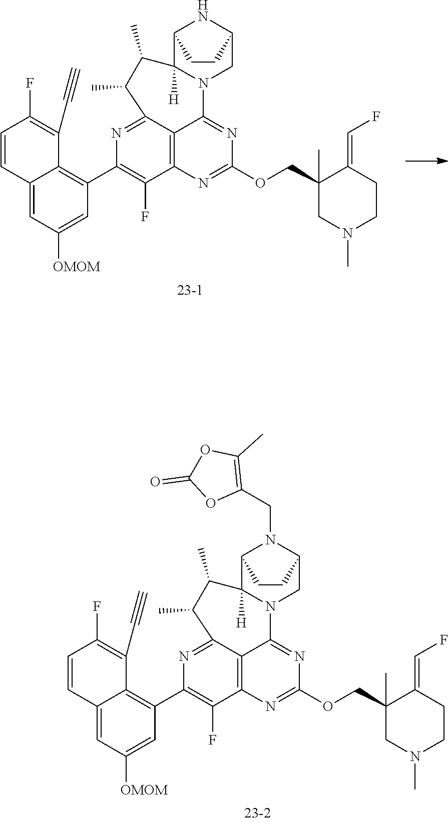

Intermediate 23-2 was synthesized in a manner similar to Intermediate 5-3 using Intermediate 23-1 instead of Intermediate 5-2. LCMS: 825.6.

### Intermediate 24-1: tert-butyl (6aR,7S,10R)-2-(8-ethyl-7-fluoro-3-(methoxymethoxy)naphthalen-1-yl)-1-fluoro-13-(((S,E)-4-(fluoromethylene)-1,3-dimethylpiperidin-3-yl)methoxy)-5,6,6a,7,8,9,10,11-octahydro-4H-3,11a,12,14,15-pentaaza-7,10-methanocyclohepta[4,5]cycloocta[1,2,3-de]naphthalene-15-carboxylate

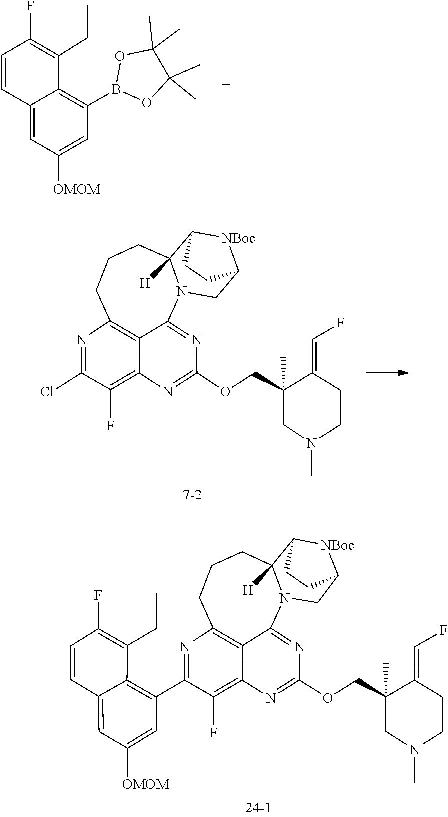

Intermediate 24-1 was synthesized in a manner similar to Intermediate 7-3 using 2-(8-ethyl-7-fluoro-3-(methoxymethoxy)naphthalen-1-yl)-4,4,5,5-tetramethyl-1,3,2-dioxaborolane instead of N-(6-fluoro-4-(4,4,5,5-tetramethyl-1,3,2-dioxaborolan-2-yl)-5-((triisopropylsilyl)ethynyl)naphthalen-2-yl)-1,1-diphenylmethanimine. LCMS: 803.5.

### Intermediate 25-1: (6aR,7S,10R)-2-(8-ethyl-7-fluoro-3-(methoxymethoxy)naphthalen-1-yl)-1-fluoro-13-(((S,E)-4-(fluoromethylene)-1,3-dimethylpiperidin-3-yl)methoxy)-5,6,6a,7,8,9,10,11-octahydro-4H-3,11a,12,14,15-pentaaza-7,10-methanocyclohepta[4,5]cycloocta[1,2,3-de]naphthalene

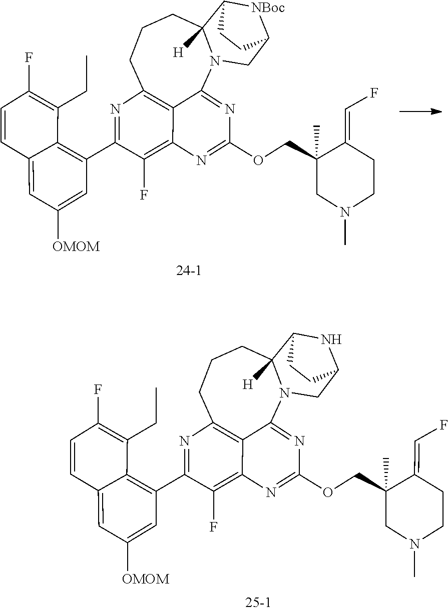

Intermediate 25-1 was synthesized in a manner similar to Intermediate 5-2 using Intermediate 24-1 instead of Intermediate 5-1. LCMS: 703.4.

### Intermediate 25-2: 4-(((6aR,7S,10R)-2-(8-ethyl-7-fluoro-3-(methoxymethoxy)naphthalen-1-yl)-1-fluoro-13-(((S,E)-4-(fluoromethylene)-1,3-dimethylpiperidin-3-yl)methoxy)-5,6,6a,7,8,9,10,11-octahydro-4H-3,11a,12,14,15-pentaaza-7,10-methanocyclohepta[4,5]cycloocta[1,2,3-de]naphthalen-15-yl)methyl)-5-methyl-1,3-dioxol-2-one

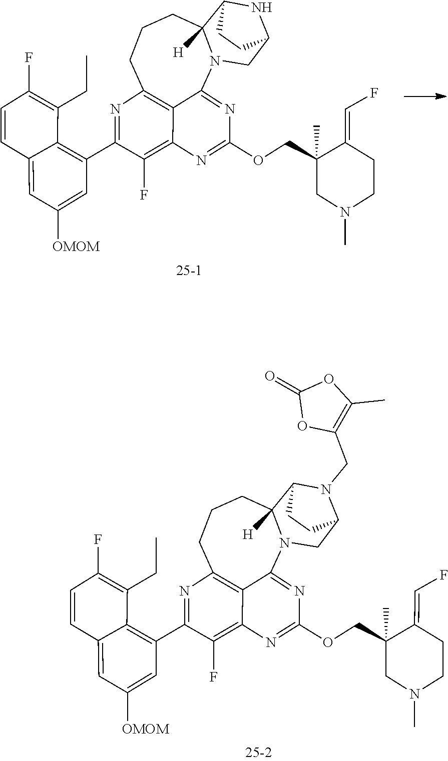

Intermediate 25-2 was synthesized in a manner similar to Intermediate 5-3 using Intermediate 25-1 instead of Intermediate 5-2. LCMS: 815.6.

### III. Compounds

### Example 1, Compound 1: 5-ethynyl-6-fluoro-4-((3aS,3bS,4S,7R,14bR)-12-fluoro-10-(((S,E)-4-(fluoromethylene)-1,3-dimethylpiperidin-3-yl)methoxy)-2,3,3a,3b,4,5,6,7,8,14b-decahydro-1H-8a,9,11,14,15-pentaaza-4,7-methanocyclopenta[d]naphtho[1,8-ab]heptalen-13-yl)naphthalen-2-ol

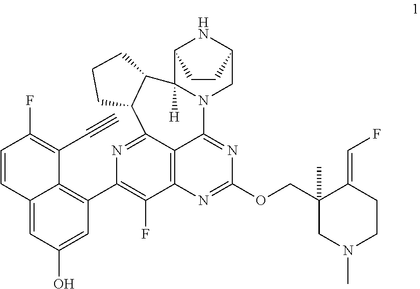

Hydrogen chloride solution (4.0 M in 1,4-dioxane, 0.50 mL, 2.0 mmol) was added via syringe to a vigorously stirred solution of Intermediate 1-13 (10.6 mg, 12.8 μmol) in acetonitrile (0.20 mL) at room temperature. After 60 min, the resulting mixture was purified by reverse phase preparative HPLC (0.1% acetic acid in acetonitrile/water) to give Example 1. ¹H NMR (400 MHz, Methanol-d4) δ 7.86 (ddd, J=9.2, 5.8, 3.5 Hz, 1H), 7.37-7.29 (m, 2.67H), 7.11 (d, J=2.6 Hz, 0.33H), 6.76 (dd, J=85.3, 4.5 Hz, 1H), 5.01 (dd, J=13.3, 2.2 Hz, 1H), 4.73-4.54 (m, 1H), 4.48-4.29 (m, 1H), 3.84 (s, 1H), 3.71-3.01 (m, 8H), 2.97-2.75 (m, 2H), 2.57-0.76 (m, 12H), 2.49 (s, 3H), 1.27 (s, 3H). LCMS: 681.2.

### Example 2, Compound 2: 5-ethynyl-6-fluoro-4-((6aR,7S,10R)-1-fluoro-13-(((S,E)-4-(fluoromethylene)-1,3-dimethylpiperidin-3-yl)methoxy)-5,6,6a,7,8,9,10,11-octahydro-4H-3,11a,12,14,15-pentaaza-7,10-methanocyclohepta[4,5]cycloocta[1,2,3-de]naphthalen-2-yl)naphthalen-2-ol

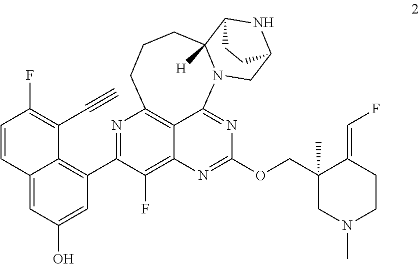

Hydrogen chloride solution (4.0 M in 1,4-dioxane, 0.065 mL, 0.26 mmol) was added via syringe to a vigorously stirred solution of Intermediate 5-1 (41.6 mg, 52.1 μmol) in acetonitrile (0.35 mL) at room temperature. After 75 min, the resulting mixture was purified by reverse phase preparative HPLC (0.1% trifluoroacetic acid in acetonitrile/water) to give Example 2. ¹H NMR (400 MHz, Methanol-d₄) δ 7.92 (dd, J=9.2, 5.4 Hz, 1H), 7.45-7.33 (m, 2H), 7.24 (dd, J=11.3, 2.6 Hz, 1H), 6.91 (dd, J=83.4, 5.3 Hz, 1H), 5.44 (t, J=11.5 Hz, 1H), 4.74-4.47 (m, 2H), 4.45-4.03 (m, 3H), 3.87-3.36 (m, 5H), 3.28-2.81 (m, 7H), 2.70 (d, J=44.7 Hz, 1H), 2.30 (s, 1H), 2.17-1.63 (m, 7H), 1.50-1.27 (m, 3H). LCMS: 655.2.

### Example 3, Compound 3: (R)-4-((1S,4R,15aR)-10,12-difluoro-8-(((S,E)-4-(fluoromethylene)-1,3-dimethylpiperidin-3-yl)methoxy)-2,3,4,5,13,14,15,15a-octahydro-1H-1,4-epiminoazepino[1′,2′:1,8]azocino[2,3,4-de]quinazolin-11-yl)-5-ethynyl-6-fluoronaphthalen-2-ol

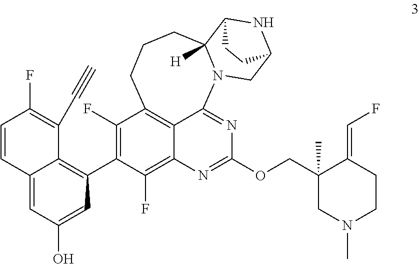

Example 3 was synthesized in a manner similar to Example 1 using Intermediate 3-7 instead of Intermediate 1-13, and using reverse phase preparative HPLC with 0.1% trifluoroacetic acid in acetonitrile/water elution for purification instead of reverse phase preparative HPLC with 0.1% acetic acid in acetonitrile/water. ¹H NMR (400 MHz, MeOD) δ 7.90 (dd, J=9.2, 5.7 Hz, 1H), 7.41-7.32 (m, 2H), 7.22-6.74 (m, 2H), 5.30 (d, J=14.8 Hz, 1H), 4.77-4.14 (m, 4H), 4.07 (dd, J=24.1, 7.8 Hz, 2H), 3.88-3.57 (m, 3H), 3.48 (s, 1H), 3.29-3.22 (m, 1H), 3.22-2.94 (m, 4H), 2.91 (s, 3H), 2.83-2.17 (m, 3H), 2.17-1.94 (m, 3H), 1.94-1.42 (m, 2H), 1.36 (s, 3H). LCMS: 672.3.

### Example 4, Compound 4: 5-ethynyl-6-fluoro-4-((7R,8S,8aS,9S,12R)-4-fluoro-2-(((S,E)-4-(fluoromethylene)-1,3-dimethylpiperidin-3-yl)methoxy)-7,8-dimethyl-7,8,8a,9,10,11,12,13-octahydro-1,3,6,13a,14-pentaaza-9,12-methanonaphtho[1,8-ab]heptalen-5-yl)naphthalen-2-ol

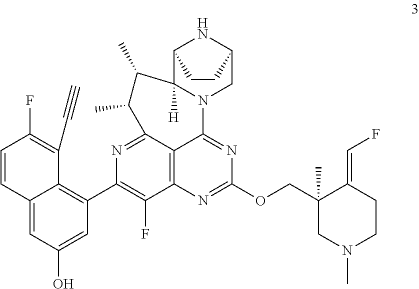

Example 4 was synthesized in a manner similar to Example 1 using Intermediate 4-5 instead of Intermediate 1-13. ¹H NMR (400 MHz, Methanol-d₄) δ 7.87 (dt, J=9.6, 5.1 Hz, 1H), 7.39-7.29 (m, 2H), 7.28 (d, J=2.6 Hz, 0.67H), 7.11 (d, J=2.6 Hz, 0.33H), 6.73 (d, J=85.5 Hz, 1H), 4.99 (d, J=13.3 Hz, 1H), 4.70 (dd, J=34.5, 10.7 Hz, 1H), 4.36 (dd, J=26.6, 10.8 Hz, 1H), 3.88-2.90 (m, 9H), 2.90-2.71 (m, 2H), 2.53-1.16 (m, 7H), 2.41 (s, 3H), 1.26 (s, 3H), 1.02 (d, J=6.5 Hz, 2H), 0.97 (d, J=6.4 Hz, 1H). LCMS: 669.2.

### Example 5, Compound 5: 4-(((6aR,7S,10R)-2-(8-ethynyl-7-fluoro-3-hydroxynaphthalen-1-yl)-1-fluoro-13-(((S,E)-4-(fluoromethylene)-1,3-dimethylpiperidin-3-yl)methoxy)-5,6,6a,7,8,9,10,11-octahydro-4H-3,11a,12,14,15-pentaaza-7,10-methanocyclohepta[4,5]cycloocta[1,2,3-de]naphthalen-15-yl)methyl)-5-methyl-1,3-dioxol-2-one

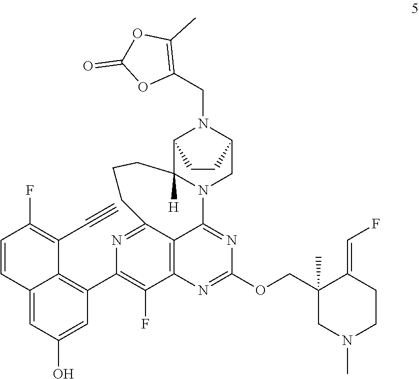

Hydrogen chloride solution (4.0 M in 1,4-dioxane, 3.12 mL, 13 mmol) was added over 1 min via syringe to a vigorously stirred solution of Intermediate 5-3 (1.03 g, 1.27 mmol) in acetonitrile (1.5 mL) at 0° C. After 120 min, sodium acetate (1.42 g, 17.4 mmol) was added, and the resulting mixture was purified by flash column chromatography on C18 reverse phase silica gel (0 to 43% acetonitrile in water, each containing 0.1% trifluoroacetic acid) to give Example 5. ¹H NMR (400 MHz, Acetonitrile-d₃) δ 7.99-7.87 (m, 1H), 7.47-7.32 (m, 2H), 7.27 (d, J=2.5 Hz, 0.67H), 7.22 (s, 0.33H), 6.84 (d, J=83.8 Hz, 1H), 5.32 (dd, J=14.9, 3.2 Hz, 1H), 4.94-4.65 (m, 1H), 4.54-4.37 (m, 1H), 4.24 (s, 1H), 4.20-3.77 (m, 5H), 3.68-3.17 (m, 3H), 3.12-1.56 (m, 14H), 2.77 (s, 3H), 2.20 (s, 3H), 1.32 (s, 3H). LCMS: 767.3.

### Example 6, Compound 6: 4-((1S,4R,11R,14S,14aS)-10,12-difluoro-8-(((S,E)-4-(fluoromethylene)-1,3-dimethylpiperidin-3-yl)methoxy)-14-methyl-1,2,3,4,5,13,14,14a-octahydro-1,4-epiminoazepino[1′,2′:1,7]azepino[2,3,4-de]quinazolin-11-yl)-5-ethynyl-6-fluoronaphthalen-2-ol

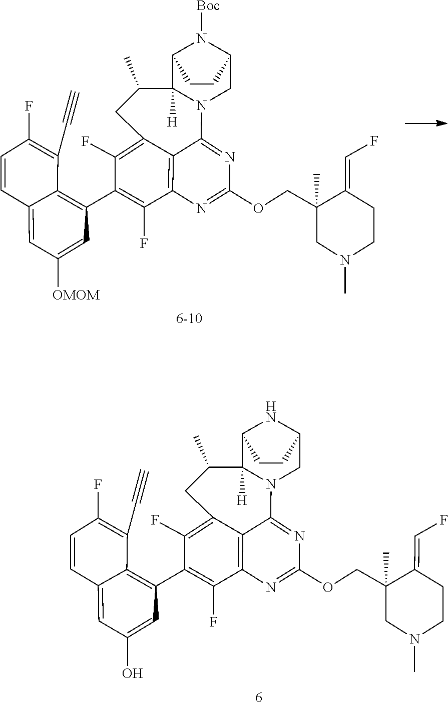

4 N HCl in dioxane (0.3 mL) was added to Intermediate 6-10 (15 mg, 0.0184 mmol) in 0.2 mL MeCN at 0° C. The reaction was stirred at 0° C. for one hour. The reaction crude was concentrated down, re-dissolve in 50% MeCN in water, purified via prep HPLC (0.1% TFA in MeCN and water) to give Example 6. ¹H NMR (400 MHz, Methanol-d4) δ 7.90 (dd, J=9.2, 5.7 Hz, 1H), 7.44-7.30 (m, 2H), 7.12 (d, J=2.5 Hz, 1H), 6.93 (d, J=83.5 Hz, 1H), 5.11 (dd, J=14.5, 2.2 Hz, 1H), 4.65 (d, J=11.4 Hz, 1H), 4.47-4.25 (m, 3H), 3.81 (dd, J=14.5, 2.2 Hz, 1H), 3.67 (s, 3H), 3.22-2.85 (m, 9H), 2.63 (m, 1H), 2.34-2.15 (m, 3H), 2.11-1.89 (m, 2H), 1.36 (s, 3H), 1.23 (d, J=6.4 Hz, 3H). LCMS (M+1): 672.3.

### Example 7, Compound 7: 5-ethynyl-6-fluoro-4-((6aR,7S,10R)-1-fluoro-13-(((S,E)-4-(fluoromethylene)-1,3-dimethylpiperidin-3-yl)methoxy)-5,6,6a,7,8,9,10,11-octahydro-4H-3,11a,12,14,15-pentaaza-7,10-methanocyclohepta[4,5]cycloocta[1,2,3-de]naphthalen-2-yl)naphthalen-2-amine

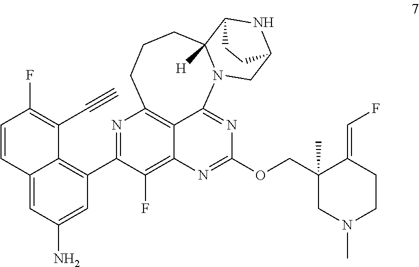

Example 7 was synthesized in a manner similar to Example 1 using Intermediate 7-4 instead of Intermediate 1-13, and and using reverse phase preparative HPLC with 0.1% trifluoroacetic acid in acetonitrile/water elution for purification instead of reverse phase preparative HPLC with 0.1% acetic acid in acetonitrile/water. ¹H NMR (400 MHz, Methanol-d₄) δ 7.83 (dd, J=9.2, 5.6 Hz, 1H), 7.40-7.14 (m, 3H), 6.92 (d, J=83.3 Hz, 1H), 5.42 (d, J=14.3 Hz, 1H), 4.74-4.46 (m, 2H), 4.33 (s, 1H), 4.19-4.01 (m, 2H), 3.64 (t, J=17.8 Hz, 3H), 2.98 (d, J=33.1 Hz, 9H), 2.64 (s, 1H), 2.31 (s, 1H), 1.86 (d, J=74.0 Hz, 7H), 1.52-1.22 (m, 3H). LCMS: 654.2.

### Example 8, Compound 8: (6aR,7S,10R)-2-(8-ethynyl-7-fluoronaphthalen-1-yl)-1-fluoro-13-(((S,E)-4-(fluoromethylene)-1,3-dimethylpiperidin-3-yl)methoxy)-5,6,6a,7,8,9,10,11-octahydro-4H-3,11a,12,14,15-pentaaza-7,10-methanocyclohepta[4,5]cycloocta[1,2,3-de]naphthalene

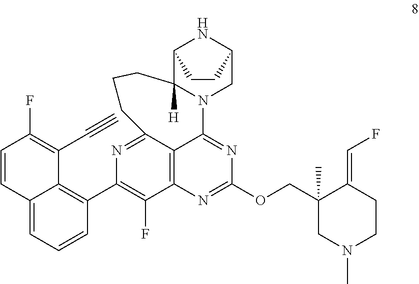

Example 8 was synthesized in a manner similar to Example 1 using Intermediate 5-1 instead of Intermediate 1-13. ¹H NMR (400 MHz, Methanol-d₄) δ 8.20-8.09 (m, 2H), 7.73-7.59 (m, 2H), 7.47 (td, J=8.9, 3.7 Hz, 1H), 6.89 (dd, J=83.6, 6.7 Hz, 1H), 5.51-5.30 (m, 1H), 4.71-4.47 (m, 2H), 4.41-4.20 (m, 1H), 4.21-4.10 (m, 1H), 4.10-4.01 (m, 1H), 3.77-3.51 (m, 3H), 3.48-1.23 (m, 15H), 2.92 (s, 3H), 1.35 (s, 3H). LCMS: 639.2.

### Example 9, Compound 9: (R)-4-((1S,4R,15aR)-10,12-difluoro-8-(((S,E)-4-(fluoromethylene)-1,3-dimethylpiperidin-3-yl)methoxy)-2,3,4,5,13,14,15,15a-octahydro-1H-1,4-epiminoazepino[1′,2′:1,8]azocino[2,3,4-de]quinazolin-11-yl)-5-ethynyl-6-fluoronaphthalen-2-amine

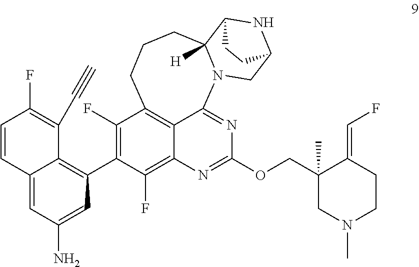

Intermediate 9-4 (5 mg) was cooled to 0° C., to it was added TFA (0.3 mL) and stirred for 0.5 hour. Upon completion, diluted it with toluene and concentrated to dryness. The residue was purified by RP-HPLC (5% to 60% 0.1% TFA in MeCN/0.1% TFA in H₂O). Fractions containing the product were pooled and lyophilized to yield the title compound. ¹H NMR (400 MHz, MeOD) δ 7.82 (dd, J=9.2, 5.8 Hz, 1H), 7.35-7.27 (m, 2H), 7.18-6.78 (m, 2H), 5.29 (d, J=14.7 Hz, 1H), 4.70 (d, J=11.4 Hz, 1H), 4.30 (t, J=32.9 Hz, 3H), 4.18-3.98 (m, 2H), 3.94-3.57 (m, 4H), 3.56-3.40 (m, 1H), 3.28-3.22 (m, 1H), 3.19-3.00 (m, 5H), 2.92 (s, 3H), 2.86-2.53 (m, 1H), 2.43-2.18 (m, 1H), 2.14-1.94 (m, 1H), 1.98-1.53 (m, 4H), 1.36 (s, 3H). LCMS (m/z): 671.4.

### Example 10, Compound 10: 2-amino-4-((1S,4R,11R,14S,14aS)-12-chloro-10-fluoro-8-(((S,E)-4-(fluoromethylene)-1,3-dimethylpiperidin-3-yl)methoxy)-14-methyl-1,2,3,4,5,13,14,14a-octahydro-1,4-epiminoazepino[1′,2′:1,7]azepino[2,3,4-de]qiuinazolin-11-yl)-7-fluorobenzo[b]thiophene-3-carbonitrile

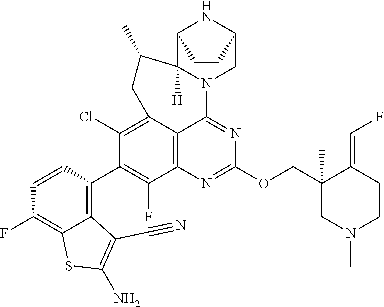

A mixture of Intermediate 10-8 (31.1 mg, 34.8 μmol) and 4 N HCl in dioxane (3 mL) was stirred at rt for 1 h followed by at 35° C. for 1 h. The reaction mixture was concentrated, and the residue was purified by preparative HPLC (Gemini 5 um C18 110A, AX, 100×30.00 mm 5 micro column) eluting 5-45% acetonitrile (0.1% TFA) in water (0.1% TFA). The product containing fractions were combined and freeze-dried to give Example 10. ¹H NMR (400 MHz, Methanol-d₄) δ 7.38-6.61 (m, 3H), 5.13 (d, J=14.4 Hz, 1H), 4.58-4.17 (m, 4H), 3.92-3.37 (m, 5H), 3.21-2.96 (m, 5H), 2.92 (s, 3H), 2.56 (s, 1H), 2.12 (d, J=41.0 Hz, 3H), 1.90 (d, J=20.5 Hz, 1H), 1.37-1.12 (m, 6H). LCMS: 694.

### Example 11, Compound 11: 4-(((6aR,7S,10R)-2-(3-amino-8-ethynyl-7-fluoronaphthalen-1-yl)-1-fluoro-13-(((S,E)-4-(fluoromethylene)-1,3-dimethylpiperidin-3-yl)methoxy)-5,6,6a,7,8,9,10,11-octahydro-4H-3,11a,12,14,15-pentaaza-7,10-methanocyclohepta[4,5]cycloocta[1,2,3-de]naphthalen-15-yl)methyl)-5-methyl-1,3-dioxol-2-one

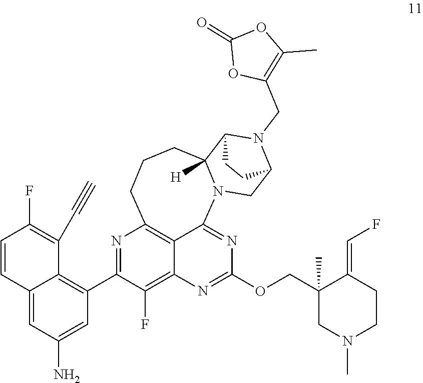

Example 11 was synthesized in a manner similar to Example 1 using Intermediate 11-2 instead of Intermediate 5-3 and using reverse phase preparative HPLC with 0.1% trifluoroacetic acid in acetonitrile/water elution for purification instead of reverse phase preparative HPLC with 0.1% acetic acid in acetonitrile/water. ¹H NMR (400 MHz, Acetonitrile-d₃) δ 7.79 (dd, J=9.2, 5.9 Hz, 1H), 7.28 (td, J=9.1, 2.8 Hz, 1H), 7.12 (dd, J=21.3, 2.4 Hz, 2H), 6.64 (d, J=86.9 Hz, 1H), 5.22 (d, J=12.7 Hz, 1H), 4.82-4.51 (m, 4H), 4.42 (dd, J=21.6, 10.5 Hz, 1H), 3.85 (d, J=10.0 Hz, 1H), 3.75-3.10 (m, 10H), 2.88-2.24 (m, 8H), 1.83-1.26 (m, 7H), 1.18 (s, 3H). LCMS: 766.1.

### Example 12, Compound 12: 5-ethynyl-6-fluoro-4-((8S,8aS,9S,12R)-4-fluoro-2-(((S,E)-4-(fluoromethylene)-1,3-dimethylpiperidin-3-yl)methoxy)-8-methyl-7,8,8a,9,10,11,12,13-octahydro-1,3,6,13a,14-pentaaza-9,12-methanonaphtho[1,8-ab]heptalen-5-yl)naphthalen-2-ol

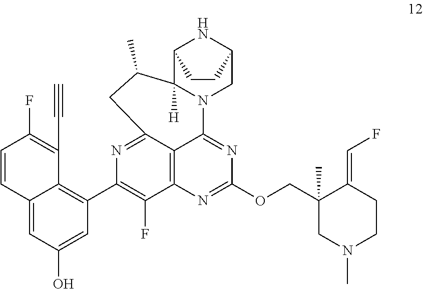

Example 12 was synthesized in a manner similar to Example 1 using Intermediate 12-1 instead of Intermediate 1-13 and using reverse phase preparative HPLC with 0.1% trifluoroacetic acid in acetonitrile/water elution for purification instead of reverse phase preparative HPLC with 0.1% acetic acid in acetonitrile/water. ¹H NMR (400 MHz, Acetonitrile-d₃) δ 7.90 (dt, J=9.2, 5.9 Hz, 1H), 7.40 (d, J=2.7 Hz, 1H), 7.35 (td, J=9.1, 2.5 Hz, 1H), 7.24 (dd, J=41.0, 2.6 Hz, 1H), 6.82 (d, J=84.0 Hz, 1H), 5.04 (dt, J=14.5, 2.5 Hz, 1H), 4.71 (d, J=11.8 Hz, 1H), 4.42-4.25 (m, 2H), 4.26-4.16 (m, 1H), 3.99-3.86 (m, 1H), 3.69-3.07 (m, 8H), 3.05-1.60 (m, 7H), 2.24-2.12 (m, 2H), 2.00-1.96 (m, 1H), 1.33-1.24 (m, 3H), 1.14 (dd, J=9.8, 6.1 Hz, 3H).

### Example 13, Compound 13: 4-ethyl-5-(((6aR,7S,10R)-2-(8-ethynyl-7-fluoro-3-hydroxynaphthalen-1-yl)-1-fluoro-13-(((S,E)-4-(fluoromethylene)-1,3-dimethylpiperidin-3-yl)methoxy)-5,6,6a,7,8,9,10,11-octahydro-4H-3,11a,12,14,15-pentaaza-7,10-methanocyclohepta[4,5]cycloocta[1,2,3-de]naphthalen-15-yl)methyl)-1,3-dioxol-2-one

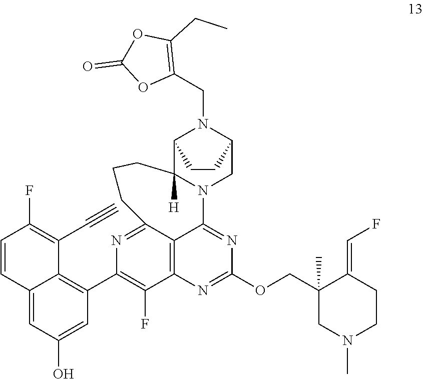

Example 13 was synthesized in a manner similar to Example 5 using 4-(bromomethyl)-5-ethyl-1,3-dioxol-2-one instead of 4-(bromomethyl)-5-methyl-1,3-dioxol-2-one and using reverse phase preparative HPLC (0.1% trifluoroacetic acid in acetonitrile/water) for purification instead of flash column chromatography on C18 reverse phase silica gel (0 to 43% acetonitrile in water, each containing 0.1% trifluoroacetic acid). ¹H NMR (400 MHz, Acetonitrile-d₃) δ 7.97-7.86 (m, 1H), 7.48-7.31 (m, 2H), 7.26 (s, 0.67H), 7.21 (s, 0.33H), 6.84 (d, J=84.1 Hz, 1H), 5.38-5.22 (m, 1H), 4.99-4.72 (m, 1H), 4.39-4.28 (m, 1H), 4.18-4.01 (m, 2H), 3.98-3.65 (m, 4H), 3.63-3.45 (m, 2H), 3.40-3.20 (m, 2H), 3.10-1.38 (m, 18H), 1.31 (s, 3H), 1.24-1.09 (m, 3H). LCMS: 781.3.

### Example 14, Compound 14: 5-ethynyl-6-fluoro-4-((6aR,7S,10R)-1-fluoro-13-(((S,E)-4-(fluoromethylene)-3-methyl-1-(methyl-d₃)piperidin-3-yl)methoxy-d2)-5,6,6a,7,8,9,10,11-octahydro-4H-3,11a,12,14,15-pentaaza-7,10-methanocyclohepta[4,5]cycloocta[1,2,3-de]naphthalen-2-yl)naphthalen-2-ol

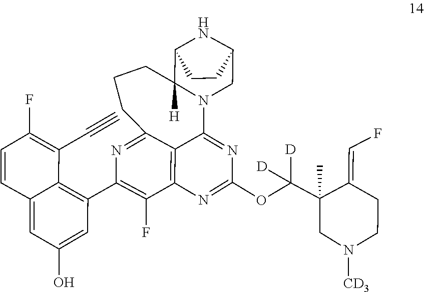

Example 14 was synthesized in a manner similar to Example 2 using Intermediate 14-2 instead of Intermediate 1-12. ¹H NMR (400 MHz, Methanol-d₄) δ 7.94-7.83 (m, 1H), 7.42-7.30 (m, 2H), 7.25-7.21 (m, 0.62H), 7.21-7.16 (m, 0.38H), 6.69 (d, J=86.0 Hz, 1H), 5.40 (d, J=13.3 Hz, 1H), 3.98 (d, J=9.9 Hz, 1H), 3.88-3.75 (m, 1H), 3.67-2.53 (m, 10H), 2.43-1.17 (m, 9H), 1.24 (s, 3H). LCMS: 660.3.

### Example 15, Compound 15: 4-(((6aR,7S,10R)-2-(8-ethynyl-7-fluoro-3-hydroxynaphthalen-1-yl)-1-fluoro-13-(((S,E)-4-(fluoromethylene)-1,3-dimethylpiperidin-3-yl)methoxy)-5,6,6a,7,8,9,10,11-octahydro-4H-3,11a,12,14,15-pentaaza-7,10-methanocyclohepta[4,5]cycloocta[1,2,3-de]naphthalen-15-yl)methyl)-5-isopropyl-1,3-dioxol-2-one

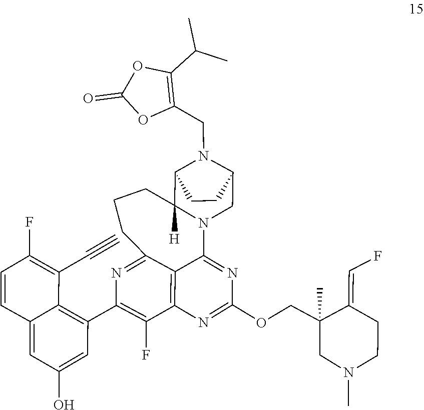

Example 15 was synthesized in a manner similar to Example 5 using 4-(bromomethyl)-5-isopropyl-1,3-dioxol-2-one instead of 4-(bromomethyl)-5-methyl-1,3-dioxol-2-one and using reverse phase preparative HPLC (0.1% trifluoroacetic acid in acetonitrile/water) for purification instead of flash column chromatography on C18 reverse phase silica gel (0 to 43% acetonitrile in water, each containing 0.1% trifluoroacetic acid). ¹H NMR (400 MHz, Acetonitrile-d₃) δ 7.99-7.87 (m, 1H), 7.47-7.33 (m, 2H), 7.29 (s, 0.67H), 7.24 (s, 0.33H), 6.86 (d, J=83.8 Hz, 1H), 5.33 (dd, J=14.2, 3.4 Hz, 1H), 5.01-4.68 (m, 1H), 4.48-4.31 (m, 1H), 4.26-4.07 (m, 2H), 4.06-3.87 (m, 2H), 3.88-3.70 (m, 2H), 3.70-3.48 (m, 2H), 3.46-3.24z (m, 2H), 3.17-1.50 (m, 15H), 1.39-1.30 (m, 3H), 1.29-1.21 (m, 6H). LCMS: 795.3.

### Example 16, Compound 16: 4-(tert-butyl)-5-(((6aR,7S,10R)-2-(8-ethynyl-7-fluoro-3-hydroxynaphthalen-1-yl)-1-fluoro-13-(((S,E)-4-(fluoromethylene)-1,3-dimethylpiperidin-3-yl)methoxy)-5,6,6a,7,8,9,10,11-octahydro-4H-3,11a,12,14,15-pentaaza-7,10-methanocyclohepta[4,5]cycloocta[1,2,3-de]naphthalen-15-yl)methyl)-1,3-dioxol-2-one

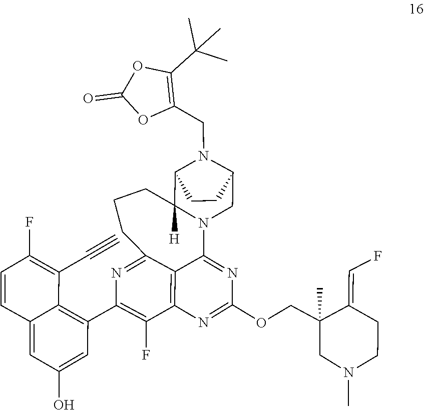

Example 16 was synthesized in a manner similar to Example 5 using 4-(bromomethyl)-5-(tert-butyl)-1,3-dioxol-2-one instead of 4-(bromomethyl)-5-methyl-1,3-dioxol-2-one and using reverse phase preparative HPLC (0.1% trifluoroacetic acid in acetonitrile/water) for purification instead of flash column chromatography on C18 reverse phase silica gel (0 to 43% acetonitrile in water, each containing 0.1% trifluoroacetic acid). ¹H NMR (400 MHz, Acetonitrile-d₃) δ 8.01-7.86 (m, 1H), 7.48-7.33 (m, 2H), 7.29 (s, 0.67H), 7.24 (s, 0.33H), 6.86 (d, J=83.7 Hz, 1H), 5.38-5.23 (m, 1H), 4.99-4.76 (m, 1H), 4.43-4.26 (m, 1H), 4.19-3.93 (m, 2H), 3.90-3.77 (m, 2H), 3.77-3.45 (m, 4H), 3.45-3.22 (m, 2H), 3.12-1.40 (m, 14H), 1.36 (s, 9H), 1.34 (s, 3H). LCMS: 809.2.

### Example 17, Compound 17: 4-(((6aR,7S,10R)-2-(8-ethynyl-7-fluoro-3-hydroxynaphthalen-1-yl)-1-fluoro-13-(((S,E)-4-(fluoromethylene)-3-methyl-1-(methyl-d₃)piperidin-3-yl)methoxy-d2)-5,6,6a,7,8,9,10,11-octahydro-4H-3,11a,12,14,15-pentaaza-7,10-methanocyclohepta[4,5]cycloocta[1,2,3-de]naphthalen-15-yl)methyl)-5-methyl-1,3-dioxol-2-one

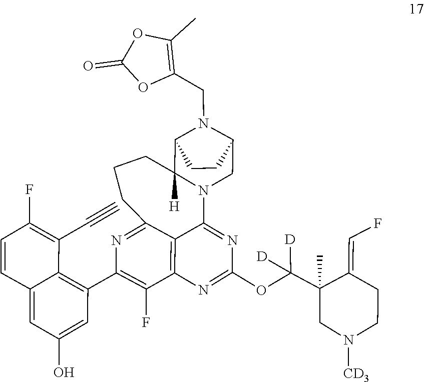

Example 17 was synthesized in a manner similar to Example 5 using Intermediate 14-2 instead of Intermediate 1-12. ¹H NMR (400 MHz, Acetonitrile-d₃) δ 7.98-7.86 (m, 1H), 7.51-7.31 (m, 2H), 7.26 (d, J=2.6 Hz, 0.67H), 7.21 (s, 0.33H), 6.83 (d, J=83.9 Hz, 1H), 5.31 (dd, J=14.6, 3.3 Hz, 1H), 4.50-4.34 (m, 1H), 4.30-4.07 (m, 1H), 4.07-3.71 (m, 4H), 3.65-3.44 (m, 2H), 3.35 (t, J=11.6 Hz, 1H), 3.13-1.38 (m, 14H), 2.19 (s, 3H), 1.36-1.28 (m, 3H). LCMS: 772.2.

### Example 18, Compound 18: 5-ethynyl-6-fluoro-4-((7R,8S,8aS,9S,12R)-4-fluoro-2-(((S,E)-4-(fluoromethylene)-3-methyl-1-(methyl-d₃)piperidin-3-yl)methoxy-d2)-7,8-dimethyl-7,8,8a,9,10,11,12,13-octahydro-1,3,6,13a,14-pentaaza-9,12-methanonaphtho[1,8-ab]heptalen-5-yl)naphthalen-2-ol

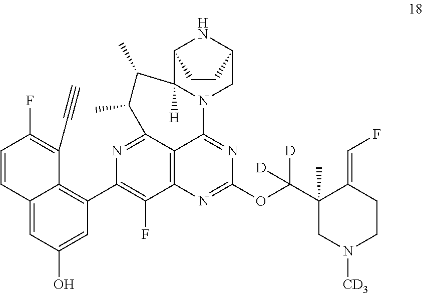

Example 18 was synthesized in a manner similar to Example 4 using Intermediate 14-2 instead of Intermediate 1-12. ¹H NMR (400 MHz, Methanol-d₄) δ 7.90-7.83 (m, 1H), 7.39-7.30 (m, 2H), 7.28 (d, J=2.6 Hz, 0.67H), 7.11 (d, J=2.6 Hz, 0.33H), 6.73 (d, J=85.7 Hz, 1H), 4.99 (d, J=13.1 Hz, 1H), 3.89-3.74 (m, 2H), 3.61 (d, J=13.0 Hz, 1H), 3.55-3.43 (m, 2.67H), 3.19 (s, 0.33H), 3.11-2.92 (m, 3H), 2.92-2.69 (m, 2H), 2.49-2.35 (m, 1H), 2.35-2.17 (m, 2H), 2.11-1.56 (m, 3H), 1.33 (d, J=6.9 Hz, 1H), 1.32 (d, J=6.9 Hz, 2H), 1.99 (s, 3H), 1.26 (s, 3H), 1.02 (d, J=6.5 Hz, 2H), 0.97 (d, J=6.5 Hz, 1H). LCMS: 674.2.

### Example 19, Compound 19: 5-ethynyl-6-fluoro-4-((8S,8aS,9S,12R)-4-fluoro-2-(((S,E)-4-(fluoromethylene)-3-methyl-1-(methyl-d₃)piperidin-3-yl)methoxy-d2)-8-methyl-7,8,8a,9,10,11,12,13-octahydro-1,3,6,13a,14-pentaaza-9,12-methanonaphtho[1,8-ab]heptalen-5-yl)naphthalen-2-ol

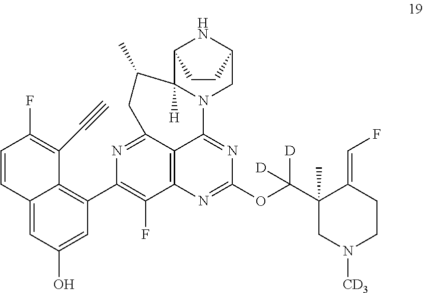

Example 19 was synthesized in a manner similar to Example 12 using Intermediate 14-2 instead of Intermediate 1-12. ¹H NMR (400 MHz, Methanol-d₄) δ 7.88 (ddd, J=9.2, 5.7, 3.5 Hz, 1H), 7.38 (t, J=1.9 Hz, 1H), 7.34 (t, J=9.0 Hz, 1H), 7.21 (dd, J=37.5, 2.6 Hz, 1H), 7.03-6.77 (m, 1H), 5.17 (dd, J=14.6, 2.2 Hz, 1H), 4.44-4.24 (m, 2H), 4.02-3.78 (m, 1H), 3.65 (d, J=17.1 Hz, 2H), 3.51-3.35 (m, 1H), 3.31-3.19 (m, 7H), 3.18-2.77 (m, 4H), 2.59 (s, 1H), 2.35-1.87 (m, 2H), 1.50-1.40 (m, 1H), 1.34 (s, 2H), 1.26 (td, J=12.5, 11.8, 5.1 Hz, 3H). LCMS: 660.2.

### Example 20, Compound 20: 5-ethynyl-6-fluoro-4-((7R,8S,8aS,9S,12R)-4-fluoro-2-(((S,E)-4-(fluoromethylene)-1,3-dimethylpiperidin-3-yl)methoxy)-7,8,14-trimethyl-7,8,8a,9,10,11,12,13-octahydro-1,3,6,13a,14-pentaaza-9,12-methanonaphtho[1,8-ab]heptalen-5-yl)naphthalen-2-ol

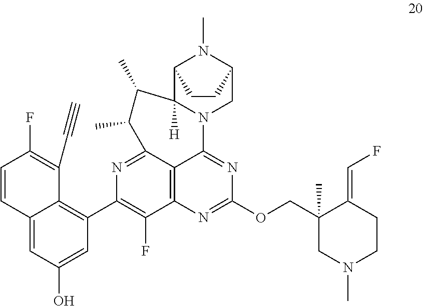

Aqeuous formaldehyde solution (37% wt, 21.3 μL, 285 μmol) was added via syringe to a stirred mixture of Example 4 (3.42 mg, 5.11 μmol), acetic acid (4.1 μL, 71 μmol), and methanol (0.30 mL) at room temperature. After 2 min, sodium cyanoborohydride solution (1.0 M in tetrahydrofuran, 107 μL, 110 μmol) was added via syringe. After 14.5 h, the resulting mixture was purified by reverse phase preparative HPLC (0.1% acetic acid in acetonitrile/water) to give Example 20. ¹H NMR (400 MHz, Methanol-d₄) δ 7.89-7.80 (m, 1H), 7.35-7.27 (m, 2H), 7.25 (d, J=2.6 Hz, 0.67H), 7.09 (d, J=2.6 Hz, 0.33H), 6.67 (d, J=85.9 Hz, 1H), 5.08-4.53 (m, 3H), 4.33 (d, J=10.6 Hz, 0.67H), 4.27 (d, J=10.6 Hz, 0.33H), 3.69-3.55 (m, 1H), 3.47-3.07 (m, 4H), 3.07-2.94 (m, 1H), 2.90 (d, J=11.5 Hz, 1H), 2.87-2.74 (m, 2H), 2.69 (d, J=14.8 Hz, 1H), 2.41-2.26 (m, 1H), 2.37 (s, 2H), 2.36 (s, 1H), 2.29 (s, 3H), 2.26-1.98 (m, 3H), 1.88-1.72 (m, 1H), 1.66-0.82 (m, 7H), 0.99 (d, J=6.5 Hz, 2H), 0.94 (d, J=6.6 Hz, 1H). LCMS: 683.3.

### Example 21, Compound 21: 6-fluoro-4-((8S,8aS,9S,12R)-4-fluoro-2-(((S,E)-4-(fluoromethylene)-1,3-dimethylpiperidin-3-yl)methoxy)-8-methyl-7,8,8a,9,10,11,12,13-octahydro-1,3,6,13a,14-pentaaza-9,12-methanonaphtho[1,8-ab]heptalen-5-yl)-5-(trifluoromethoxy)naphthalen-2-ol

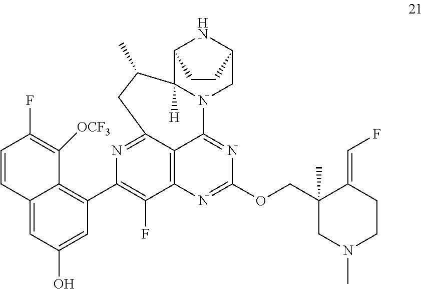

Example 21 was synthesized in a manner similar to Example 12 using Intermediate 21-1 instead of Intermediate 12-1 and using reverse phase preparative HPLC (0.1% trifluoroacetic acid in acetonitrile/water) for purification. ¹H NMR (400 MHz, Acetonitrile-d₃) δ 7.89 (td, J=9.0, 5.1 Hz, 1H), 7.51-7.23 (m, 3H), 6.81 (dd, J=83.9, 6.6 Hz, 1H), 5.03 (dt, J=14.5, 2.7 Hz, 1H), 4.73 (d, J=12.1 Hz, 1H), 4.35 (d, J=5.7 Hz, 1H), 4.29-4.13 (m, 2H), 3.94 (dd, J=14.4, 2.2 Hz, 1H), 3.80-3.32 (m, 5H), 3.26-3.07 (m, 1H), 3.00 (dd, J=14.5, 8.4 Hz, 2H), 2.94-2.73 (m, 5H), 2.69-2.45 (m, 1H), 2.26-2.08 (m, 2H), 2.05-1.96 (m, 1H), 1.88-1.72 (m, 1H), 1.40 (t, J=8.4 Hz, 1H), 1.29 (s, 3H), 1.14 (d, J=6.5 Hz, 3H). LCMS: 715.3.

### Example 22, Compound 22: 4-(((1S,4R,11R,15aR)-11-(8-ethynyl-7-fluoro-3-hydroxynaphthalen-1-yl)-10,12-difluoro-8-(((S,E)-4-(fluoromethylene)-1,3-dimethylpiperidin-3-yl)methoxy)-2,3,4,5,13,14,15,15a-octahydro-1H-1,4-epiminoazepino[1′,2′:1,8]azocino[2,3,4-de]quinazolin-16-yl)methyl)-5-methyl-1,3-dioxol-2-one

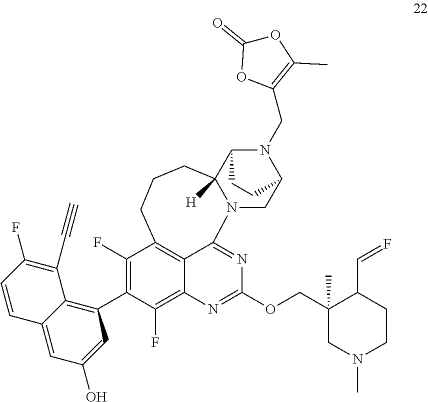

Example 22 was synthesized in a manner similar to Example 5 using Intermediate 22-2 instead of Intermediate 5-3. ¹H NMR (400 MHz, CD3CN) δ 10.16 (s, 1H), 7.96 (dd, J=9.2, 5.8 Hz, 1H), 7.49-7.36 (m, 2H), 7.21 (d, J=2.6 Hz, 1H), 6.87 (d, J=84.2 Hz, 1H), 5.25 (dd, J=14.9, 3.0 Hz, 1H), 4.85 (d, J=12.1 Hz, 1H), 4.43 (d, J=8.0 Hz, 1H), 4.37-4.27 (m, 1H), 4.11 (dd, J=13.4, 9.4 Hz, 3H), 4.07-3.83 (m, 3H), 3.59 (d, J=10.2 Hz, 1H), 3.50 (d, J=13.1 Hz, 1H), 3.25 (s, 1H), 3.18 (d, J=13.1 Hz, 1H), 3.11-2.99 (m, 3H), 2.83 (d, J=12.8 Hz, 1H), 2.61 (t, J=15.1 Hz, 1H), 2.44-2.03 (m, 6H), 1.94 (s, 3H), 1.67 (s, 4H), 1.34 (s, 3H). LCMS: 784.4.

### Example 23, Compound 23: 4-(((7R,8S,8aS,9S,12R)-5-(8-ethynyl-7-fluoro-3-hydroxynaphthalen-1-yl)-4-fluoro-2-(((S,E)-4-(fluoromethylene)-1,3-dimethylpiperidin-3-yl)methoxy)-7,8-dimethyl-7,8,8a,9,10,11,12,13-octahydro-1,3,6,13a,14-pentaaza-9,12-methanonaphtho[1,8-ab]heptalen-14-yl)methyl)-5-methyl-1,3-dioxol-2-one

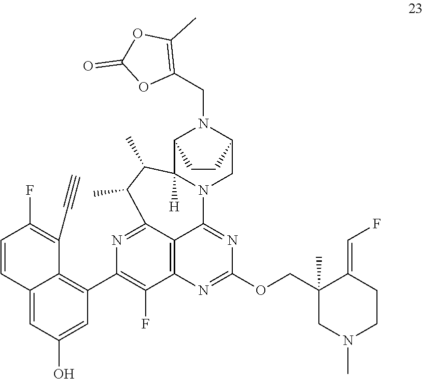

Example 23 was synthesized in a manner similar to Example 5 using Intermediate 23-2 instead of Intermediate 5-3. ¹H NMR (400 MHz, Acetonitrile-d₃) δ 7.91 (dt, J=9.1, 6.5 Hz, 1H), 7.42 (t, J=2.4 Hz, 1H), 7.41-7.37 (m, 1H), 7.37-7.32 (m, 1H), 6.83 (d, J=83.9 Hz, 1H), 5.06-4.88 (m, 1H), 4.73 (t, J=13.7 Hz, 1H), 4.25 (dd, J=31.5, 11.9 Hz, 1H), 3.91 (d, J=23.6 Hz, 1H), 3.84-3.72 (m, 2H), 3.66 (d, J=4.6 Hz, 1H), 3.60-3.51 (m, 1H), 3.43 (tt, J=11.0, 5.1 Hz, 1H), 3.34-3.01 (m, 6H), 2.99-2.82 (m, 6H), 2.76 (d, J=6.1 Hz, 2H), 2.24-2.07 (m, 5H), 1.91-1.78 (m, 2H), 1.28 (d, J=9.3 Hz, 2H), 1.23-1.11 (m, 3H), 0.94-0.82 (m, 3H). LCMS: 781.3.

### Example 24, Compound 24: 5-ethyl-6-fluoro-4-((6aR,7S,10R)-1-fluoro-13-(((S,E)-4-(fluoromethylene)-1,3-dimethylpiperidin-3-yl)methoxy)-5,6,6a,7,8,9,10,11-octahydro-4H-3,11a,12,14,15-pentaaza-7,10-methanocyclohepta[4,5]cycloocta[1,2,3-de]naphthalen-2-yl)naphthalen-2-ol

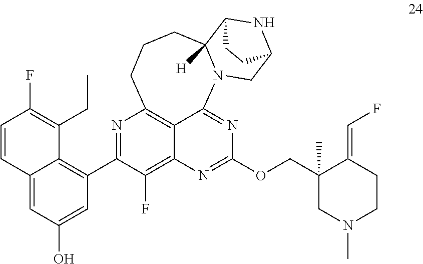

Example 24 was synthesized in a manner similar to Example 1 using Intermediate 24-1 instead of Intermediate 1-13 and using reverse phase preparative HPLC with 0.1% trifluoroacetic acid in acetonitrile/water elution for purification instead of reverse phase preparative HPLC with 0.1% acetic acid in acetonitrile/water. ¹H NMR (400 MHz, Acetonitrile-d₃) δ 7.71 (dd, J=9.1, 5.9 Hz, 1H), 7.35 (d, J=2.6 Hz, 1H), 7.29 (t, J=9.4 Hz, 1H), 7.07 (dd, J=28.0, 2.7 Hz, 1H), 6.81 (d, J=84.0 Hz, 1H), 5.40-5.24 (m, 1H), 4.74 (d, J=11.7 Hz, 1H), 4.41-4.18 (m, 3H), 4.18-3.91 (m, 1H), 3.72 (d, J=14.7 Hz, 1H), 3.58 (s, 2H), 3.35 (td, J=12.8, 4.6 Hz, 1H), 2.96-2.33 (m, 16H), 2.22 (dd, J=15.6, 8.1 Hz, 1H), 1.82-1.52 (m, 3H), 1.39-1.18 (m, 3H), 0.75 (dt, J=21.6, 7.4 Hz, 3H). LCMS: 659.2.

### Example 25, Compound 25: 4-(((6aR,7S,10R)-2-(8-ethyl-7-fluoro-3-hydroxynaphthalen-1-yl)-1-fluoro-13-(((S,E)-4-(fluoromethylene)-1,3-dimethylpiperidin-3-yl)methoxy)-5,6,6a,7,8,9,10,11-octahydro-4H-3,11a,12,14,15-pentaaza-7,10-methanocyclohepta[4,5]cycloocta[1,2,3-de]naphthalen-15-yl)methyl)-5-methyl-1,3-dioxol-2-one

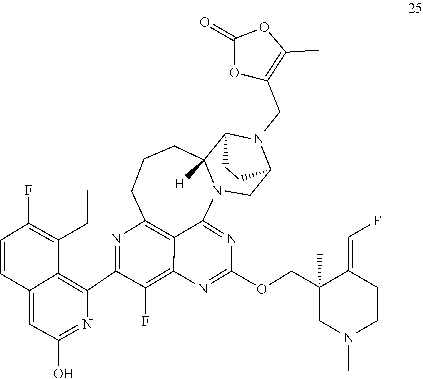

Example 25 was synthesized in a manner similar to Example 5 using Intermediate 25-2 instead of Intermediate 5-3. ¹H NMR (400 MHz, Acetonitrile-d₃) δ 7.72 (dd, J=9.1, 5.9 Hz, 1H), 7.35 (d, J=2.7 Hz, 1H), 7.29 (t, J=9.4 Hz, 1H), 7.07 (dd, J=26.4, 2.7 Hz, 1H), 6.83 (d, J=84.1 Hz, 1H), 5.35 (ddd, J=15.3, 7.8, 3.0 Hz, 1H), 4.76 (d, J=11.7 Hz, 1H), 4.59-4.38 (m, 1H), 4.37-4.13 (m, 2H), 4.13-3.91 (m, 3H), 3.86 (d, J=14.7 Hz, 1H), 3.65-3.07 (m, 12H), 3.05-2.70 (m, 7H), 2.70-2.24 (m, 1H), 2.19 (s, 3H), 1.86-1.53 (m, 2H), 1.30 (s, 3H), 0.75 (dt, J=21.6, 7.4 Hz, 3H). LCMS: 771.5.

### IV. Biological Examples

### Example A. KRAS G12D (GDP) Biochemical Assay

Compounds were tested for binding to GDP-loaded KRAS G12D in a 384-well assay format using a TR-FRET probe displacement assay in buffer consisting of 50 mM Hepes (pH 7.4), 150 mM NaCl, 5 mM MgCl₂ and 0.005% Tween-20. 0.5 nM enzyme was used in this assay with 0.25 nM Eu-streptavidin and 200 nM (2×K_{D}) Cy-5 labelled probe. Compounds were serially diluted (1:3) in DMSO. The LabCyte ECHO Acoustic dispenser system was used to pre-spot the assay plates (384-well Non-Binding Surface plates, Corning, Catalog #3824) with 50 nL of compound. The compounds were pre-incubated with 5 μL of 2× final enzyme concentration for 30 minutes before adding 5 μL of 2× final concentration of Eu-streptavidin and TR-FRET probe (10 p L final reaction volume). The plates were incubated at room temperature for 2 hours before measuring TR-FRET ratio on the Envision plate reader. IC₅₀ values were defined as the compound concentration that causes a 50% decrease in TR-FRET ratio and were calculated using a sigmoidal dose-response model to generate curve fits.

### Example B. 2D Cell Viability Assay

Compounds were tested in a 384-well format for their ability to inhibit the viability of GP2D (KRAS G12D) cells in 2D assays. Compounds were serially diluted (1:3) in DMSO. The LabCyte ECHO Acoustic dispenser system was used to pre-spot assay plates with 200 nL of test molecule per well. 1000 cells/well (40 p L volume per well) in RPMI medium with 10% FBS, and Penicillin-Streptomycin-Glutamine were plated in pre-spotted 384-well plates (Greiner, Catalog #781076) on the BioTEK EL406 liquid dispenser with a 5 μL dispensing cassette (BioTek 7170011). The plates were incubated at 37° C., 5% CO₂ for 4 days before addition of CellTiter-Glo (CTG) reagent and measurement of luminescence signal. EC₅₀ values were defined as the compound concentration that causes a 50% decrease in luminescence signal and were calculated using a sigmoidal dose-response model to generate curve fits.

### Example C. Pharmacokinetic Profiling!

### Rat Oral Pharmacokinetics

Test compounds were formulated in solution formulations (10 w/v % Sulfobutylether-β-cyclodextrin; 90% 50 mM Citrate) and administered orally at 7.5 or 15 mg-eq/kg to a group of three Male Sprague Dawley rats. The animals were fasted overnight prior to dose administration and up to four hours after dosing. The test articles were administrated by oral gavage at a dose volume of 5 mL-eq/kg.

Blood samples were collected at predose and 0.25, 0.5, 1, 2, 4, 6, 8, 12, 24, 48 and 72 hours post dose into K₂EDTA tubes and stored on wet ice until processed. Whole blood was processed to plasma by centrifugation (3500 rpm for 10 minutes at 5° C.) within 30 minutes of collection. Plasma samples were placed in Micronic 96 well tubes and stored at −80° C. until transferred.

Plasma concentration of the test compounds was determined by LC-MS/MS. Pharmacokinetic parameters were estimated using non-compartment model analysis. The maximum observed concentration (Cₘₐₓ) and area under the curve from time of dosing to last measured concentration (AUCₗₐₛₜ) were calculated and reported.

### Mouse Oral Cassette Pharmacokinetics

Test compounds (1-3 compounds per cassette) were formulated in solution formulations (10 w/v % Captisol in 50 mM Citrate buffer) and administered orally at 30 mg-eq./kg each compound to a group of four male CD-1 mice. The test articles were administrated by oral gavage at a dose volume of 10 mL-eq/kg.

Blood samples were collected at 0.5, 1, 3, 6, 8, and 24 hours post dose into K₂EDTA tubes and stored on wet ice until processed. Whole blood was processed to plasma by centrifugation (3500 rpm for 10 minutes at 5° C.) within 30 minutes of collection. Plasma samples were placed in Micronic 96 well tubes and stored at −80° C. until transferred. Plasma concentration of the test compounds was determined by LC-MS/MS. Pharmacokinetic parameters were estimated using non-compartment model analysis. The maximum observed concentration (Cₘₐₓ) and area under the curve from time of dosing to last measured concentration (AUCₗₐₛₜ) were calculated and reported.

### Biological Data

Provided below in Table 2 is data related to compounds disclosed herein.

TABLE 2
Biological Data for Compounds Disclosed Herein

| Example | G12D | G12D 2D |
|---|---|---|
| No. | IC₅₀ (nM) | EC₅₀ (nM) |
| 1 | 0.49 | 3.2 |
| 2 | 0.04 | 0.083 |
| 3 | 0.045 | 0.34 |
| 4 | 0.31 | 0.21 |
| 5 | 2.6 | 0.077 |
| 6 | 0.19 | 0.4 |
| 7 | 0.07 | 0.9 |
| 8 | 0.15 | 7.6 |
| 9 | 0.11 | 4.2 |
| 10 | 0.27 | 1 |
| 11 | 1.9 | 0.44 |
| 12 | 0.13 | 0.16 |
| 13 | 6.9 | 0.093 |
| 14 | 0.03 | 0.054 |
| 15 | 7.9 | 0.062 |
| 16 | 18 | 0.48 |
| 17 | 2.7 | 0.067 |
| 18 | 0.25 | 0.12 |
| 19 | 0.07 | 0.14 |
| 20 | 83 | 130 |
| 21 | 9.6 | 0.55 |
| 22 | 59 | 0.19 |
| 23 | 12 | 0.18 |
| 24 | 0.069 | 0.3 |
| 25 | 4.2 | 0.33 |

The following compounds were evaluated according to procedures described in Example C.

TABLE 3
Pharmacokinetic Data for Compounds Disclosed Herein

| | Rat AUC | Rat Dose | Rat |
|---|---|---|---|
| Example No. | (nM*h) | (mpk) | (% F) |
| 4 | 1,810 | 7.5 | 19 |
| 8 | 3,260 | 7.5 | 12 |
| 12 | 212 | 7.5 | 2.1 |

The present disclosure provides reference to various embodiments and techniques. However, it should be understood that many variations and modifications can be made while remaining within the spirit and scope of the present disclosure. The description is made with the understanding that it is to be considered an exemplification of the claimed subject matter, and is not intended to limit the appended claims to the specific embodiments illustrated.

1. A compound of Formula (Ib): 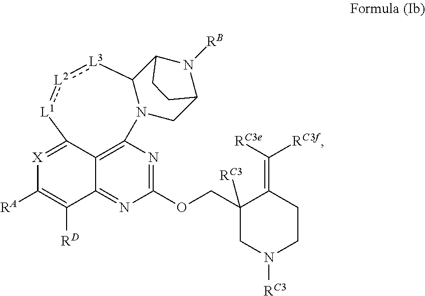
or a pharmaceutically acceptable salt thereof,
wherein
X is N, CH, or CR^{x};
R^{x} is C₁₋₃ alkyl, C₂₋₄ alkenyl, C₁₋₃ alkoxy, C₁₋₃ thioalkyl, —CN, (CH₂)ₘCN, halo, C₁₋₃ haloalkyl, or C₃₋₆ cycloalkyl;
m is 0, 1, 2 or 3;
R¹, R², R³, and R⁴ are each independently H or C₁-C₃ alkyl;
L¹ is O, S, CR^{1a}R^{1b}, C(═CR^{1c}R^{1d}), C(═O), or —C(R^{1e})=;
R^{1a} and R^{1b} are each independently H, C₁-C₃ alkyl, C₁-C₃ alkoxy, halo, C₁-C₆ haloalkyl, C₁-C₆ haloalkoxy, —OH, —CN, C₁-C₃ cyanoalkyl, or C₃-C₆ cycloalkyl;
alternatively, R^{1a} and R^{1b} can combine with the atom to which they are attached to form a C₃-C₆ cycloalkyl or a 3 to 6 membered heterocyclyl having 1 heteroatom that is O, wherein each cycloalkyl and heterocyclyl is substituted with 0, 1, 2, or 3 R^{1x};
R^{1c} and R^{1d} are each independently H, C₁-C₃ alkyl, halo, C₁-C₆ haloalkyl, —CN, C₁-C₃ cyanoalkyl, or C₃-C₆ cycloalkyl;
alternatively, R^{1c} and R^{1d} can combine with the atom to which they are attached to form a C₃-C₆ cycloalkyl or a 3 to 6 membered heterocyclyl having 1 heteroatom that is O, wherein each cycloalkyl and heterocyclyl is substituted with 0, 1, 2, or 3 R^{1x};
R^{1e} is H, C₁-C₃ alkyl, halo, C₁-C₆ haloalkyl, —CN, C₁-C₃ cyanoalkyl, or C₃-C₆ cycloalkyl;
each R^{1x} is independently C₁-C₃ alkyl, C₁-C₃ alkoxy, halo, C₁-C₆ haloalkyl, C₁-C₆ haloalkoxy, or —OH;
L² is absent, O, S, CR^{2a}R^{2b}, C(═CR^{2c}R^{2d}), C(═O), or =C(R^{2e})—, such that when L² is O or S, then L¹ is CR^{1a}R^{1b};
R^{2a} and R^{2b} are each independently H, C₁-C₃ alkyl, C₁-C₃ alkoxy, halo, C₁-C₆ haloalkyl, C₁-C₆ haloalkoxy, —OH, —CN, C₁-C₃ cyanoalkyl, or C₃-C₆ cycloalkyl;
alternatively, R^{2a} and R^{2b} can combine with the atom to which they are attached to form a C₃-C₆ cycloalkyl;
alternatively, R^{1b} and R^{2b} can combine with the atoms to which they are attached to form a C₃-C₆ cycloalkyl, 4- to 10-membered heterocyclyl, C₆-C₁₀ aryl, or 5- to 14-membered heteroaryl, wherein each cycloalkyl, heterocyclyl, aryl and heteroaryl is substituted with 0, 1, 2, or 3 R^{2x};
R^{2e} and R^{2d} are each independently H, C₁-C₃ alkyl, halo, C₁-C₆ haloalkyl, —CN, C₁-C₃ cyanoalkyl, or C₃-C₆ cycloalkyl;
alternatively, R^{2e} and R^{2d} can combine with the atom to which they are attached to form a C₃-C₆ cycloalkyl or a 3 to 6 membered heterocyclyl having 1 heteroatom that is O, wherein each cycloalkyl and heterocyclyl is substituted with 0, 1, 2, or 3 R^{2x};
R^{2e} is H, C₁-C₃ alkyl, halo, C₁-C₆ haloalkyl, —CN, C₁-C₃ cyanoalkyl, or C₃-C₆ cycloalkyl;
alternatively, R^{1e} and R^{2e} can combine with the atoms to which they are attached to form a C₅-C₆ cycloalkyl, 5- to 10-membered heterocyclyl, C₆-C₁₀ aryl, or 5- to 14-membered heteroaryl, wherein each cycloalkyl, heterocyclyl, aryl and heteroaryl is substituted with 0, 1, 2, or 3 R^{2x};
each R^{2x} is independently C₁-C₃ alkyl, C₁-C₃ alkoxy, halo, C₁-C₆ haloalkyl, C₁-C₆ haloalkoxy, or —OH;
L³ is absent, CR^{3a}R^{3b}, C(═CR^{3c}R^{3d}), C(═O), or =C(R^{3e})—;
R^{3a} and R^{3b} are each independently H, C₁-C₃ alkyl, C₁-C₃ alkoxy, halo, C₁-C₆ haloalkyl, C₁-C₆ haloalkoxy, —OH, —CN, C₁-C₃ cyanoalkyl, or C₃-C₆ cycloalkyl;
alternatively, R^{3a} and R^{3b} can combine with the atom to which they are attached to form a C₃-C₆ cycloalkyl;
alternatively, R^{2b} and R^{3b} can combine with the atoms to which they are attached to form a C₃-C₆ cycloalkyl;
R^{3e} and R^{3d} are each independently H, C₁-C₃ alkyl, halo, C₁-C₆ haloalkyl, —CN, C₁-C₃ cyanoalkyl, or C₃-C₆ cycloalkyl;
alternatively, R^{3e} and R^{3d} can combine with the atom to which they are attached to form a C₃-C₆ cycloalkyl or a 3 to 6 membered heterocyclyl having 1 heteroatom that is O, wherein each cycloalkyl and heterocyclyl is substituted with 0, 1, 2, or 3 R^{3x};
each R^{3x} is independently C₁-C₃ alkyl, C₁-C₃ alkoxy, halo, C₁-C₆ haloalkyl, C₁-C₆ haloalkoxy, or —OH;
R^{3e} is H, C₁-C₃ alkyl, halo, C₁-C₆ haloalkyl, —CN, C₁-C₃ cyanoalkyl, or C₃-C₆ cycloalkyl;
alternatively, R^{2e} and R^{3e} can combine with the atoms to which they are attached to form a C₅-C₆ cycloalkyl, 5- to 10-membered heterocyclyl, C₆-C₁₀ aryl, or 5- to 14-membered heteroaryl;
such that when L² is =C(R^{2e})— then L¹ is —C(R^{1e})═ or L³ is =C(R^{3e}), and when L³ is =C(R^{3e})— then L² is +C(R^{2e});
R^{A} is phenyl, naphthyl, or 5- to 14-membered heteroaryl, wherein the phenyl, naphthyl, and heteroaryl is substituted with 0, 1, 2, 3, 4, or 5 R^{A2};
each R^{A2} is independently —OH, C₁-C₁₀ alkyl, C₂-C₆ alkenyl, C₂-C₆ alkynyl, C₁-C₁₀ alkoxy, C₁-C₁₀ hydroxyalkyl, C₂-C₁₀ alkoxyalkyl, C₁-C₆ alkyl-N(R^{A2a})(R^{A2b}), C₁-C₁₀ thioalkyl, halo, C₁-C₆ haloalkyl, —CN, —C(O)R^{A2a}, —C(O)OR^{A2}a, —C(O)R^{A2a}, —C(O)OR^{A2a}, —C(O)N(R^{A2a})(R^{A2b}), —N(R^{A2a})C(O)(R^{A2b}), —OC(O)N(R^{A2a})(R^{A2b}), —N(R^{A2a})C(O)(OR^{A2b}), oxo, —OR^{A2a}, —R^{A2x}, —SR^{A2a}, —S(O)₂R^{A2a}, —S(O)₂OR^{A2a}, —N(R^{A2a})(R^{A2b}), —(C₀-C₃ alkyl)-SF₅, —OP(O)(OR^{A2a})(OR^{A2b}), C₃-C₈ cycloalkyl, —(C₁-C₆ alkyl)-(C₃-C₈ cycloalkyl), 3- to 14-membered heterocyclyl, —(C₁-C₆ alkyl)-(3- to 14-membered heterocyclyl), C₆-C₁₄ aryl, —(C₁-C₆ alkyl)-(C₆-C₁₄ aryl), 5- to 14-membered heteroaryl, or —(C₁-C₆ alkyl)-(5- to 14-membered heteroaryl), wherein each alkyl, alkenyl, alkynyl, alkoxy, hydroxyalkyl, and haloalkyl is substituted with 0, 1, 2, or 3 R^{A3}, and wherein each cycloalkyl, alkyl-cycloalkyl, heterocyclyl, alkyl-heterocyclyl, aryl, alkyl-aryl, heteroaryl, and alkyl-heteroaryl is substituted with 0, 1, 2, or 3 R^{A4};
each R^{A2a} and R^{A2b} is independently H, C₁-C₁₀ alkyl, C₁-C₆ haloalkyl, or C₃-C₈ cycloalkyl;
each R^{A3} is independently halo, —CN, OR^{A3a}, SR^{A3a}, —N(R^{A3a})(R^{A3b}), C₃-C₈ cycloalkyl, or 5- to 14-membered heteroaryl;
each R^{A3a} and R^{A3b} is independently H, C₁-C₁₀ alkyl, C₁-C₆ haloalkyl, or C₃-C₈ cycloalkyl;
each R^{A4} is independently C₁-C₆ alkoxy, C₁-C₆ hydroxyalkyl, C₂-C₆ alkoxyalkyl, C₁-C₆ haloalkyl, C₁-C₆ haloalkoxy, C₁-C₆ haloalkylthio, C₃-C₈ cycloalkyl, —(C₁-C₆ alkyl)-(C₆-C₁₀ aryl), halo, —CN, —OH, or —N(R^{A4a})(R^{A4b});
each R^{A4a} and R^{A4b} is independently H or C₁-C₆ alkyl;
alternatively, two R^{A2} can combine to form a C₃-C₁₀ cycloalkyl, C₆-C₁₀ aryl, a 3- to 10-membered heterocyclyl, or 5- to 14-membered heteroaryl on two adjacent atoms on R^{A}, wherein each cycloalkyl, aryl, heterocyclyl, and heteroaryl is substituted with 0, 1, 2, or 3 R^{A5};
each R^{A5} is independently C₁-C₁₀ alkyl, C₂-C₁₀ alkenyl, C₂-C₁₀ alkynyl, halo, C₁-C₆ haloalkyl, —CN, or C₃-C₈ cycloalkyl;
R^{B} is H, Me, or R^{B1};
R^{B1} and R^{A2x} are each independently: 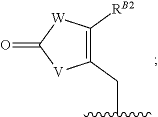
W and V are each independently NR^{B2}, O, or S;
each R^{B2} is independently C₁-C₁₂ alkyl, C₁-C₆ alkoxy, C₂-C₆ alkoxyalkyl, C₁-C₆ alkyl-N(R^{B2a})(R^{B2b}), C₁-C₁₀ thioalkyl, C₁-C₆ haloalkyl, C₁-C₆ haloalkoxy, C₃-C₈ cycloalkyl, C₆-C₁₄ aryl, or 5- to 14-membered heteroaryl, wherein the cycloalkyl, aryl, and heteroaryl are substituted with 0, 1, 2, or 3 R^{B2c};
each R^{B2a} and R^{B2b} is independently H, C₁-C₆ alkyl, C₁-C₆ haloalkyl, or C₃-C₈ cycloalkyl;
each R^{B2c} is independently C₁-C₆ alkyl, C₁-C₆ alkoxy, C₂-C₆ alkoxyalkyl, halo, C₁-C₆ haloalkyl, C₁-C₆ haloalkoxy, oxo, —OH, —CN, or C₃-C₁₀ cycloalkyl;
R^{C3e} is H, C₁, C₁-C₆ alkyl, C₁-C₆ haloalkyl, or C₃-C₆ cycloalkyl;
R^{C3f} is F, C₁, C₁-C₆ alkyl, C₁-C₆ haloalkyl, or C₃-C₆ cycloalkyl;
each R^{C3} is independently C₁-C₆ alkyl, C₂-C₆ alkenyl, C₂-C₈ alkynyl, C₁-C₆ alkoxyalkyl, C₁-C₆ hydroxyalkyl, halo, C₁-C₆ haloalkyl, C₁-C₆ heteroalkyl, —(C₁-C₆ alkyl)-N(R^{C3a})(R^{C3b}), —CN, —C(O)R^{C3a}, —C(O)OR^{C3a}, —C(O)N(R^{C3a})(R^{C3b}), —N(R^{C3a})C(O)(R^{C3b}), —OC(O)N(R^{C3a})(R^{C3b}), —N(R^{C3a})C(O)(OR^{C3b}), =CH₂, =CHF, =CF₂, oxo, —OR^{C3a}, —SR^{C3a}, —N(R^{C3a})(R^{C3b}), —N₃, SF₅, C₃-C₈ cycloalkyl, —(C₁-C₆ alkyl)-(C₃-C₈ cycloalkyl), 3- to 10-membered heterocyclyl, —(C₁-C₆ alkyl)-(3- to 10-membered heterocyclyl), C₆-C₁₀ aryl, —(C₁-C₆ alkyl)-(C₆-C₁₀ aryl), 5- to 10-membered heteroaryl, or —(C₁-C₆ alkyl)-(5- to 10-membered heteroaryl),
wherein each alkyl is substituted with 0, 1, 2, or 3-CN, —C(O)OR^{C3a1}, —C(O)N(R^{C3a1})(R^{C3a2}), —N(R^{C3a1})C(O)(R^{C3a2}), —OC(O)N(R^{C3a1})(R^{C3a2}), —OR^{C3a1}, —SR^{C3a1}, N₃, SF₅, or 3- to 10-membered heterocyclyl substituted with 0, 1, 2, or 3 R^{C3a2}each cycloalkyl, alkyl-cycloalkyl, heterocyclyl, alkyl-heterocyclyl, aryl, alkyl-aryl, heteroaryl, and alkyl-heteroaryl is substituted with 0, 1, 2, or 3 halo, —CN, or R^{C3a2}each alkenyl is substituted with 0, 1, 2, or 3 halo, and
each alkoxyalkyl and alkynyl is substituted with 0, 1, 2, or 3 C₁-C₆ alkyl, C₁-C₆ haloalkyl, C₃-C₈ cycloalkyl substituted with 0 or 1 C₁-C₆ haloalkyl, C₆-C₁₀ aryl, or 5- to 10-membered heteroaryl;
each R^{C3a} and R^{C3b} is independently H, C₁-C₁₀ alkyl, C₁-C₆ haloalkyl, C₆-C₁₀ aryl, C₃-C₆ cycloalkyl, 3- to 6-membered heterocyclyl, or 5- to 10-membered heteroaryl, wherein each aryl and heteroaryl is substituted with 0, 1, 2, or 3 halo, —CN, or R^{C3a2};
alternatively, R^{C3a} and R^{C3b} together with the N to which they are attached form a 3- to 8-membered heterocycle;
each R^{C3a1} and R^{C3a2} is independently C₁-C₃ alkyl, halo, C₁-C₆ haloalkyl, C₃-C₈ cycloalkyl, —(C₁-C₃ alkyl)-(C₃-C₈ cycloalkyl), 3- to 10-membered heterocyclyl, —(C₁-C₃ alkyl)-(3- to 10-membered heterocyclyl), C₆-C₁₀ aryl, —(C₁-C₃ alkyl)-(C₆-C₁₀ aryl), —(C₂-C₄ alkynyl)-(C₆-C₁₀ aryl), 5- to 10-membered heteroaryl, —(C₁-C₃ alkyl)-(5- to 10-membered heteroaryl), or SF₅, wherein each cycloalkyl, alkyl-cycloalkyl, heterocyclyl, alkyl-heterocyclyl, aryl, alkyl-aryl, alkynyl-aryl, heteroaryl, and alkyl-heteroaryl is substituted with 0, 1, 2, or 3 halo, C₁-C₃ haloalkyl, C₁-C₃ haloalkoxy, or SF₅;
alternatively, R^{C3a1} and R^{C3a2} together with the N to which they are attached form a 3- to 8-membered heterocycle;
R^{D} is halo;
each heterocyclyl has 1, 2, 3, or 4 heteroatoms selected from N, O, S, and Si; and
each heteroaryl has 1, 2, 3, or 4 heteroatoms selected from N, O, and S.

2. (canceled)

3. The compound of claim 1, or a pharmaceutically acceptable salt thereof, wherein X is N, C—F, or C—Cl.

4-6. (canceled)

7. The compound of claim 1, or a pharmaceutically acceptable salt thereof, wherein L¹ is CHR^{1b}, R^{1b} is H, C₁-C₃ alkyl, or halo.

8. The compound of claim 1, or a pharmaceutically acceptable salt thereof, wherein L¹ is CHR^{1b}, R^{1b} is H or methyl.

9-12. (canceled)

13. The compound of claim 1, or a pharmaceutically acceptable salt thereof, wherein L² is CHR^{2b}, R^{2b} is H or C₁-C₃ alkyl.

14-17. (canceled)

18. The compound of claim 1, or a pharmaceutically acceptable salt thereof, wherein L³ is a bond.

19. The compound of claim 1, or a pharmaceutically acceptable salt thereof, wherein L³ is CR^{3a}R^{3b}: R^{3a} and R^{3b} are H.

20. (canceled)

21. (canceled)

22. The compound of claim 1, or a pharmaceutically acceptable salt thereof, wherein
R^{A} is naphthyl substituted with 0, 1, 2, 3, 4, or 5 R^{A2};
each R^{A2} is independently C₁-C₆ alkyl, —OH, C₂-C₆ alkenyl, C₂-C₆ alkynyl, halo, C₁-C₆ haloalkyl, —OR^{A2a}—SR^{A2a}, or —(C₁-C₆ alkyl)-(C₃-C₈ cycloalkyl; and
each R^{A2a} is independently C₁-C₆ haloalkyl, or C₃-C₈ cycloalkyl.

23. The compound of claim 1, or a pharmaceutically acceptable salt thereof, wherein R^{A} is pyridyl, benzothienyl, benzothiazolyl, or isoquinolinyl, each substituted with 0, 1, 2, 3, 4, or 5 R^{A}2.

24. (canceled)

25. (canceled)

26. The compound of claim 1, or a pharmaceutically acceptable salt thereof, wherein R^{A} is

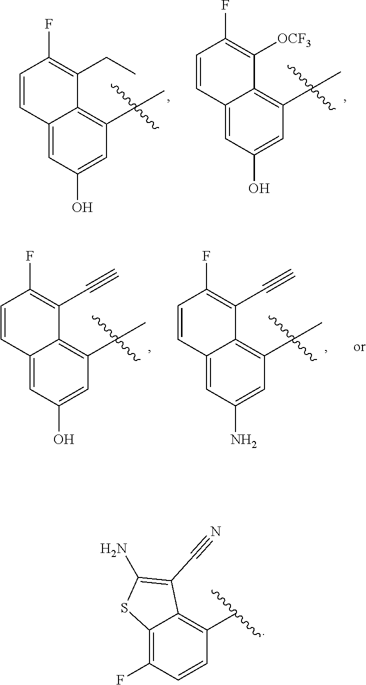 [SMILES: *c1cc(O)cc2ccc(F)c(CC)c12 ; *c1cc(O)cc2ccc(F)c(OC(F)(F)F)c12 ; *c1cc(O)cc2ccc(F)c(C#C)c12 ; *c1cc(N)cc2ccc(F)c(C#C)c12 ; *c1ccc(F)c2sc(N)c(C#N)c12]

27-29. (canceled)

30. The compound of claim 1, or a pharmaceutically acceptable salt thereof, wherein
R^{B1} is: 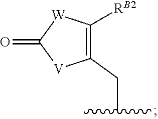
R^{B2} is C₁-C₄ alkyl.

31-33. (canceled)

34. The compound of claim 1, or a pharmaceutically acceptable salt thereof, wherein R^{B1} is

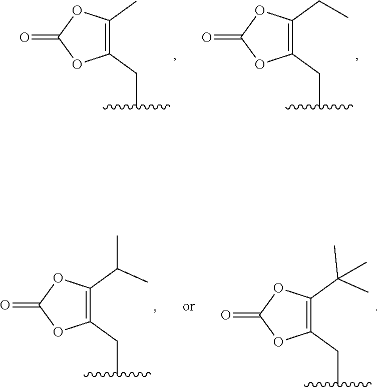

35-40. (canceled)

41. The compound of claim 1, or a pharmaceutically acceptable salt thereof, having the structure of Formula (Id):

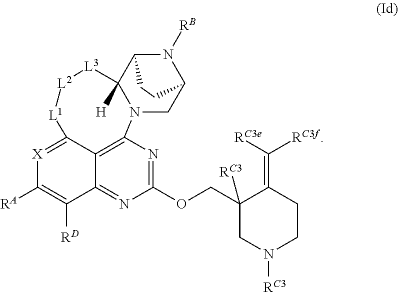

42. The compound of claim 1, wherein
each R^{C3} is C₁-C₆ alkyl;
R^{C3e} is H; and
R^{C3f} is F.

43. (canceled)

44. (canceled)

45. The compound of claim 1, or a pharmaceutically acceptable salt thereof, wherein

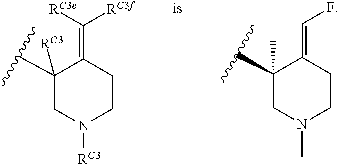

46. The compound of claim 1, or a pharmaceutically acceptable salt thereof, wherein R^{D} is F.

47. A compound of having the structure: 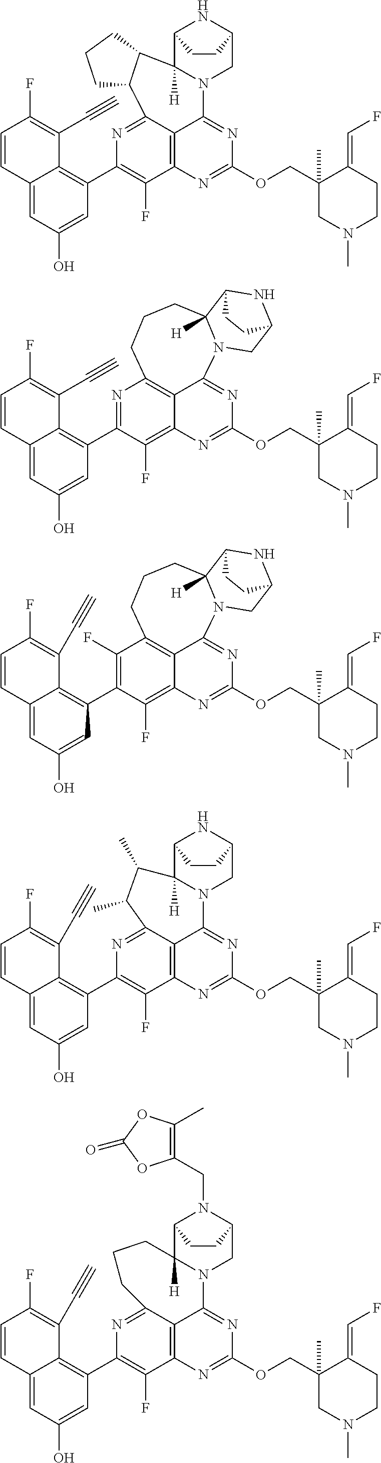 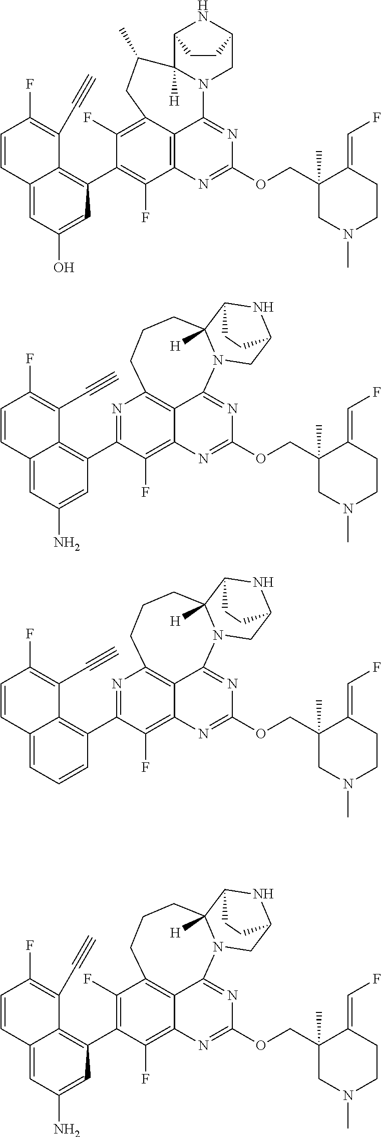 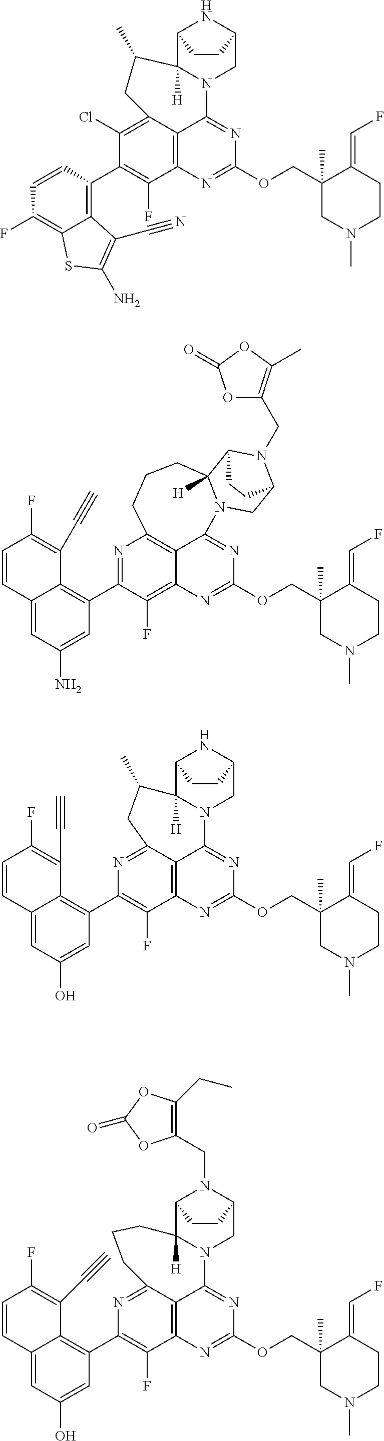 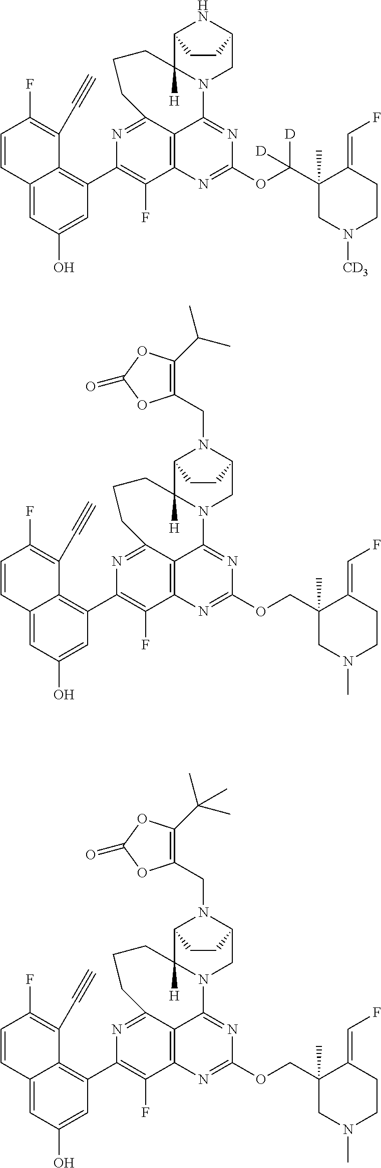 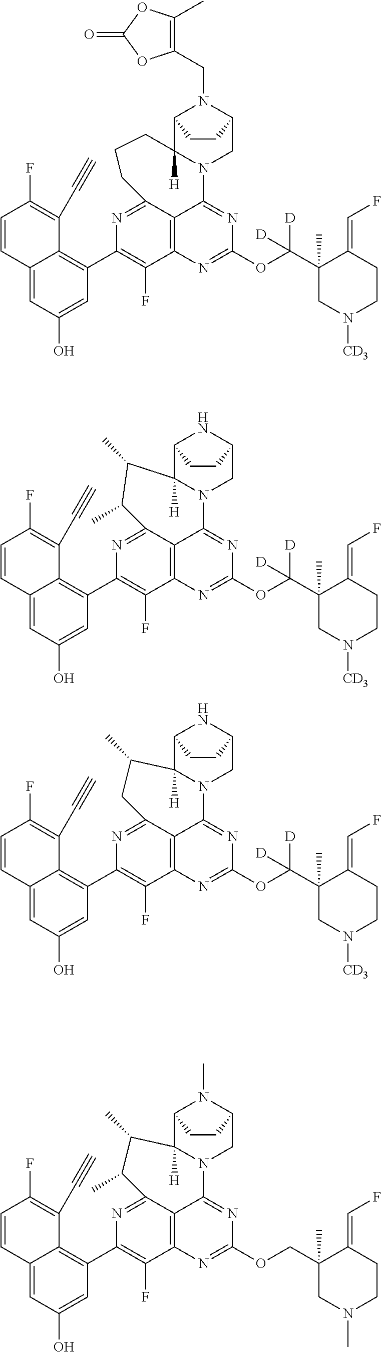 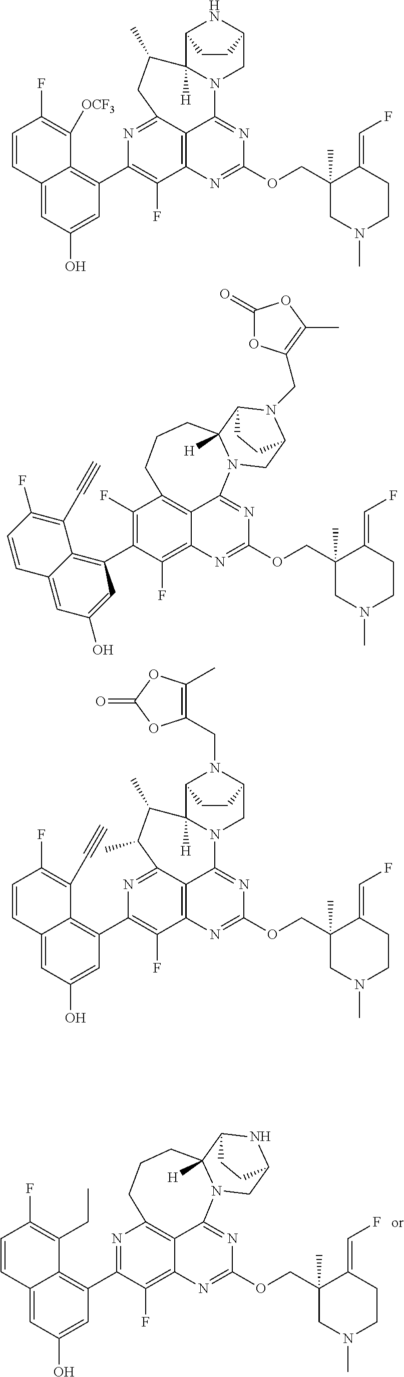 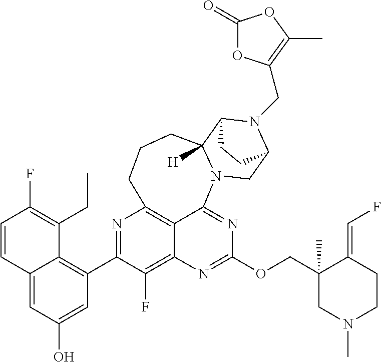
or a pharmaceutically acceptable salt thereof.

48. A pharmaceutical composition comprising a compound of claim 1, or pharmaceutically acceptable salt thereof, and a pharmaceutically acceptable excipient.

49. (canceled)

50. (canceled)

51. A method of treating cancer in a subject in need thereof, the method comprising administering to the subject a therapeutically effective amount of a compound of claim 1, or a pharmaceutically acceptable salt thereof.

52-91. (canceled)
